(19) United States
(12) Patent Application Publication
(10) Pub. No.: US 2026/0092103 A1
(43) Pub. Date: Apr. 2, 2026

(54) MONOCLONAL ANTIBODIES TO ARABINOMANNAN (AM), GROEL2, AND LPRG AND METHODS OF USE

(71) Applicant: Albert Einstein College of Medicine, Bronx, NY (US)

(72) Inventors: Jacqueline M. Achkar, Brooklyn, NY (US); Tingting Chen, Bronx, NY (US); Yanyan Liu, Bronx, NY (US)

(21) Appl. No.: 19/110,451

(22) PCT Filed: Sep. 11, 2023
(86) PCT No.: PCT/US2023/073841

§ 371 Date: Mar. 11, 2025

Related U.S. Application Data
(60) Provisional application No. 63/375,273, filed on Sep. 12, 2022.

(51) Int. Cl. C07K 16/12; A61K 39/00; A61P 31/06; G01N 33/569

(52) U.S. Cl. CPC C07K 16/1289; A61P 31/06; G01N 33/5695; A61K 2039/505; C07K 2317/24; C07K 2317/92

(57) ABSTRACT

Provided are antibodies and antigen-binding fragments thereof binding to Mycobacterium tuberculosis arabinomannan, to GroEL2, or to LprG, as well as methods of use and devices employing such antibodies and/or antigen-binding fragments.

### CROSS-REFERENCE TO RELATED APPLICATIONS

This application claims the benefit under 35 U.S.C. § 119(e) of the earlier filing date of U.S. Provisional Patent No. 63/375,273, filed Sep. 12, 2022, which is hereby incorporated by reference in its entirety.

### STATEMENT OF GOVERNMENT SUPPORT

This invention was made with government support under grant number 1 RO1 AI146329 awarded by National Institutes of Health/National Institute of Allergy and Infectious Diseases. The government has certain rights in the invention.

### REFERENCE TO AN ELECTRONIC SEQUENCE LISTING

The contents of the electronic sequence listing (SeqList_182219-00195.xml; Size: 175,858 bytes; and Date of Creation: Sep. 8, 2023) is herein incorporated by reference in its entirety.

### FIELD

This disclosure relates generally to antibodies against Mycobacterium tuberculosis (Mtb) surface polysaccharide arabinomannan (AM), antibodies against GroEL2, antibodies against LprG, and methods of use thereof.

### BACKGROUND

With over 10 million cases per year and 1.5 million associated deaths, active tuberculosis (TB), caused by the facultative intracellularMtb, is, after COVID-19, the leading cause of death from a single infectious agent. While an estimated quarter of the world is latently infected with Mtb, TB is caused by uncontrolled infection leading to a predominantly respiratory and transmissible disease.

The capsule of microorganisms, including Mtb, is an important virulence factor. Antibodies (Abs) to capsular and surface polysaccharides are protective against infections with encapsulated extra- and intracellular pathogens. Some successful vaccines are based on inducing Abs to capsular polysaccharides. The mechanisms by which Abs protect against Mtb have been insufficiently studied because of the general belief that Mtb, a predominantly intracellular organism, is outside the reach of extracellularly located Abs. However, Abs contribute to the defense against many intracellular pathogens, including Mtb, through various functions, including interactions with Fc receptors (FcR) and the modulation of innate and other immune responses.

The majority of the mycobacterial capsule is composed of polysaccharides and proteins; lipids are a minor component of the capsule. The three major capsular polysaccharides are α-glucan, arabinomannan (AM), and mannan. The 13-20 kDa, immunogenic polysaccharide AM can be isolated from the capsule of Mtb. The cell walls and membranes of mycobacteria also comprise lipoarabinomannan (LAM), a glycolipid that comprises AM. Both AM and LAM are very immunogenic. LAM/AM varies structurally within and between Mtb complex species and non-tuberculous mycobacteria. Some, but not all, murine monoclonal antibodies (mAbs) to AM/LAM show protective in vivo efficacy, and immunization with AM/LAM-protein conjugates improves the outcome of Mtb infected mice. Mtb sheds LAM and AM in cell culture and during infection by various means, such as extracellular vesicles and disassociation of capsular material from the bacterium.

Accordingly, to combat the major global public health problem caused by TB, ongoing development of additional tools for both research and clinical care is critical to meet the continuing urgent need for the rapid detection, treatment, and prevention of Mtb infection. Beyond their potential to inform vaccine and immunotherapy development, antibodies are versatile and indispensable tools in a plethora of applications in medicine and research, including the detection of pathogens and their antigens.

Additionally, there is an urgent need for a simple, yet highly sensitive non-sputum-based point-of-care (POC) test, a World Health Organization (WHO) priority. To be able to impact global TB control, a non-sputum-based POC test needs to be not only accurate and robust, but also suitable for the use of community health care providers in resource-limited settings. The most promising approach to achieve these criteria is the detection of the Mtb surface glycolipid LAM in urine (referred to as U-LAM) via a lateral flow or other simple point-of-care (POC) formats. Existing tests such as the “Determine TB LAM Ag dipstick test” (formerly known as “Alere Determine TB LAM Ag test”), however, have a low sensitivity (<50% in HIV-associated TB, <20% in non-HIV TB for Determine) with an inverse correlation between sensitivity and CD4 cells. As such, the WHO recommends Determines' use only in people living with HIV (PLHIV) and CD4 counts below 100 cells/μl. The test's reliance on polyclonal rabbit sera for LAM capture and detection is another limitation. The recently developed next generation “FujiLAM test” uses two anti-LAM mAbs (rabbit mAB S4-20 (capture antibody) and human mAb A194-01 (detection antibody)) and has higher sensitivity as compared to Determine. However, the FujiLAM test is less simple, requires an extra silver amplification step, and its sensitivity for TB remains <50% in PLHIV with >200 CD4 cells (˜50% in non-HIV TB). Further, antibody S4-20 recognizes only MTXiManx motifs (see FIG. 3). Antibody A194-01 reacts with a several arabinan termini and Man cap motifs and is a good LAM detection antibody, but not a good capture antibody. Finally, as of to date, the FujiLAM test is not commercially available. Therefore, additional tools such as high-affinity mAbs that bind to AM/LAM for simple POC diagnosis of TB are urgently needed.

Similarly, there is also a need for the diagnosis of diseases caused by nontuberculous mycobacteria (NTMs). While some of the NTMs can mimic TB, most present clinically differently from TB. Diseases caused by NTMs are typically challenging to treat (up to years on several drugs), and there is an urgent need for better ways of monitoring of treatment success than sputum microscopy and cultures. Recent studies suggest that U-LAM detection can be a useful tool for both the diagnosis and treatment monitoring of disease caused by NTMs. As of to date, most anti-LAM mAbs do not bind strongly to Mycobacterium avium complex or Mycobacterium abscessus, the mycobacteria causing most NTM disease. Although cross-reactivity with NTMs would be less desired for a capture mAb used for TB diagnostic LAM detection, it would be a necessary attribute for the diagnosis and treatment monitoring of NTM disease. Therefore, additional tools that allow the detection of NTMs are urgently needed.

### BRIEF SUMMARY

Provided herein is an isolated anti-Mycobacterium tuberculosis arabinomannan (anti-Mtb AM) antibody, or Mycobacterium tuberculosis arabinomannan-binding fragment (Mtb AM-binding fragment) thereof, wherein the anti-Mtb AM antibody, or Mtb AM-binding fragment thereof, comprises a heavy chain variable region and a light chain variable region, wherein each of the heavy chain and the light chain variable regions comprises a CDR1, CDR2, and CDR3, and wherein:
(a) CDR1H comprises SEQ ID NO:1, CDR2H comprises SEQ ID NO:2, CDR3H comprises SEQ ID NO:3, CDR1L comprises SEQ ID NO:4, CDR2L comprises sequence AVT, and CDR3L comprises SEQ ID NO:5;
(b) CDR1H comprises SEQ ID NO:10, CDR2H comprises SEQ ID NO:11, CDR3H comprises SEQ ID NO:12, CDR1L comprises SEQ ID NO:13, CDR2L comprises sequence DVT, and CDR3L comprises SEQ ID NO:14;
(c) CDR1H comprises SEQ ID NO:19, CDR2H comprises SEQ ID NO:20, CDR3H comprises SEQ ID NO:21, CDR1L comprises SEQ ID NO:22, CDR2L comprises sequence KIS, and CDR3L comprises SEQ ID NO:23;
(d) CDR1H comprises SEQ ID NO:28, CDR2H comprises SEQ ID NO:29, CDR3H comprises SEQ ID NO:30, CDR1L comprises SEQ ID NO:31, CDR2L comprises sequence GAS, and CDR3L comprises SEQ ID NO:32;
(e) CDR1H comprises SEQ ID NO:37, CDR2H comprises SEQ ID NO:38, CDR3H comprises SEQ ID NO:39, CDR1L comprises SEQ ID NO:40, CDR2L comprises sequence EVS, and CDR3L comprises SEQ ID NO:41; or
(f) CDR1H comprises SEQ ID NO:161, CDR2H comprises SEQ ID NO:162, CDR3H comprises SEQ ID NO:163, CDR1L comprises SEQ ID NO:164, CDR2L comprises sequence KSD, and CDR3L comprises SEQ ID NO:165.

In embodiments, an anti-Mtb AM antibody, or Mtb AM-binding fragment thereof is provided, wherein the anti-Mtb AM antibody, or Mtb AM-binding fragment thereof, comprises:
(a) a heavy chain variable region that comprises a sequence that is at least 80% identical to SEQ ID NO:6 and a light chain variable region that comprises a sequence that is at least 80% identical to SEQ ID NO:7;
(b) a heavy chain variable region that comprises a sequence that is at least 80% identical to SEQ ID NO:15 and a light chain variable region that comprises a sequence that is at least 80% identical to SEQ ID NO:16;
(c) a heavy chain variable region that comprises a sequence that is at least 80% identical to SEQ ID NO:24 and a light chain variable region that comprises a sequence that is at least 80% identical to SEQ ID NO:25;
(d) a heavy chain variable region that comprises a sequence that is at least 80% identical to SEQ ID NO:33 and a light chain variable region that comprises a sequence that is at least 80% identical to SEQ ID NO:34;
(e) a heavy chain variable region that comprises a sequence that is at least 80% identical to SEQ ID NO:42 and a light chain variable region that comprises a sequence that is at least 80% identical to SEQ ID NO:43; or
(f) a heavy chain variable region that comprises a sequence that is at least 80% identical to SEQ ID NO:166 and a light chain variable region that comprises a sequence that is at least 80% identical to SEQ ID NO:167.

In embodiments, an anti-Mtb AM antibody, or Mtb AM-binding fragment thereof is provided, wherein the anti-Mtb AM antibody, or Mtb AM-binding fragment thereof, comprises:
(a) a heavy chain variable region that comprises a sequence that is at least 90% identical to SEQ ID NO:6 and a light chain variable region that comprises a sequence that is at least 90% identical to SEQ ID NO:7;
(b) a heavy chain variable region that comprises a sequence that is at least 90% identical to SEQ ID NO:15 and a light chain variable region that comprises a sequence that is at least 90% identical to SEQ ID NO:16;
(c) a heavy chain variable region that comprises a sequence that is at least 90% identical to SEQ ID NO:24 and a light chain variable region that comprises a sequence that is at least 90% identical to SEQ ID NO:25;
(d) a heavy chain variable region that comprises a sequence that is at least 90% identical to SEQ ID NO:33 and a light chain variable region that comprises a sequence that is at least 90% identical to SEQ ID NO:34;
(e) a heavy chain variable region that comprises a sequence that is at least 90% identical to SEQ ID NO:42 and a light chain variable region that comprises a sequence that is at least 90% identical to SEQ ID NO:43; or
(f) a heavy chain variable region that comprises a sequence that is at least 90% identical to SEQ ID NO:166 and a light chain variable region that comprises a sequence that is at least 90% identical to SEQ ID NO:167.

In embodiments, an anti-Mtb AM antibody, or Mtb AM-binding fragment thereof, is provided, wherein the anti-Mtb AM antibody, or Mtb AM-binding fragment thereof, comprises:
(a) a heavy chain variable region that comprises SEQ ID NO:6 and a light chain variable region that comprises SEQ ID NO:7;
(b) a heavy chain variable region that comprises SEQ ID NO:15 and a light chain variable region that comprises SEQ ID NO:16;
(c) a heavy chain variable region that comprises SEQ ID NO:24 and a light chain variable region that comprises SEQ ID NO:25;
(d) a heavy chain variable region that comprises SEQ ID NO:33 and a light chain variable region that comprises SEQ ID NO:34;
(e) a heavy chain variable region that comprises SEQ ID NO:42 and a light chain variable region that comprises SEQ ID NO:43; or
(f) a heavy chain variable region that comprises SEQ ID NO:166 and a light chain variable region that comprises SEQ ID NO:167.

In one embodiment, the anti-Mtb AM antibody is a monoclonal antibody.

In one embodiment, the anti-Mtb AM antibody is a recombinant antibody.

In one embodiment, the anti-Mtb AM antibody, or Mtb AM-binding fragment thereof, comprises a human framework region or a modified human framework region.

In one embodiment, the anti-Mtb AM antibody, or Mtb AM-binding fragment thereof, comprises a human constant region or a modified human constant region.

In one embodiment, the Mtb AM-binding fragment is an scFv, Fv, Fab′, Fab, F(ab′)₂, or diabody.

In one embodiment, the anti-Mtb AM antibody, or Mtb AM-binding fragment thereof, is deglycosylated.

In one embodiment, the anti-Mtb AM antibody, or Mtb AM-binding fragment thereof, is conjugated to one or more of a cytotoxin, a fluorescent label, and an imaging agent.

Provided herein is a nucleic acid molecule encoding an anti-Mtb AM antibody, or Mtb AM-binding fragment thereof, disclosed herein. In one embodiment, nucleic acid molecule is an isolated nucleic acid molecule. In an embodiment, the nucleic acid molecule comprises:
(a) SEQ ID NO:8 and/or SEQ ID NO: 9;
(b) SEQ ID NO:17 and/or SEQ ID NO: 18;
(c) SEQ ID NO:26 and/or SEQ ID NO:27;
(d) SEQ ID NO:35 and/or SEQ ID NO:36;
(e) SEQ ID NO:44 and/or SEQ ID NO:45; or
(f) SEQ ID NO:168 and/or SEQ ID NO:169.

Provided herein is a vector or set of vectors comprising one or more nucleic acid molecules disclosed herein. Provided herein is a vector or set of vectors comprising a nucleic acid molecule encoding an anti-Mtb AM antibody, or Mtb AM-binding fragment thereof, disclosed herein.

Provided herein is a host cell comprising a nucleic acid molecule or a vector disclosed herein. Provided herein is a host cell comprising a nucleic acid molecule encoding an anti-Mtb AM antibody, or Mtb AM-binding fragment thereof, disclosed herein. Provided herein is a host cell comprising vector comprising a nucleic acid molecule encoding an anti-Mtb AM antibody, orMtb AM-binding fragment thereof, disclosed herein. The cell may be isolated.

Provided herein is a pharmaceutical composition comprising an anti-Mtb AM antibody, or Mtb AM-binding fragment thereof, disclosed herein and a pharmaceutically acceptable excipient.

In one aspect, provided is a method of producing an anti-Mtb AM antibody, or Mtb AM-binding fragment thereof, comprising culturing a host cell comprising vector comprising a nucleic acid molecule encoding an anti-Mtb AM antibody, or Mtb AM-binding fragment thereof, disclosed herein, under conditions wherein the anti-Mtb AM antibody, or Mtb AM-binding fragment thereof, is produced by the host cell.

In one aspect, provided is a method of reducing an activity of Mycobacterium tuberculosis AM in a subject in need thereof, the method comprising administering to said subject an anti-Mtb AM antibody, or Mtb AM-binding fragment thereof, disclosed herein.

In one aspect, provided is a method of reducing an activity of Mycobacterium tuberculosis AM in a subject in need thereof, the method comprising administering to said subject a pharmaceutical composition, wherein the pharmaceutical composition comprises an anti-Mtb AM antibody, or Mtb AM-binding fragment thereof, disclosed herein, and a pharmaceutically acceptable excipient.

In one aspect, provided is a method of treating a Mycobacterium tuberculosis infection in a subject, the method comprising administering to the subject an amount of an anti-Mtb AM antibody, or Mtb AM-binding fragment thereof, disclosed herein. In one aspect, provided is a method of treating a Mycobacterium tuberculosis infection in a subject, the method comprising administering to the subject an amount of an anti-Mtb AM antibody, or Mtb AM-binding fragment thereof, disclosed herein, wherein the amount is effective to treat a Mycobacterium tuberculosis infection.

In one aspect, provided is a method of treating a Mycobacterium tuberculosis infection in a subject, the method comprising administering to the subject an amount of a pharmaceutical composition, wherein the amount is effective to treat a Mycobacterium tuberculosis infection, and wherein the pharmaceutical composition comprises an anti-Mtb AM antibody, or Mtb AM-binding fragment thereof, disclosed herein, and a pharmaceutically acceptable excipient.

In one aspect, provided is a method of reducing the likelihood of a Mycobacterium tuberculosis infection in a subject who does not have a Mycobacterium tuberculosis infection, the method comprising administering to the subject an amount of an anti-Mtb AM antibody, or Mtb AM-binding fragment thereof, disclosed herein, wherein the amount is effective to reduce the likelihood of a Mycobacterium tuberculosis infection.

In one aspect, provided is a method of reducing the likelihood of a Mycobacterium tuberculosis infection in a subject who does not have a Mycobacterium tuberculosis infection, the method comprising administering to the subject an amount of a pharmaceutical composition, wherein the pharmaceutical composition comprises an anti-Mtb AM antibody, or Mtb AM-binding fragment thereof, disclosed herein, and a pharmaceutically acceptable excipient, and wherein the amount is effective to reduce the likelihood of a Mycobacterium tuberculosis infection.

In one aspect, provided is a method of treating a disease, disorder, or condition mediated by, or related to increased activity of Mycobacterium tuberculosis in a subject, the method comprising administering to said subject an anti-Mtb AM antibody, or Mtb AM-binding fragment thereof, disclosed herein.

In one aspect, provided is a method of treating a disease, disorder, or condition mediated by, or related to increased activity of Mycobacterium tuberculosis in a subject, the method comprising administering to said subject a pharmaceutical composition, wherein the pharmaceutical composition comprises an anti-Mtb AM antibody, or Mtb AM-binding fragment thereof, disclosed herein, and a pharmaceutically acceptable excipient.

An assay device is provided for selectively detecting AM, an AM fragment, LAM and/or an AM-comprising fragment of LAM in a biological sample comprising: a first portion comprising a first plurality of anti-Mtb AM antibodies, or Mtb AM-binding fragments thereof, disclosed herein, wherein the anti-Mtb AM antibodies, or Mtb AM-binding fragments thereof, are attached to a reporting entity; and a second portion comprising a second plurality of anti-Mtb AM antibodies, or Mtb AM-binding fragments thereof, or anti-mycobacterial AM-antibodies of antigen-binding fragments thereof.

An assay device is provided for selectively detecting Mycobacterium tuberculosis complex (MTC) AM, an AM fragment, LAM and/or an AM-comprising fragment of LAM and/or one or more bacteria from the MTC group in a biological sample comprising: a first portion comprising a first plurality of anti-Mtb AM-antibodies, or Mtb AM-binding fragments thereof, as described herein, or anti-mycobacterial AM antibodies, wherein the antibodies or fragments are each attached to their own reporting entity; and a second portion comprising a second plurality of anti-Mtb AM-antibodies, or Mtb AM-binding fragments thereof, or anti-mycobacterial AM-antibodies.

In one embodiment, the reporting entity comprises a gold nanoparticle. In one embodiment, the reporting entity comprises an enzyme. In some embodiments, the enzyme is horseradish peroxidase (HRP) or alkaline phosphatase (AP).

In some embodiments, the second plurality of anti-Mtb AM-antibodies, or Mtb AM-binding fragments thereof, or anti-mycobacterial AM-antibodies is affixed to a solid support of the device.

In some embodiments, the first plurality of second plurality of anti-Mtb AM-antibodies, or Mtb AM-binding fragments thereof, or anti-mycobacterial AM-antibodies is not affixed to a solid support of the device.

In some embodiments, the solid support comprises nitrocellulose.

In some embodiments, the assay device further comprises a fluid sample pad prior in sequential order to the first and second portions. In some embodiments, the assay device further comprises a control portion subsequent in sequential order to the first and second portions.

In some embodiments, the control portion comprises a third plurality of antibodies, immobilized on a solid support of the device, and which third plurality of antibodies are capable of binding the first plurality of anti-Mtb AM-antibodies, or Mtb AM-binding fragments thereof, or anti-mycobacterial AM-antibodies each attached to their own reporting molecule.

In some embodiments, assay device further comprises a fluid-absorbent wicking pad subsequent in sequential order to the first and second portions, and third portion if present.

In some embodiments, the assay device is a lateral flow assay device.

In one aspect, provided is a method of detecting AM, an AM fragment, LAM and/or an AM-comprising fragment of LAM in a biological sample comprising: (a) contacting the device disclosed herein with the biological sample; and (b) observing if the AM, AM fragment, LAM and/or AM-comprising fragment of LAM bind to the second plurality of anti-Mtb AM antibodies, or Mtb AM-binding fragments thereof, or anti-mycobacterial AM-antibodies, or antigen-binding fragments thereof, wherein if the AM, AM fragment, LAM and/or AM-comprising fragment of LAM bind to the second plurality of anti-Mtb AM antibodies, or Mtb AM-binding fragments thereof, or anti-mycobacterial AM-antibodies, or antigen-binding fragments thereof, then AM, AM fragment, LAM and/or AM-comprising fragment of LAM have been detected in the biological sample; and wherein if no AM, AM fragment, LAM and/or AM-comprising fragment of LAM bind to the second plurality of anti-Mtb AM antibodies, or Mtb AM-binding fragments thereof, or anti-mycobacterial AM-antibodies, or antigen-binding fragments thereof, then AM, AM fragment, LAM and/or AM-comprising fragment of LAM have not been detected in the biological sample.

In one aspect, provided is a method of detecting MTC AM, an AM fragment, LAM and/or an AM-comprising fragment of LAM and/or one or more bacteria from the MTC group in a biological sample comprising: (a) contacting an assay device disclosed herein with the sample; and (b) observing if MTC AM, an AM fragment, LAM and/or an AM-comprising fragment of LAM and/or one or more bacteria from the MTC group bind to the second plurality of anti-Mtb AM antibodies, or Mtb AM-binding fragments thereof, or anti-mycobacterial AM-antibodies; wherein if MTC AM, an AM fragment, LAM and/or an AM-comprising fragment of LAM and/or one or more bacteria from the MTC group bind to the second plurality of anti-Mtb AM antibodies, or Mtb AM-binding fragments thereof, or anti-mycobacterial AM-antibodies, then MTC AM, an AM fragment, LAM and/or an AM-comprising fragment of LAM and/or one or more bacteria from the MTC group have been detected in the biological sample; and wherein if no MTC AM, an AM fragment, LAM and/or an AM-comprising fragment of LAM and no bacteria from the MTC group bind to the second plurality of anti-Mtb AM antibodies, or Mtb AM-binding fragments thereof, or anti-mycobacterial AM-antibodies, then MTC AM, an AM fragment, LAM and/or an AM-comprising fragment of LAM and/or bacteria from the MTC group have not been detected in the biological sample. In some embodiments, the method further comprises obtaining the sample from asubject.

In some embodiments, the sample is urine, cerebrospinal fluid, pleural fluid, peritoneal fluid, sputum, saliva, a mucosal lining fluid (including but not limited to nasal or oral mucosal lining fluid), breath or exhaled breath condensate, whole blood, serum, plasma, or any other blood component, a tissue sample, or a fine-needle aspirate.

In some embodiments, the subject is a mammal. In some embodiments, the subject is human.

In one aspect, provided is an isolated anti-GroEL2 antibody, or GroEL2-binding fragment thereof, wherein the anti-GroEL2 antibody, or GroEL2-binding fragment thereof, comprises a heavy chain variable region and a light chain variable region, wherein each of the heavy chain and the light chain variable regions comprises a CDR1, CDR2, and CDR3, and wherein:
(a) CDR1H comprises SEQ ID NO:59, CDR2H comprises SEQ ID NO:60, CDR3H comprises SEQ ID NO:61, CDR1L comprises SEQ ID NO:62, CDR2L comprises sequence LVS, and CDR3L comprises SEQ ID NO:63;
(b) CDR1H comprises SEQ ID NO:72, CDR2H comprises SEQ ID NO:73, CDR3H comprises SEQ ID NO:74, CDR1L comprises SEQ ID NO:75, CDR2L comprises sequence DTS, and CDR3L comprises SEQ ID NO:76;
(c) CDR1H comprises SEQ ID NO:85, CDR2H comprises SEQ ID NO:86, CDR3H comprises SEQ ID NO:87, CDR1L comprises SEQ ID NO:88, CDR2L comprises sequence SAS, and CDR3L comprises SEQ ID NO:89;
(d) CDR1H comprises SEQ ID NO:98, CDR2H comprises SEQ ID NO:99, CDR3H comprises SEQ ID NO:100, CDR1L comprises SEQ ID NO:101, CDR2L comprises sequence AAS, and CDR3L comprises SEQ ID NO:102; or
(e) CDR1H comprises SEQ ID NO:111, CDR2H comprises SEQ ID NO:112, CDR3H comprises SEQ ID NO:113, CDR1L comprises SEQ ID NO:114, CDR2L comprises sequence GTS, and CDR3L comprises SEQ ID NO:115.

In some embodiments, the anti-GroEL2 antibody, or GroEL2-binding fragment thereof, comprises:
(a) a heavy chain variable region that comprises a sequence that is at least 80% identical to SEQ ID NO:64 and a light chain variable region that comprises a sequence that is at least 80% identical to SEQ ID NO:65;
(b) a heavy chain variable region that comprises a sequence that is at least 80% identical to SEQ ID NO:77 and a light chain variable region that comprises a sequence that is at least 80% identical to SEQ ID NO:78;
(c) a heavy chain variable region that comprises a sequence that is at least 80% identical to SEQ ID NO:90 and a light chain variable region that comprises a sequence that is at least 80% identical to SEQ ID NO:91;
(d) a heavy chain variable region that comprises a sequence that is at least 80% identical to SEQ ID NO:103 and a light chain variable region that comprises a sequence that is at least 80% identical to SEQ ID NO:104; or
(e) a heavy chain variable region that comprises a sequence that is at least 80% identical to SEQ ID NO:116 and a light chain variable region that comprises a sequence that is at least 80% identical to SEQ ID NO:117.

In some embodiments, the anti-GroEL2 antibody, or GroEL2-binding fragment thereof, of comprises:
(a) a heavy chain variable region that comprises a sequence that is at least 90% identical to SEQ ID NO:64 and a light chain variable region that comprises a sequence that is at least 90% identical to SEQ ID NO:65;
(b) a heavy chain variable region that comprises a sequence that is at least 90% identical to SEQ ID NO:77 and a light chain variable region that comprises a sequence that is at least 90% identical to SEQ ID NO:78;
(c) a heavy chain variable region that comprises a sequence that is at least 90% identical to SEQ ID NO:90 and a light chain variable region that comprises a sequence that is at least 90% identical to SEQ ID NO:91;
(d) a heavy chain variable region that comprises a sequence that is at least 90% identical to SEQ ID NO:103 and a light chain variable region that comprises a sequence that is at least 90% identical to SEQ ID NO:104; or
(e) a heavy chain variable region that comprises a sequence that is at least 90% identical to SEQ ID NO:116 and a light chain variable region that comprises a sequence that is at least 90% identical to SEQ ID NO:117.

In some embodiments, the anti-GroEL2 antibody, or GroEL2-binding fragment thereof, of comprises:
(a) a heavy chain variable region that comprises SEQ ID NO:64 and a light chain variable region that comprises SEQ ID NO:65;
(b) a heavy chain variable region that comprises SEQ ID NO:77 and a light chain variable region that comprises SEQ ID NO:78;
(c) a heavy chain variable region that comprises SEQ ID NO:90 and a light chain variable region that comprises SEQ ID NO:91;
(d) a heavy chain variable region that comprises SEQ ID NO:103 and a light chain variable region that comprises SEQ ID NO:104; or
(e) a heavy chain variable region that comprises SEQ ID NO:116 and a light chain variable region that comprises SEQ ID NO:117.

In some embodiments, the anti-GroEL2 antibody is a monoclonal antibody.

In some embodiments, the anti-GroEL2 antibody is a recombinant antibody.

In some embodiments, the anti-GroEL2 antibody, or GroEL2-binding fragment thereof, comprises a human framework region or a modified human framework region. In some embodiments, the anti-GroEL2 antibody, or GroEL2-binding fragment thereof, comprises a human constant region or a modified human constant region.

In some embodiments, the GroEL2-binding fragment is an is an scFv, Fv, Fab′, Fab, F(ab′)₂, or diabody.

In some embodiments, the anti-GroEL2 antibody, or GroEL2-binding fragment thereof, is deglycosylated.

In some embodiments, the anti-GroEL2 antibody, or GroEL2-binding fragment thereof, is conjugated to one or more of a cytotoxin, a fluorescent label, and an imaging agent.

Provided herein is a nucleic acid molecule encoding an anti-GroEL2 antibody, or GroEL2-binding fragment thereof, disclosed herein. In embodiments, the nucleic acid molecule is isolated. In embodiments, the nucleic acid molecule comprises
(a) SEQ ID NO:66 and/or SEQ ID NO:67;
(b) SEQ ID NO:79 and/or SEQ ID NO:80;
(c) SEQ ID NO:92 and/or SEQ ID NO:93;
(d) SEQ ID NO:105 and/or SEQ ID NO:106; or
(e) SEQ ID NO:118 and/or SEQ ID NO:119.

Provided herein is a vector or set of vectors comprising one or more nucleic acid molecules disclosed herein. Provided herein is a vector or set of vectors comprising a nucleic acid molecule encoding an anti-GroEL2 antibody, or GroEL2-binding fragment thereof, disclosed herein.

Provided herein is a host cell comprising a nucleic acid molecule or a vector disclosed herein. Provided herein is a host cell comprising a nucleic acid molecule encoding an anti-GroEL2 antibody, or GroEL2-binding fragment thereof, disclosed herein. Provided herein is a host cell comprising vector comprising a nucleic acid molecule encoding an anti-GroEL2 antibody, or GroEL2-binding fragment thereof, disclosed herein. The cell may be isolated.

Provided herein is a pharmaceutical composition comprising an anti-GroEL2 antibody, or GroEL2-binding fragment thereof, disclosed herein and a pharmaceutically acceptable excipient.

Provided herein is a method of producing an anti-GroEL2 antibody, or GroEL2-binding fragment thereof, comprising culturing a host cell disclosed herein, under conditions wherein the anti-GroEL2 antibody, or GroEL2-binding fragment thereof, is produced by the host cell.

In one aspect, provided is an isolated anti-LprG antibody, or LprG-binding fragment thereof, wherein the anti-LprG antibody, or LprG-binding fragment thereof, comprises a heavy chain variable region and a light chain variable region, wherein each of the heavy chain and the light chain variable regions comprises a CDR1, CDR2, and CDR3, and wherein CDR1H comprises SEQ ID NO:124, CDR2H comprises SEQ ID NO:125, CDR3H comprises SEQ ID NO:126, CDR1L comprises SEQ ID NO:127, CDR2L comprises sequence LVS, and CDR3L comprises SEQ ID NO:128.

In embodiments, the anti-LprG antibody, or LprG-binding fragment thereof, of comprises a heavy chain variable region that comprises a sequence that is at least 80% identical to SEQ ID NO:129 and a light chain variable region that comprises a sequence that is at least 80% identical to SEQ ID NO:130.

In embodiments, the anti-LprG antibody, or LprG-binding fragment thereof, of comprises a heavy chain variable region that comprises a sequence that is at least 90% identical to SEQ ID NO:129 and a light chain variable region that comprises a sequence that is at least 90% identical to SEQ ID NO:130.

In embodiments, the anti-LprG antibody, or LprG-binding fragment thereof, of comprises a heavy chain variable region that comprises SEQ ID NO:129 and a light chain variable region that comprises SEQ ID NO:130.

In some embodiments, the anti-LprG antibody is a monoclonal antibody.

In some embodiments, the anti-LprG antibody is a recombinant antibody.

In some embodiments, the anti-LprG antibody, or LprG-binding fragment thereof, comprises a human framework region or a modified human framework region. In some embodiments, the anti-LprG antibody, or LprG-binding fragment thereof, comprises a human constant region or a modified human constant region.

In some embodiments, the LprG-binding fragment is an is an scFv, Fv, Fab′, Fab, F(ab′)₂, or diabody.

In some embodiments, the anti-LprG antibody, or LprG-binding fragment thereof, is deglycosylated.

In some embodiments, the anti-LprG antibody, or LprG-binding fragment thereof, is conjugated to one or more of a cytotoxin, a fluorescent label, and an imaging agent.

Provided herein is a nucleic acid molecule encoding an anti-LprG antibody, or LprG-binding fragment thereof, disclosed herein. In embodiments, the nucleic acid molecule is isolated. In embodiments, the nucleic acid molecule comprises SEQ ID NO:131 and/or SEQ ID NO:132.

Provided herein is a vector or set of vectors comprising one or more nucleic acid molecules disclosed herein. Provided herein is a vector or set of vectors comprising a nucleic acid molecule encoding an anti-LprG antibody, or LprG-binding fragment thereof, disclosed herein.

Provided herein is a host cell comprising a nucleic acid molecule or a vector disclosed herein. Provided herein is a host cell comprising a nucleic acid molecule encoding an anti-LprG antibody, or LprG-binding fragment thereof, disclosed herein. Provided herein is a host cell comprising a vector comprising a nucleic acid molecule encoding an anti-LprG antibody, or LprG-binding fragment thereof, disclosed herein. The cell may be isolated.

Provided herein is a pharmaceutical composition comprising an anti-LprG antibody, or LprG-binding fragment thereof, disclosed herein and a pharmaceutically acceptable excipient.

Provided herein is a method of producing an anti-LprG antibody, or LprG-binding fragment thereof, comprising culturing a host cell disclosed herein, under conditions wherein the anti-LprG antibody, or LprG-binding fragment thereof, is produced by the host cell.

### BRIEF DESCRIPTION OF THE DRAWINGS

FIGS. 1A, 1B, 1C, 1D, 1E, IF, 1G, 1H, 1I, and 1J illustrate the binding of anti-AM mAb to AM from different strains. X axis shows antibody concentration in ng/ml. FIG. 1A: Clinical Mtb strain CDC1551. Traces from left to right: T2AM11, T2AM02, P1AM25, A1AM23, T2AM15. FIG. 1B: Clinical Mtb strain CDC1551. Traces from left to right: T2AM11, T2AM02, P1AM25, T1AM65, A2AM10/AlAM23, A2AM30. FIG. 1C: Clinical Mtb strain Beijing HN878. Traces from left to right: T2AM02, T2AM11, P1AM25, T2AM15, A1AM23. FIG. 1D: Clinical Mtb strain Beijing HN878. Traces from left to right: T2AM11, T2AM02, T1AM65/P1AM25, A2AM10, A1AM23, A2AM30. FIG. 1E: Laboratory Mtb strain H37Rv. Traces from left to right: T2AM02, T2AM11, A1AM23, P1AM25, T2AM15. FIG. 1F: Laboratory Mtb strain H37Rv. Traces from left to right: T2AM11, T2AM02, A1AM23, P1AM25, T1AM65, A2AM10, A2AM30. FIG. 1G: Avirulent Mtb strain H37Ra. Traces from left to right: T2AM11, T2AM02, P1AM25/A1AM23, T1AM65, A2AM10, A2AM30. FIG. 1H: Avirulent M bovis strain BCG. Traces from left to right: T2AM11, T2AM02, P1AM25, T1AM65/A1AM23, A2AM10, A2AM30. FIG. 1I: NTM strainM abscessus. Traces from left to right: T2AM02/A1AM23, P1AM25, T2AM15, T2AM11. FIG. 1J: NTM strainM. abscessus. Traces from left to right: A1AM23, P1AM25/T2AM02, A2AM30, T1AM65, T2AM11/A2AM10. FIGS. 1A, 1B, 1C, 1E, and 11: Five-fold serial diluted mAbs were applied as primary antibodies. Binding was detected using an HRP-conjugated anti-human IgG. FIGS. 1D, 1F, 1G, and 1H: Binding curves were obtained by ELISA (AM coated at 10 μg/ml) and four-fold serial dilution of mAbs starting at 20 μg/ml. Human IgG1 mAb 5M16 was used at 20 μg/ml as negative control.

FIGS. 2A, 2B, 2C, 2D, and 2E illustrate binding of anti-AM mAbs to whole cells from different strains. FIG. 2A. Clinical strain CDC1551. Traces from left to right: T2AM11, T2AM02, P1AM25, A1AM23, T2AM15, L1AM04. FIG. 2B: Clinical Mtb strain Beijing. Traces from left to right: T2AM02, T2AM11, P1AM25, T2AM15, A1AM23, L1AM04. FIG. 2C: Laboratory Mtb strain H37Rv. Traces from left to right: T2AM02, T2AM11, P1AM25, A1AM23, T2AM15, L1AMO4. FIG. 2D: NTM strainM. abscessus. Traces from left to right: T2AM02, A1AM23, P1AM25, T2AM15, L1AMO4, T2AM11. FIG. 2E: NTM strainM. avium. Traces from left to right: T2AM02, P1AM25, T2AM11, A1AM23, T2AM15, L1AMO4.

FIG. 3 shows the synthetically generated AM/LAM motifs used for epitope determination. The motifs are organized into seven groups based on their structural/chemical similarities. The oligosaccharide (OS) motifs bound by control antibodies P1AM25, T1AM09, and L1AMO4 are circled (solid lines represent strong binding, dotted lines represent weak binding), and their binding sites are also illustrated on the AM/LAM molecule shown in the middle.

FIGS. 4A, 4B, 4C, 4D, 4E, 4F, 4G, 4H, 4I, 4J, and 4K illustrate the OS mapping strategy to identify glycan epitopes recognized by anti-AM mAbs. FIGS. 4A, 4B, 4C, 4D, 4E, and 4F: Binding of the indicated antibodies to the AM/LAM motifs shown in FIG. 3. FIG. 4G: Binding of mAbs to AM OS. Heatmap shows median fluorescent intensity (MFI) for IgG reactivity to 30 synthetic OS motifs assessed by glycan array and organized according to clustering results with polyclonal IgG. Numbers correspond to position on glycan array.

FIG. 4H: Prediction of epitopes for anti-AM mAbs. FIG. 4I: Verification of predicted epitopes by OS ELISA. FIG. 4J: Binding of T2AM11 and A2AM10 to core mannan motifs by OS ELISA. FIG. 4K: Confirmation of tetra-mannoside epitope for T2AM11 and A2AM10 (identified in this disclosure). Biotin- or BSA-conjugated OS motifs coated at 2-4 μg/ml and probed with anti-AM mAbs at 1 μg/ml.

FIG. 5 illustrates that co-incubation of anti-AM mAbs with bacteria (avirulent Mtb strain H37Ra) increases uptake of bacteria into neutrophils. Neutrophils were infected with fluorescein isothiocyanate-(FITC) labeled Mtb strain H37Ra in the presence of anti-LAM IgG1 mAbs and heat-inactivated (HI) fetal bovine serum (FBS). The rate of Mtb phagocytosis was assessed using flow cytometry. Human IgG1 monoclonal antibodies (5M16 and C144) targeting a non-Mtb antigens served as negative controls.

FIGS. 6A and 6B illustrate that an anti-AM mAb pair shows high sensitivity for urinary LAM detection. FIG. 6A: Standard curve for anti-AM mAb pair (A1AM23 as the capture antibody; P1AM25 as the detection antibody) for detection of serial dilutions of cultured LAM from Mtb CDC 1551 spiked into urine from a healthy volunteer. Dotted line without spiked LAM. FIG. 6B: Detection of LAM in urine from paucibacillary HIV negative culture-confirmed pulmonary TB patients with sputum smear negative (n=2) or 1+ positive (n=3). Dotted line shows value for urine from healthy control.

FIGS. 7A, 7B, and 7C illustrate binding of anti-GroEL2 antibodies to recombinant Mtb GroEL2 protein andMtb fractions. FIG. 7A: Binding of anti-GroEL2 antibodies to recombinant GroEL2. From left to right: Antibodies 63.4/111.6, 80.8/128.16, 238.11, 131.14, 103.11.

FIG. 7B: Binding of anti-GroEL2 antibodies to different Mtb fractions. From left to right: Antibodies 64.4, 131.14, 80.8,128.16, 103.11, 111.6, 238.11. FIG. 7C: Binding of anti-GroEL2 antibodies to M. bovis Bacillus Calmette-Guerin (BCG) (intact bacteria) with or without capsule. From left to right: Antibodies 64.4, 131.14, 80.8,128.16, 103.11, 111.6, 238.11.

FIG. 8 illustrates the results from a competition assay for antibody binding to GroEL2.

FIG. 9 illustrates that a polyclonal anti-GroEL2 antibody reduces lung mycobacterial burden in Mtb-infected mice.

### DETAILED DESCRIPTION

The disclosure provides antibodies and antigen-binding fragments thereof that bind to Mtb AM and the AM portion of LAM, as well as methods of using such antibodies and an antigen-binding fragments thereof. Provided herein are antibodies or antigen-binding fragments thereof disclosed herein bind to bacteria from the Mycobacterium tuberculosis complex (MTC) group or AM/LAM from bacteria from MTC. Provided herein are antibodies or antigen-binding fragments thereof disclosed herein bind to nontuberculous mycobacteria (NTMs) or AM/LAM from NTMs.

The disclosure provides antibodies and an antigen-binding fragments thereof that bind to GroEL2. In embodiments, antibodies and an antigen-binding fragments thereof bind to mycobacterial GroEL2. Chaperone GroEL2 is a major immunogenic capsular protein and is essential to the survival of Mtb. GroEL2 is further located in the cytoplasm and promotes the proper assembly of unfolded proteins within the bacteria. GroEL2 is known to facilitate Mtb association with mouse bone marrow-derived macrophages (BMMΦ) through CD43, a transmembrane glycoprotein found on all hematopoietic cell surfaces. The use of anti-GroEL2 F(ab′)2 to block GroEL2 on the bacterial surface leads to decreased binding of Mtb to murine BMM(D. Moreover, the addition of GroEL2 protein inhibits binding of Mtb to these cells, plausibly via competitive binding to the Mtb receptor CD43 on macrophages. Furthermore, a DNA vaccine containing GroEL2 and IL-12 delivered by a virus envelope has been shown to reduce bacterial burden in lung of Mtb infected mice and prolong survival of Mtb infected monkeys by inducing T-cell mediated responses.

The disclosure provides antibodies and an antigen-binding fragments thereof that bind to lipoarabinomannan carrier protein LprG. In embodiments, antibodies and an antigen-binding fragments thereof that bind to mycobacterial LprG. LprG is a mycobacterial surface lipoprotein that is essential for Mtb virulence. LprG can increase Mtb virulence through several mechanisms such as inhibiting antigen processing in human macrophages and binding LAM which can facilitate LAM-mediated immune evasion.

Because the Mtb capsule is shed in vivo and in vitro, protein GroEL2 and other surface proteins can be found in body fluids. Thus, monoclonal antibodies targeting GroEL2 and other surface proteins have value for TB diagnosis via detection of Mtb proteins.

### Antibodies

The term “antibody” is used in the broadest sense and includes monoclonal antibodies (including full length or intact monoclonal antibodies), polyclonal antibodies, multivalent antibodies, multispecific antibodies (e.g., bispecific antibodies).

As used herein, “antibody variable domain,” “variable chain,” or the like, refers to the portions of the light and heavy chains of antibody molecules that include amino acid sequences of complementarity determining regions (CDRs; i.e., CDR1, CDR2, and CDR3), and framework regions. VH refers to the variable domain of the heavy chain. V_{L} refers to the variable domain of the light chain. The term “framework regions” refers to those variable domain residues other than the CDR residues. Further, the “light chains” of antibodies (immunoglobulins) from any vertebrate species can be assigned to one of two clearly distinct types, called kappa (κ) and lambda (λ), based on the amino acid sequences of their constant domains.

As used herein, the term “Complementarity Determining Regions” (CDRs) refers to portions of an antibody variable domain that are (typically) involved in antigen binding. Each variable domain typically has three CDR regions identified as CDR1, CDR2 and CDR3. Each CDR can comprise amino acid residues from a CDR as defined by e.g., Kabat (i.e., about residues 24-34 (L1), 50-56 (L2) and 89-97 (L3) in the light chain variable domain and 31-35 (H1), 50-65 (H2) and 95-102 (H3) in the heavy chain variable domain (Kabat et al., Sequences of Proteins of Immunological Interest, 5th Ed. Public Health Service, National Institutes of Health, Bethesda, Md. (1987, 1991)). Each CDR can also comprise amino acid residues from a “hypervariable loop” (i.e., about residues 26-32 (LI), 50-52 (L2) and 91-96 (L3) in the light chain variable domain and 26-32 (H1), 53-55 (H2) and 96-101 (H3) in the heavy chain variable domain (Chothia & Lesk 196 J. Mol. Biol. 901 (1987)). In some instances, a CDR can include amino acids from both a CDR region defined according to Kabat and a hypervariable loop. The Kabat residue designations do not always correspond directly with the linear numbering of the amino acid residues (primary amino acid sequence). The actual linear amino acid sequence may contain fewer or additional amino acids than in the strict Kabat numbering corresponding to a shortening of, or insertion into, a structural component, whether framework or CDR, of the basic variable domain structure. The correct Kabat numbering of residues may be determined for a given antibody or antigen-binding fragment thereof by alignment of residues of homology in the sequence of the antibody or antigen-binding fragment thereof with a “standard” Kabat numbered sequence. Alternatively, a CDR can be defined according to the ImMunoGeneTics (IMGT) system (Lefranc, M.-P. et al., Dev. Comp. Immunol., 27, 55-77 (2003)).

As used herein, the term “constant region” refers to a region of an immunoglobulin light chain or heavy chain that is distinct from the variable region. The constant domain of the heavy chain generally comprises at least one of: a CH1 domain, a hinge (e.g., upper, middle, and/or lower hinge region), a CH2 domain, and a CH3 domain. For example, an antibody described herein may comprise a polypeptide comprising a CH1 domain; a polypeptide comprising a CH1 domain, at least a portion of a hinge domain, and a CH2 domain; a polypeptide comprising a CH1 domain and a CH3 domain; a polypeptide comprising a CH1 domain, at least a portion of a hinge domain, and a CH3 domain, or a polypeptide comprising a CH1 domain, at least a portion of a hinge domain, a CH2 domain, and a CH3 domain. In some embodiments, a polypeptide comprises a polypeptide chain comprising a CH3 domain. The constant domain of a light chain can be a kappa (κ) or lambda (λ) constant region. However, it will be understood by one of ordinary skill in the art that these constant domains (e.g., the heavy chain or light chain) may be modified such that they vary in amino acid sequence from the naturally occurring immunoglobulin molecule. HC refers to the heavy chain, including the V_{H} and the constant region. LC refers to the light chain, including the V_{L} and the constant region. In embodiments, an antibody disclosed herein comprises a sequence that is at least 90%, at least 95%, at least 96%, at least 97%, at least 98%, or at least 99% identical to any one of SEQ ID NOs:56-58. In embodiments, an antibody disclosed herein comprises a sequence selected from any one of SEQ ID NOs:56-58.

As used herein, the term “Fc region” or “Fc portion” refers to the C terminal region of an immunoglobulin heavy chain. The Fc region can be a native-sequence Fc region or a non-naturally occurring variant Fc region. Generally, the Fc region of an immunoglobulin comprises constant domains CH2 and CH3. Although the boundaries of the Fc region can vary, in some embodiments, the human IgG heavy chain Fc region can be defined to extend from an amino acid residue at position C226 or from P230 to the carboxy terminus thereof. In some embodiments, the “CH2 domain” of a human IgG Fc region, usually extends from about amino acid residue 231 to about amino acid residue 340. In some embodiments, N-linked carbohydrate chains are interposed between the two CH2 domains of an intact native IgG molecule. In some embodiments, the CH3 domain” of a human IgG Fc region comprises residues C-terminal to the CH2 domain, e.g., from about amino acid residue 341 to about amino acid residue 447 of the Fc region.

### Anti-Mtb AM Antibodies and Mtb AM-Binding Fragments Thereof

Provided herein is an anti-Mycobacterium tuberculosis arabinomannan (anti-Mtb AM) antibody, or Mtb AM-binding fragment thereof, wherein said antibody or fragment thereof comprises a heavy chain variable region and a light chain variable region, wherein each of the heavy chain and the light chain variable regions comprises a CDR1, CDR2, and CDR3; and wherein CDR1H comprises SEQ ID NO:1, CDR2H comprises SEQ ID NO:2, CDR3H comprises SEQ ID NO:3, CDR1L comprises SEQ ID NO:4, CDR2L comprises sequence AVT, and CDR3L comprises SEQ ID NO:5.

Provided herein is an anti-Mtb AM antibody, or Mtb AM-binding fragment thereof, wherein said antibody or fragment thereof comprises a heavy chain variable region and a light chain variable region, wherein each of the heavy chain and the light chain variable regions comprises a CDR1, CDR2, and CDR3; and wherein CDR1H comprises SEQ ID NO:10, CDR2H comprises SEQ ID NO:11, CDR3H comprises SEQ ID NO:12, CDR1L comprises SEQ ID NO:13, CDR2L comprises sequence DVT, and CDR3L comprises SEQ ID NO:14.

Provided herein is an anti-Mtb AM antibody, or Mtb AM-binding fragment thereof, wherein said antibody or fragment thereof comprises a heavy chain variable region and a light chain variable region, wherein each of the heavy chain and the light chain variable regions comprises a CDR1, CDR2, and CDR3; and wherein CDR1H comprises SEQ ID NO:19, CDR2H comprises SEQ ID NO:20, CDR3H comprises SEQ ID NO:21, CDR1L comprises SEQ ID NO:22, CDR2L comprises sequence KIS, and CDR3L comprises SEQ ID NO:23.

Provided herein is an anti-Mtb AM antibody, or Mtb AM-binding fragment thereof, wherein said antibody or fragment thereof comprises a heavy chain variable region and a light chain variable region, wherein each of the heavy chain and the light chain variable regions comprises a CDR1, CDR2, and CDR3; and wherein CDR1H comprises SEQ ID NO:28, CDR2H comprises SEQ ID NO:29, CDR3H comprises SEQ ID NO:30, CDR1L comprises SEQ ID NO:31, CDR2L comprises sequence GAS, and CDR3L comprises SEQ ID NO:32.

Provided herein is an anti-Mtb AM antibody, or Mtb AM-binding fragment thereof, wherein said antibody or fragment thereof comprises a heavy chain variable region and a light chain variable region, wherein each of the heavy chain and the light chain variable regions comprises a CDR1, CDR2, and CDR3; and wherein CDR1H comprises SEQ ID NO:37, CDR2H comprises SEQ ID NO:38, CDR3H comprises SEQ ID NO:39, CDR1L comprises SEQ ID NO:40, CDR2L comprises sequence EVS, and CDR3L comprises SEQ ID NO:41.

Provided herein is an anti-Mtb AM antibody, or Mtb AM-binding fragment thereof, wherein said antibody or fragment thereof comprises a heavy chain variable region and a light chain variable region, wherein each of the heavy chain and the light chain variable regions comprises a CDR1, CDR2, and CDR3; and wherein CDR1H comprises SEQ ID NO:46, CDR2H comprises SEQ ID NO:47, CDR3H comprises SEQ ID NO:48, CDR1L comprises SEQ ID NO:49, CDR2L comprises sequence KIS, and CDR3L comprises SEQ ID NO:50.

Provided herein is an anti-Mtb AM antibody, or Mtb AM-binding fragment thereof, wherein said antibody or fragment thereof comprises a heavy chain variable region and a light chain variable region, wherein each of the heavy chain and the light chain variable regions comprises a CDR1, CDR2, and CDR3; and wherein CDR1H comprises SEQ ID NO:161, CDR2H comprises SEQ ID NO:162, CDR3H comprises SEQ ID NO:163, CDR1L comprises SEQ ID NO:164, CDR2L comprises sequence KSD, and CDR3L comprises SEQ ID NO:165.

### Anti-GroEL2 Antibodies and GroEL2-Binding Fragments Thereof

Provided herein is an anti-GroEL2 antibody, or GroEL2-binding fragment thereof, wherein said antibody or fragment thereof comprises a heavy chain variable region and a light chain variable region, wherein each of the heavy chain and the light chain variable regions comprises a CDR1, CDR2, and CDR3; and wherein CDR1H comprises SEQ ID NO:59, CDR2H comprises SEQ ID NO:60, CDR3H comprises SEQ ID NO:61, CDR1L comprises SEQ ID NO:62, CDR2L comprises sequence LVS, and CDR3L comprises SEQ ID NO:63.

Provided herein is an anti-GroEL2 antibody, or GroEL2-binding fragment thereof, wherein said antibody or fragment thereof comprises a heavy chain variable region and a light chain variable region, wherein each of the heavy chain and the light chain variable regions comprises a CDR1, CDR2, and CDR3; and wherein CDR1H comprises SEQ ID NO:72, CDR2H comprises SEQ ID NO:73, CDR3H comprises SEQ ID NO:74, CDR1L comprises SEQ ID NO:75, CDR2L comprises sequence DTS, and CDR3L comprises SEQ ID NO:76.

Provided herein is an anti-GroEL2 antibody, or GroEL2-binding fragment thereof, wherein said antibody or fragment thereof comprises a heavy chain variable region and a light chain variable region, wherein each of the heavy chain and the light chain variable regions comprises a CDR1, CDR2, and CDR3; and wherein CDR1H comprises SEQ ID NO:85, CDR2H comprises SEQ ID NO:86, CDR3H comprises SEQ ID NO:87, CDR1L comprises SEQ ID NO:88, CDR2L comprises sequence SAS, and CDR3L comprises SEQ ID NO:89.

Provided herein is an anti-GroEL2 antibody, or GroEL2-binding fragment thereof, wherein said antibody or fragment thereof comprises a heavy chain variable region and a light chain variable region, wherein each of the heavy chain and the light chain variable regions comprises a CDR1, CDR2, and CDR3; and wherein CDR1H comprises SEQ ID NO:98, CDR2H comprises SEQ ID NO:99, CDR3H comprises SEQ ID NO:100, CDR1L comprises SEQ ID NO:101, CDR2L comprises sequence AAS, and CDR3L comprises SEQ ID NO:102.

Provided herein is an anti-GroEL2 antibody, or GroEL2-binding fragment thereof, wherein said antibody or fragment thereof comprises a heavy chain variable region and a light chain variable region, wherein each of the heavy chain and the light chain variable regions comprises a CDR1, CDR2, and CDR3; and wherein CDR1H comprises SEQ ID NO:111, CDR2H comprises SEQ ID NO:112, CDR3H comprises SEQ ID NO:113, CDR1L comprises SEQ ID NO:114, CDR2L comprises sequence GTS, and CDR3L comprises SEQ ID NO:115.

### Anti-LprG Antibodies and LprG-Binding Fragments Thereof

Provided herein is an anti-LprG antibody, or LprG-binding fragment thereof, wherein said antibody or fragment thereof comprises a heavy chain variable region and a light chain variable region, wherein each of the heavy chain and the light chain variable regions comprises a CDR1, CDR2, and CDR3; and wherein CDR1H comprises SEQ ID NO:124, CDR2H comprises SEQ ID NO:125, CDR3H comprises SEQ ID NO:126, CDR1L comprises SEQ ID NO:127, CDR2L comprises sequence LVS, and CDR3L comprises SEQ ID NO:128.

According to certain embodiments, the contemplated antibodies and antigen-binding fragments thereof disclosed herein also feature humanized frameworks for reduced immunogenicity. In certain embodiments, the CDRs of the contemplated antibody or antigen-binding fragment thereof are located in frameworks obtained from a human antibody or antigen-binding fragment thereof. In other embodiments, surface-exposed framework residues of the contemplated antibody or antigen-binding fragment thereof are replaced with framework residues of a human antibody or antigen-binding fragment thereof. The CDRs may also be located in murine or humanized frameworks linked to human constant regions (i.e., chimeric antibodies). In one embodiment, the antibody or antigen-binding fragment thereof comprises a murine variable region and human constant regions. In one embodiment, the antibody or antigen-binding fragment thereof comprises a murine variable region, a murine CH1 region and human CH2 and CH3 constant regions. In one embodiment, the CDRs of a contemplated antibody or antigen-binding fragment thereof are located in frameworks that are a composite of two or more human antibodies. In such embodiments, the contemplated antibodies or antigen-binding fragments thereof comprise two or more sequence segments (“composites”) derived from V-regions of unrelated human antibodies that are selected to maintain monoclonal antibody sequences important for antigen binding of the starting precursor monoclonal antibody, and which have all been filtered for the presence of potential T cell epitopes using “in silico tools” (see, e.g., Holgate & Baker, IDrugs. 2009 April; 12(4):233-7). The close fit of human sequence segments with all sections of the starting antibody V regions and the elimination of CD4⁺T cell epitopes prior to synthesis of the antibody or antigen-binding fragment thereof allow this technology to circumvent immunogenicity while maintaining optimal affinity and specificity through the prior analysis of sequences necessary for antigen-specificity (Holgate & Baker, 2009).

Provided herein are variants of antibodies or antigen-binding fragments disclosed herein, where the variants share certain percentage sequence identity with antibodies or antigen-binding fragments disclosed herein. As used herein, the term “identity” refers to sequence identity between two nucleic acid molecules or polypeptides. Identity can be determined by comparing a position in each sequence which may be aligned for purposes of comparison. For example, when a position in the compared nucleotide sequence is occupied by the same base, then the molecules are identical at that position. A degree identity between nucleic acid or amino acid sequences is a function of the number of identical or matching nucleotides or amino acids at shared positions. For example, polypeptides having at least 85%, 90%, 95%, 98%, or 99% identity to specific polypeptides described herein and preferably exhibiting substantially the same functions, as well as polynucleotides encoding such polypeptides, are contemplated. Methods and computer programs for determining both sequence identity and similarity are publicly available, including, but not limited to, the GCG program package (Devereux et al., Nucleic Acids Research 12: 387, 1984), BLASTP, BLASTN, FASTA (Altschul et al., J. Mol. Biol. 215:403 (1990), and the ALIGN program (version 2.0). The well-known Smith Waterman algorithm may also be used to determine similarity. The BLAST program is publicly available from NCBI and other sources (BLAST Manual, Altschul, et al., NCBI NLM NIH, Bethesda, Md. 20894; BLAST 2.0 at http://www.ncbi.nlm.nih.gov/blast/). In comparing sequences, these methods account for various substitutions, deletions, and other modifications.

### Anti-Mtb AM Antibodies and Mtb AM-Binding Fragments Thereof

Provided herein are (i) variable heavy chain sequences that comprise a sequence that is similar, but not identical to a sequence of any one of SEQ ID NOs:6, 15, 24, 33, 42, 51, and 166 (ii) variable light chains sequences that comprise a sequence that is similar, but not identical to a sequence of any one of SEQ ID NOs:7, 16, 25, 34, 43, 52, and 167, and/or (iii) pairings of variable heavy chain sequences that comprise a sequence that is similar, but not identical to a sequence of any one of SEQ ID NOs:6, 15, 24, 33, 42, 51, and 166 and variable light chains sequences that comprise a sequence that is similar, but not identical to a sequence of any one of SEQ ID NOs:7, 16, 25, 34, 43, 52, and 167.

In one embodiment, the anti-Mtb AM antibody, or Mtb AM-binding fragment thereof, comprises a heavy chain variable domain comprising a sequence that is at least 80%, at least 85%, at least 90%, at least 95%, at least 96%, at least 97%, at least 98%, or at least 99% identical to SEQ ID NO:6. In one embodiment, the anti-Mtb AM antibody, or Mtb AM-binding fragment thereof, comprises a light chain variable domain comprising a sequence that is at least 80%, at least 85%, at least 90%, at least 95%, at least 96%, at least 97%, at least 98%, or at least 99% identical to SEQ ID NO:7.

In one embodiment, the anti-Mtb AM antibody, or Mtb AM-binding fragment thereof, comprises
(i) a heavy chain variable domain comprising a sequence that is at least 80%, at least 85%, at least 90%, at least 95%, at least 96%, at least 97%, at least 98%, or at least 99% identical to SEQ ID NO:6; and
(ii) a light chain variable domain comprising a sequence that is at least 80%, at least 85%, at least 90%, at least 95%, at least 96%, at least 97%, at least 98%, or at least 99% identical to SEQ ID NO:7.

In one embodiment, the anti-Mtb AM antibody, or Mtb AM-binding fragment thereof, comprises
(i) a heavy chain variable domain comprising SEQ ID NO:6; and/or
(ii) a light chain variable domain comprising SEQ ID NO:7.

In one embodiment, the anti-Mtb AM antibody, or Mtb AM-binding fragment thereof, comprises
(i) a heavy chain variable domain comprising SEQ ID NO:6; and
(ii) a light chain variable domain comprising SEQ ID NO:7.

In one embodiment, the anti-Mtb AM antibody, or Mtb AM-binding fragment thereof, comprises a heavy chain variable domain comprising a sequence that is at least 80%, at least 85%, at least 90%, at least 95%, at least 96%, at least 97%, at least 98%, or at least 99% identical to SEQ ID NO:15. In one embodiment, the anti-Mtb AM antibody, or Mtb AM-binding fragment thereof, comprises a light chain variable domain comprising a sequence that is at least 80%, at least 85%, at least 90%, at least 95%, at least 96%, at least 97%, at least 98%, or at least 99% identical to SEQ ID NO:16.

In one embodiment, the anti-Mtb AM antibody, or Mtb AM-binding fragment thereof, comprises
(i) a heavy chain variable domain comprising a sequence that is at least 80%, at least 85%, at least 90%, at least 95%, at least 96%, at least 97%, at least 98%, or at least 99% identical to SEQ ID NO:15; and
(ii) a light chain variable domain comprising a sequence that is at least 80%, at least 85%, at least 90%, at least 95%, at least 96%, at least 97%, at least 98%, or at least 99% identical to SEQ ID NO:16.

In one embodiment, the anti-Mtb AM antibody, or Mtb AM-binding fragment thereof, comprises
(i) a heavy chain variable domain comprising SEQ ID NO: 15; and/or
(ii) a light chain variable domain comprising SEQ ID NO: 16.

In one embodiment, the anti-Mtb AM antibody, or Mtb AM-binding fragment thereof, comprises
(i) a heavy chain variable domain comprising SEQ ID NO: 15; and
(ii) a light chain variable domain comprising SEQ ID NO: 16.

In one embodiment, the anti-Mtb AM antibody, or Mtb AM-binding fragment thereof, comprises a heavy chain variable domain comprising a sequence that is at least 80%, at least 85%, at least 90%, at least 95%, at least 96%, at least 97%, at least 98%, or at least 99% identical to SEQ ID NO:24. In one embodiment, the anti-Mtb AM antibody, or Mtb AM-binding fragment thereof, comprises a light chain variable domain comprising a sequence that is at least 80%, at least 85%, at least 90%, at least 95%, at least 96%, at least 97%, at least 98%, or at least 99% identical to SEQ ID NO:25.

In one embodiment, the anti-Mtb AM antibody, or Mtb AM-binding fragment thereof, comprises
(i) a heavy chain variable domain comprising a sequence that is at least 80%, at least 85%, at least 90%, at least 95%, at least 96%, at least 97%, at least 98%, or at least 99% identical to SEQ ID NO:24; and
(ii) a light chain variable domain comprising a sequence that is at least 80%, at least 85%, at least 90%, at least 95%, at least 96%, at least 97%, at least 98%, or at least 99% identical to SEQ ID NO:25.

In one embodiment, the anti-Mtb AM antibody, or Mtb AM-binding fragment thereof, comprises
(i) a heavy chain variable domain comprising SEQ ID NO:24; and/or
(ii) a light chain variable domain comprising SEQ ID NO:25.

In one embodiment, the anti-Mtb AM antibody, or Mtb AM-binding fragment thereof, comprises
(i) a heavy chain variable domain comprising SEQ ID NO:24; and
(ii) a light chain variable domain comprising SEQ ID NO:25.

In one embodiment, the anti-Mtb AM antibody, or Mtb AM-binding fragment thereof, comprises a heavy chain variable domain comprising a sequence that is at least 80%, at least 85%, at least 90%, at least 95%, at least 96%, at least 97%, at least 98%, or at least 99% identical to SEQ ID NO:33. In one embodiment, the anti-Mtb AM antibody, or Mtb AM-binding fragment thereof, comprises a light chain variable domain comprising a sequence that is at least 80%, at least 85%, at least 90%, at least 95%, at least 96%, at least 97%, at least 98%, or at least 99% identical to SEQ ID NO:34.

In one embodiment, the anti-Mtb AM antibody, or Mtb AM-binding fragment thereof, comprises
(i) a heavy chain variable domain comprising a sequence that is at least 80%, at least 85%, at least 90%, at least 95%, at least 96%, at least 97%, at least 98%, or at least 99% identical to SEQ ID NO:33; and
(ii) a light chain variable domain comprising a sequence that is at least 80%, at least 85%, at least 90%, at least 95%, at least 96%, at least 97%, at least 98%, or at least 99% identical to SEQ ID NO:34.

In one embodiment, the anti-Mtb AM antibody, or Mtb AM-binding fragment thereof, comprises
(i) a heavy chain variable domain comprising SEQ ID NO:33; and/or
(ii) a light chain variable domain comprising SEQ ID NO:34.

In one embodiment, the anti-Mtb AM antibody, or Mtb AM-binding fragment thereof, comprises
(i) a heavy chain variable domain comprising SEQ ID NO:33; and
(ii) a light chain variable domain comprising SEQ ID NO:34.

In one embodiment, the anti-Mtb AM antibody, or Mtb AM-binding fragment thereof, comprises a heavy chain variable domain comprising a sequence that is at least 80%, at least 85%, at least 90%, at least 95%, at least 96%, at least 97%, at least 98%, or at least 99% identical to SEQ ID NO:42. In one embodiment, the anti-Mtb AM antibody, or Mtb AM-binding fragment thereof, comprises a light chain variable domain comprising a sequence that is at least 80%, at least 85%, at least 90%, at least 95%, at least 96%, at least 97%, at least 98%, or at least 99% identical to SEQ ID NO:43.

In one embodiment, the anti-Mtb AM antibody, or Mtb AM-binding fragment thereof, comprises
(i) a heavy chain variable domain comprising a sequence that is at least 80%, at least 85%, at least 90%, at least 95%, at least 96%, at least 97%, at least 98%, or at least 99% identical to SEQ ID NO:42; and
(ii) a light chain variable domain comprising a sequence that is at least 80%, at least 85%, at least 90%, at least 95%, at least 96%, at least 97%, at least 98%, or at least 99% identical to SEQ ID NO:43.

In one embodiment, the anti-Mtb AM antibody, or Mtb AM-binding fragment thereof, comprises
(i) a heavy chain variable domain comprising SEQ ID NO:42; and/or
(ii) a light chain variable domain comprising SEQ ID NO:43.

In one embodiment, the anti-Mtb AM antibody, or Mtb AM-binding fragment thereof, comprises
(i) a heavy chain variable domain comprising SEQ ID NO:42; and
(ii) a light chain variable domain comprising SEQ ID NO:43.

In one embodiment, the anti-Mtb AM antibody, or Mtb AM-binding fragment thereof, comprises a heavy chain variable domain comprising a sequence that is at least 80%, at least 85%, at least 90%, at least 95%, at least 96%, at least 97%, at least 98%, or at least 99% identical to SEQ ID NO:51. In one embodiment, the anti-Mtb AM antibody, or Mtb AM-binding fragment thereof, comprises a light chain variable domain comprising a sequence that is at least 80%, at least 85%, at least 90%, at least 95%, at least 96%, at least 97%, at least 98%, or at least 99% identical to SEQ ID NO:52.

In one embodiment, the anti-Mtb AM antibody, or Mtb AM-binding fragment thereof, comprises
(i) a heavy chain variable domain comprising a sequence that is at least 80%, at least 85%, at least 90%, at least 95%, at least 96%, at least 97%, at least 98%, or at least 99% identical to SEQ ID NO:51; and
(ii) a light chain variable domain comprising a sequence that is at least 80%, at least 85%, at least 90%, at least 95%, at least 96%, at least 97%, at least 98%, or at least 99% identical to SEQ ID NO:52.

In one embodiment, the anti-Mtb AM antibody, or Mtb AM-binding fragment thereof, comprises
(i) a heavy chain variable domain comprising SEQ ID NO:51; and/or
(ii) a light chain variable domain comprising SEQ ID NO:52.

In one embodiment, the anti-Mtb AM antibody, or Mtb AM-binding fragment thereof, comprises
(i) a heavy chain variable domain comprising SEQ ID NO:51; and
(ii) a light chain variable domain comprising SEQ ID NO:52.

In one embodiment, the anti-Mtb AM antibody, or Mtb AM-binding fragment thereof, comprises a heavy chain variable domain comprising a sequence that is at least 80%, at least 85%, at least 90%, at least 95%, at least 96%, at least 97%, at least 98%, or at least 99% identical to SEQ ID NO:166. In one embodiment, the anti-Mtb AM antibody, or Mtb AM-binding fragment thereof, comprises a light chain variable domain comprising a sequence that is at least 80%, at least 85%, at least 90%, at least 95%, at least 96%, at least 97%, at least 98%, or at least 99% identical to SEQ ID NO:167.

In one embodiment, the anti-Mtb AM antibody, or Mtb AM-binding fragment thereof, comprises
(i) a heavy chain variable domain comprising a sequence that is at least 80%, at least 85%, at least 90%, at least 95%, at least 96%, at least 97%, at least 98%, or at least 99% identical to SEQ ID NO:166; and
(ii) a light chain variable domain comprising a sequence that is at least 80%, at least 85%, at least 90%, at least 95%, at least 96%, at least 97%, at least 98%, or at least 99% identical to SEQ ID NO:167.

In one embodiment, the anti-Mtb AM antibody, or Mtb AM-binding fragment thereof, comprises
(i) a heavy chain variable domain comprising SEQ ID NO: 166; and/or
(ii) a light chain variable domain comprising SEQ ID NO: 167.

In one embodiment, the anti-Mtb AM antibody, or Mtb AM-binding fragment thereof, comprises
(i) a heavy chain variable domain comprising SEQ ID NO: 166; and
(ii) a light chain variable domain comprising SEQ ID NO:167.

Provided herein is an anti-Mtb AM antibody, or Mtb AM-binding fragment thereof, wherein said antibody or fragment thereof, wherein the anti-Mtb AM antibody, or Mtb AM-binding fragment thereof, comprises a heavy chain variable region and a light chain variable region, wherein each of the heavy chain and the light chain variable regions comprises a CDR1, CDR2, and CDR3; and wherein:
(a) CDR1H comprises SEQ ID NO:1, CDR2H comprises SEQ ID NO:2, CDR3H comprises SEQ ID NO:3, CDR1L comprises SEQ ID NO:4, CDR2L comprises sequence AVT, and CDR3L comprises SEQ ID NO:5;
(b) the heavy chain variable region comprises a sequence that is at least 80%, at least 85%, at least 90%, at least 95%, at least 96%, at least 97%, at least 98%, or at least 99% identical to SEQ ID NO:6; and
(c) the light chain variable region comprises a sequence that is at least 80%, at least 85%, at least 90%, at least 95%, at least 96%, at least 97%, at least 98%, or at least 99% identical to SEQ ID NO:7.

Provided herein is an anti-Mtb AM antibody, or Mtb AM-binding fragment thereof, wherein said antibody or fragment thereof, wherein the anti-Mtb AM antibody, or Mtb AM-binding fragment thereof, comprises a heavy chain variable region and a light chain variable region, wherein each of the heavy chain and the light chain variable regions comprises a CDR1, CDR2, and CDR3; and wherein:
(a) CDR1H comprises SEQ ID NO:10, CDR2H comprises SEQ ID NO:11, CDR3H comprises SEQ ID NO:12, CDR1L comprises SEQ ID NO:13, CDR2L comprises sequence DVT, and CDR3L comprises SEQ ID NO:14;
(b) the heavy chain variable region comprises a sequence that is at least 80%, at least 85%, at least 90%, at least 95%, at least 96%, at least 97%, at least 98%, or at least 99% identical to SEQ ID NO:15; and
(c) the light chain variable region comprises a sequence that is at least 80%, at least 85%, at least 90%, at least 95%, at least 96%, at least 97%, at least 98%, or at least 99% identical to SEQ ID NO:16.

Provided herein is an anti-Mtb AM antibody, or Mtb AM-binding fragment thereof, wherein said antibody or fragment thereof, wherein the anti-Mtb AM antibody, or Mtb AM-binding fragment thereof, comprises a heavy chain variable region and a light chain variable region, wherein each of the heavy chain and the light chain variable regions comprises a CDR1, CDR2, and CDR3; and wherein:
(a) CDR1H comprises SEQ ID NO:19, CDR2H comprises SEQ ID NO:20, CDR3H comprises SEQ ID NO:21, CDR1L comprises SEQ ID NO:22, CDR2L comprises sequence KIS, and CDR3L comprises SEQ ID NO:23;
(b) the heavy chain variable region comprises a sequence that is at least 80%, at least 85%, at least 90%, at least 95%, at least 96%, at least 97%, at least 98%, or at least 99% identical to SEQ ID NO:24; and
(c) the light chain variable region comprises a sequence that is at least 80%, at least 85%, at least 90%, at least 95%, at least 96%, at least 97%, at least 98%, or at least 99% identical to SEQ ID NO:25.

Provided herein is an anti-Mtb AM antibody, or Mtb AM-binding fragment thereof, wherein said antibody or fragment thereof, wherein the anti-Mtb AM antibody, or Mtb AM-binding fragment thereof, comprises a heavy chain variable region and a light chain variable region, wherein each of the heavy chain and the light chain variable regions comprises a CDR1, CDR2, and CDR3; and wherein:
(a) CDR1H comprises SEQ ID NO:28, CDR2H comprises SEQ ID NO:29, CDR3H comprises SEQ ID NO:30, CDR1L comprises SEQ ID NO:31, CDR2L comprises sequence GAS, and CDR3L comprises SEQ ID NO:32;
(b) the heavy chain variable region comprises a sequence that is at least 80%, at least 85%, at least 90%, at least 95%, at least 96%, at least 97%, at least 98%, or at least 99% identical to SEQ ID NO:33; and
(c) the light chain variable region comprises a sequence that is at least 80%, at least 85%, at least 90%, at least 95%, at least 96%, at least 97%, at least 98%, or at least 99% identical to SEQ ID NO:34.

Provided herein is an anti-Mtb AM antibody, or Mtb AM-binding fragment thereof, wherein said antibody or fragment thereof, wherein the anti-Mtb AM antibody, or Mtb AM-binding fragment thereof, comprises a heavy chain variable region and a light chain variable region, wherein each of the heavy chain and the light chain variable regions comprises a CDR1, CDR2, and CDR3; and wherein:
(a) CDR1H comprises SEQ ID NO:37, CDR2H comprises SEQ ID NO:38, CDR3H comprises SEQ ID NO:39, CDR1L comprises SEQ ID NO:40, CDR2L comprises sequence EVS, and CDR3L comprises SEQ ID NO:41;
(b) the heavy chain variable region comprises a sequence that is at least 80%, at least 85%, at least 90%, at least 95%, at least 96%, at least 97%, at least 98%, or at least 99% identical to SEQ ID NO:42; and
(c) the light chain variable region comprises a sequence that is at least 80%, at least 85%, at least 90%, at least 95%, at least 96%, at least 97%, at least 98%, or at least 99% identical to SEQ ID NO:43.

Provided herein is an anti-Mtb AM antibody, or Mtb AM-binding fragment thereof, wherein said antibody or fragment thereof, wherein the anti-Mtb AM antibody, or Mtb AM-binding fragment thereof, comprises a heavy chain variable region and a light chain variable region, wherein each of the heavy chain and the light chain variable regions comprises a CDR1, CDR2, and CDR3; and wherein:
(a) CDR1H comprises SEQ ID NO:46, CDR2H comprises SEQ ID NO:37, CDR3H comprises SEQ ID NO:48, CDR1L comprises SEQ ID NO:49, CDR2L comprises sequence KIS, and CDR3L comprises SEQ ID NO:50;
(b) the heavy chain variable region comprises a sequence that is at least 80%, at least 85%, at least 90%, at least 95%, at least 96%, at least 97%, at least 98%, or at least 99% identical to SEQ ID NO:51; and
(c) the light chain variable region comprises a sequence that is at least 80%, at least 85%, at least 90%, at least 95%, at least 96%, at least 97%, at least 98%, or at least 99% identical to SEQ ID NO:52.

Provided herein is an anti-Mtb AM antibody, or Mtb AM-binding fragment thereof, wherein said antibody or fragment thereof, wherein the anti-Mtb AM antibody, or Mtb AM-binding fragment thereof, comprises a heavy chain variable region and a light chain variable region, wherein each of the heavy chain and the light chain variable regions comprises a CDR1, CDR2, and CDR3; and wherein:
(a) CDR1H comprises SEQ ID NO:161, CDR2H comprises SEQ ID NO:162, CDR3H comprises SEQ ID NO:163, CDR1L comprises SEQ ID NO:164, CDR2L comprises sequence KSD, and CDR3L comprises SEQ ID NO:165;
(b) the heavy chain variable region comprises a sequence that is at least 80%, at least 85%, at least 90%, at least 95%, at least 96%, at least 97%, at least 98%, or at least 99% identical to SEQ ID NO:166; and
the light chain variable region comprises a sequence that is at least 80%, at least 85%, at least 90%, at least 95%, at least 96%, at least 97%, at least 98%, or at least 99% identical to SEQ ID NO:167.

Provided herein is an anti-Mtb AM antibody, or Mtb AM-binding fragment thereof, wherein said antibody or fragment thereof, wherein the anti-Mtb AM antibody, or Mtb AM-binding fragment thereof, comprises a heavy chain variable region and a light chain variable region, wherein each of the heavy chain and the light chain variable regions comprises a CDR1, CDR2, and CDR3; and wherein:
(a) CDR1H comprises SEQ ID NO:1, CDR2H comprises SEQ ID NO:2, CDR3H comprises SEQ ID NO:3, CDR1L comprises SEQ ID NO:4, CDR2L comprises sequence AVT, and CDR3L comprises SEQ ID NO:5;
(b) the heavy chain variable region comprises SEQ ID NO:6; and
(c) the light chain variable region comprises SEQ ID NO:7.

Provided herein is an anti-Mtb AM antibody, or Mtb AM-binding fragment thereof, wherein said antibody or fragment thereof, wherein the anti-Mtb AM antibody, or Mtb AM-binding fragment thereof, comprises a heavy chain variable region and a light chain variable region, wherein each of the heavy chain and the light chain variable regions comprises a CDR1, CDR2, and CDR3; and wherein:
(a) CDR1H comprises SEQ ID NO:10, CDR2H comprises SEQ ID NO:11, CDR3H comprises SEQ ID NO:12, CDR1L comprises SEQ ID NO:13, CDR2L comprises sequence DVT, and CDR3L comprises SEQ ID NO:14;
(b) the heavy chain variable region comprises SEQ ID NO:15; and
(c) the light chain variable region comprises SEQ ID NO: 16.

Provided herein is an anti-Mtb AM antibody, or Mtb AM-binding fragment thereof, wherein said antibody or fragment thereof, wherein the anti-Mtb AM antibody, or Mtb AM-binding fragment thereof, comprises a heavy chain variable region and a light chain variable region, wherein each of the heavy chain and the light chain variable regions comprises a CDR1, CDR2, and CDR3; and wherein:
(a) CDR1H comprises SEQ ID NO:19, CDR2H comprises SEQ ID NO:20, CDR3H comprises SEQ ID NO:21, CDR1L comprises SEQ ID NO:22, CDR2L comprises sequence KIS, and CDR3L comprises SEQ ID NO:23;
(b) the heavy chain variable region comprises SEQ ID NO:24; and
(c) the light chain variable region comprises SEQ ID NO:25.

Provided herein is an anti-Mtb AM antibody, or Mtb AM-binding fragment thereof, wherein said antibody or fragment thereof, wherein the anti-Mtb AM antibody, or Mtb AM-binding fragment thereof, comprises a heavy chain variable region and a light chain variable region, wherein each of the heavy chain and the light chain variable regions comprises a CDR1, CDR2, and CDR3; and wherein:
(a) CDR1H comprises SEQ ID NO:28, CDR2H comprises SEQ ID NO:29, CDR3H comprises SEQ ID NO:30, CDR1L comprises SEQ ID NO:31, CDR2L comprises sequence GAS, and CDR3L comprises SEQ ID NO:32;
(b) the heavy chain variable region comprises SEQ ID NO:33; and
(c) the light chain variable region comprises SEQ ID NO:34.

Provided herein is an anti-Mtb AM antibody, or Mtb AM-binding fragment thereof, wherein said antibody or fragment thereof, wherein the anti-Mtb AM antibody, or Mtb AM-binding fragment thereof, comprises a heavy chain variable region and a light chain variable region, wherein each of the heavy chain and the light chain variable regions comprises a CDR1, CDR2, and CDR3; and wherein:
(a) CDR1H comprises SEQ ID NO:37, CDR2H comprises SEQ ID NO:38, CDR3H comprises SEQ ID NO:39, CDR1L comprises SEQ ID NO:40, CDR2L comprises sequence EVS, and CDR3L comprises SEQ ID NO:41;
(b) the heavy chain variable region comprises SEQ ID NO:42; and
(c) the light chain variable region comprises SEQ ID NO:43.

Provided herein is an anti-Mtb AM antibody, or Mtb AM-binding fragment thereof, wherein said antibody or fragment thereof, wherein the anti-Mtb AM antibody, or Mtb AM-binding fragment thereof, comprises a heavy chain variable region and a light chain variable region, wherein each of the heavy chain and the light chain variable regions comprises a CDR1, CDR2, and CDR3; and wherein:
(a) CDR1H comprises SEQ ID NO:46, CDR2H comprises SEQ ID NO:37, CDR3H comprises SEQ ID NO:48, CDR1L comprises SEQ ID NO:49, CDR2L comprises sequence KIS, and CDR3L comprises SEQ ID NO:50;
(b) the heavy chain variable region comprises SEQ ID NO:51; and
(c) the light chain variable region comprises SEQ ID NO: 52.

Provided herein is an anti-Mtb AM antibody, or Mtb AM-binding fragment thereof, wherein said antibody or fragment thereof, wherein the anti-Mtb AM antibody, or Mtb AM-binding fragment thereof, comprises a heavy chain variable region and a light chain variable region, wherein each of the heavy chain and the light chain variable regions comprises a CDR1, CDR2, and CDR3; and wherein:
(a) CDR1H comprises SEQ ID NO:161, CDR2H comprises SEQ ID NO:162, CDR3H comprises SEQ ID NO:163, CDR1L comprises SEQ ID NO:164, CDR2L comprises sequence KSD, and CDR3L comprises SEQ ID NO:165;
(b) the heavy chain variable region comprises SEQ ID NO:166; and
(c) the light chain variable region comprises SEQ ID NO:167.

### Anti-GroEL2 Antibodies and GroEL2-Binding Fragments Thereof

Also provided herein are (i) variable heavy chain sequences that comprise a sequence that is similar, but not identical to a sequence of any one of SEQ ID NOs:64, 77, 90, 103, and 116, (ii) variable light chains sequences that comprise a sequence that is similar, but not identical to a sequence of any one of SEQ ID NOs:65, 78, 91, 104, 117, and/or (iii) pairings of variable heavy chain sequences that comprise a sequence that is similar, but not identical to a sequence of any one of SEQ ID NOs:64, 77, 90, 103, and 116 and variable light chains sequences that comprise a sequence that is similar, but not identical to a sequence of any one of SEQ ID NOs:65, 78, 91, 104, 117.

In one embodiment, the anti-GroEL2 antibody, or GroEL2-binding fragment thereof, comprises a heavy chain variable domain comprising a sequence that is at least 80%, at least 85%, at least 90%, at least 95%, at least 96%, at least 97%, at least 98%, or at least 99% identical to SEQ ID NO:64. In one embodiment, the anti-GroEL2 antibody, or GroEL2-binding fragment thereof, comprises a light chain variable domain comprising a sequence that is at least 80%, at least 85%, at least 90%, at least 95%, at least 96%, at least 97%, at least 98%, or at least 99% identical to SEQ ID NO:65.

In one embodiment, the anti-GroEL2 antibody, or GroEL2-binding fragment thereof, comprises
(i) a heavy chain variable domain comprising a sequence that is at least 80%, at least 85%, at least 90%, at least 95%, at least 96%, at least 97%, at least 98%, or at least 99% identical to SEQ ID NO:64; and
(ii) a light chain variable domain comprising a sequence that is at least 80%, at least 85%, at least 90%, at least 95%, at least 96%, at least 97%, at least 98%, or at least 99% identical to SEQ ID NO:65.

In one embodiment, the anti-GroEL2 antibody, or GroEL2-binding fragment thereof, comprises
(i) a heavy chain variable domain comprising SEQ ID NO:64; and/or
(ii) a light chain variable domain comprising SEQ ID NO:65.

In one embodiment, the anti-GroEL2 antibody, or GroEL2-binding fragment thereof, comprises
(i) a heavy chain variable domain comprising SEQ ID NO:64; and
(ii) a light chain variable domain comprising SEQ ID NO:65.

In one embodiment, the anti-GroEL2 antibody, or GroEL2-binding fragment thereof, comprises a heavy chain variable domain comprising a sequence that is at least 80%, at least 85%, at least 90%, at least 95%, at least 96%, at least 97%, at least 98%, or at least 99% identical to SEQ ID NO:77. In one embodiment, the anti-GroEL2 antibody, or GroEL2-binding fragment thereof, comprises a light chain variable domain comprising a sequence that is at least 80%, at least 85%, at least 90%, at least 95%, at least 96%, at least 97%, at least 98%, or at least 99% identical to SEQ ID NO:78.

In one embodiment, the anti-GroEL2 antibody, or GroEL2-binding fragment thereof, comprises
(i) a heavy chain variable domain comprising a sequence that is at least 80%, at least 85%, at least 90%, at least 95%, at least 96%, at least 97%, at least 98%, or at least 99% identical to SEQ ID NO:77; and
(ii) a light chain variable domain comprising a sequence that is at least 80%, at least 85%, at least 90%, at least 95%, at least 96%, at least 97%, at least 98%, or at least 99% identical to SEQ ID NO:78.

In one embodiment, the anti-GroEL2 antibody, or GroEL2-binding fragment thereof, comprises
(i) a heavy chain variable domain comprising SEQ ID NO:77; and/or
(ii) a light chain variable domain comprising SEQ ID NO:78.

In one embodiment, the anti-GroEL2 antibody, or GroEL2-binding fragment thereof, comprises
(i) a heavy chain variable domain comprising SEQ ID NO:77; and
(ii) a light chain variable domain comprising SEQ ID NO:78.

In one embodiment, the anti-GroEL2 antibody, or GroEL2-binding fragment thereof, comprises a heavy chain variable domain comprising a sequence that is at least 80%, at least 85%, at least 90%, at least 95%, at least 96%, at least 97%, at least 98%, or at least 99% identical to SEQ ID NO:90. In one embodiment, anti-GroEL2 antibody, or GroEL2-binding fragment thereof, comprises a light chain variable domain comprising a sequence that is at least 80%, at least 85%, at least 90%, at least 95%, at least 96%, at least 97%, at least 98%, or at least 99% identical to SEQ ID NO:91.

In one embodiment, the anti-GroEL2 antibody, or GroEL2-binding fragment thereof, comprises
(i) a heavy chain variable domain comprising a sequence that is at least 80%, at least 85%, at least 90%, at least 95%, at least 96%, at least 97%, at least 98%, or at least 99% identical to SEQ ID NO:90; and
(ii) a light chain variable domain comprising a sequence that is at least 80%, at least 85%, at least 90%, at least 95%, at least 96%, at least 97%, at least 98%, or at least 99% identical to SEQ ID NO:91.

In one embodiment, the anti-GroEL2 antibody, or GroEL2-binding fragment thereof, comprises
(i) a heavy chain variable domain comprising SEQ ID NO:90; and/or
(ii) a light chain variable domain comprising SEQ ID NO:91.

In one embodiment, the anti-GroEL2 antibody, or GroEL2-binding fragment thereof, comprises
(i) a heavy chain variable domain comprising SEQ ID NO:90; and
(ii) a light chain variable domain comprising SEQ ID NO:91.

In one embodiment, the anti-GroEL2 antibody, or GroEL2-binding fragment thereof, comprises a heavy chain variable domain comprising a sequence that is at least 80%, at least 85%, at least 90%, at least 95%, at least 96%, at least 97%, at least 98%, or at least 99% identical to SEQ ID NO:103. In one embodiment, anti-GroEL2 antibody, or GroEL2-binding fragment thereof, comprises a light chain variable domain comprising a sequence that is at least 80%, at least 85%, at least 90%, at least 95%, at least 96%, at least 97%, at least 98%, or at least 99% identical to SEQ ID NO:104.

In one embodiment, the anti-GroEL2 antibody, or GroEL2-binding fragment thereof, comprises
(i) a heavy chain variable domain comprising a sequence that is at least 80%, at least 85%, at least 90%, at least 95%, at least 96%, at least 97%, at least 98%, or at least 99% identical to SEQ ID NO:103; and
(ii) a light chain variable domain comprising a sequence that is at least 80%, at least 85%, at least 90%, at least 95%, at least 96%, at least 97%, at least 98%, or at least 99% identical to SEQ ID NO:104.

In one embodiment, the anti-GroEL2 antibody, or GroEL2-binding fragment thereof, comprises
(i) a heavy chain variable domain comprising SEQ ID NO:103; and/or
(ii) a light chain variable domain comprising SEQ ID NO:104.

In one embodiment, the anti-GroEL2 antibody, or GroEL2-binding fragment thereof, comprises
(i) a heavy chain variable domain comprising SEQ ID NO: 103; and
(ii) a light chain variable domain comprising SEQ ID NO: 104.

In one embodiment, the anti-GroEL2 antibody, or GroEL2-binding fragment thereof, comprises a heavy chain variable domain comprising a sequence that is at least 80%, at least 85%, at least 90%, at least 95%, at least 96%, at least 97%, at least 98%, or at least 99% identical to SEQ ID NO:116. In one embodiment, anti-GroEL2 antibody, or GroEL2-binding fragment thereof, comprises a light chain variable domain comprising a sequence that is at least 80%, at least 85%, at least 90%, at least 95%, at least 96%, at least 97%, at least 98%, or at least 99% identical to SEQ ID NO:117.

In one embodiment, the anti-GroEL2 antibody, or GroEL2-binding fragment thereof, comprises
(i) a heavy chain variable domain comprising a sequence that is at least 80%, at least 85%, at least 90%, at least 95%, at least 96%, at least 97%, at least 98%, or at least 99% identical to SEQ ID NO:116; and
(ii) a light chain variable domain comprising a sequence that is at least 80%, at least 85%, at least 90%, at least 95%, at least 96%, at least 97%, at least 98%, or at least 99% identical to SEQ ID NO:117.

In one embodiment, the anti-GroEL2 antibody, or GroEL2-binding fragment thereof, comprises
(i) a heavy chain variable domain comprising SEQ ID NO: 116; and/or
(ii) a light chain variable domain comprising SEQ ID NO:117.

In one embodiment, the anti-GroEL2 antibody, or GroEL2-binding fragment thereof, comprises
(i) a heavy chain variable domain comprising SEQ ID NO: 116; and
(ii) a light chain variable domain comprising SEQ ID NO:117.

Provided herein is an anti-GroEL2 antibody, or GroEL2-binding fragment thereof, wherein said antibody or fragment thereof, wherein the anti-GroEL2 antibody, or GroEL2-binding fragment thereof, comprises a heavy chain variable region and a light chain variable region, wherein each of the heavy chain and the light chain variable regions comprises a CDR1, CDR2, and CDR3; and wherein:
(a) CDR1H comprises SEQ ID NO:59, CDR2H comprises SEQ ID NO:60, CDR3H comprises SEQ ID NO:61, CDR1L comprises SEQ ID NO:62, CDR2L comprises sequence LVS, and CDR3L comprises SEQ ID NO:63;
(b) the heavy chain variable region comprises a sequence that is at least 80%, at least 85%, at least 90%, at least 95%, at least 96%, at least 97%, at least 98%, or at least 99% identical to SEQ ID NO:64; and
(c) the light chain variable region comprises a sequence that is at least 80%, at least 85%, at least 90%, at least 95%, at least 96%, at least 97%, at least 98%, or at least 99% identical to SEQ ID NO:65.

Provided herein is an anti-GroEL2 antibody, or GroEL2-binding fragment thereof, wherein said antibody or fragment thereof, wherein the anti-GroEL2 antibody, or GroEL2-binding fragment thereof, comprises a heavy chain variable region and a light chain variable region, wherein each of the heavy chain and the light chain variable regions comprises a CDR1, CDR2, and CDR3; and wherein:
(a) CDR1H comprises SEQ ID NO:72, CDR2H comprises SEQ ID NO:73, CDR3H comprises SEQ ID NO:74, CDR1L comprises SEQ ID NO:75, CDR2L comprises sequence DTS, and CDR3L comprises SEQ ID NO:76;
(b) the heavy chain variable region comprises a sequence that is at least 80%, at least 85%, at least 90%, at least 95%, at least 96%, at least 97%, at least 98%, or at least 99% identical to SEQ ID NO:77; and
(c) the light chain variable region comprises a sequence that is at least 80%, at least 85%, at least 90%, at least 95%, at least 96%, at least 97%, at least 98%, or at least 99% identical to SEQ ID NO:78.

Provided herein is an anti-GroEL2 antibody, or GroEL2-binding fragment thereof, wherein said antibody or fragment thereof, wherein the anti-GroEL2 antibody, or GroEL2-binding fragment thereof, comprises a heavy chain variable region and a light chain variable region, wherein each of the heavy chain and the light chain variable regions comprises a CDR1, CDR2, and CDR3; and wherein:
(a) CDR1H comprises SEQ ID NO:85, CDR2H comprises SEQ ID NO:86, CDR3H comprises SEQ ID NO:87, CDR1L comprises SEQ ID NO:88, CDR2L comprises sequence SAS, and CDR3L comprises SEQ ID NO:89;
(b) the heavy chain variable region comprises a sequence that is at least 80%, at least 85%, at least 90%, at least 95%, at least 96%, at least 97%, at least 98%, or at least 99% identical to SEQ ID NO:90; and
(c) the light chain variable region comprises a sequence that is at least 80%, at least 85%, at least 90%, at least 95%, at least 96%, at least 97%, at least 98%, or at least 99% identical to SEQ ID NO:91.

Provided herein is an anti-GroEL2 antibody, or GroEL2-binding fragment thereof, wherein said antibody or fragment thereof, wherein the anti-GroEL2 antibody, or GroEL2-binding fragment thereof, comprises a heavy chain variable region and a light chain variable region, wherein each of the heavy chain and the light chain variable regions comprises a CDR1, CDR2, and CDR3; and wherein:
(a) CDR1H comprises SEQ ID NO:98, CDR2H comprises SEQ ID NO:99, CDR3H comprises SEQ ID NO:100, CDR1L comprises SEQ ID NO:101, CDR2L comprises sequence AAS, and CDR3L comprises SEQ ID NO:102;
(b) the heavy chain variable region comprises a sequence that is at least 80%, at least 85%, at least 90%, at least 95%, at least 96%, at least 97%, at least 98%, or at least 99% identical to SEQ ID NO: 103; and
(c) the light chain variable region comprises a sequence that is at least 80%, at least 85%, at least 90%, at least 95%, at least 96%, at least 97%, at least 98%, or at least 99% identical to SEQ ID NO: 104.

Provided herein is an anti-GroEL2 antibody, or GroEL2-binding fragment thereof, wherein said antibody or fragment thereof, wherein the anti-GroEL2 antibody, or GroEL2-binding fragment thereof, comprises a heavy chain variable region and a light chain variable region, wherein each of the heavy chain and the light chain variable regions comprises a CDR1, CDR2, and CDR3; and wherein:
(a) CDR1H comprises SEQ ID NO:111, CDR2H comprises SEQ ID NO:112, CDR3H comprises SEQ ID NO:113, CDR1L comprises SEQ ID NO:114, CDR2L comprises sequence GTS, and CDR3L comprises SEQ ID NO:115;
(b) the heavy chain variable region comprises a sequence that is at least 80%, at least 85%, at least 90%, at least 95%, at least 96%, at least 97%, at least 98%, or at least 99% identical to SEQ ID NO: 116; and
(c) the light chain variable region comprises a sequence that is at least 80%, at least 85%, at least 90%, at least 95%, at least 96%, at least 97%, at least 98%, or at least 99% identical to SEQ ID NO: 117.

Provided herein is an anti-GroEL2 antibody, or GroEL2-binding fragment thereof, wherein said antibody or fragment thereof, wherein the anti-GroEL2 antibody, or GroEL2-binding fragment thereof, comprises a heavy chain variable region and a light chain variable region, wherein each of the heavy chain and the light chain variable regions comprises a CDR1, CDR2, and CDR3; and wherein:
(a) CDR1H comprises SEQ ID NO:59, CDR2H comprises SEQ ID NO:60, CDR3H comprises SEQ ID NO:61, CDR1L comprises SEQ ID NO:62, CDR2L comprises sequence LVS, and CDR3L comprises SEQ ID NO:63;
(b) the heavy chain variable region comprises SEQ ID NO:64; and
(c) the light chain variable region comprises SEQ ID NO:65.

Provided herein is an anti-GroEL2 antibody, or GroEL2-binding fragment thereof, wherein said antibody or fragment thereof, wherein the anti-GroEL2 antibody, or GroEL2-binding fragment thereof, comprises a heavy chain variable region and a light chain variable region, wherein each of the heavy chain and the light chain variable regions comprises a CDR1, CDR2, and CDR3; and wherein:
(a) CDR1H comprises SEQ ID NO:72, CDR2H comprises SEQ ID NO:73, CDR3H comprises SEQ ID NO:74, CDR1L comprises SEQ ID NO:75, CDR2L comprises sequence DTS, and CDR3L comprises SEQ ID NO:76;
(b) the heavy chain variable region comprises SEQ ID NO:77; and
(c) the light chain variable region comprises SEQ ID NO:78.

Provided herein is an anti-GroEL2 antibody, or GroEL2-binding fragment thereof, wherein said antibody or fragment thereof, wherein the anti-GroEL2 antibody, or GroEL2-binding fragment thereof, comprises a heavy chain variable region and a light chain variable region, wherein each of the heavy chain and the light chain variable regions comprises a CDR1, CDR2, and CDR3; and wherein:
(a) CDR1H comprises SEQ ID NO:85, CDR2H comprises SEQ ID NO:86, CDR3H comprises SEQ ID NO:87, CDR1L comprises SEQ ID NO:88, CDR2L comprises sequence SAS, and CDR3L comprises SEQ ID NO:89;
(b) the heavy chain variable region comprises SEQ ID NO:90; and
(c) the light chain variable region comprises SEQ ID NO:91.

Provided herein is an anti-GroEL2 antibody, or GroEL2-binding fragment thereof, wherein said antibody or fragment thereof, wherein the anti-GroEL2 antibody, or GroEL2-binding fragment thereof, comprises a heavy chain variable region and a light chain variable region, wherein each of the heavy chain and the light chain variable regions comprises a CDR1, CDR2, and CDR3; and wherein:
(a) CDR1H comprises SEQ ID NO:98, CDR2H comprises SEQ ID NO:99, CDR3H comprises SEQ ID NO:100, CDR1L comprises SEQ ID NO:101, CDR2L comprises sequence AAS, and CDR3L comprises SEQ ID NO:102;
(b) the heavy chain variable region comprises SEQ ID NO:103; and
(c) the light chain variable region comprises SEQ ID NO:104.

Provided herein is an anti-GroEL2 antibody, or GroEL2-binding fragment thereof, wherein said antibody or fragment thereof, wherein the anti-GroEL2 antibody, or GroEL2-binding fragment thereof, comprises a heavy chain variable region and a light chain variable region, wherein each of the heavy chain and the light chain variable regions comprises a CDR1, CDR2, and CDR3; and wherein:
(a) CDR1H comprises SEQ ID NO:111, CDR2H comprises SEQ ID NO:112, CDR3H comprises SEQ ID NO:113, CDR1L comprises SEQ ID NO:114, CDR2L comprises sequence GTS, and CDR3L comprises SEQ ID NO:115;
(b) the heavy chain variable region comprises SEQ ID NO:116; and
(c) the light chain variable region comprises SEQ ID NO:117.

In one embodiment, the anti-GroEL2 antibody, or GroEL2-binding fragment thereof, comprises
(i) a heavy chain comprising a sequence that is at least 80%, at least 85%, at least 90%, at least 95%, at least 96%, at least 97%, at least 98%, or at least 99% identical to SEQ ID NO:68; and
(ii) a light chain comprising a sequence that is at least 80%, at least 85%, at least 90%, at least 95%, at least 96%, at least 97%, at least 98%, or at least 99% identical to SEQ ID NO:69.

In one embodiment, the anti-GroEL2 antibody, or GroEL2-binding fragment thereof, comprises
(i) a heavy chain comprising a sequence that is at least 80%, at least 85%, at least 90%, at least 95%, at least 96%, at least 97%, at least 98%, or at least 99% identical to SEQ ID NO:81; and
(ii) a light chain comprising a sequence that is at least 80%, at least 85%, at least 90%, at least 95%, at least 96%, at least 97%, at least 98%, or at least 99% identical to SEQ ID NO:82.

In one embodiment, the anti-GroEL2 antibody, or GroEL2-binding fragment thereof, comprises
(i) a heavy chain comprising a sequence that is at least 80%, at least 85%, at least 90%, at least 95%, at least 96%, at least 97%, at least 98%, or at least 99% identical to SEQ ID NO:94; and
(ii) a light chain comprising a sequence that is at least 80%, at least 85%, at least 90%, at least 95%, at least 96%, at least 97%, at least 98%, or at least 99% identical to SEQ ID NO:95.

In one embodiment, the anti-GroEL2 antibody, or GroEL2-binding fragment thereof, comprises
(i) a heavy chain comprising a sequence that is at least 80%, at least 85%, at least 90%, at least 95%, at least 96%, at least 97%, at least 98%, or at least 99% identical to SEQ ID NO:107; and
(ii) a light chain comprising a sequence that is at least 80%, at least 85%, at least 90%, at least 95%, at least 96%, at least 97%, at least 98%, or at least 99% identical to SEQ ID NO:108.

In one embodiment, the anti-GroEL2 antibody, or GroEL2-binding fragment thereof, comprises
(i) a heavy chain comprising a sequence that is at least 80%, at least 85%, at least 90%, at least 95%, at least 96%, at least 97%, at least 98%, or at least 99% identical to SEQ ID NO:120; and
(ii) a light chain comprising a sequence that is at least 80%, at least 85%, at least 90%, at least 95%, at least 96%, at least 97%, at least 98%, or at least 99% identical to SEQ ID NO:121.

### Anti-LprG Antibodies and LprG-Binding Fragments Thereof

Also provided herein are (i) variable heavy chain sequences that comprise a sequence that is similar, but not identical to a sequence of SEQ ID NO:129, (ii) variable light chains sequences that comprise a sequence that is similar, but not identical to a sequence of SEQ ID NO:130, and/or (iii) pairings of variable heavy chain sequences that comprise a sequence that is similar, but not identical to a sequence of sequence of SEQ ID NO:129 and variable light chains sequences that comprise a sequence that is similar, but not identical to a sequence of sequence of SEQ ID NO:130.

In one embodiment, the anti-LprG antibody, or LprG-binding fragment thereof, comprises a heavy chain variable domain comprising a sequence that is at least 80%, at least 85%, at least 90%, at least 95%, at least 96%, at least 97%, at least 98%, or at least 99% identical to SEQ ID NO:129. In one embodiment, anti-LprG antibody, or LprG-binding fragment thereof, comprises a light chain variable domain comprising a sequence that is at least 80%, at least 85%, at least 90%, at least 95%, at least 96%, at least 97%, at least 98%, or at least 99% identical to SEQ ID NO:130.

In one embodiment, the anti-LprG antibody, or LprG-binding fragment thereof, comprises
(i) a heavy chain variable domain comprising a sequence that is at least 80%, at least 85%, at least 90%, at least 95%, at least 96%, at least 97%, at least 98%, or at least 99% identical to SEQ ID NO:129; and
(ii) a light chain variable domain comprising a sequence that is at least 80%, at least 85%, at least 90%, at least 95%, at least 96%, at least 97%, at least 98%, or at least 99% identical to SEQ ID NO:130.

In one embodiment, the anti-LprG antibody, or LprG-binding fragment thereof, comprises
(i) a heavy chain variable domain comprising SEQ ID NO: 129; and/or
(ii) a light chain variable domain comprising SEQ ID NO:130.

In one embodiment, the anti-LprG antibody, or LprG-binding fragment thereof, comprises
(i) a heavy chain variable domain comprising SEQ ID NO: 129; and
(ii) a light chain variable domain comprising SEQ ID NO:130.
of the heavy chain and the light chain variable regions comprises a CDR1, CDR2, and CDR3; and wherein:
(a) CDR1H comprises SEQ ID NO:124, CDR2H comprises SEQ ID NO:125, CDR3H comprises SEQ ID NO:126, CDR1L comprises SEQ ID NO:127, CDR2L comprises sequence LVS, and CDR3L comprises SEQ ID NO:128;
(b) the heavy chain variable region comprises a sequence that is at least 80%, at least 85%, at least 90%, at least 95%, at least 96%, at least 97%, at least 98%, or at least 99% identical to SEQ ID NO:129; and
(c) the light chain variable region comprises a sequence that is at least 80%, at least 85%, at least 90%, at least 95%, at least 96%, at least 97%, at least 98%, or at least 99% identical to SEQ ID NO:130.

Provided herein is an anti-LprG antibody, or LprG-binding fragment thereof, wherein said antibody or fragment thereof, wherein the anti-LprG antibody, or LprG-binding fragment thereof, comprises a heavy chain variable region and a light chain variable region, wherein each of the heavy chain and the light chain variable regions comprises a CDR1, CDR2, and CDR3; and wherein:
(a) CDR1H comprises SEQ ID NO:124, CDR2H comprises SEQ ID NO:125, CDR3H comprises SEQ ID NO:126, CDR1L comprises SEQ ID NO:127, CDR2L comprises sequence LVS, and CDR3L comprises SEQ ID NO:128;
(b) the heavy chain variable region comprises SEQ ID NO:129; and
(c) the light chain variable region comprises SEQ ID NO: 130.

In one embodiment, the anti-LprG antibody, or LprG-binding fragment thereof, comprises
(i) a heavy chain comprising a sequence that is at least 80%, at least 85%, at least 90%, at least 95%, at least 96%, at least 97%, at least 98%, or at least 99% identical to SEQ ID NO:133; and
(ii) a light chain comprising a sequence that is at least 80%, at least 85%, at least 90%, at least 95%, at least 96%, at least 97%, at least 98%, or at least 99% identical to SEQ ID NO:134.

The antibodies disclosed herein may comprises modifications in their variable and their constant regions. For example, provides herein are antibodies comprising modified, but functionally equivalent variable regions and/or CDRs. In some embodiments, the modification does not significantly affect the properties of the antibody or antigen-binding fragment thereof. In some embodiments, the modification leads to enhanced or decreased activity and/or affinity. For example, the amino acid sequence of an anti-Mtb AM antibody or Mtb AM-binding fragment thereof may be mutated to obtain an antibody with the desired binding affinity to Mtb AM. The amino acid sequence of an anti-GroEL2 antibody or GroEL2-binding fragment thereof may be mutated to obtain an antibody with the desired binding affinity to GroEL2. The amino acid sequence of an anti-LprG antibody or LprG-binding fragment thereof may be mutated to obtain an antibody with the desired binding affinity to LprG. Modification of polypeptides is routine practice in the art and need not be described in detail herein. Examples of modified polypeptides include polypeptides with conservative substitutions of amino acid residues, one or more deletions or additions of amino acids which do not significantly deleteriously change the functional activity, or which mature (enhance) the affinity of the polypeptide for its ligand, or use of chemical analogs.

Amino acid sequence insertions include amino- and/or carboxyl-terminal fusions ranging in length from one residue to polypeptides containing a hundred or more residues, as well as intra-sequence insertions of single or multiple amino acid residues. Examples of terminal insertions include an antibody with an N-terminal methionyl residue or the antibody fused to an epitope tag. Other insertional variants of the antibody molecule include the fusion to the N- or C-terminus of the antibody or antigen-binding fragment thereof with an enzyme or a polypeptide which increases the half-life of the antibody in the blood circulation.

Substitution variants of the antibodies and antigen-binding fragments thereof have at least one amino acid residue in the antibody molecule removed and a different residue inserted in its place. Substitutional mutagenesis may be directed to the hypervariable regions, but framework alterations are also contemplated. Examples of conservative substitutions are shown in Table 1.

TABLE 1
Conservative amino acid substitutions.

| Original Residue | Conservative Substitutions |
|---|---|
| Ala (A) | Val |
| Arg (R) | Lys |
| Asn (N) | Gln |
| Asp (D) | Glu |
| Cys (C) | Ser |
| Gln (Q) | Asn |
| Glu (E) | Asp |
| Gly (G) | Ala |
| His (H) | Arg |
| Ile (I) | Leu |
| Leu (L) | Ile |
| Lys (K) | Arg |
| Met (M) | Leu |
| Phe (F) | Tyr |
| Pro (P) | Ala |
| Ser (S) | Thr |
| Thr (T) | Ser |
| Trp (W) | Tyr |
| Tyr (Y) | Phe |
| Val (V) | Leu |

Naturally occurring residues are divided into groups based on common side-chain properties:
(1) Non-polar: Norleucine, Met, Ala, Val, Leu, Ile;
(2) Polar without charge: Cys, Ser, Thr, Asn, Gln;
(3) Acidic (negatively charged): Asp, Glu;
(4) Basic (positively charged): Lys, Arg,
(5) Residues that influence chain orientation: Gly, Pro; and
(6) Aromatic: Trp, Tyr, Phe, His.

Conservative substitutions can also be made by exchanging a member of one of these classes for another member of the class.

One type of substitution, for example, that may be made is to change one or more cysteines in the antibody, which may be chemically reactive, to another residue, such as, without limitation, alanine or serine. For example, there can be a substitution of a non-canonical cysteine. The substitution can be made in a CDR or framework region of a variable domain or in the constant region of an antibody. In some embodiments, the cysteine is canonical. Any cysteine residue not involved in maintaining the proper conformation of the antibody also may be substituted, generally with serine, to improve the oxidative stability of the molecule and prevent aberrant cross-linking. Conversely, cysteine bond(s) may be added to the antibody to improve its stability, particularly where the antibody is an antibody fragment such as an Fv fragment.

In embodiments, the antibody disclosed is a monoclonal antibody or the antigen-binding fragment thereof is a fragment of a monoclonal antibody. The term “monoclonal antibody” as used herein refers to an antibody member of a population of substantially homogeneous antibodies, i.e., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds a target on Mtb capsular AM, wherein the target-binding polypeptide sequence was obtained by a process that includes the selection of a single target binding polypeptide sequence from a plurality of polypeptide sequences. In certain embodiments, such a monoclonal antibody typically includes an antibody comprising a polypeptide sequence that binds a target on GroEL2, wherein the target-binding polypeptide sequence was obtained by a process that includes the selection of a single target binding polypeptide sequence from a plurality of polypeptide sequences. In certain embodiments, such a monoclonal antibody typically includes an antibody comprising a polypeptide sequence that binds a target on LprG, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. In contrast to polyclonal antibody preparations, which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins. Thus, an identified monoclonal antibody can be produced by non-hybridoma techniques, e.g., by appropriate recombinant means once the sequence thereof is identified.

In some embodiments, the antibody disclosed herein is a human antibody and/or the antigen-binding fragment thereof is a fragment of a human antibody. As used herein, a “human antibody” is one whose sequences correspond to (i.e., are identical in sequence to) an antibody that could be produced by a human and/or has been made using any of the techniques for making human antibodies as disclosed herein, but not one which has been made in a human. A “human antibody” as used herein can be produced using various techniques known in the art, including phage-display libraries (Hoogenboom and Winter, J. Mol. Biol., 227:381 (1991); Marks et al., J. Mol. Biol., 222:581 (1991), hereby incorporated by reference in their entireties, by methods described in Cole et al., Monoclonal Antibodies and Cancer Therapy, Alan R. Liss, p. 77 (1985) hereby incorporated by reference in its entirety; Boerner et al., J. Immunol., 147(1):86-95 (1991) hereby incorporated by reference in its entirety, van Dijk and van de Winkel, Curr. Opin. Pharmacol., 5: 368-74 (2001) hereby incorporated by reference in its entirety, and by administering the antigen (e.g., Mtb capsular AM or an entity comprising such; GroEL2 or an entity comprising such; or LprG or an entity comprising such) to a transgenic animal that has been modified to produce such antibodies in response to antigenic challenge, but whose endogenous loci have been disabled, e.g., immunized xenomice (see, e.g., U.S. Pat. Nos. 5,939,598; 6,075,181; 6,114,598; 6,150,584 and 6,162,963 to Kucherlapati et al. regarding Xenomouse® technology, each of which patents are hereby incorporated by reference in their entireties), e.g., VelocImmune® (Regeneron, Tarrytown, NY), e.g., UltiMab® platform (Medarex, now Bristol Myers Squibb, Princeton, NJ). See also, for example, Li et al., Proc. Natl. Acad. Sci. USA, 103:3557-3562 (2006) regarding human antibodies generated via a human B-cell hybridoma technology. See also KM Mouse® system, described in PCT Publication WO 02/43478 by Ishida et al., in which the mouse carries a human heavy chain transchromosome and a human light chain transgene, and the TC mouse system, described in Tomizuka et al. (2000) Proc. Natl. Acad. Sci. USA 97:722-727, in which the mouse carries both a human heavy chain transchromosome and a human light chain transchromosome, both of which are hereby incorporated by reference in their entireties. In each of these systems, the transgenes and/or transchromosomes carried by the mice comprise human immunoglobulin variable and constant region sequences. The term “human antibody”, as used herein, is intended to include antibodies having variable regions in which both the framework and CDR regions are sequences of human origin or identical thereto other than antibodies naturally occurring in a human or made in a human. Furthermore, if the antibody (e.g., an intact antibody rather than, for example, a Fab fragment) contains a constant region, the constant region also is derived from such human sequences, e.g., human germline sequences, or mutated versions of human germline sequences. The human antibodies disclosed herein may include amino acid residues not encoded by human sequences (e.g., mutations introduced by random or site-specific mutagenesis in vitro or by somatic mutation in vivo). In one non-limiting embodiment, where the human antibodies are human monoclonal antibodies, such antibodies can be produced by a hybridoma which includes a B cell obtained from a transgenic nonhuman animal, e.g., a transgenic mouse, having a genome comprising a human heavy chain transgene and a light chain transgene fused to an immortalized cell.

In embodiments, the antibody, or antigen-binding fragment thereof, is a recombinant antibody. The term “recombinant human antibody”, as used herein, includes all human antibodies that are prepared, expressed, created or isolated by recombinant means, such as antibodies isolated from an animal (e.g., a mouse) that is transgenic or transchromosomal for human immunoglobulin genes or a hybridoma prepared therefrom, antibodies isolated from a host cell transformed to express the human antibody, e.g., from a transfectoma, antibodies isolated from a recombinant, combinatorial human antibody library, and antibodies prepared, expressed, created, or isolated by any other means that involve splicing of all or a portion of a human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

In some embodiments, the antibody, or antigen-binding fragment thereof, is isolated. As used herein, the term “isolated antibody” refers to an antibody that by virtue of its origin or source of derivation meets one, two, three or four of the following criteria: (1) is not substantially associated with naturally associated components that accompany it in its native state, (2) is substantially free of other proteins from the same species, (3) is expressed by a cell from a different species, and (4) does not occur in nature.

In embodiments, the antibody, or antigen-binding fragment thereof, comprises a human constant region or modified constant region. In some embodiments, the antibody, or antigen-binding fragment thereof, has a non-human constant region or a modified non-human constant region. In one embodiment, antibody, or antigen-binding fragment thereof, has murine constant region or modified murine constant region. In one embodiment, the antibody, or antigen-binding fragment thereof, has a non-human primate constant region or modified non-human primate constant region. In some embodiments, the antibody, or antigen-binding fragment thereof, has a non-human constant region or a modified non-human constant region. In some embodiments, constant region is from a non-human primate, a mouse, a rat, a sheep, a goat, or a rabbit.

Depending on the amino acid sequences of the constant domains of their heavy chains, antibodies (immunoglobulins) can be assigned to different classes. The antibody or antigen-binding fragment can be, e.g., any of an IgG, IgD, IgE, IgA or IgM antibody or fragment thereof, respectively. In an embodiment the antibody is an immunoglobulin G. In an embodiment the antibody fragment is a fragment of an immunoglobulin G. In an embodiment the antibody is an IgG1, IgG2, IgG2a, IgG2b, IgG3 or IgG4. In an embodiment the antibody comprises sequences from a mammalian IgG1, mammalian IgG2, mammalian IgG3 or mammalian IgG4. In an embodiment the antibody comprises sequences from a human IgG1, human IgG2, human IgG3 or human IgG4. A combination of any of these antibodies subtypes can also be used. One consideration in selecting the type of antibody to be used is the desired serum half-life of the antibody. For example, an IgG generally has a serum half-life of 23 days, IgA 6 days, IgM 5 days, IgD 3 days, and IgE 2 days. (Abbas AK, Lichtman AH, Pober JS. Cellular and Molecular Immunology, 4th edition, W.B. Saunders Co., Philadelphia, 2000, hereby incorporated by reference in its entirety). The antibody, or antigen-binding fragment thereof, may comprise sequences from more than one class or isotype.

In an embodiment, the antibody, or antigen-binding fragment thereof, comprises an Fc domain that has the same sequence or 99% or greater sequence similarity with a human IgG1 Fc domain. In an embodiment, the antibody, or antigen-binding fragment thereof, comprises an Fc domain that has the same sequence or 99% or greater sequence similarity with a human IgG2 Fc domain. In an embodiment, the antibody, or antigen-binding fragment thereof, comprises an Fc domain that has the same sequence or 99% or greater sequence similarity with a human IgG3 Fc domain. In an embodiment, the antibody, or antigen-binding fragment thereof, comprises an Fc domain that has the same sequence or 99% or greater sequence similarity with a human IgG4 Fc domain. In an embodiment, the Fc domain is not mutated.

In an embodiment, the Fc domain is mutated at the CH2-CH3 domain interface to increase the affinity of IgG for FcRn at acidic but not neutral pH. In an embodiment, the antibody, or antigen-binding fragment thereof, comprises an Fc domain that has the same sequence as a human IgG1 Fc domain.

In some embodiment, the antibody, or antigen-binding fragment thereof, has a modified IgG Fc region. Modified IgG Fc regions are well known in the art. For example, see any of the mutations listed in Table 1 of Wang et al. Protein Cell (2018), 9(1):63-73. In embodiments, the modified Fc region, relative to the unmodified Fc region, has enhanced complement-based effector function, increased or decreased FcγR-based effector function, reduced effector function, enhanced coengagement of antigen and FcγRs, and/or increased serum half-life.

Non-limiting examples of Fc modifications to modulate antibody effector function for IgG1 (see Wang et al. Protein Cell (2018), 9(1):63-73) include are:
(a) Increased FcγRIIIa binding: F243L/R292P/Y300L/V305I/P396L
(b) Increased FcγRIIIa binding: S239D/I332E
(c) Increased FcγRIIIa binding: decreased FcγRIIb binding S239D/I332E/A330L
(d) Increased FcγRIIIa binding: S298A/E333A/K334A.
   i. For example, in one heavy chain: L234Y/L235Q/G236W/S239M/H268D/D270E/S298A
   ii. For example, in the opposing heavy chain: D270E/K326D/A330M/K334E
(e) Increased FcγRIIa binding, increased FcγRIIIa binding: G236A/S239D/I332E
(f) Enhanced CDC, increased C1q binding: K326W/E333S
(g) Increased C1q binding: S267E/H268F/S324T
(h) Increased C1q binding, IgG1/IgG3 cross subclass Hexamerization: E345R/E430G/S440Y
(i) Reduced effector function: Aglycosylated N297A or N297Q or N297G
(j) Reduced FcγR and C1q binding: L235E
(k) Reduced FcγR and C1q binding
   i. IgG1: L234A/L235A
   ii. IgG4: F234A/L235A
(l) Reduced FcγR and C1q binding, IgG2/I gG4 cross isotype
(m) Reduced FcγR and C1q binding IgG2: H268Q/V309L/A330S/P331S
(n) Reduced FcγR and C1q binding IgG2: V234A/G237A/P238S/H268A/V309L/A330S/P331S
(o) Increased FcRn binding at pH 6.0: M252Y/S254T/T256E
(p) Increased FcRn binding at pH 6.0: M428L/N434S
(q) Increased FcγRIIb binding: S267E/L328F
(r) Increased FcγRIIa binding, decreased FcγRIIIa binding: N325S/L328F.

### Antibody Binding

In embodiments, the anti-Mtb AM antibody, or Mtb AM-binding fragment thereof, binds Mtb AM with a binding affinity (K_{D}) of from about 1×10⁻⁶ M to about 1×10⁻¹⁰ M, from about 1×10⁻⁷ M to about 1×10⁻¹⁰ M, from about 1×10⁻⁸ M to about 1×10⁻¹⁰ M, from about 1×10⁻⁸ M to about 1×10⁻¹⁹ M. In embodiments, the anti-Mtb AM antibody, or Mtb AM-binding fragment thereof, binds Mtb AM with a binding affinity (K_{D}) of about 1×10⁻⁷ M, about 1×10⁻⁸ M, about 1×10⁻⁹ M, or about 1×10⁻¹⁰ M.

In embodiments, the anti-GroEL2 antibody, or GroEL2-binding fragment thereof, binds GroEL2 with a binding affinity (K_{D}) of from about 1×10⁻⁶ M to about 1×10⁻¹⁰ M, from about 1×10⁻⁷ M to about 1×10⁻¹⁰ M, from about 1×10⁻⁸ M to about 1×10⁻¹⁰ M, from about 1×10⁻⁸ M to about 1×10⁻¹⁹ M. In embodiments, the anti-GroEL2 antibody, or GroEL2-binding fragment thereof, binds GroEL2 with a binding affinity (K_{D}) of about 1×10⁻⁷ M, about 1×10⁻⁸ M, about 1×10⁻⁹ M, or about 1×10⁻¹⁰ M.

In embodiments, the anti-LprG antibody, or LprG-binding fragment thereof, binds LprG with a binding affinity (K_{D}) of from about 1×10⁻⁶ M to about 1×10⁻¹⁰ M, from about 1×10⁻⁷ M to about 1×10⁻¹⁰ M, from about 1×10⁻⁸ M to about 1×10⁻¹⁰ M, from about 1×10⁻⁸ M to about 1×10⁻¹⁹ M. In embodiments, the anti-LprG antibody, or LprG-binding fragment thereof, binds LprG with a binding affinity (K_{D}) of about 1×10⁻⁷ M, about 1×10⁻⁸ M, about 1×10⁻⁹ M, or about 1×10⁻¹⁰ M.

The term “K_{D}”, as used herein, is intended to refer to the dissociation constant of an antibody-antigen interaction. One way of determining the K_{D} or binding affinity of antibodies to their antigen is by measuring binding affinity Using a Dip and Read assay using an immobilized antigen and monoclonal antibodies (Octet Red96 ForteBio, Fremont, CA). (The affinity constant is the inverted dissociation constant). Biotinylated antigen can be diluted into PBS+0.1% BSA, 0.02% Tween 20 and 0.05% sodium azide (Kinetics Buffer, ForteBio) and dipped into wells containing serial diluted mAbs starting from 37.75 nM. The concentrations of the Fab proteins are determined by ELISA and/or SDS-PAGE electrophoresis using an IgG1 standard monoclonal antibody of known concentration as a standard. Kinetic association rates (kₒₙ) and dissociation rates (k_{off}) are obtained simultaneously by fitting the data to a 1:1 Langmuir binding model (Karlsson, R. Roos, H. Fagerstam, L. Petersson, B. (1994). Methods Enzymology 6. 99-110, the content of which is hereby incorporated in its entirety) using the BIA evaluation program. Equilibrium dissociation constant (K_{D}) values are calculated as k_{off}/kₒₙ. This protocol is suitable for use in determining binding affinity of an antibody or fragment to any antigen. Other protocols known in the art may also be used. For example, ELISA of antigen with mAb can be used to determine the K_{D} values.

In an embodiment, the anti-Mtb AM antibody, or Mtb AM-binding fragment thereof, described herein is capable of specifically binding or specifically binds an Mtb capsular AM. In an embodiment, the anti-GroEL2 antibody, or GroEL2-binding fragment thereof, described herein is capable of specifically binding or specifically binds to GroEL2. In an embodiment, the anti-LprG antibody, or LprG-binding fragment thereof, described herein is capable of specifically binding or specifically binds to LprG. As used herein, the terms “is capable of specifically binding” or “specifically binds” refers to the property of an antibody or fragment of binding to the specified antigen with a dissociation constant that is <1 μM, preferably <1 nM and most preferably <10 μM.

In an embodiment, the K_{D} of the anti-Mtb AM antibody, or Mtb AM-binding fragment thereof, for Mtb capsular AM is less than 100 nM. In an embodiment, the K_{D} of the anti-Mtb AM antibody, or Mtb AM-binding fragment thereof, for Mtb capsular AM is less than 10 nM. In an embodiment, the K_{D} of the anti-Mtb AM antibody, or Mtb AM-binding fragment thereof, for Mtb capsular AM is less than 1.0 nM. In an embodiment, the K_{D} of the anti-Mtb AM antibody, or Mtb AM-binding fragment thereof, for avirulent Mtb capsular AM is a lower affinity than its K_{D} for virulent Mtb capsular AM. In an embodiment, the K_{D} of the anti-GroEL2 antibody, or GroEL2-binding fragment thereof, for GroEL2 is less than 100 nM. In an embodiment, the K_{D} of the anti-GroEL2 antibody, or GroEL2-binding fragment thereof, for GroEL2 is less than 10 nM. In an embodiment, the K_{D} of the anti-GroEL2 antibody, or GroEL2-binding fragment thereof, for GroEL2 is less than 1.0 nM. In an embodiment, the K_{D}of the anti-LprG antibody, or LprG-binding fragment thereof, for LprG is less than 100 nM. In an embodiment, the K_{D} of the anti-LprG antibody, or LprG-binding fragment thereof, for LprG is less than 10 nM. In an embodiment, the K_{D} of the anti-LprG antibody, or LprG-binding fragment thereof, for LprG is less than 1.0 nM.

An epitope that “specifically binds” to an antibody or a polypeptide is a term well understood in the art. A molecular entity is said to exhibit “specific binding” or “preferential binding” if it reacts or associates more frequently, more rapidly, with greater duration and/or with greater affinity with a particular cell or substance than it does with alternative cells or substances. An antibody “specifically binds” or “preferentially binds” to a target if it binds with greater affinity, avidity, more readily, and/or with greater duration than it binds to other substances. For example, an antibody that specifically or preferentially binds to anMtb capsular AM conformational epitope is an antibody that binds this epitope with greater affinity, avidity, more readily, and/or with greater duration than it binds to other Mtb capsular AM epitopes or non-Mtb capsular AM epitopes. An antibody that specifically or preferentially binds to a GroEL2 conformational epitope is an antibody that binds this epitope with greater affinity, avidity, more readily, and/or with greater duration than it binds to other GroEL2 epitopes. An antibody that specifically or preferentially binds to a LprG conformational epitope is an antibody that binds this epitope with greater affinity, avidity, more readily, and/or with greater duration than it binds to other LprG epitopes. The specific epitope of an antibody may be relevant for the therapeutic or diagnostic value of the antibody.

It is also understood by reading this definition that, for example, an antibody (or moiety or epitope) that specifically or preferentially binds to a first target may or may not specifically or preferentially bind to a second target. As such, “specific binding” or “preferential binding” does not necessarily require, although it can include, exclusive binding.

In embodiments, the antibody, or antigen-binding fragment thereof, binds to a linear epitope. In embodiments, the antibody, or antigen-binding fragment thereof, binds to an oligosaccharide fragment.

In one embodiment, an antibody, or antigen-binding fragment thereof, disclosed herein binds to its antigen with one, two, three four, five, or six CDRs.

The term “compete”, as used herein with regard to an antibody, means that a first antibody, or an antigen-binding portion thereof, binds to an epitope in a manner sufficiently similar to the binding of a second antibody, or an antigen-binding portion thereof, such that the result of binding of the first antibody with its cognate epitope is detectably decreased in the presence of the second antibody compared to the binding of the first antibody in the absence of the second antibody. The alternative, where the binding of the second antibody to its epitope is also detectably decreased in the presence of the first antibody, can, but need not be the case. That is, a first antibody can inhibit the binding of a second antibody to its epitope without that second antibody inhibiting the binding of the first antibody to its respective epitope. However, where each antibody detectably inhibits the binding of the other antibody with its cognate epitope or ligand, whether to the same, greater, or lesser extent, the antibodies are said to “cross-compete” with each other for binding of their respective epitope(s). Both competing and cross-competing antibodies are disclosed herein. Regardless of the mechanism by which such competition or cross-competition occurs (e.g., steric hindrance, conformational change, or binding to a common epitope, or portion thereof), the skilled artisan would appreciate, based upon the teachings provided herein, that such competing and/or cross-competing antibodies are encompassed and can be useful for the methods disclosed herein.

Provided herein are antibodies that compete with any of the antibodies disclosed herein.

Provided herein are bispecific antibodies comprising any of the CDR and/or variable light chain disclosed herein. As used herein, a bispecific antibody refers to an antibody that is capable of binding two antigens simultaneously. In embodiments, at least one of the antigens is AM, an AM fragment, LAM, or an AM-comprising LAM fragment. In embodiments, at least one of the antigens is GroEL2. In embodiments, at least one of the antigens is LprG. In embodiments, the bispecific antibody or antigen-binding fragment thereof bind to a first AM, an AM fragment, LAM, or an AM-comprising LAM fragment antigen and a second AM, an AM fragment, LAM, or an AM-comprising LAM fragment antigen, wherein the first and the second antigens are different.

### Antibody Fragments

As used herein, the term “antigen-binding portion” or “antigen-binding fragment” may be a fragment comprising a Fab, Fab′, F(ab′)2, Fd, Fv, domain antibodies (dAbs such as shark and camel antibodies), ScFv, a maxibody, a minibody, a nanobody, an intrabody, a diabody, a triabody, a tetrabody, a v-NAR and a bis-scFv, or a polypeptide that contain at least certain portions of an immunoglobulin sufficient to confer specific antigen-binding to the polypeptide.

Antibody fragments can be prepared, for example, by cleaving an intact antibody or by recombinant means. See generally, Fundamental Immunology, Ch. 7 (Paul, W., ed., 2nd ed. Raven Press, N.Y. (1989), hereby incorporated by reference in its entirety). Antigen-binding fragments may be produced by recombinant DNA techniques or by enzymatic or chemical cleavage of intact antibodies or by molecular biology techniques.

In some embodiments of the aspects described herein, the antibody disclosed herein is a Fab fragment, which comprises or consist essentially of a variable (VL) and constant (CL) domain of the light chain and a variable domain (VH) and the first constant domain (CH1) of the heavy chain.

In some embodiments of the aspects described herein, the antibody fragment is a Fab′ fragment, which refers to a Fab fragment having one or more cysteine residues at the C-terminus of the CH1 domain.

In some embodiments of the aspects described herein, the antibody fragment is an Fd fragment comprising or consisting essentially of VH and CH1 domains.

In some embodiments of the aspects described herein, the antibody fragment is an Fd′ fragment comprising VH and CH1 domains and one or more cysteine residues at the C-terminus of the CH1 domain.

Single-chain Fv or scFv antibody fragments comprise or consist essentially of the VH and VL domains of an antibody, such that these domains are present in a single polypeptide chain. Generally, an Fv polypeptide further comprises a polypeptide linker between the VH and VL domains, which allows the scFv to form the desired structure for antigen-binding. Accordingly, in some embodiments of the aspects described herein, the antibody fragment is a Fv fragment comprising or consisting essentially of the VL and VH domains of a single arm of an antibody.

In some embodiments of the aspects described herein, the antibody fragment is a diabody comprising two antigen-binding sites, comprising a heavy chain variable domain (VH) connected to a light chain variable domain (VL) in the same polypeptide chain.

In some embodiments of the aspects described herein, the antibody fragment is a dAb fragment comprising or consisting essentially of a VH domain.

In some embodiments of the aspects described herein, the antibody fragment is a F(ab′)2 fragment, which comprises a bivalent fragment comprising two Fab′ fragments linked by a disulfide bridge at the hinge region.

In some embodiments of the aspects described herein, the antibody fragment is a linear antibody comprising a pair of tandem Fd segments (VH-CH1-VH—CH1) which, together with complementary light chain polypeptides, form a pair of antigen-binding regions.

A person skilled in the arts can use various techniques that have been developed and are available for the production of antibody fragments. Traditionally, these fragments were derived via proteolytic digestion of intact antibodies. However, F(ab′)2 fragments can be isolated directly from recombinant host cell culture. Other techniques for the production of antibody fragments will be apparent to the skilled practitioner. In other embodiments, the antibody fragment of choice is a single chain Fv fragment (scFv). See, for example, WO 93/16185. Alternatively, these fragments can also be produced directly by recombinant host cells. For example, antibody fragments can be isolated from the antibody phage libraries discussed herein. In another approach, Fab′-SH fragments can be directly recovered from E. coli and chemically coupled to form F(ab′)2 fragments (Carter et al., 1992).

### Antibody Conjugates

In some embodiments, an antibody, or antigen-binding fragment thereof, disclosed herein may include a modification, including, but not limited to glycosylation, acetylation, pegylation, phosphorylation, phosphorylation, amidation, derivatization by known protecting/blocking groups, proteolytic cleavage, linkage to a cellular ligand or other protein, etc. The process of making chemical modifications is known in the art, and may include, but are not limited to, specific chemical cleavage, acetylation, formylation, metabolic synthesis of tunicamycin, etc. Additionally, the molecules may contain one or more non-classical amino acids.

In some embodiments of the aspects described herein, an antibody, or antigen-binding fragment thereof, is conjugated to a functional moiety. Examples of useful functional moieties include, but are not limited to, a blocking moiety, a detectable moiety, a diagnostic moiety, a targeting moiety, and a therapeutic moiety.

Exemplary blocking moieties include moieties of sufficient steric bulk and/or charge such that reduced glycosylation occurs, for example, by blocking the ability of a glycosidase to glycosylate the antibody or antigen-binding fragment thereof. The blocking moiety may, additionally or alternatively, reduce effector function, for example, by inhibiting the ability of the Fc region to bind a receptor or complement protein. Preferred blocking moieties include cysteine adducts and PEG moieties.

In one embodiment, the blocking moiety is a cysteine, preferably a cysteine that has associated with a free cysteine, e.g., during or subsequent to the translation of the Fc containing polypeptide, e.g., in cell culture. Other blocking cysteine adducts include cystine, mixed disulfide adducts, or disulfide linkages.

In another embodiment, the blocking moiety is a polyalkylene glycol moiety, for example, a PEG moiety and preferably a PEG-maleimide moiety. Preferred pegylation moieties (or related polymers) can be, for example, polyethylene glycol (“PEG”), polypropylene glycol (“PPG”), polyoxyethylated glycerol (“POG”) and other polyoxyethylated polyols, polyvinyl alcohol (“PVA”) and other polyalkylene oxides, polyoxyethylated sorbitol, or polyoxyethylated glu-cose. The polymer can be a homopolymer, a random or block copolymer, a terpolymer based on the monomers listed above, straight chain or branched, substituted or unsubstituted as long as it has at least one active sulfone moiety. The polymeric portion can be of any length or molecular weight but these characteristics can affect the biological properties. Polymer average molecular weights particularly useful for decreasing clearance rates in pharmaceutical applications are in the range of 2,000 to 35,000 Daltons. In addition, if two groups are linked to the polymer, one at each end, the length of the polymer can impact upon the effective distance, and other spatial relationships, between the two groups. Thus, one skilled in the art can vary the length of the polymer to optimize or confer the desired biological activity. PEG is useful in biological applications for several reasons. PEG typically is clear, colorless, odorless, soluble in water, stable to heat, inert to many chemical agents, does not hydrolyze, and is nontoxic. Pegylation can im-prove pharmacokinetic performance of a molecule by increasing the molecule's apparent molecular weight. The increased apparent molecular weight reduces the rate of clearance from the body following subcutaneous or systemic administration. In many cases, pegylation can decrease antigenicity and immunogenicity. In addition, pegylation can increase the solubility of a biologically-active molecule.

Examples of detectable moieties for the detection of the antibodies and antigen-binding fragments thereof disclosed herein include fluorescent moieties or labels, imaging agents, radioisotopic moieties, radiopaque moieties, and the like, e.g. detectable labels such as biotin, fluorophores, chromophores, spin resonance probes, or radiolabels. Exemplary fluorophores include fluorescent dyes (e.g. fluorescein, rhodamine, and the like) and other luminescent molecules (e.g. luminol). A fluorophore may be environmentally-sensitive such that its fluorescence changes if it is located close to one or more residues in the modified protein that undergo structural changes upon binding a substrate (e.g. dansyl probes). Exemplary radiolabels include small molecules containing atoms with one or more low sensitivity nuclei (¹³C, ¹⁵N, ²H, ¹²⁵I, ¹²³J, ⁹⁹Tc, ⁴³K, ⁵²Fe, ⁶⁷Ga, ⁶⁸Ga, ¹¹In and the like). Other useful moieties are known in the art.

Examples of therapeutic moieties include anti-tuberculosis agents. Anti-tuberculosis agents include, but are not limited to, ethambutol, pyrazinamide, streptomycin, isoniazid, moxifloxacin rifampicin, levofloxacin, moxifloxacin, clofazimine, bedaquiline, cycloserine, terizidone, delamanid, linezolid, pyrazinamide, imipenem-cilastatin (Ipm-Cln) or Meropenem, amikacin, streptomycin, ethionamide, Prothionamide, and p-aminosalicylic acid.

The functional moiety may also have one or more of the above-mentioned functions.

To increase the half-life of the antibodies or polypeptides containing the amino acid sequences described herein, one can attach a salvage receptor binding epitope to an antibody, or antigen-binding fragment thereof, (especially an antibody fragment), as described, e.g., in U.S. Pat. No. 5,739,277. The term “salvage receptor binding epitope” may refer to an epitope of the Fc region of an IgG molecule (e.g., IgG1, IgG2, IgG3, or IgG4) that is responsible for in-creasing the in vivo serum half-life of the IgG molecule (e.g., Ghetie et al., 18 Ann. Rev. Immunol. 739 (2000). Antibodies with substitutions in an Fc region thereof and increased serum half-lives are also described in WO 00/42072, WO 02/060919; Shields et al., 276 J. Biol. Chem. 6591 (2001); Hinton, 279 J. Biol. Chem. 6213-6216 (2004). For example, a nucleic acid molecule encoding the salvage receptor binding epitope can be linked in frame to a nucleic acid encoding a polypeptide sequence described herein so that the fusion protein expressed by the engineered nucleic acid molecule comprises the salvage receptor binding epitope and a polypeptide sequence described herein. In another embodiment, the serum half-life can also be in-creased, for example, by attaching other polypeptide sequences.

Other types of functional moieties are known in the art and can be readily used in the methods and compositions of the present disclosure based on the teachings contained herein.

### Nucleic Acids

Also provided herein are nucleic acids encoding the antibodies, or antigen-binding fragments thereof, disclosed herein, as well as vectors, host cells, and expression systems. The term “nucleic acid” as used herein refers to a polymeric form of nucleotides of any length, either ribonucleotides or desoxyribonucleotides. Thus, this term includes, but is not limited to, single-, double- or multi-stranded DNA or RNA, genomic DNA, cDNA, DNA-RNA hybrids, or a polymer comprising purine and pyrimidine bases, or other natural, chemically or biochemically modified, non-natural, or derivatized nucleotide bases.

### Nucleic Acids Encoding Anti-Mtb AM Antibodies or Mtb AM-Binding Fragments Thereof

In some embodiments, provided is a nucleic acid sequence encoding a heavy chain variable region, the nucleic acid sequence comprising a sequence that is at least 80%, at least 85%, at least 90%, at least 95%, at least 96%, at least 97%, at least 98%, or at least 99% identical to any one of SEQ ID NOs:8, 17, 26, 35, 44, 53, and 168. In some embodiments, provided is a nucleic acid sequence encoding a heavy chain variable region, the nucleic acid sequence comprising any one of SEQ ID NOs:8, 17, 26, 35, 44, 53, and 168.

In some embodiments, provided is a nucleic acid sequence encoding a light chain variable region, the nucleic acid sequence comprising a sequence that is at least 80%, at least 85%, at least 90%, at least 95%, at least 96%, at least 97%, at least 98%, or at least 99% identical to any one of SEQ ID NOs:9, 18, 27, 36, 45, 54, and 169. In some embodiments, provided is a nucleic acid sequence encoding a light chain variable region, the nucleic acid sequence comprising any one of SEQ ID NOs:9, 18, 27, 36, 45, 54, and 169.

### Nucleic Acids Encoding Anti-GroEL2 Antibodies or GroEL2-Bindin Fragments Thereof

In some embodiments, provided is a nucleic acid sequence encoding a heavy chain variable region, the nucleic acid sequence comprising a sequence that is at least 80%, at least 85%, at least 90%, at least 95%, at least 96%, at least 97%, at least 98%, or at least 99% identical to any one of SEQ ID NOs:66, 79, 92, 105, and 118. In some embodiments, provided is a nucleic acid sequence encoding a heavy chain variable region, the nucleic acid sequence comprising any one of SEQ ID NOs:66, 79, 92, 105, and 118.

In some embodiments, provided is a nucleic acid sequence encoding a light chain variable region, the nucleic acid sequence comprising a sequence that is at least 80%, at least 85%, at least 90%, at least 95%, at least 96%, at least 97%, at least 98%, or at least 99% identical to any one of SEQ ID NOs:67, 80, 93, 106, and 119. In some embodiments, provided is a nucleic acid sequence encoding a light chain variable region, the nucleic acid sequence comprising any one of SEQ ID NOs:67, 80, 93, 106, and 119.

In some embodiments, provided is a nucleic acid sequence encoding a heavy chain region, the nucleic acid sequence comprising a sequence that is at least 80%, at least 85%, at least 90%, at least 95%, at least 96%, at least 97%, at least 98%, or at least 99% identical to any one of SEQ ID NOs:70, 83, 96, 109, and 122. In some embodiments, provided is a nucleic acid sequence encoding a heavy chain variable region, the nucleic acid sequence comprising any one of SEQ ID NOs:70, 83, 96, 109, and 122.

In some embodiments, provided is a nucleic acid sequence encoding a light chain region, the nucleic acid sequence comprising a sequence that is at least 80%, at least 85%, at least 90%, at least 95%, at least 96%, at least 97%, at least 98%, or at least 99% identical to any one of SEQ ID NOs:71, 84, 97, 110, and 123. In some embodiments, provided is a nucleic acid sequence encoding a heavy chain variable region, the nucleic acid sequence comprising any one of SEQ ID NOs:71, 84, 97, 110, and 123.

### Nucleic Acids Encoding Anti-LprG Antibodies or LprG-Binding Fragments Thereof

In some embodiments, provided is a nucleic acid sequence encoding a heavy chain variable region, the nucleic acid sequence comprising a sequence that is at least 80%, at least 85%, at least 90%, at least 95%, at least 96%, at least 97%, at least 98%, or at least 99% identical to SEQ ID NO:131. In some embodiments, provided is a nucleic acid sequence encoding a heavy chain variable region, the nucleic acid sequence comprising SEQ ID NO:131.

In some embodiments, provided is a nucleic acid sequence encoding a light chain variable region, the nucleic acid sequence comprising a sequence that is at least 80%, at least 85%, at least 90%, at least 95%, at least 96%, at least 97%, at least 98%, or at least 99% identical to SEQ ID NO:132. In some embodiments, provided is a nucleic acid sequence encoding a light chain variable region, the nucleic acid sequence comprising SEQ ID NO:132.

In some embodiments, provided is a nucleic acid sequence encoding a heavy chain region, the nucleic acid sequence comprising a sequence that is at least 80%, at least 85%, at least 90%, at least 95%, at least 96%, at least 97%, at least 98%, or at least 99% identical to SEQ ID NO:135. In some embodiments, provided is a nucleic acid sequence encoding a heavy chain variable region, the nucleic acid sequence comprising SEQ ID NO:135.

In some embodiments, provided is a nucleic acid sequence encoding a light chain region, the nucleic acid sequence comprising a sequence that is at least 80%, at least 85%, at least 90%, at least 95%, at least 96%, at least 97%, at least 98%, or at least 99% identical to SEQ ID NO:136. In some embodiments, provided is a nucleic acid sequence encoding a heavy chain variable region, the nucleic acid sequence comprising SEQ ID NO:136.

The nucleic acids encoding the antibody, or antigen-binding fragment thereof, disclosed herein may be, e.g., DNA, cDNA, RNA, synthetically produced DNA or RNA, or a recombinantly produced chimeric nucleic acid molecule comprising any of those polynucleotides either alone or in combination. For example, provided is an expression vector or set of expression vectors comprising a polynucleotide sequence encoding an antibody, or antigen-binding fragment thereof, described herein operably linked to expression control sequences suitable for expression in a eukaryotic and/or prokaryotic host cell.

The term “vector” refers to a nucleic acid molecule capable of transporting another nucleic acid to which it has been linked. A “vector” includes, but is not limited to, a viral vector, a plasmid, an RNA vector or a linear or circular DNA or RNA molecule which may consists of a chromosomal, non-chromosomal, semi-synthetic or synthetic nucleic acids. In some embodiments, the employed vectors are those capable of autonomous replication (episomal vector) and/or expression of nucleic acids to which they are linked (expression vectors). Large numbers of suitable vectors are known to those of skill in the art and commercially available. Viral vectors include retrovirus, adenovirus, parvovirus (e.g., adeno associated viruses, AAV), coronavirus, negative strand RNA viruses such as orthomyxovirus (e.g., influenza virus), rhabdovirus (e. g., rabies and vesicular stomatitis virus), paramyxovirus (e.g., measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses, D type viruses, HTLV-BLV group, lentivirus, and spumavirus.

### Vectors Encoding Anti-Mtb AM Antibodies or Mtb AM-Binding Fragments Thereof

In some embodiments, provided is a vector comprising a nucleic acid sequence encoding a heavy chain variable region, the nucleic acid sequence comprising a sequence that is at least 80%, at least 85%, at least 90%, at least 95%, at least 96%, at least 97%, at least 98%, or at least 99% identical to any one of SEQ ID NOs:8, 17, 26, 35, 44, 53, and 168. In some embodiments, provided is a vector comprising a nucleic acid sequence encoding a heavy chain variable region, the nucleic acid sequence comprising any one of SEQ ID NOs:8, 17, 26, 35, 44, 53, and 168.

In some embodiments, provided is a vector comprising a nucleic acid sequence encoding a light chain variable region, the nucleic acid sequence comprising a sequence that is at least 80%, at least 85%, at least 90%, at least 95%, at least 96%, at least 97%, at least 98%, or at least 99% identical to any one of SEQ ID NOs:9, 18, 27, 36, 45, 54, and 169. In some embodiments, provided is a vector comprising a nucleic acid sequence encoding a light chain variable region, the nucleic acid sequence comprising any one of SEQ ID NOs:9, 18, 27, 36, 45, 54, and 169.

In some embodiments, provided is a vector or a set of vectors comprising (1) a nucleic acid sequence comprising a sequence that is at least 80%, at least 85%, at least 90%, at least 95%, at least 96%, at least 97%, at least 98%, or at least 99% identical to any one of SEQ ID NOs:8, 17, 26, 35, 44, 53, and 168 and (2) a nucleic acid sequence comprising a sequence that is at least 80%, at least 85%, at least 90%, at least 95%, at least 96%, at least 97%, at least 98%, or at least 99% identical to any one of SEQ ID NOs:9, 18, 27, 36, 45, 54, and 169.

In some embodiments, provided is a vector or a set of vectors comprising (1) a nucleic acid sequence comprising any one of SEQ ID NOs:8, 17, 26, 35, 44, 53, and 168 and (2) a nucleic acid sequence comprising any one of SEQ ID NOs:9, 18, 27, 36, 45, 54, and 169.

In one embodiment, provided is a vector or a set of vectors comprising
(a) SEQ ID NO:8 and/or SEQ ID NO:9;
(b) SEQ ID NO:17 and/or SEQ ID NO:18;
(c) SEQ ID NO:26 and/or SEQ ID NO:27;
(d) SEQ ID NO:35 and/or SEQ ID NO:36;
(e) SEQ ID NO:44 and/or SEQ ID NO:45;
(f) SEQ ID NO:53 and/or SEQ ID NO:54; or
(g) SEQ ID NO:168 and/or SEQ ID NO:169.

### Vectors Encoding Anti-GroEL2 Antibodies or GroEL2-Binding Fragments Thereof

In some embodiments, provided is a vector comprising a nucleic acid sequence encoding a heavy chain variable region, the nucleic acid sequence comprising a sequence that is at least 80%, at least 85%, at least 90%, at least 95%, at least 96%, at least 97%, at least 98%, or at least 99% identical to any one of SEQ ID NOs:66, 79, 92, 105, and 118. In some embodiments, provided is a vector comprising a nucleic acid sequence encoding a heavy chain variable region, the nucleic acid sequence comprising any one of SEQ ID NOs:66, 79, 92, 105, and 118.

In some embodiments, provided is a vector comprising a nucleic acid sequence encoding a light chain variable region, the nucleic acid sequence comprising a sequence that is at least 80%, at least 85%, at least 90%, at least 95%, at least 96%, at least 97%, at least 98%, or at least 99% identical to any one of SEQ ID NOs:67, 80, 93, 106, and 119. In some embodiments, provided is a vector comprising a nucleic acid sequence encoding a light chain variable region, the nucleic acid sequence comprising any one of SEQ ID NOs:67, 80, 93, 106, and 119.

In some embodiments, provided is a vector comprising a nucleic acid sequence encoding a heavy chain region, the nucleic acid sequence comprising a sequence that is at least 80%, at least 85%, at least 90%, at least 95%, at least 96%, at least 97%, at least 98%, or at least 99% identical to any one of SEQ ID NOs:70, 83, 96, 109, and 122. In some embodiments, provided is a vector comprising a nucleic acid sequence encoding a heavy chain variable region, the nucleic acid sequence comprising any one of SEQ ID NOs:70, 83, 96, 109, and 122.

In some embodiments, provided is a vector comprising a nucleic acid sequence encoding a light chain region, the nucleic acid sequence comprising a sequence that is at least 80%, at least 85%, at least 90%, at least 95%, at least 96%, at least 97%, at least 98%, or at least 99% identical to any one of SEQ ID NOs:71, 84, 97, 110, and 123. In some embodiments, provided is a vector comprising a nucleic acid sequence encoding a heavy chain variable region, the nucleic acid sequence comprising any one of SEQ ID NOs:71, 84, 97, 110, and 123.

In some embodiments, provided is a vector or a set of vectors comprising (1) a nucleic acid sequence comprising any one of SEQ ID NOs:66, 79, 92, 105, and 118 and (2) a nucleic acid sequence comprising any one of SEQ ID NOs:67, 80, 93, 106, and 119.

In one embodiment, provided is a vector or a set of vectors comprising
(a) SEQ ID NO:66 and/or SEQ ID NO:67;
(b) SEQ ID NO:79 and/or SEQ ID NO:80;
(c) SEQ ID NO:92 and/or SEQ ID NO:93;
(d) SEQ ID NO:105 and/or SEQ ID NO:106; or
(e) SEQ ID NO:118 and/or SEQ ID NO:119.

### Vectors Encoding Anti-LprG Antibodies or LprG-Binding Fragments Thereof

In some embodiments, provided is a vector comprising a nucleic acid sequence encoding a heavy chain variable region, the nucleic acid sequence comprising a sequence that is at least 80%, at least 85%, at least 90%, at least 95%, at least 96%, at least 97%, at least 98%, or at least 99% identical to SEQ ID NO:131. In some embodiments, provided is a vector comprising a nucleic acid sequence encoding a heavy chain variable region, the nucleic acid sequence comprising SEQ ID NO:131.

In some embodiments, provided is a vector comprising a nucleic acid sequence encoding a light chain variable region, the nucleic acid sequence comprising a sequence that is at least 80%, at least 85%, at least 90%, at least 95%, at least 96%, at least 97%, at least 98%, or at least 99% identical to SEQ ID NO:132. In some embodiments, provided is a vector comprising a nucleic acid sequence encoding a light chain variable region, the nucleic acid sequence comprising SEQ ID NO:132.

In some embodiments, provided is a vector comprising a nucleic acid sequence encoding a heavy chain region, the nucleic acid sequence comprising a sequence that is at least 80%, at least 85%, at least 90%, at least 95%, at least 96%, at least 97%, at least 98%, or at least 99% identical to SEQ ID NO:135. In some embodiments, provided is a vector comprising a nucleic acid sequence encoding a heavy chain variable region, the nucleic acid sequence comprising SEQ ID NO:135.

In some embodiments, provided is a vector comprising a nucleic acid sequence encoding a light chain region, the nucleic acid sequence comprising a sequence that is at least 80%, at least 85%, at least 90%, at least 95%, at least 96%, at least 97%, at least 98%, or at least 99% identical to SEQ ID NO:136. In some embodiments, provided is a vector comprising a nucleic acid sequence encoding a heavy chain variable region, the nucleic acid sequence comprising SEQ ID NO: 136.

In some embodiments, provided is a vector or a set of vectors comprising a nucleic acid sequence comprising SEQ ID NO:131 and a nucleic acid sequence comprising SEQ ID NO:132.

A variety of expression vectors have been developed for the efficient synthesis of an antibody, or antigen-binding fragment thereof, in prokaryotic cells such as bacteria and in eukaryotic systems, including but not limited to yeast and mammalian cell culture systems have been developed. The vectors can comprise segments of chromosomal, non-chromosomal and synthetic DNA sequences. Also provided are cells comprising expression vectors for the expression of the disclosed the antibody, or antigen-binding fragment thereof.

In an embodiment, a host cell comprising a nucleic acid molecule described herein, or a vector described herein, is provided.

### Antibody Production

In an embodiment, provided is a method of producing an antibody, or antigen-binding fragment thereof, disclosed herein comprising culturing a cell comprising one or more nucleic acid molecules encoding for an antibody, or antigen-binding fragment thereof, disclosed herein under conditions whereby the antibody, or antigen-binding fragment thereof, is produced by the host cell.

### Methods for Producing Anti-Mtb AM Antibodies or Mtb AM-Binding Fragments Thereof

Provided herein is a method of making an anti-Mtb AM antibody, or Mtb AM-binding fragment thereof, disclosed herein, the method comprising:
(a) cultivating a cell comprising one or more nucleic acid molecules encoding an anti-Mtb AM antibody, or Mtb AM-binding fragment thereof, under conditions whereby the anti-Mtb AM antibody, or Mtb AM-binding fragment thereof, is produced by the host cell, wherein the anti-Mtb AM antibody, or Mtb AM-binding fragment thereof, comprises a heavy chain variable region and a light chain variable region, wherein each of the heavy chain and the light chain variable regions comprises a CDR1, CDR2, and CDR3; and wherein CDR1H comprises SEQ ID NO:1, CDR2H comprises SEQ ID NO:2, CDR3H comprises SEQ ID NO:3, CDR1L comprises SEQ ID NO:4, CDR2L comprises sequence AVT, and CDR3L comprises SEQ ID NO:5, and
(b) optionally, substantially isolating the anti-Mtb AM antibody, or Mtb AM-binding fragment thereof.

Provided herein is a method of making an anti-Mtb AM antibody, or Mtb AM-binding fragment thereof, disclosed herein, the method comprising:
(a) cultivating a cell comprising one or more nucleic acid molecules encoding an anti-Mtb AM antibody, or Mtb AM-binding fragment thereof, under conditions whereby the anti-Mtb AM antibody, or Mtb AM-binding fragment thereof, is produced by the host cell, wherein the anti-Mtb AM antibody, or Mtb AM-binding fragment thereof, comprises a heavy chain variable region and a light chain variable region, wherein each of the heavy chain and the light chain variable regions comprises a CDR1, CDR2, and CDR3; and wherein CDR1H comprises SEQ ID NO:10, CDR2H comprises SEQ ID NO:11, CDR3H comprises SEQ ID NO:12, CDR1L comprises SEQ ID NO:13, CDR2L comprises sequence DVT, and CDR3L comprises SEQ ID NO:14, and
(b) optionally, substantially isolating the anti-Mtb AM antibody, or Mtb AM-binding fragment thereof.

Provided herein is a method of making an anti-Mtb AM antibody, or Mtb AM-binding fragment thereof, disclosed herein, the method comprising:
(a) cultivating a cell comprising one or more nucleic acid molecules encoding an anti-Mtb AM antibody, or Mtb AM-binding fragment thereof, under conditions whereby the anti-Mtb AM antibody, or Mtb AM-binding fragment thereof, is produced by the host cell, wherein the anti-Mtb AM antibody, or Mtb AM-binding fragment thereof, comprises a heavy chain variable region and a light chain variable region, wherein each of the heavy chain and the light chain variable regions comprises a CDR1, CDR2, and CDR3; and wherein CDR1H comprises SEQ ID NO:19, CDR2H comprises SEQ ID NO:20, CDR3H comprises SEQ ID NO:21, CDR1L comprises SEQ ID NO:22, CDR2L comprises sequence KIS, and CDR3L comprises SEQ ID NO:23, and
(b) optionally, substantially isolating the anti-Mtb AM antibody, or Mtb AM-binding fragment thereof.

Provided herein is a method of making an anti-Mtb AM antibody, or Mtb AM-binding fragment thereof, disclosed herein, the method comprising:
(a) cultivating a cell comprising one or more nucleic acid molecules encoding an anti-Mtb AM antibody, or Mtb AM-binding fragment thereof, under conditions whereby the anti-Mtb AM antibody, or Mtb AM-binding fragment thereof, is produced by the host cell, wherein the anti-Mtb AM antibody, or Mtb AM-binding fragment thereof, comprises a heavy chain variable region and a light chain variable region, wherein each of the heavy chain and the light chain variable regions comprises a CDR1, CDR2, and CDR3; and wherein CDR1H comprises SEQ ID NO:28, CDR2H comprises SEQ ID NO:29, CDR3H comprises SEQ ID NO:30, CDR1L comprises SEQ ID NO:31, CDR2L comprises sequence GAS, and CDR3L comprises SEQ ID NO:32, and
(b) optionally, substantially isolating the anti-Mtb AM antibody, or Mtb AM-binding fragment thereof.

Provided herein is a method of making an anti-Mtb AM antibody, or Mtb AM-binding fragment thereof, disclosed herein, the method comprising:
(a) cultivating a cell comprising one or more nucleic acid molecules encoding an anti-Mtb AM antibody, or Mtb AM-binding fragment thereof, under conditions whereby the anti-Mtb AM antibody, or Mtb AM-binding fragment thereof, is produced by the host cell, wherein the anti-Mtb AM antibody, or Mtb AM-binding fragment thereof, comprises a heavy chain variable region and a light chain variable region, wherein each of the heavy chain and the light chain variable regions comprises a CDR1, CDR2, and CDR3; and wherein CDR1H comprises SEQ ID NO:37, CDR2H comprises SEQ ID NO:38, CDR3H comprises SEQ ID NO:39, CDR1L comprises SEQ ID NO:40, CDR2L comprises sequence EVS, and CDR3L comprises SEQ ID NO:41, and
(b) optionally, substantially isolating the anti-Mtb AM antibody, or Mtb AM-binding fragment thereof.

Provided herein is a method of making an anti-Mtb AM antibody, or Mtb AM-binding fragment thereof, disclosed herein, the method comprising:
(a) cultivating a cell comprising one or more nucleic acid molecules encoding an anti-Mtb AM antibody, or Mtb AM-binding fragment thereof, under conditions whereby the anti-Mtb AM antibody, or Mtb AM-binding fragment thereof, is produced by the host cell, wherein the anti-Mtb AM antibody, or Mtb AM-binding fragment thereof, comprises a heavy chain variable region and a light chain variable region, wherein each of the heavy chain and the light chain variable regions comprises a CDR1, CDR2, and CDR3; and wherein CDR1H comprises SEQ ID NO:46, CDR2H comprises SEQ ID NO:37, CDR3H comprises SEQ ID NO:48, CDR1L comprises SEQ ID NO:49, CDR2L comprises sequence KIS, and CDR3L comprises SEQ ID NO:50, and
(b) optionally, substantially isolating the anti-Mtb AM antibody, or Mtb AM-binding fragment thereof.

Provided herein is a method of making an anti-Mtb AM antibody, or Mtb AM-binding fragment thereof, disclosed herein, the method comprising:
(a) cultivating a cell comprising one or more nucleic acid molecules encoding an anti-Mtb AM antibody, or Mtb AM-binding fragment thereof, under conditions whereby the anti-Mtb AM antibody, or Mtb AM-binding fragment thereof, is produced by the host cell, wherein the anti-Mtb AM antibody, or Mtb AM-binding fragment thereof, comprises a heavy chain variable region and a light chain variable region, wherein each of the heavy chain and the light chain variable regions comprises a CDR1, CDR2, and CDR3; and wherein CDR1H comprises SEQ ID NO:161, CDR2H comprises SEQ ID NO:162, CDR3H comprises SEQ ID NO:163, CDR1L comprises SEQ ID NO:164, CDR2L comprises sequence KSD, and CDR3L comprises SEQ ID NO:165, and
(b) optionally, substantially isolating the anti-Mtb AM antibody, or Mtb AM-binding fragment thereof.

### Methods for Producing Anti-GroEL2 Antibodies or GroEL2-Binding Fragments Thereof

Provided herein is a method of making an anti-GroEL2 antibody, or GroEL2-binding fragment thereof, disclosed herein, the method comprising:
(a) cultivating a cell comprising one or more nucleic acid molecules encoding an anti-GroEL2 antibody, or GroEL2-binding fragment thereof, under conditions whereby the anti-GroEL2 antibody, or GroEL2-binding fragment thereof, is produced by the host cell, wherein the anti-GroEL2 antibody, or GroEL2-binding fragment thereof, comprises a heavy chain variable region and a light chain variable region, wherein each of the heavy chain and the light chain variable regions comprises a CDR1, CDR2, and CDR3; and wherein CDR1H comprises SEQ ID NO:59, CDR2H comprises SEQ ID NO:60, CDR3H comprises SEQ ID NO:61, CDR1L comprises SEQ ID NO:62, CDR2L comprises sequence LVS, and CDR3L comprises SEQ ID NO:63, and
(b) optionally, substantially isolating the anti-GroEL2 antibody, or GroEL2-binding fragment thereof.

Provided herein is a method of making an anti-GroEL2 antibody, or GroEL2-binding fragment thereof, disclosed herein, the method comprising:
(a) cultivating a cell comprising one or more nucleic acid molecules encoding an anti-GroEL2 antibody, or GroEL2-binding fragment thereof, under conditions whereby the anti-GroEL2 antibody, or GroEL2-binding fragment thereof, is produced by the host cell, wherein the anti-GroEL2 antibody, or GroEL2-binding fragment thereof, comprises a heavy chain variable region and a light chain variable region, wherein each of the heavy chain and the light chain variable regions comprises a CDR1, CDR2, and CDR3; and wherein CDR1, CDR2, and CDR3; and wherein CDR1H comprises SEQ ID NO:72, CDR2H comprises SEQ ID NO:73, CDR3H comprises SEQ ID NO:74, CDR1L comprises SEQ ID NO:75, CDR2L comprises sequence DTS, and CDR3L comprises SEQ ID NO:76, and
(b) optionally, substantially isolating the anti-GroEL2 antibody, or GroEL2-binding fragment thereof.

Provided herein is a method of making an anti-GroEL2 antibody, or GroEL2-binding fragment thereof, disclosed herein, the method comprising:
(a) cultivating a cell comprising one or more nucleic acid molecules encoding an anti-GroEL2 antibody, or GroEL2-binding fragment thereof, under conditions whereby the anti-GroEL2 antibody, or GroEL2-binding fragment thereof, is produced by the host cell, wherein the anti-GroEL2 antibody, or GroEL2-binding fragment thereof, comprises a heavy chain variable region and a light chain variable region, wherein each of the heavy chain and the light chain variable regions comprises a CDR1, CDR2, and CDR3; and wherein CDR1, CDR2, and CDR3; and wherein CDR1H comprises SEQ ID NO:85, CDR2H comprises SEQ ID NO:86, CDR3H comprises SEQ ID NO:87, CDR1L comprises SEQ ID NO:88, CDR2L comprises sequence SAS, and CDR3L comprises SEQ ID NO:89, and
(b) optionally, substantially isolating the anti-GroEL2 antibody, or GroEL2-binding fragment thereof.

Provided herein is a method of making an anti-GroEL2 antibody, or GroEL2-binding fragment thereof, disclosed herein, the method comprising:
(a) cultivating a cell comprising one or more nucleic acid molecules encoding an anti-GroEL2 antibody, or GroEL2-binding fragment thereof, under conditions whereby the anti-GroEL2 antibody, or GroEL2-binding fragment thereof, is produced by the host cell, wherein the anti-GroEL2 antibody, or GroEL2-binding fragment thereof, comprises a heavy chain variable region and a light chain variable region, wherein each of the heavy chain and the light chain variable regions comprises a CDR1, CDR2, and CDR3; and CDR1, CDR2, and CDR3; and wherein CDR1H comprises SEQ ID NO:98, CDR2H comprises SEQ ID NO:99, CDR3H comprises SEQ ID NO:100, CDR1L comprises SEQ ID NO:101, CDR2L comprises sequence AAS, and CDR3L comprises SEQ ID NO:102, and
(b) optionally, substantially isolating the anti-GroEL2 antibody, or GroEL2-binding fragment thereof.

Provided herein is a method of making an anti-GroEL2 antibody, or GroEL2-binding fragment thereof, disclosed herein, the method comprising:
(a) cultivating a cell comprising one or more nucleic acid molecules encoding an anti-GroEL2 antibody, or GroEL2-binding fragment thereof, under conditions whereby the anti-GroEL2 antibody, or GroEL2-binding fragment thereof, is produced by the host cell, wherein the anti-GroEL2 antibody, or GroEL2-binding fragment thereof, comprises a heavy chain variable region and a light chain variable region, wherein each of the heavy chain and the light chain variable regions comprises a CDR1, CDR2, and CDR3; and wherein CDR1, CDR2, and CDR3; and wherein CDR1H comprises SEQ ID NO:111, CDR2H comprises SEQ ID NO:112, CDR3H comprises SEQ ID NO:113, CDR1L comprises SEQ ID NO:114, CDR2L comprises sequence GTS, and CDR3L comprises SEQ ID NO:115, and
(b) optionally, substantially isolating the anti-GroEL2 antibody, or GroEL2-binding fragment thereof.

### Methods for Producing Anti-LprG Antibodies or LprG-Binding Fragments Thereof

Provided herein is a method of making an anti-LprG antibody, or LprG-binding fragment thereof, disclosed herein, the method comprising:
(a) cultivating a cell comprising one or more nucleic acid molecules encoding an anti-LprG antibody, or LprG-binding fragment thereof, under conditions whereby the anti-LprG antibody, or LprG-binding fragment thereof, is produced by the host cell, wherein the anti-LprG antibody, or LprG-binding fragment thereof, comprises a heavy chain variable region and a light chain variable region, wherein each of the heavy chain and the light chain variable regions comprises a CDR1, CDR2, and CDR3; and wherein CDR1H comprises SEQ ID NO:124, CDR2H comprises SEQ ID NO:125, CDR3H comprises SEQ ID NO:126, CDR1L comprises SEQ ID NO:127, CDR2L comprises sequence LVS, and CDR3L comprises SEQ ID NO:128, and
(b) optionally, substantially isolating the anti-LprG antibody, or LprG-binding fragment thereof.

The antibodies, or antigen-binding fragments thereof, disclosed herein are typically produced by recombinant expression. Nucleic acids encoding light and heavy chain variable regions, optionally linked to constant regions, may be inserted into the same expression vectors. Alternatively, the nucleic acids encoding light and heavy chain variable regions, optionally linked to constant regions, are inserted into different expression vectors. The expression vector may further comprise one or more expression control sequences, which include, but are not limited to, promoters (e.g., homologous or heterologous promoters), signal sequences, enhancer elements, and transcription termination sequences. Preferably, the expression control sequences are eukaryotic promoter systems in vectors capable of transforming or transfecting eukaryotic host cells. Typically, the host is maintained under conditions suitable for high-level expression of the nucleotide sequences, and the collection and purification of the cross-reacting antibodies after the vector is incorporated into the appropriate host.

Commonly, expression vectors contain selection markers (e.g., ampicillin-resistance, hygromycin-resistance, tetracycline resistance or neomycin resistance) to permit detection of those cells transformed with the desired DNA sequences.

The host used to express the antibodies, or antigen-binding fragments thereof, disclosed herein can be a prokaryotic or eukaryotic host. Examples of suitable hosts include bacterial or eukaryotic hosts, including yeast, insects, fungi, bird and mammalian cells either in vivo, or in situ, or host cells of mammalian, insect, bird, or yeast origin. The mammalian cell or tissue can be of human, primate, hamster, rabbit, rodent, cow, pig, sheep, horse, goat, dog, or cat origin, but any other mammalian cell may be used.

Examples of bacterial hosts that can be used to express the antibodies, antigen-binding fragments disclosed herein can be E. coli, bacilli, such as Bacillus subtilus, and other enterobacteriaceae, such as Salmonella, Serratia, and various Pseudomonas species.

Yeasts may also be used as hosts for expressing the express the antibodies, antigen-binding fragments or the fusion protein disclosed herein. Saccharomyces and Pichia are exemplary yeast hosts, with suitable vectors having expression control sequences (e.g., promoters), an origin of replication, termination sequences, and the like as desired. Typical promoters include 3-phosphoglycerate kinase and other glycolytic enzymes. Inducible yeast promoters include, among others, promoters from alcohol dehydrogenase, isocytochrome C, and enzymes responsible for methanol, maltose, and galactose utilization.

Mammalian cells in culture may also be used as host cells for expressing the antibodies, antigen-binding fragments or the fusion proteins disclosed herein. Examples of suitable host cell lines capable of secreting heterologous proteins (e.g., intact immunoglobulins) which are well known in the art, include CHO cell lines, various COS cell lines, HeLa cells, 293 cells, myeloma cell lines, transformed B-cells, and hybridomas. Expression vectors for these cells can include expression control sequences, such as an origin of replication, a promoter, an enhancer and necessary processing information sites such as ribosome binding site, RNA splice site and/or transcriptional terminator sequences. Examples of expression control sequences include SV40, adenovirus, bovine papilloma virus, cytomegalovirus and the like.

The antibodies, or antigen-binding fragments thereof, disclosed herein can be expressed using a single expression construct or vector or multiple expression constructs or vectors (e.g., two or three expression constructs). When the antibody heavy and light chains are cloned on separate expression vectors, the vectors are co-transfected to obtain expression and assembly of intact immunoglobulins. Once expressed, the whole antibodies, their dimers, individual light and heavy chains, or other immunoglobulin forms disclosed herein can be purified according to standard procedures of the art, including ammonium sulfate precipitation, affinity columns, column chromatography, HPLC purification, gel electrophoresis, and the like (see generally Scopes, Protein Purification (Springer-Verlag, N.Y., (1982)). Substantially pure immunoglobulins of at least about 90 to 95% homogeneity are preferred, and 98 to 99% or more homogeneity most preferred, for pharmaceutical uses.

The antibodies, or antigen-binding fragments thereof, disclosed herein can be made by any method known in the art.

### Pharmaceutical Compositions

In an embodiment, a pharmaceutical composition is provided comprising an anti-Mtb AM antibody, or Mtb AM-binding fragment thereof as described herein. In an embodiment, a pharmaceutical composition is provided comprising an anti-GroEL2 antibody, or GroEL2-binding fragment thereof as described herein. In an embodiment, a pharmaceutical composition is provided comprising an anti-LprG antibody, or LprG-binding fragment thereof as described herein.

In an embodiment, a pharmaceutical composition comprising an antibody, or antigen-binding fragment thereof, described herein, and a pharmaceutically acceptable excipient, is provided. The pharmaceutically acceptable excipient can be a pharmaceutically-acceptable material, composition or vehicle, such as a liquid or solid filler, diluent, manufacturing aid (e.g., lubricant, talc magnesium, calcium or zinc stearate, or steric acid), solvent or encapsulating material, involved in carrying or transporting the therapeutic compound for administration to the subject, bulking agent, salt, surfactant and/or a preservative. Some examples of materials which can serve as pharmaceutically-acceptable excipients include: sugars, such as lactose, glucose and sucrose; starches, such as corn starch and potato starch; cellulose and its derivatives, such as sodium carboxymethyl cellulose, ethyl cellulose and cellulose acetate; gelatin; talc; waxes; oils, such as peanut oil, cottonseed oil, safflower oil, sesame oil, olive oil, corn oil and soybean oil; glycols, such as ethylene glycol and propylene glycol; polyols, such as glycerin, sorbitol, mannitol and polyethylene glycol; esters, such as ethyl oleate and ethyl laurate; agar; buffering agents; water; isotonic saline; pH buffered solutions; and other non-toxic compatible substances employed in pharmaceutical formulations.

Compositions or pharmaceutical compositions comprising the antibody, or antigen-binding fragment thereof, disclosed herein comprise stabilizers to prevent loss of activity or structural integrity of the protein due to the effects of denaturation, oxidation, or aggregation over a period of time during storage and transportation prior to use. The compositions or pharmaceutical compositions can comprise one or more of any combination of salts, surfactants, pH and tonicity agents such as sugars that contribute to overcoming aggregation problems. Where a composition or pharmaceutical composition disclosed herein is used as an injection, the composition may have a pH value in an approximately neutral pH range. In some embodiments, surfactant levels are minimized to avoid bubbles in the formulation which are detrimental for injection into subjects. In an embodiment, the composition or pharmaceutical composition is in liquid form and stably supports high concentrations of bioactive antibody in solution and is suitable for inhalational or parenteral administration. In an embodiment, the composition or pharmaceutical composition is suitable for intravenous, intramuscular, intraperitoneal, intradermal and/or subcutaneous injection. In an embodiment, the composition or pharmaceutical composition is in liquid form and has minimized risk of bubble formation and anaphylactoid side effects. In an embodiment, the composition or pharmaceutical composition is isotonic. In an embodiment, the composition or pharmaceutical composition has a pH or 6.8 to 7.4.

In one embodiment, the antibody, or antigen-binding fragment thereof, disclosed herein is lyophilized and/or freeze dried and is reconstituted for use.

The antibodies, or fragments of antibodies, or compositions, or pharmaceutical compositions described herein can also be lyophilized or provided in any suitable forms including, but not limited to, injectable solutions or inhalable solutions, gel forms, and tablet forms.

### Methods of Using the Antibodies Described Herein

### Methods of Using Anti-Mtb AM Antibodies, and Mtb AM-Binding Fragments Thereof

Provided herein is a method of reducing the activity of Mtb AM in a subject in need thereof, the method comprising administering to said subject a therapeutically effective amount of (1) an anti-Mtb AM antibody, or Mtb AM-binding fragment thereof, disclosed herein or (2) a pharmaceutical composition comprising an anti-Mtb AM antibody, or Mtb AM-binding fragment thereof, disclosed herein. Provided herein is (1) an anti-Mtb AM antibody, or Mtb AM-binding fragment thereof, disclosed herein or (2) a pharmaceutical composition comprising an anti-Mtb AM antibody, or Mtb AM-binding fragment thereof, for use in reducing the activity of Mtb AM in a subject in need thereof. Provided herein is the use of an anti-Mtb AM antibody, or Mtb AM-binding fragment thereof, disclosed herein in the manufacture of a medicament for reducing the activity of Mtb AM in a subject in need thereof.

Provided herein is a method of treating a Mycobacterium tuberculosis infection in a subject in need thereof, the method comprising administering to said subject a therapeutically effective amount of (1) an anti-Mtb AM antibody, or Mtb AM-binding fragment thereof, disclosed herein or (2) a pharmaceutical composition comprising an anti-Mtb AM antibody, or Mtb AM-binding fragment thereof, disclosed herein. Provided herein is (1) an anti-Mtb AM antibody, or Mtb AM-binding fragment thereof, disclosed herein or (2) a pharmaceutical composition comprising an anti-Mtb AM antibody, or Mtb AM-binding fragment thereof, for use in treating a Mycobacterium tuberculosis infection in a subject in need thereof. Provided herein is the use of an anti-Mtb AM antibody, or Mtb AM-binding fragment thereof, disclosed herein in the manufacture of a medicament for treating a Mycobacterium tuberculosis infection in a subject in need thereof.

In an embodiment, the disclosure provides a method of reducing the likelihood of a Mycobacterium tuberculosis infection in a subject in need thereof who does not have a Mycobacterium tuberculosis infection, the method comprising administering to the subject a prophylactically effective amount of (1) an anti-Mtb AM antibody, or Mtb AM-binding fragment thereof, disclosed herein or (2) a pharmaceutical composition comprising an anti-Mtb AM antibody, or Mtb AM-binding fragment thereof, disclosed herein. Provided herein is (1) an anti-Mtb AM antibody, or Mtb AM-binding fragment thereof, disclosed herein or (2) a pharmaceutical composition comprising an anti-Mtb AM antibody, or Mtb AM-binding fragment thereof, for use in reducing the likelihood of a Mycobacterium tuberculosis infection in a subject in need thereof. Provided herein is the use of an anti-Mtb AM antibody, or Mtb AM-binding fragment thereof, disclosed herein in the manufacture of a medicament for reducing the likelihood of a Mycobacterium tuberculosis infection in a subject in need thereof.

In an embodiment, the disclosure provides a method of treating a disease, disorder, or condition mediated by, or related to increased activity of Mycobacterium tuberculosis in a subject in need thereof, the method comprising administering to said subject a therapeutically effective amount of (1) an anti-Mtb AM antibody, or Mtb AM-binding fragment thereof, disclosed herein or (2) a pharmaceutical composition comprising an anti-Mtb AM antibody, or Mtb AM-binding fragment thereof, disclosed herein. Provided herein is (1) an anti-Mtb AM antibody, or Mtb AM-binding fragment thereof, disclosed herein or (2) a pharmaceutical composition comprising an anti-Mtb AM antibody, or Mtb AM-binding fragment thereof, for use in treating a disease, disorder, or condition mediated by, or related to increased activity of Mycobacterium tuberculosis in a subject in need thereof. Provided herein is the use of an anti-Mtb AM antibody, or Mtb AM-binding fragment thereof, disclosed herein in the manufacture of a medicament for treating a disease, disorder, or condition mediated by, or related to increased activity of Mycobacterium tuberculosis in a subject in need thereof.

### Methods of Using Anti-GroEL2 Antibodies, and GroEL2-Binding Fragments Thereof

Provided herein is a method of reducing the activity of GroEL2 in a subject in need thereof, the method comprising administering to said subject a therapeutically effective amount of (1) an anti-GroEL2 antibody, or GroEL2-binding fragment thereof, disclosed herein or (2) a pharmaceutical composition comprising an anti-GroEL2 antibody, or GroEL2-binding fragment thereof, disclosed herein. Provided herein is (1) an anti-GroEL2 antibody, or GroEL2-binding fragment thereof, disclosed herein or (2) a pharmaceutical composition comprising an anti-GroEL2 antibody, or GroEL2-binding fragment thereof, for use in reducing the activity of GroEL2 in a subject in need thereof. Provided herein is the use of an anti-GroEL2 antibody, or GroEL2-binding fragment thereof, disclosed herein in the manufacture of a medicament for reducing the activity of GroEL2 in a subject in need thereof.

Provided herein is a method of treating a Mycobacterium tuberculosis infection in a subject in need thereof, the method comprising administering to said subject a therapeutically effective amount of (1) an anti-GroEL2 antibody, or GroEL2-binding fragment thereof, disclosed herein or (2) a pharmaceutical composition comprising an anti-GroEL2 antibody, or GroEL2-binding fragment thereof, disclosed herein. Provided herein is (1) an anti-GroEL2 antibody, or GroEL2-binding fragment thereof, disclosed herein or (2) a pharmaceutical composition comprising an anti-GroEL2 antibody, or GroEL2-binding fragment thereof, for use in treating a Mycobacterium tuberculosis infection in a subject in need thereof. Provided herein is the use of an anti-GroEL2 antibody, or GroEL2-binding fragment thereof, disclosed herein in the manufacture of a medicament for treating a Mycobacterium tuberculosis infection in a subject in need thereof.

In an embodiment, the disclosure provides a method of reducing the likelihood of a Mycobacterium tuberculosis infection in a subject in need thereof who does not have a Mycobacterium tuberculosis infection, the method comprising administering to the subject a prophylactically effective amount of (1) an anti-GroEL2 antibody, or GroEL2-binding fragment thereof, disclosed herein or (2) a pharmaceutical composition comprising an anti-GroEL2 antibody, or GroEL2-binding fragment thereof, disclosed herein. Provided herein is (1) an anti-GroEL2 antibody, or GroEL2-binding fragment thereof, disclosed herein or (2) a pharmaceutical composition comprising an anti-GroEL2 antibody, or GroEL2-binding fragment thereof, for use in reducing the likelihood of a Mycobacterium tuberculosis infection in a subject in need thereof. Provided herein is the use of an anti-GroEL2 antibody, or GroEL2-binding fragment thereof, disclosed herein in the manufacture of a medicament for reducing the likelihood of a Mycobacterium tuberculosis infection in a subject in need thereof.

In an embodiment, the disclosure provides a method of treating a disease, disorder, or condition mediated by, or related to increased activity of Mycobacterium tuberculosis in a subject in need thereof, the method comprising administering to said subject a therapeutically effective amount of (1) an anti-GroEL2 antibody, or GroEL2-binding fragment thereof, disclosed herein or (2) a pharmaceutical composition comprising an anti-GroEL2 antibody, or GroEL2-binding fragment thereof, disclosed herein. Provided herein is (1) an anti-GroEL2 antibody, or GroEL2-binding fragment thereof, disclosed herein or (2) a pharmaceutical composition comprising an anti-GroEL2 antibody, or GroEL2-binding fragment thereof, for use in treating a disease, disorder, or condition mediated by, or related to increased activity of Mycobacterium tuberculosis in a subject in need thereof. Provided herein is the use of an anti-GroEL2 antibody, or GroEL2-binding fragment thereof, disclosed herein in the manufacture of a medicament for treating a disease, disorder, or condition mediated by, or related to increased activity of Mycobacterium tuberculosis in a subject in need thereof.

### Methods of Using Anti-LprG Antibodies, and LprG-Binding Fragments Thereof

Provided herein is a method of reducing the activity of LprG in a subject in need thereof, the method comprising administering to said subject a therapeutically effective amount of (1) an anti-LprG antibody, or LprG-binding fragment thereof, disclosed herein or (2) a pharmaceutical composition comprising an anti-LprG antibody, or LprG-binding fragment thereof, disclosed herein. Provided herein is (1) an anti-LprG antibody, or LprG-binding fragment thereof, disclosed herein or (2) a pharmaceutical composition comprising an anti-LprG antibody, or LprG-binding fragment thereof, for use in reducing the activity of LprG in a subject in need thereof. Provided herein is the use of an anti-LprG antibody, or LprG-binding fragment thereof, disclosed herein in the manufacture of a medicament for reducing the activity of LprG in a subject in need thereof.

Provided herein is a method of treating a Mycobacterium tuberculosis infection in a subject in need thereof, the method comprising administering to said subject a therapeutically effective amount of (1) an anti-LprG antibody, or LprG-binding fragment thereof, disclosed herein or (2) a pharmaceutical composition comprising an anti-LprG antibody, or LprG-binding fragment thereof, disclosed herein. Provided herein is (1) an anti-LprG antibody, or LprG-binding fragment thereof, disclosed herein or (2) a pharmaceutical composition comprising an anti-LprG antibody, or LprG-binding fragment thereof, for use in treating a Mycobacterium tuberculosis infection in a subject in need thereof. Provided herein is the use of an anti-LprG antibody, or LprG-binding fragment thereof, disclosed herein in the manufacture of a medicament for treating a Mycobacterium tuberculosis infection in a subject in need thereof.

In an embodiment, the disclosure provides a method of reducing the likelihood of a Mycobacterium tuberculosis infection in a subject in need thereof who does not have a Mycobacterium tuberculosis infection, the method comprising administering to the subject a prophylactically effective amount of (1) an anti-LprG antibody, or LprG-binding fragment thereof, disclosed herein or (2) a pharmaceutical composition comprising an anti-LprG antibody, or LprG-binding fragment thereof, disclosed herein. Provided herein is (1) an anti-LprG antibody, or LprG-binding fragment thereof, disclosed herein or (2) a pharmaceutical composition comprising an anti-LprG antibody, or LprG-binding fragment thereof, for use in reducing the likelihood of a Mycobacterium tuberculosis infection in a subject in need thereof. Provided herein is the use of an anti-LprG antibody, or LprG-binding fragment thereof, disclosed herein in the manufacture of a medicament for reducing the likelihood of a Mycobacterium tuberculosis infection in a subject in need thereof.

In an embodiment, the disclosure provides a method of treating a disease, disorder, or condition mediated by, or related to increased activity of Mycobacterium tuberculosis in a subject in need thereof, the method comprising administering to said subject a therapeutically effective amount of (1) an anti-LprG antibody, or LprG-binding fragment thereof, disclosed herein or (2) a pharmaceutical composition comprising an anti-LprG antibody, or LprG-binding fragment thereof, disclosed herein. Provided herein is (1) an anti-LprG antibody, or LprG-binding fragment thereof, disclosed herein or (2) a pharmaceutical composition comprising an anti-LprG antibody, or LprG-binding fragment thereof, for use in treating a disease, disorder, or condition mediated by, or related to increased activity of Mycobacterium tuberculosis in a subject in need thereof. Provided herein is the use of an anti-LprG antibody, or LprG-binding fragment thereof, disclosed herein in the manufacture of a medicament for treating a disease, disorder, or condition mediated by, or related to increased activity of Mycobacterium tuberculosis in a subject in need thereof.

In embodiments, the subject is an animal. In embodiments, the subject is a mammal. In some embodiment, the subject is a non-human mammal. In embodiments, the subject is a non-primate (e.g., cows, buffalos, pigs, horses, cats, dogs, rats, mouse, guinea pigs, sheep, goats etc.) or a primate (e.g., monkey and human). In one embodiment, the subject is human. Individuals and patients are also subjects herein.

The antibodies, or antigen-binding fragments thereof, disclosed herein can be administered with to one or more additional therapeutic agents. Suitable additional therapeutic agents in this regard include, but are not limited to, drugs, toxins, and derivatives thereof. Non-limiting examples of additional therapeutic agents include isoniazid, rifampin, ethambutol, pyrazinamide, bedaquiline, linezolid, and pretomanid.

### Assay Devices and Methods of Detection and Diagnosis

Provided herein are devices and methods for the detection of Mtb AM, AM fragments, LAM, LAM fragments, GroEL, LprG, and/or Mtb bacteria. In embodiments, the devices provided herein comprise one or more of the antibodies or antigen-binding fragments disclosed herein. Also provided are devices and methods for diagnosing TB in a subject by detectingMtb AM, AM fragments, LAM, LAM fragments, GroEL, LprG, and/or Mtb bacteria in a sample from the subject. Detection of Mtb AM, AM fragments, LAM, LAM fragments, GroEL, LprG, and/or Mtb bacteria in a sample from the subject can assist medical professional to triage subjects for further TB assessment and/or treatment. In embodiments, the subject is an animal. In embodiments, the subject is a mammal. In embodiments, the subject is a non-primate (e.g., cows, pigs, horses, cats, dogs, rats, mouse, etc.) or a primate (e.g., monkey and human). In one embodiment, the subject is a human. Also provided herein are devices and methods for the detection of NTM AM, AM fragments, LAM, LAM fragments, and/or NTM bacteria. In embodiments, the devices provided herein comprise one or more of the antibodies disclosed herein. Also provided are devices and methods for diagnosing infection with NTM in a subject by detecting NTM AM, AM fragments, LAM, LAM fragments, and/or NTM bacteria in a sample from the subject.

In embodiments, the devices and methods for the detection of Mtb AM, AM fragments, LAM, LAM fragments, GroEL, LprG, and/orMtb bacteria employ antibodies that exhibit high affinity, low background in an assay, high sensitivity, high stability in a device, and/or that react with clinically relevant LAM motifs.

In embodiments, the antigen to be detected is immobilized directly onto a surface of a multi-well microtiter and is then contacted with an antibody or antigen-binding fragment thereof, disclosed herein, wherein the antibody or antigen-binding fragment thereof is equipped with a detection moiety.

In embodiments, the antigen to be detected is immobilized directly onto a surface of a multi-well microtiter and is then contacted with an antibody or antigen-binding fragment thereof, disclosed herein. The antibody or antigen-binding fragment thereof, is detected by a secondary antibody that binds to the antibody or antigen-binding fragment thereof and that is equipped with a detection moiety.

In embodiments, the antibodies or antigen-binding fragments herein are used in a sandwich ELISA or variation thereof. In a sandwich ELISA, the analyte to be measured is bound between two primary antibodies, each detecting a different epitope of the antigen. The first primary antibody, the capture antibody, is often immobilized on a surface. The second primary antibody is often equipped with a detection moiety. However, the second primary antibody may also be unlabeled, and may in turn be detected by a secondary antibody that binds to the second primary antibody and that is equipped with a detection moiety. In embodiments, the antibodies or antigen-binding fragments herein are used as a first primary antibody. In embodiments, the antibodies or antigen-binding fragments herein are used as a second primary antibody.

### Assay Devices and Methods of Detection and Diagnosis Using Anti-Mtb AM Antibodies, or Mtb AM-Binding Fragments Thereof

Provided herein are devices and methods for the detection of Mtb AM, AM fragments, LAM, LAM fragments, and/or Mtb bacteria by using one or more anti-Mtb AM antibodies, or Mtb AM-binding fragments thereof, as described herein.

In embodiments, a pair of antibodies is used for the detection of Mtb AM, AM fragments, LAM, LAM fragments, and/or Mtb bacteria, wherein one of the two antibodies or both antibodies are antibodies disclosed herein. In one embodiments, a pair of antibodies A1AM23 and P1Am25, or antigen-binding fragments thereof, is used.

Provided herein is a method of detecting Mtb AM, an AM fragment, LAM (which comprises AM), and/or an AM-comprising fragment of LAM, the method comprising contacting a biological sample with an anti-Mtb AM antibody, or Mtb AM-binding fragment thereof, as described herein and detecting binding of the anti-Mtb AM antibody, or Mtb AM-binding fragment thereof. Binding of the anti-Mtb AM antibody, or Mtb AM-binding fragment thereof, to its antigen can be detected in numerous ways, including, but not limited to (1) attaching a reporting moiety to the anti-Mtb AM antibody, or Mtb AM-binding fragment thereof, (2) contacting the Mtb AM, AM fragment, LAM (which comprises AM), and/or AM-comprising fragment of LAM bound by a first anti-Mtb AM antibody, or Mtb AM-binding fragment thereof, with a second anti-Mtb AM antibody, or Mtb AM-binding fragments thereof (which in turn may be attached to a reporting moiety), or (3) contacting the second anti-Mtb AM antibody, or Mtb AM-binding fragment thereof, with a third antibody (which in turn may be attached to a reporting moiety) that recognizes an antigen located on the second Mtb AM antibody, or Mtb AM-binding fragment thereof, such as the constant region of the second anti-Mtb AM antibody, or Mtb AM-binding fragment thereof.

In an embodiment, an assay device is provided for selectively detecting MTC AM, an AM fragment, LAM (which comprises AM), and/or an AM-comprising fragment of LAM and/or one or more bacteria from the MTC group in a biological sample comprising: a first portion comprising a first plurality of anti-Mtb AM antibodies, or Mtb AM-binding fragments thereof, as described herein, or anti-mycobacterial AM-antibodies, wherein the antibodies or fragments are each attached to their own reporting entity; and a second portion comprising a second plurality of anti-Mtb AM antibodies, or Mtb AM-binding fragments thereof, as described herein, or anti-mycobacterial AM-antibodies. As used herein, an anti-mycobacterial AM-antibody binds to AM, an AM fragment, LAM (which comprises AM), and/or to an AM-comprising fragment of LAM.

In an embodiment, an assay device is provided for selectively detecting Mtb AM, an AM fragment, LAM and/or an AM-comprising fragment of LAM in a biological sample comprising: a first portion comprising a first plurality of anti-Mtb AM antibodies, or Mtb AM-binding fragments thereof, as described herein, or anti-mycobacterial AM-antibodies, wherein the antibodies or fragments are each attached to their own reporting entity; and a second portion comprising a second plurality of anti-Mtb AM antibodies, or Mtb AM-binding fragments thereof, as described herein, or anti-mycobacterial AM-antibodies.

In an embodiment, an assay device is provided for selectively detecting Mtb AM, an AM fragment, LAM and/or an AM-comprising fragment of LAM from MTC in a biological sample comprising: a first portion comprising a first plurality of anti-Mtb AM antibodies, or Mtb AM-binding fragments thereof, as described herein, or anti-mycobacterial AM-antibodies, wherein the antibodies or fragments are each attached to their own reporting entity; and a second portion comprising a second plurality of anti-Mtb AM antibodies, or Mtb AM-binding fragments thereof, as described herein, or anti-mycobacterial AM-antibodies.

MTC is a genetically related group of Mycobacterium species that can cause tuberculosis in humans or other animals and includes Mycobacterium tuberculosis, M africanum, M. canettii, M bovis, M. microti, M. orygis, M. caprae, M pinnipedii, M. suricattae, and M. mungi.

In an embodiment, the reporting entity comprises an enzyme. In an embodiment, the enzyme is horseradish peroxidase (HRP) or alkaline phosphatase (AP).

In one embodiment, the reporting entity comprises a gold nanoparticle.

In embodiments, the second plurality of anti-Mtb AM-antibodies, or Mtb AM-binding fragments thereof, or anti-mycobacterial AM-antibodies, is affixed to a solid support of the device.

In embodiments, the first plurality of anti-Mtb AM-antibodies, or Mtb AM-binding fragments thereof, or anti-mycobacterial AM-antibodies, is not affixed to a solid support of the device.

In embodiments, the solid support comprises nitrocellulose.

In embodiments, the device further comprises a fluid sample pad prior in sequential order to the first and second portions.

In embodiments, the device further comprises a control portion subsequent in sequential order to the first and second portions.

In embodiments, the control portion comprises a third plurality of antibodies, wherein the third plurality of antibodies are capable of binding the first plurality of anti-Mtb AM-antibodies, or Mtb AM-binding fragments thereof, or anti-mycobacterial AM-antibodies each attached to their own reporting molecule. The immobilized third plurality of antibodies may be immobilized on a solid support of the device. In embodiments, the third plurality of antibodies comprises an antibody disclosed herein.

In embodiments, the device further comprises a fluid-absorbent wicking pad subsequent in sequential order to the first and second portions, and third portion if present.

In embodiments, the device is a lateral flow device.

Provided is a lateral flow assay device for detecting a Mycobacterium tuberculosis in a biological sample comprising:
(a) a first portion comprising a first plurality of anti-Mtb AM-antibodies, or Mtb AM-binding fragments thereof, as disclosed herein, wherein the antibodies or fragments are each attached to their own reporting entity; and
(b) a second plurality of anti-Mtb AM-antibodies, or Mtb AM-binding fragments thereof, or anti-mycobacterial AM-antibodies.

Provided is a lateral flow assay device for detecting Mtb AM, an AM fragment, LAM and/or an AM-comprising fragment of LAM in a biological sample comprising:
(a) a first portion comprising a first plurality of anti-Mtb AM-antibodies, or Mtb AM-binding fragments thereof, as disclosed herein, wherein the antibodies or fragments are each attached to their own reporting entity; and
(b) a second plurality of anti-Mtb AM-antibodies, or Mtb AM-binding fragments thereof, or anti-mycobacterial AM-antibodies.

In some embodiments, the first or second plurality of anti-Mtb AM-antibodies, or Mtb AM-binding fragments thereof, or anti-mycobacterial AM-antibodies comprises an antibody comprising the CDR sequences of an antibody in Table 2. In some embodiments, the second plurality of anti-Mtb AM-antibodies, or Mtb AM-binding fragments thereof, or anti-mycobacterial AM-antibodies comprises an antibody comprising a heavy and/or light variable chain sequence that is at least 80%, at least 85%, at least 90%, at least 95%, at least 96%, at least 97%, at least 98%, or at least 99% identical to a sequence in in Table 3. In some embodiments, the second plurality of anti-Mtb AM-antibodies, or Mtb AM-binding fragments thereof, or anti-mycobacterial AM-antibodies comprises an antibody comprising a sequence in Table 3. In some embodiments, the second plurality of anti-Mtb AM-antibodies, or Mtb AM-binding fragments thereof, or anti-mycobacterial AM-antibodies comprises antibody L1AMO4, T1AM09, or P1AM25 or a variant of antibody L1AMO4, T1AM09, or P1AM25. Antibody T1AM09 is disclosed in U.S. application Ser. No. 17/247,532, which is herein incorporated by reference in its entirety. L1AMO4 and P1AM25 are disclosed in PCT Application No. PCT/US2021/063461, which is herein incorporated by reference in its entirety.

TABLE 2
CDR sequences of anti-AM antibodies L1AM04, T1AM09, and P1AM25.
CDR nomenclature based on Kabat.

| Antibody | CDR | SEQ ID NO: | Amino acid sequence |
|---|---|---|---|
| L1AM04 | CDRH1 | 55 | SYAMS |
| L1AM04 | CDRH2 | 137 | TLSDSGGATHYADSVQG |
| L1AM04 | CDRH3 | 138 | DVPPKTAGPMFDY |
| L1AM04 | CRDL1 | 139 | RSSESLVHSNGNTYLS |
| L1AM04 | CDRL2 | 140 | EISKRYSG |
| L1AM04 | CDRL3 | 141 | AQVSHFPRT |
| T1AM09 | CDRH1 | 142 | TYWIH |
| T1AM09 | CDRH2 | 143 | WIIPKSGGTNYAQKFQG |
| T1AM09 | CDRH3 | 144 | GILLNGIGAFDY |
| T1AM09 | CRDL1 | 145 | RTSQTVSSNLN |
| T1AM09 | CDRL2 | 146 | GISDLHS |
| T1AM09 | CDRL3 | 147 | QQSYSLPRT |
| P1AM25 | CDRH1 | 148 | SYYLS |
| P1AM25 | CDRH2 | 149 | YMFHRGSTNRNPSLRS |
| P1AM25 | CDRH3 | 150 | HLGIVGVTNAAFDI |
| P1AM25 | CRDL1 | 151 | SGSSSNIGNNYVS |
| P1AM25 | CDRL2 | 152 | QNDKRPS |
| P1AM25 | CDRL3 | 153 | GTWDRSLNADV |

TABLE 3
Heavy and light variable chains sequences of
anti-AM antibodies L1AM04, T1AM09, and P1AM25.

| | | SEQ | |
|---|---|---|---|
| Anti- | Se- | ID | |
| body | quence | NO: | Sequence |
| L1AM04 | VH | 154 | EVQLLESGGGLVQPGGSQRLFCEASGFT |
| | | | FASYAMSWVRLAPGKGLEWVSTLSDSGG |
| | | | ATHYADSVQGRFTISRDNSNNVVYLQMN |
| | | | SLRAEDTAVYYCARDVPPKTAGPMFDYW |
| | | | GLGTLVTVSS |
| L1AM04 | VL | 155 | DIVMTQTPLSSPVSLGQPASISCRSSES |
| | | | LVHSNGNTYLSWLHQRPGQPPRLLIYEI |
| | | | SKRYSGVSDRFSGSGAGTDFTLKISRVE |
| | | | AEDVGIYFCAQVSHFPRTFGQGTKVEIK |
| T1AM09 | VH-1 | 156 | QVQLVESGAEVKKPGASVKVSCKASGYT |
| | | | FSTYWIHWMRQAPGQGPEWMGWIIPKSG |
| | | | GTNYAQKFQGRVAMTRDTSLNTVYMELS |
| | | | RLTSDDTAVYYCARGILLNGIGAFDYWG |
| | | | QGTLVTVSS |
| T1AM09 | VH-2 | 157 | QVQLVESGAEVKKPGASVKVSCKASGYT |
| | | | FATYWIHWMRQAPGQGPEWMGWIIPKSG |
| | | | GTNYAQKFQGRVAMTRDTSLNTVYMELS |
| | | | RLTSDDTAVYYCARGILLNGIGAFDYWG |
| | | | QGTLVTVSS |
| T1AM09 | VL | 158 | DIVMTQSPSSLSASVGDRVTITCRTSQT |
| | | | VSSNLNWYQQRPGKAPKLLISGISDLHS |
| | | | GVPSRFSGSGSGTDFTLTISSLQPEDSA |
| | | | TYYCQQSYSLPRTFGQGTKVEIK |
| P1AM25 | VH | 159 | QVQLQESGPGLVEPSETLSLTCTVSGGS |
| | | | IRSYYLSWIRQSPKKGLEWIGYMFHRGS |
| | | | TNRNPSLRSRVTMSLDTSKNQFSLKLTS |
| | | | VTAADTAVYYCATHLGIVGVTNAAFDIW |
| | | | GQGTMVTVSS |
| P1AM25 | VL | 160 | QSVLTQPPSVSAAPGQKVTISCSGSSSN |
| | | | IGNNYVSWYQLLPGTAPKLLIYQNDKRP |
| | | | SDIPDRFSGSKSGTSATLGITGLQTGDE |
| | | | ADYYCGTWDRSLNADVFGGGTKLTVL |

In some embodiments, the assay device comprises one or more pluralities of anti-Mtb AM antibodies, or Mtb AM-binding fragments thereof, or anti-mycobacterial AM-antibodies, wherein at least one of the pluralities of anti-Mtb AM antibodies, or Mtb AM-binding fragments thereof, or anti-mycobacterial AM-antibodies, comprises a non-human constant region or a modified non-human constant region. In some embodiments, the assay device comprises one or more pluralities of anti-Mtb AM antibodies, or Mtb AM-binding fragments thereof, or anti-mycobacterial AM-antibodies, wherein at least one of the pluralities of anti-Mtb AM antibodies, or Mtb AM-binding fragments thereof, or anti-mycobacterial AM-antibodies, comprises a murine constant region or a modified murine constant region.

In an embodiment, the reporting entity comprises an enzyme. In an embodiment, the enzyme is horseradish peroxidase (HRP) or alkaline phosphatase (AP). In an embodiment, the reporting entity comprises a gold nanoparticle.

In an embodiment, the second plurality of anti-Mtb AM antibodies, or Mtb AM-binding fragments thereof, or anti-mycobacterial AM-antibodies is affixed to a solid support of the device.

In an embodiment, the first plurality of anti-Mtb AM antibodies, or Mtb AM-binding fragments thereof, or anti-mycobacterial AM-antibodies is not affixed to a solid support of the device.

In an embodiment, the solid support comprises nitrocellulose.

In an embodiment, the device further comprises a fluid sample pad prior in sequential order to the first and second portions.

In an embodiment, the device further comprises a control portion subsequent in sequential order to the first and second portions.

In an embodiment, the control portion comprises a third plurality of antibodies, immobilized on a solid support of the device, and which third plurality of antibodies are capable of binding the first plurality of anti-Mtb AM antibodies, or Mtb AM-binding fragments thereof, or anti-mycobacterial AM-antibodies, each attached to their own reporting molecule.

In an embodiment, the device further comprises a fluid-absorbent wicking pad subsequent in sequential order to the first and second portions, and third portion if present.

In embodiments, the second plurality of anti-Mtb AM antibodies, or Mtb AM-binding fragments thereof, comprises anti-Mtb AM antibodies, or Mtb AM-binding fragments thereof, described herein.

In an embodiment, a method is provided for detecting one or more bacteria from the MTC group in a biological sample comprising:
(a) contacting a device described herein with the biological sample; and
(b) observing if one or more bacteria from the MTC group bind to the second plurality of anti-Mtb AM antibodies, or Mtb AM-binding fragments thereof, or anti-mycobacterial AM-antibodies, wherein if such anti-Mtb AM antibodies, or Mtb AM-binding fragments thereof, or anti-mycobacterial AM-antibodies, bind, then one or more bacteria from the MTC group have been detected in the biological sample; and if no anti-Mtb AM antibodies, or Mtb AM-binding fragments thereof, or anti-mycobacterial AM-antibodies, bind to the second plurality of anti-Mtb AM antibodies, or Mtb AM-binding fragments thereof, or anti-mycobacterial AM-antibodies, then bacteria from the MTC group have not been detected in the biological sample.

In an embodiment, a method is provided for selectively detecting Mtb AM, an AM fragment, LAM and/or an AM-comprising fragment of LAM in a biological sample comprising:
(a) contacting a device described herein with the sample; and
(b) observing if the Mtb AM, AM fragment, LAM and/or AM-comprising fragment of LAM bind to the second plurality of anti-Mtb AM antibodies, or Mtb AM-binding fragments thereof, or anti-mycobacterial AM-antibodies, wherein if such anti-Mtb AM antibodies, or Mtb AM-binding fragments thereof, or anti-mycobacterial AM-antibodies, bind, then Mtb AM, an AM fragment, LAM and/or an AM-comprising fragment of LAM has been detected in the biological sample; and if no anti-Mtb AM antibodies, or Mtb AM-binding fragments thereof, or anti-mycobacterial AM-antibodies, bind to the second plurality of anti-Mtb AM antibodies, or Mtb AM-binding fragments thereof, or anti-mycobacterial AM-antibodies, then Mtb AM, an AM fragment, LAM and/or an AM-comprising fragment of LAM have not been detected in the biological sample.

Provided herein are anti-Mtb AM antibodies that react with (1) an AM fragment, LAM and/or an AM-comprising fragment of LAM from mycobacterial strains from the MTC group and/or MTC bacteria and (2) an AM fragment, LAM and/or an AM-comprising fragment of LAM from non-tuberculous mycobacteria (NTM) and/or NTM bacteria. In embodiments, provided herein are antibodies and antigen-binding fragments thereof that (1) bind to an AM fragment, LAM and/or an AM-comprising fragment of LAM from MTC and/or to MTC bacteria and (2) show significantly decreased or no binding to an AM fragment, LAM and/or an AM-comprising fragment of LAM from NTM and/or to NTM bacteria. Accordingly, provided is a method of discriminating between (1) an AM fragment, LAM and/or an AM-comprising fragment of LAM from MTC and/or to MTC bacteria and (2) an AM fragment, LAM and/or an AM-comprising fragment of LAM from NTM and/or to NTM bacteria using anti-Mtb AM antibodies, or Mtb AM-binding fragments thereof, or anti-mycobacterial AM-antibodies, including anti-Mtb AM antibodies, or Mtb AM-binding fragments thereof, disclosed herein. Diagnostic tests based on such methods that are able to discriminate between virulent and non-virulent mycobacterium infections, or between (1) an AM fragment, LAM and/or an AM-comprising fragment of LAM from MTC and/or to MTC bacteria and (2) an AM fragment, LAM and/or an AM-comprising fragment of LAM from NTM and/or to NTM bacteria, represent a major advance that will greatly reduce wasted effort and consequent costs resulting from further diagnostic tests and/or therapeutic procedures.

In one embodiment, provided is a method of detecting and/or distinguishing between (1) an AM fragment, LAM and/or an AM-comprising fragment of LAM from MTC and/or to MTC bacteria and (2) an AM fragment, LAM and/or an AM-comprising fragment of LAM from NTM and/or to NTM bacteria in a biological sample, the method comprising:
(a) providing a biological sample comprising an Mtb antigen;
(b) contacting the sample with a first anti-Mtb AM antibody, or Mtb AM-binding fragment thereof, or anti-mycobacterial AM-antibody, wherein the first anti-Mtb AM antibody, or Mtb AM-binding fragment thereof, or anti-mycobacterial AM-antibody, binds an AM fragment, LAM and/or an AM-comprising fragment of LAM from MTC and/or to MTC bacteria, but does not substantially bind to an AM fragment, LAM and/or an AM-comprising fragment of LAM from NTM and/or to NTM bacteria;
(c) observing if the Mtb antigen in the biological sample binds to the first anti-Mtb AM antibody, or Mtb AM-binding fragment thereof, or anti-mycobacterial AM-antibody;
(d) contacting the sample with a second anti-Mtb antibody, or Mtb-binding fragment thereof, or anti-mycobacterial AM-antibody, wherein the second anti-Mtb AM antibody, or Mtb AM-binding fragment thereof, or anti-mycobacterial AM-antibody binds to AM fragments, LAM and/or an AM-comprising fragments of LAM from MTC or NTM and/or to MTC bacteria and to NTM bacteria;
(e) observing if the Mtb antigen in the biological sample binds to the second anti-Mtb AM antibody, or Mtb AM-binding fragment thereof, or anti-mycobacterial AM-antibody; wherein if the Mtb antigen in the biological sample binds to the first and the second anti-Mtb AM antibodies, or Mtb AM-binding fragments thereof, or anti-mycobacterial AM-antibody, then an AM fragment, LAM and/or an AM-comprising fragment of LAM from MTC and/or to MTC bacteria have been detected in the biological sample; and
wherein if the Mtb antigen in the biological sampl e binds to the second, but not the first anti-Mtb AM antibody, or Mtb AM-binding fragment thereof, or anti-mycobacterial AM-antibody, then an AM fragment, LAM and/or an AM-comprising fragment of LAM from NTM and/or to NTM bacteria have been detected in the biological sample.

Provided herein is an assay device for selectively detecting one or more nontuberculous mycobacteria (NTMs) in a biological sample comprising:
(a) a first portion comprising a first plurality of anti-NTM AM antibodies, or NTM AM-binding fragments thereof, disclosed herein, wherein the anti-NTM AM antibodies, or NTM AM-binding fragments thereof, are attached to a reporting entity; and
(b) a second portion comprising a second plurality of anti-NTM AM antibodies, or NTM AM-binding fragments thereof, disclosed herein.

In some embodiments, the NTM is Mycobacterium avium or Mycobacterium abscessus.

In embodiments, the method further comprises obtaining the sample from a subject.

In embodiments, the sample is blood, blood plasma, blood serum, cerebrospinal fluid, bile acid, saliva, mucosal lining fluid (including but not limited to nasal or oral mucosal lining fluid), whole blood, serum, plasma, or any other blood component, breath or exhaled breath condensate, synovial fluid, pleural fluid, pericardial fluid, peritoneal fluid, feces, nasal fluid, ocular fluid, intracellular fluid, intercellular fluid, lymph fluid, urine, tissue, sputum, bladder washings, oral washings, tissue samples, touch preps, or fine-needle aspirates. The biological sample can be concentrated prior to use.

In embodiments, the sample is treated with substances that make LAM structures more accessible and/or enhance LAM detection. Such substances include, but are not limited to, enzymes (such as alpha-mannosidase, proteinase-K) and chloroform.

In embodiments, the subject is an animal. In embodiments, the subject is a mammal. In some embodiment, the subject is a non-human mammal. In embodiments, the subject is a non-primate (e.g., cows, buffalos, pigs, horses, cats, dogs, rats, mouse, guinea pigs, sheep, goats etc.) or a primate (e.g., monkey and human). In one embodiment, the subject is human. In some embodiments, the antibodies and antigen-binding fragments thereof disclosed herein bind to one or more strains of the MTC group, which comprises Mycobacterium tuberculosis, M africanum, M. canettii, M bovis, M. microti, M. orygis, M. caprae, M pinnipedii, M. suricattae, and M. mungi. In some embodiments, the antibodies and antigen-binding fragments thereof disclosed herein bind to M bovis.

Provided herein is a method of diagnosing TB in a subject, the method comprising (1) contacting a sample from the subject with an anti-Mtb AM antibody, or Mtb AM-binding fragment thereof, disclosed herein, and (2) determining binding of the anti-Mtb AM antibody, or Mtb AM-binding fragment thereof, to Mtb AM, an AM fragment, LAM, and/or an AM-comprising fragment of LAM, wherein if binding of the anti-Mtb AM antibody, or Mtb AM-binding fragment thereof, to Mtb AM, the AM fragment, LAM, and/or the AM-comprising fragment of LAM is detected, the subject is diagnosed with TB. Provided herein is a method of diagnosing TB in a subject, the method comprising detecting Mtb AM, an AM fragment, LAM, and/or an AM-comprising fragment of LAM in a sample using an assay device disclosed herein. Provided herein is a method of determining that a subject likely has TB, the method comprising (1) contacting a sample from the subject with an anti-Mtb AM antibody, or Mtb AM-binding fragment thereof, disclosed herein, and (2) determining binding of the anti-Mtb AM antibody, or Mtb AM-binding fragment thereof, to Mtb AM, an AM fragment, LAM, and/or an AM-comprising fragment of LAM, wherein if binding of the anti-Mtb AM antibody, or Mtb AM-binding fragment thereof, to Mtb AM, the AM fragment, LAM, and/or the AM-comprising fragment of LAM is detected, the subject is determined to likely have TB. In embodiments, the subject that has been determined to likely have TB is subjected to further Mtb/TB testing. In embodiments, the sample used for testing has been previously concentrated to increase the concentration of Mtb AM, an AM fragment, LAM, and/or an AM-comprising fragment of LAM in the sample.

Provided herein is a method of concentrating Mtb AM, AM fragments, LAM, and/or AM-comprising LAM fragments in a sample using an anti-Mtb AM antibody, or Mtb AM-binding fragment thereof, disclosed herein Methods of concentrating antigen using an antibody binding to the antigen are known in the art. In some embodiments, an anti-Mtb AM antibody, or Mtb AM-binding fragment thereof, disclosed herein is immobilized on a surface. The anti-Mtb AM antibody, or Mtb AM-binding fragment thereof, is then contacted with a sample of a first volume comprising Mtb AM, AM fragments, LAM, and/or AM-comprising LAM fragments, causing binding of the anti-Mtb AM antibody, or Mtb AM-binding fragment thereof, to the Mtb AM, AM fragments, LAM, and/or AM-comprising LAM fragments. Subsequently, binding is disrupted, e.g., by contacting the bound Mtb AM, AM fragments, LAM, and/or AM-comprising LAM fragments with a second (smaller) volume of a liquid that disfavors binding between the anti-Mtb AM antibody, or Mtb AM-binding fragment thereof, and the Mtb AM, AM fragments, LAM, and/or AM-comprising LAM fragments.

### Assay Devices and Methods of Detection and Diagnosis Using Anti-GroEL2 Antibodies, or GroEL2-Binding Fragments Thereof

Provided herein are devices and methods for the detection of Groel2 by using one or more anti-GroEL2 antibodies, or GroEL2-binding fragments thereof, as described herein.

Provided herein is a method of detecting Groel2, the method comprising contacting a biological sample with an anti-GroEL2 antibody, or GroEL2-binding fragment thereof, as described herein and detecting binding of the anti-GroEL2 antibody, or GroEL2-binding fragment thereof. Binding of the anti-GroEL2 antibody, or GroEL2-binding fragment thereof, to its antigen can be detected in numerous ways, including, but not limited to (1) attaching a reporting moiety to the anti-GroEL2 antibody, or GroEL2-binding fragment thereof, (2) contacting the GroEL2 bound by a first anti-GroEL2 antibody, or GroEL2-binding fragment thereof, with a second anti-GroEL2 antibody, or GroEL2-binding fragment thereof (which in turn may be attached to a reporting moiety), or (3) contacting the second anti-GroEL2 antibody, or GroEL2-binding fragment thereof, with a third antibody (which in turn may be attached to a reporting moiety) that recognizes an antigen located on the second anti-GroEL2 antibody, or GroEL2-binding fragment thereof, such as the constant region of the anti-GroEL2 antibody, or GroEL2-binding fragment thereof.

In an embodiment, an assay device is provided for selectively detecting GroEL2 in a biological sample comprising: a first portion comprising a first plurality of anti-GroEL2 antibodies, or GroEL2-binding fragments thereof, as described herein, or anti-mycobacterial GroEL2-antibodies, wherein the antibodies or fragments are each attached to their own reporting entity; and a second portion comprising a second plurality of anti-GroEL2 antibodies, or GroEL2-binding fragments thereof, as described herein, or anti-mycobacterial GroEL2-antibodies.

In an embodiment, an assay device is provided for selectively detecting GroEL2 from the MTC group in a biological sample comprising: a first portion comprising a first plurality of anti-GroEL2 antibodies, or GroEL2-binding fragments thereof, as described herein, or anti-mycobacterial GroEL2-antibodies, wherein the antibodies or fragments are each attached to their own reporting entity; and a second portion comprising a second plurality of anti-GroEL2 antibodies, or GroEL2-binding fragments thereof, as described herein, or anti-mycobacterial GroEL2-antibodies. As used herein, an anti-mycobacterial GroEL2-antibody binds to GroEL2 derived from a mycobacterium.

MTC is a genetically related group of Mycobacterium species that can cause tuberculosis in humans or other animals and includes Mycobacterium tuberculosis, M africanum, M. canettii, M bovis, M. microti, M. orygis, M. caprae, M pinnipedii, M. suricattae, and M. mungi.

In an embodiment, the reporting entity comprises an enzyme. In an embodiment, the enzyme is horseradish peroxidase (HRP) or alkaline phosphatase (AP).

In one embodiment, the reporting entity comprises a gold nanoparticle.

In embodiments, the second plurality of anti-GroEL2-antibodies, or GroEL2-binding fragments thereof, or anti-mycobacterial GroEL2-antibodies, is affixed to a solid support of the device.

In embodiments, the first plurality of anti-GroEL2-antibodies, or GroEL2-binding fragments thereof, or anti-mycobacterial GroEL2-antibodies, is not affixed to a solid support of the device.

In embodiments, the solid support comprises nitrocellulose.

In embodiments, the device further comprises a fluid sample pad prior in sequential order to the first and second portions.

In embodiments, the device further comprises a control portion subsequent in sequential order to the first and second portions.

In embodiments, the control portion comprises a third plurality of antibodies, wherein the third plurality of antibodies are capable of binding the first plurality of anti-GroEL2-antibodies, or GroEL2-binding fragments thereof, or anti-mycobacterial GroEL2-antibodies each attached to their own reporting molecule. The third plurality of antibodies may be immobilized on a solid support of the device. In embodiments, the third plurality of antibodies comprises an antibody disclosed herein.

In embodiments, the device further comprises a fluid-absorbent wicking pad subsequent in sequential order to the first and second portions, and third portion if present.

In embodiments, the device is a lateral flow device.

Provided is a lateral flow assay device for detecting Mtb GroE12 in a biological sample comprising:
(a) a first portion comprising a first plurality of anti-GroEL2-antibodies, or GroEL2-binding fragments thereof, as disclosed herein, wherein the antibodies or fragments are each attached to their own reporting entity; and
(b) a second plurality of anti-GroEL2-antibodies, or GroEL2-binding fragments thereof, or anti-mycobacterial GroEL2-antibodies.

Provided is a lateral flow assay device for detecting GroEL2 in a biological sample comprising:
(a) a first portion comprising a first plurality of anti-GroEL2-antibodies, or GroEL2-binding fragments thereof, as disclosed herein, wherein the antibodies or fragments are each attached to their own reporting entity; and
(b) a second plurality of anti-GroEL2-antibodies, or GroEL2-binding fragments thereof, or anti-mycobacterial GroEL2-antibodies.

In some embodiments, the assay device comprises one or more pluralities of anti-GroEL2 antibodies, or GroEL2-binding fragments thereof, or anti-mycobacterial GroEL2-antibodies, wherein at least one of the pluralities of anti-GroEL2 antibodies, or GroEL2-binding fragments thereof, or anti-mycobacterial GroEL2-antibodies, comprises a non-human constant region or a modified non-human constant region. In some embodiments, the assay device comprises one or more pluralities of anti-GroEL2 antibodies, or GroEL2-binding fragments thereof, or anti-mycobacterial GroEL2-antibodies, wherein at least one of the pluralities of anti-GroEL2 antibodies, or GroEL2-binding fragments thereof, or anti-mycobacterial GroEL2-antibodies, comprises a murine constant region or a modified murine constant region.

In an embodiment, the reporting entity comprises an enzyme. In an embodiment, the enzyme is horseradish peroxidase (HRP) or alkaline phosphatase (AP). In an embodiment, the reporting entity comprises a gold nanoparticle.

In an embodiment, the second plurality of anti-GroEL2 antibodies, or GroEL2-binding fragments thereof, or anti-mycobacterial GroEL2-antibodies is affixed to a solid support of the device.

In an embodiment, the first plurality of anti-GroEL2 antibodies, or GroEL2-binding fragments thereof, or anti-mycobacterial GroEL2-antibodies is not affixed to a solid support of the device.

In an embodiment, the solid support comprises nitrocellulose.

In an embodiment, the device further comprises a fluid sample pad prior in sequential order to the first and second portions.

In an embodiment, the device further comprises a control portion subsequent in sequential order to the first and second portions.

In an embodiment, the control portion comprises a third plurality of antibodies, immobilized on a solid support of the device, and which third plurality of antibodies are capable of binding the first plurality of anti-GroEL2 antibodies, or GroEL2-binding fragments thereof, or anti-mycobacterial GroEL2-antibodies, each attached to their own reporting molecule.

In an embodiment, the device further comprises a fluid-absorbent wicking pad subsequent in sequential order to the first and second portions, and third portion if present.

In embodiments, the device is a lateral flow device.

In an embodiment, a method is provided for detecting MTC GroEL2 and/or one or more bacteria from the MTC group in a biological sample comprising:
(a) contacting a device described herein with the biological sample; and
(b) observing if MTC GroEL2 binds to the second plurality of anti-GroEL2 antibodies, or GroEL2-binding fragments thereof, or anti-mycobacterial GroEL2-antibodies, wherein if such anti-GroEL2 antibodies, or GroEL2-binding fragments thereof, or anti-mycobacterial GroEL2-antibodies, bind, then MTC GroEL2 and/or one or more bacteria from the MTC group have been detected in the biological sample; and if no anti-GroEL2 antibodies, or GroEL2-binding fragments thereof, or anti-mycobacterial GroEL2-antibodies, bind to the second plurality of anti-GroEL2 antibodies, or GroEL2-binding fragments thereof, or anti-mycobacterial GroEL2-antibodies, then MTC GroEL2 and bacteria from the MTC group have not been detected in the biological sample.

In embodiments, the method further comprises obtaining the sample from a subject.

In embodiments, the sample is blood, blood plasma, blood serum, cerebrospinal fluid, bile acid, saliva, mucosal lining fluid (including but not limited to nasal or oral mucosal lining fluid), whole blood, serum, plasma, or any other blood component, breath or exhaled breath condensate, synovial fluid, pleural fluid, pericardial fluid, peritoneal fluid, feces, nasal fluid, ocular fluid, intracellular fluid, intercellular fluid, lymph fluid, urine, tissue, sputum, bladder washings, oral washings, tissue samples, touch preps, or fine-needle aspirates. The biological sample can be concentrated prior to use.

In embodiments, the sample is treated with substances that make LAM structures more accessible and/or enhance LAM detection. Such substances include, but are not limited to, enzymes (such as alpha-mannosidase, proteinase-K) and chloroform.

In embodiments, the subject is an animal. In embodiments, the subject is a mammal. In some embodiment, the subject is a non-human mammal. In embodiments, the subject is a non-primate (e.g., cows, buffalos, pigs, horses, cats, dogs, rats, mouse, guinea pigs, sheep, goats etc.) or a primate (e.g., monkey and human). In one embodiment, the subject is human. In some embodiments, the antibodies and antigen-binding fragments thereof disclosed herein bind to one or more strains of the MTC group, which comprises Mycobacterium tuberculosis, M africanum, M. canettii, M bovis, M. microti, M. orygis, M. caprae, M pinnipedii, M. suricattae, and M. mungi. In some embodiments, the antibodies and antigen-binding fragments thereof disclosed herein bind to M bovis.

Provided herein is a method of diagnosing TB in a subject, the method comprising (1) contacting a sample from the subject with an anti-GroEL2 antibody, or GroEL2-binding fragment thereof, disclosed herein, and (2) determining binding of the anti-GroEL2 antibody, or GroEL2-binding fragment thereof, to GroEL2, wherein if binding of the anti-GroEL2 antibody, or GroEL2-binding fragment thereof, to GroEL2 is detected, the subject is diagnosed with TB. Provided herein is a method of diagnosing TB in a subject, the method comprising detecting GroEL2 in a sample using an assay device disclosed herein. Provided herein is a method of determining that a subject likely has TB, the method comprising (1) contacting a sample from the subject with an anti-GroEL2 antibody, or GroEL2-binding fragment thereof, disclosed herein, and (2) determining binding of the anti-GroEL2 antibody, or GroEL2-binding fragment thereof, to GroEL2, wherein if binding of the anti-GroEL2 antibody, or GroEL2-binding fragment thereof, to GroEL2 is detected, the subject is determined to likely have TB. In embodiments, the subject that has been determined to likely have TB is subjected to further Mtb/TB testing. In embodiments, the sample used for testing has been previously concentrated to increase the concentration of GroEL2 in the sample.

Provided herein is a method of concentrating GroEL2 in a sample using an anti-GroEL2 antibody, or GroEL2-binding fragment thereof, disclosed herein. Methods of concentrating antigen using an antibody binding to the antigen are known in the art. In some embodiments, an anti-GroEL2 antibody, or GroEL2-binding fragment thereof, disclosed herein is immobilized on a surface. The anti-GroEL2 antibody, or GroEL2-binding fragment thereof, is then contacted with a sample of a first volume comprising GroEL2, causing binding of the anti-GroEL2 antibody, or GroEL2-binding fragment thereof, to GroEL2. Subsequently, binding is disrupted, e.g., by contacting the bound GroEL2 with a second (smaller) volume of a liquid that disfavors binding between the anti-GroEL2 antibody, or GroEL2-binding fragment thereof, and GroEL2.

### Assay Devices and Methods of Detection and Diagnosis Using Anti-LprG Antibodies, or LprG-Binding Fragments Thereof

Provided herein are devices and methods for the detection of LprG by using one or more anti-LprG antibodies, or LprG-binding fragments thereof, as described herein.

Provided herein is a method of detecting LprG, the method comprising contacting a biological sample with an anti-LprG antibody, or LprG-binding fragment thereof, as described herein and detecting binding of the anti-LprG antibody, or LprG-binding fragment thereof. Binding of the anti-LprG antibody, or LprG-binding fragment thereof, to its antigen can be detected in numerous ways, including, but not limited to (1) attaching a reporting moiety to the anti-LprG antibody, or LprG-binding fragment thereof, (2) contacting the LprG bound by a first anti-LprG antibody, or LprG-binding fragment thereof, with a second anti-LprG antibody, or LprG-binding fragment thereof (which in turn may be attached to a reporting moiety), or (3) contacting the second anti-LprG antibody, or LprG-binding fragment thereof, with a third antibody (which in turn may be attached to a reporting moiety) that recognizes an antigen located on the second anti-LprG antibody, or LprG-binding fragment thereof, such as the constant region of the anti-LprG antibody, or LprG-binding fragment thereof.

In an embodiment, an assay device is provided for selectively detecting LprG in a biological sample comprising: a first portion comprising a first plurality of anti-LprG antibodies, or LprG-binding fragments thereof, as described herein, or anti-mycobacterial LprG-antibodies, wherein the antibodies or fragments are each attached to their own reporting entity; and a second portion comprising a second plurality of anti-LprG antibodies, or LprG-binding fragments thereof, as described herein, or anti-mycobacterial LprG-antibodies.

In an embodiment, an assay device is provided for selectively detecting MTC LprG and/or one or more bacteria from the MTC group in a biological sample comprising: a first portion comprising a first plurality of anti-LprG antibodies, or LprG-binding fragments thereof, as described herein, or anti-mycobacterial LprG-antibodies, wherein the antibodies or fragments are each attached to their own reporting entity; and a second portion comprising a second plurality of anti-LprG antibodies, or LprG-binding fragments thereof, as described herein, or anti-mycobacterial LprG-antibodies. As used herein, an anti-mycobacterial LprG-antibody binds to LprG derived from a mycobacterium.

MTC is a genetically related group of Mycobacterium species that can cause tuberculosis in humans or other animals and includes Mycobacterium tuberculosis, M africanum, M. canettii, M bovis, M. microti, M. orygis, M. caprae, M pinnipedii, M. suricattae, and M. mungi.

In an embodiment, the reporting entity comprises an enzyme. In an embodiment, the enzyme is horseradish peroxidase (HRP) or alkaline phosphatase (AP).

In one embodiment, the reporting entity comprises a gold nanoparticle.

In embodiments, the second plurality of anti-LprG-antibodies, or LprG-binding fragments thereof, or anti-mycobacterial LprG-antibodies, is affixed to a solid support of the device.

In embodiments, the first plurality of anti-LprG-antibodies, or LprG-binding fragments thereof, or anti-mycobacterial LprG-antibodies, is not affixed to a solid support of the device.

In embodiments, the solid support comprises nitrocellulose.

In embodiments, the device further comprises a fluid sample pad prior in sequential order to the first and second portions.

In embodiments, the device further comprises a control portion subsequent in sequential order to the first and second portions.

In embodiments, the control portion comprises a third plurality of antibodies, wherein the third plurality of antibodies are capable of binding the first plurality of anti-LprG-antibodies, or LprG-binding fragments thereof, or anti-mycobacterial LprG-antibodies each attached to their own reporting molecule. The third plurality of antibodies may be immobilized on a solid support of the device. In embodiments, the third plurality of antibodies comprises an antibody disclosed herein.

In embodiments, the device further comprises a fluid-absorbent wicking pad subsequent in sequential order to the first and second portions, and third portion if present.

In embodiments, the device is a lateral flow device.

Provided is a lateral flow assay device for detecting Mtb LprG and/orMtb bacteria in a biological sample comprising:
(a) a first portion comprising a first plurality of anti-LprG-antibodies, or LprG-binding fragments thereof, as disclosed herein, wherein the antibodies or fragments are each attached to their own reporting entity; and
(b) a second plurality of anti-LprG-antibodies, or LprG-binding fragments thereof, or anti-mycobacterial LprG-antibodies.

Provided is a lateral flow assay device for detecting LprG in a biological sample comprising:
(a) a first portion comprising a first plurality of anti-LprG-antibodies, or LprG-binding fragments thereof, as disclosed herein, wherein the antibodies or fragments are each attached to their own reporting entity; and
(b) a second plurality of anti-LprG-antibodies, or LprG-binding fragments thereof, or anti-mycobacterial LprG-antibodies.

In some embodiments, the assay device comprises one or more pluralities of anti-LprG antibodies, or LprG-binding fragments thereof, or anti-mycobacterial LprG-antibodies, wherein at least one of the pluralities of anti-LprG antibodies, or LprG-binding fragments thereof, or anti-mycobacterial LprG-antibodies, comprises a non-human constant region or a modified non-human constant region. In some embodiments, the assay device comprises one or more pluralities of anti-LprG antibodies, or LprG-binding fragments thereof, or anti-mycobacterial LprG-antibodies, wherein at least one of the pluralities of anti-LprG antibodies, or LprG-binding fragments thereof, or anti-mycobacterial LprG-antibodies, comprises a murine constant region or a modified murine constant region.

In an embodiment, the reporting entity comprises an enzyme. In an embodiment, the enzyme is horseradish peroxidase (HRP) or alkaline phosphatase (AP). In an embodiment, the reporting entity comprises a gold nanoparticle.

In an embodiment, the second plurality of anti-LprG antibodies, or LprG-binding fragments thereof, or anti-mycobacterial LprG-antibodies is affixed to a solid support of the device.

In an embodiment, the first plurality of anti-LprG antibodies, or LprG-binding fragments thereof, or anti-mycobacterial LprG-antibodies is not affixed to a solid support of the device.

In an embodiment, the solid support comprises nitrocellulose.

In an embodiment, the device further comprises a fluid sample pad prior in sequential order to the first and second portions.

In an embodiment, the device further comprises a control portion subsequent in sequential order to the first and second portions.

In an embodiment, the control portion comprises a third plurality of antibodies, immobilized on a solid support of the device, and which third plurality of antibodies are capable of binding the first plurality of anti-LprG antibodies, or LprG-binding fragments thereof, or anti-mycobacterial LprG-antibodies, each attached to their own reporting molecule.

In an embodiment, the device further comprises a fluid-absorbent wicking pad subsequent in sequential order to the first and second portions, and third portion if present.

In embodiments, the device is a lateral flow device.

In an embodiment, a method is provided for detecting MTC LprG and/or one or more bacteria from the MTC group in a biological sample comprising:
(a) contacting a device described herein with the biological sample; and
(b) observing if MTC LprG binds to the second plurality of anti-LprG antibodies, or LprG-binding fragments thereof, or anti-mycobacterial LprG-antibodies, wherein if such anti-LprG antibodies, or LprG-binding fragments thereof, or anti-mycobacterial LprG-antibodies, bind, then MTC LprG and/or one or more bacteria from the MTC group have been detected in the biological sample; and if no anti-LprG antibodies, or LprG-binding fragments thereof, or anti-mycobacterial LprG-antibodies, bind to the second plurality of anti-LprG antibodies, or LprG-binding fragments thereof, or anti-mycobacterial LprG-antibodies, then MTC LprG and bacteria from the MTC group have not been detected in the biological sample.

In embodiments, the method further comprises obtaining the sample from a subject.

In embodiments, the sample is blood, blood plasma, blood serum, cerebrospinal fluid, bile acid, saliva, mucosal lining fluid (including but not limited to nasal or oral mucosal lining fluid), whole blood, serum, plasma, or any other blood component, breath or exhaled breath condensate, synovial fluid, pleural fluid, pericardial fluid, peritoneal fluid, feces, nasal fluid, ocular fluid, intracellular fluid, intercellular fluid, lymph fluid, urine, tissue, sputum, bladder washings, oral washings, tissue samples, touch preps, or fine-needle aspirates. The biological sample can be concentrated prior to use.

In embodiments, the sample is treated with substances that make LAM structures more accessible and/or enhance LAM detection. Such substances include, but are not limited to, enzymes (such as alpha-mannosidase, proteinase-K) and chloroform.

In embodiments, the subject is an animal. In embodiments, the subject is a mammal. In some embodiment, the subject is a non-human mammal. In embodiments, the subject is a non-primate (e.g., cows, buffalos, pigs, horses, cats, dogs, rats, mouse, guinea pigs, sheep, goats etc.) or a primate (e.g., monkey and human). In one embodiment, the subject is human. In some embodiments, the antibodies and antigen-binding fragments thereof disclosed herein bind to one or more strains of the MTC group, which comprises Mycobacterium tuberculosis, M africanum, M. canettii, M bovis, M. microti, M. orygis, M. caprae, M pinnipedii, M. suricattae, and M. mungi. In some embodiments, the antibodies and antigen-binding fragments thereof disclosed herein bind to M bovis.

Provided herein is a method of diagnosing TB in a subject, the method comprising (1) contacting a sample from the subject with an anti-LprG antibody, or LprG-binding fragment thereof, disclosed herein, and (2) determining binding of the anti-LprG antibody, or LprG-binding fragment thereof, to LprG, wherein if binding of the anti-LprG antibody, or LprG-binding fragment thereof, to LprG is detected, the subject is diagnosed with TB. Provided herein is a method of diagnosing TB in a subject, the method comprising detecting LprG in a sample using an assay device disclosed herein. Provided herein is a method of determining that a subject likely has TB, the method comprising (1) contacting a sample from the subject with an anti-LprG antibody, or LprG-binding fragment thereof, disclosed herein, and (2) determining binding of the anti-LprG antibody, or LprG-binding fragment thereof, to LprG, wherein if binding of the anti-LprG antibody, or LprG-binding fragment thereof, to LprG is detected, the subject is determined to likely have TB. In embodiments, the subject that has been determined to likely have TB is subjected to further Mtb/TB testing. In embodiments, the sample used for testing has been previously concentrated to increase the concentration of LprG in the sample.

Provided herein is a method of concentrating LprG in a sample using an anti-LprG antibody, or LprG-binding fragment thereof, disclosed herein. Methods of concentrating antigen using an antibody binding to the antigen are known in the art. In some embodiments, an anti-LprG antibody, or LprG-binding fragment thereof, disclosed herein is immobilized on a surface. The anti-LprG antibody, or LprG-binding fragment thereof, is then contacted with a sample of a first volume comprising LprG, causing binding of the anti-LprG antibody, or LprG-binding fragment thereof, to LprG. Subsequently, binding is disrupted, e.g., by contacting the bound LprG with a second (smaller) volume of a liquid that disfavors binding between the anti-LprG antibody, or LprG-binding fragment thereof, and LprG.

### Kits

Provided herein is a kit containing one or more of the antibodies or antigen-binding fragments disclosed herein and one or more buffers.

“And/or” as used herein, for example, with option A and/or option B, encompasses the separate embodiments of (i) option A, (ii) option B, and (iii) option A plus option B.

All combinations of the various elements described herein are within the scope of the disclosure unless otherwise indicated herein or otherwise clearly contradicted by context.

All references cited herein are incorporated by reference herein in their entireties.

The following non-limiting example serves to further illustrate embodiments of the disclosure.

### EXAMPLES

### Material and Methods for the Examples Below

### Donor Information

Donors were selected based on a history of exposure to or infection with Mycobacterium tuberculosis, persistently high serum antibody IgG titers to AM, and desired reactivities to AM oligosaccharide motifs.

Donor T1/V57: 36 y/o asymptomatic male, HIV negative, immigrated to the US from India around 6 years prior to blood draw, reported exposure to family member with TB, Tuberculin skin-test (TST) positive

Donor T2/V58: 31 y/o asymptomatic male, HIV negative, immigrated to the US from Saudi Arabia around 6 years prior to blood draw, reported exposure to family member with TB, TST+.

Donor A1/NCB32: 51 y/o female, HIV negative, immigrated to the US from the Philippines around 30 years prior to blood draw, diagnosed with active tuberculosis (TB) with positive sputum microscopy and cultures for Mycobacterium tuberculosis, pulmonary and disseminated manifestations, was on antituberculous treatment for 6 weeks prior to blood draw for PBMCs.

Donor A2/WH37: 58 y/o male, HIV negative, immigrated to the US from Guyana around 3 years prior to blood draw, diagnosed with active tuberculosis (TB) with positive sputum microscopy and cultures for Mycobacterium tuberculosis, pulmonary and disseminated manifestations, was on antituberculous treatment for 8 weeks prior to blood draw for PBMCs.

### Culturing of Mycobacteria and Generation of Mycobacterial Capsular Arabinomannan (AM4)

Various strains of the Mycobacterium tuberculosis (Mtb) complex group comprised of laboratory strains (H37Rv, Erdman) and clinical strains (CDC1551, Beijing) were pre-cultured in Middlebrook 7H9 broth supplemented with 0.05% (v/v) tyloxapol and 10% (v/v) oleic albumin dextrose catalase enrichment to reach stationary growth phase (OD600 of 0.5-1.0). Similarly, non-tuberculous mycobacterial strains (M abscesses and M. avium) were cultured. To allow for mycobacterial capsule formation, the pre-culture strains were inoculated in minimal medium without detergent at 37° C. for 3 weeks. Capsular polysaccharides were isolated by physical extraction of capsular material of cells using glass beads, followed by clarification, and tetrafluorobor. AM was separated from the other capsular components using chloroform:methanol:water extraction (1:1:0.9) and was isolated and purified by size exclusion chromatography. Collected fractions were assayed for carbohydrate content by the phenol-sulfuric acid assay. AM-positive fractions were tested using a standard ELISA and positive control antibody (murine mAb CS-35, BEI NR-13811). The concentrations of AM and LAM were confirmed by GC-MS calculating the ratio of arabinose to mannose.

### Isolation of AM-Speci Fic B Cells

To identify arabinomannan (AM)-positive B cells by standard B cell immunophenotyping and fluorescence activated cell sorting (FACS), AM was activated by 1-cyano-4-dimethylaminopyridinium tetrafluoroborate (CDAP) and conjugated to biotin (1:20). For the isolation of mAb A2AM30, OS6 was synthetically generated and coupled to biotin. The biotinylated AM or OS6 was conjugated to Streptavidin labeled Alexa Fluor 647 (A647). The AM-A647 probe was then used with the antibody cocktail for cell staining. Lymphocytes were first identified, then live B-cells were further enriched based on CD19+CD27 or only CD19+ Cells stained with the same antibody cocktail but with A647 alone were used to set the sorting gate for AM+B cells. IgM- and antigen⁺ were sorted into a 96-well plate into RNAse-inhibiting lysis buffer.

Recombinant generation of mAbs from isolated single B cells

Reverse transcription PCR (RT-PCR) of lysed single B cells was used to synthesize complementary DNA (cDNA). Immunoglobulin genes for the heavy and light chains of the variable (V) region (Ig V_{H}, V_{κ}, and V_{L}) were amplified using random hexamer primers and sequenced. Then nested PCR was used to clone Ig genes into IgG heavy- and light-chain expression vectors and co-expressed by transfection of 293 HEK cells.

### Antibody Binding Assays

ELISA experiments were used to determine binding and estimate affinity of mAbs to isolated capsular AM, cell wall LAM, recombinant Mtb proteins, Mtb fractions, as well whole cells from different mycobacteria strains.

### Synthetic AM Glycan Microarray

A glycan microarray comprised of 30 synthetically generated AM oligosaccharide fragments was used to determine mAb reactivity to glycan epitopes. Briefly, microarray slides were blocked with 3% BSA in PBS at 4° C. overnight, then mAbs were incubated at 5 μg/ml for 4 hrs at 37° C. After washing with PBST, the slides were first incubated with Alexa 647 Goat Anti-Human IgG (Jackson Immunoresearch) at 37° C. for 2 hrs. The GenePix 4000 Microarray scanner system (Molecular Devices, CA) was used for scanning. Images were analyzed by using GenePix Pro 7.3.0.0 to measure median pixel intensity (MPI) and neighboring background pixel intensity (BPI) of individual spots. The median fluorescence intensity (MFI), representing AM-epitope specific mAb reactivity, was calculated using the MPI minus the BPI and averaged from the triplicate spots. The median fluorescent intensity (MFI), representing AM-epitope specific mAb reactivity, was the MPI minus the BPI. The final MFI was averaged from the triplicates.

### Example 1: Generation of anti-AM antibodies T2AM02, T2AM11, T2AM15, A1AM23, T1AM65, A2AM30, and A2AM10

Monoclonal mAbs were generated using a targeted, flow cytometry-based strategy. Initially, subjects from a range of Mtb exposure and infection along the clinical spectrum were tested for their serum anti-AM IgG responses. The donors were selected based on having available PBMCs and anti-AM IgG titers. CD3⁻, CD14⁻, CD19⁺, CD27⁺, IgM⁻, AM⁺ memory B cells were isolated from a sample of peripheral blood mononuclear cells (PBMC) from the respective donors using flow cytometry. The variable heavy and light chains of the B cell receptors of the isolated B cells were recovered by nested RT-PCR and an initial panel of recombinant human mAbs were expressed in an IgG1 vector in HEK293 cells. For the expression of the heavy chains, cloning vector AbVec-hIgG1 was used (GenBank: FJ475055.1; Di Niro R, Mesin L, Raki M, Zheng N Y, Lund-Johansen F, Lundin K E, Charpilienne A, Poncet D, Wilson P C, Sollid L M. Rapid generation of rotavirus-specific human monoclonal antibodies from small-intestinal mucosa. J Immunol. 2010 Nov. 1; 185(9):5377-83). For the expression of λ light chains, a pBR322 based IG-lambda expression vector was used (GenBank: FJ517647.1). For the expression of the κ light chains, cloning vector AbVec-hIgKappa was used (GenBank: FJ475056.1; Di Niro R, Mesin L, Raki M, Zheng N Y, Lund-Johansen F, Lundin K E, Charpilienne A, Poncet D, Wilson P C, Sollid L M. Rapid generation of rotavirus-specific human monoclonal antibodies from small-intestinal mucosa. J Immunol. 2010 Nov. 1; 185(9):5377-83). See Table 11 for sequences of the heavy and light constant regions.

Antibodies T2AM02, T2AM11, and T2AM15 were generated from B cells isolated from donor T2. Antibody A1AM23 was generated from B cells isolated from donor A1. T1AM65 was generated from B cells isolated from donor T1. A2AM10 and A2AM30 were generated from B cells isolated from donor A2.

The CDR and variable chain sequences of antibodies T2AM02, T2AM1 1, T2AM15, A1AM23, T1AM65, A2AM30, A2AM1O are provided in Tables 4-10.

TABLE 4
CDR sequences of anti-AM antibody T2AM02
(CDR nomenclature using IMGT).

| SEQ | | |
|---|---|---|
| ID | Fea- | Amino acid or nucleic acid |
| NO: | ture | sequence |
| 1 | CDRH1 | GFRFSDYW |
| 2 | CDRH2 | IKQDGSER |
| 3 | CDRH3 | ARDFGMATPGGMFY |
| 4 | CRDL1 | SSDVGRYNL |
| n/a | CDRL2 | AVT |
| 5 | CDRL3 | FSYAGGDNWM |
| 6 | VH | EVQLVESGGDLVQPGGSLRLSCVAS |
| | (CDRs | GFRFSDYWMTWVRQAPGKGLEWVAN |
| | in | IKQDGSERHYVDSVKGRFTVSRDNA |
| | bold) | ENSVYLQMNSLRAEDTAMYYCARDF |
| | | GMATPGGMFYWGQGALVTVSS |
| 7 | VL | QSALTQPPSASGSPGQSVTISCSGT |
| | (λ) | SSDVGRYNLVSWFQRHPGKAPKLMI |
| | (CDRs | YAVTKRPSGVPARFSGSKSGNTASL |
| | in | TVSGLQADDEGDYYCFSYAGGDNWM |
| | bold) | FGGGTKLTVL |
| 8 | VH | GAGGTGCAGCTGGTGGAGTCAGGGG |
| | | GAGACTTGGTCCAGCCGGGGGGGTC |
| | | CCTGAGACTCTCCTGTGTTGCCTCT |
| | | GGATTCAGGTTTAGTGACTACTGGA |
| | | TGACGTGGGTCCGCCAGGCTCCAGG |
| | | GAAGGGGCTGGAGTGGGTGGCCAAC |
| | | ATAAAGCAAGATGGAAGTGAGAGGC |
| | | ACTATGTGGACTCTGTGAAGGGCCG |
| | | GTTCACCGTCTCCAGAGACAACGCC |
| | | GAGAACTCAGTCTATCTACAAATGA |
| | | ACAGCCTGAGAGCCGAAGACACGGC |
| | | TATGTATTATTGTGCGCGAGATTTT |
| | | GGTATGGCCACCCCGGGAGGGATGT |
| | | TCTACTGGGGCCAGGGAGCCCTGGT |
| | | CACCGTCTCCTCAG |
| 9 | VL | CAGTCTGCCCTGACTCAGCCTCCCT |
| | (λ) | CCGCGTCCGGGTCTCCTGGACAGTC |
| | | AGTCACCATCTCCTGCTCTGGAACC |
| | | AGCAGTGACGTTGGTCGTTATAACC |
| | | TTGTCTCCTGGTTCCAGCGGCACCC |
| | | AGGCAAAGCCCCCAAACTCATGATT |
| | | TATGCGGTCACTAAGCGGCCCTCAG |
| | | GGGTCCCTGCTCGCTTCTCTGGCTC |
| | | CAAGTCTGGCAACACGGCCTCCCTG |
| | | ACCGTCTCTGGGCTCCAGGCTGACG |
| | | ATGAGGGTGATTATTACTGCTTCTC |
| | | ATATGCCGGCGGCGACAATTGGATG |
| | | TTCGGCGGGGGGACCAAACTGACCG |
| | | TCCTGG |

TABLE 5
CDR sequences of anti-AM antibody T2AM11
(CDR nomenclature using IMGT).

| SEQ | | |
|---|---|---|
| ID | Fea- | Amino acid or nucleic acid |
| NO: | ture | sequence |
| 10 | CDRH1 | GFTFTNAW |
| 11 | CDRH2 | VKSEGDGGTT |
| 12 | CDRH3 | ATGGLKYEA |
| 13 | CRDL1 | SSDVGGHNL |
| n/a | CDRL2 | DVT |
| 14 | CDRL3 | SSYRSSDTLVV |
| 15 | VH | EVQLVESGGGLVKPGGSLRLSCVVS |
| | (CDRs | GFTFTNAWMNWVRQAPGKGLEWVGR |
| | in | VKSEGDGGTTEYAASVKGRFIISRD |
| | bold) | DSKDTLYLQMNSLKREDTALYYCAT |
| | | GGLKYEAWGQGTMVTVSS |
| 16 | VL | QSALTQPASVSGSPGQSITISCTGT |
| | (λ) | SSDVGGHNLVAWYQQHPGDAPKLMI |
| | (CDRs | FDVTRRPSGVSNRFSGSKSGNTASL |
| | in | TISGLQAEDEADYYCSSYRSSDTLV |
| | bold) | VLGGGTRLTVL |
| 17 | VH | GAGGTGCAGCTGGTGGAGTCGGGGG |
| | | GAGGCTTGGTCAAGCCTGGGGGGTC |
| | | CCTTAGACTCTCCTGTGTAGTCTCT |
| | | GGATTCACTTTCACTAACGCCTGGA |
| | | TGAACTGGGTCCGCCAGGCTCCAGG |
| | | GAAGGGGCTGGAGTGGGTTGGCCGT |
| | | GTTAAAAGTGAAGGTGACGGTGGGA |
| | | CAACAGAGTACGCTGCATCCGTGAA |
| | | AGGCAGATTCATCATCTCAAGAGAT |
| | | GATTCAAAAGACACTCTGTATCTGC |
| | | AAATGAACAGTCTGAAAAGGGAGGA |
| | | CACAGCCCTGTATTATTGTGCCACA |
| | | GGAGGGTTAAAATACGAAGCCTGGG |
| | | GCCAAGGGACAATGGTCACCGTCTC |
| | | TTCAG |
| 18 | VL | CAGTCTGCCCTGACTCAGCCTGCCT |
| | (λ) | CCGTGTCTGGGTCTCCTGGACAGTC |
| | | GATCACCATCTCCTGCACTGGAACC |
| | | AGCAGTGACGTTGGAGGGCATAACC |
| | | TCGTCGCCTGGTACCAACAACACCC |
| | | AGGCGATGCCCCCAAACTCATGATT |
| | | TTTGATGTCACTAGACGGCCCTCAG |
| | | GGGTTTCTAATCGCTTCTCTGGCTC |
| | | CAAGTCTGGCAACACGGCCTCCCTG |
| | | ACCATCTCTGGGCTCCAGGCTGAGG |
| | | ACGAGGCTGATTATTATTGCAGCTC |
| | | ATATAGAAGCAGCGACACTCTCGTG |
| | | GTGCTCGGCGGAGGGACCAGGTTGA |
| | | CCGTCCTAG |

TABLE 6
CDR sequences of anti-AM antibody T2AM15
(CDR nomenclature using IMGT).

| SEQ | | |
|---|---|---|
| ID | Fea- | Amino acid or nucleic acid |
| NO: | ture | sequence |
| 19 | CDRH1 | GFAFVSGM |
| 20 | CDRH2 | SGDGHAT |
| 21 | CDRH3 | ALWLQVHFDN |
| 22 | CRDL1 | EVLVHSDGNTY |
| n/a | CDRL2 | KIS |
| 23 | CDRL3 | MQVTHFPWT |
| 24 | VH | EVQLLESGGALVQPGGSLRLSCAAS |
| | (CDRs | GFAFVSGMSWVRQAPGKGLEWVSTI |
| | in | SGDGHATHYADSVKGRFTISREDSK |
| | bold) | STLFLDMSNLRAEDTALYYCALWLQ |
| | | VHFDNWGQGTLVTVSS |
| 25 | VL | DIVMTQTPLSSPVALGQSASISCRS |
| | (κ) | SEVLVHSDGNTYMTWFHQRPGQPPR |
| | (CDRs | LLIYKISRRFSGVPDRFSGSGAGTD |
| | in | FTLKISRVEAEDVGVYYCMQVTFP |
| | bold) | HWTFGRGTKVEIK |
| 26 | VH | GAGGTGCAGCTGTTGGAGTCTGGGG |
| | | GAGCCTTGGTACAGCCTGGGGGGTC |
| | | CCTGAGACTCTCCTGTGCAGCCTCT |
| | | GGATTCGCCTTTGTGTCTGGCATGA |
| | | GTTGGGTCCGCCAGGCTCCAGGGAA |
| | | GGGGCTGGAGTGGGTCTCAACTATT |
| | | AGTGGTGATGGTCATGCTACACATT |
| | | ATGCAGACTCCGTGAAGGGCCGGTT |
| | | CACCATCTCCAGAGAGGATTCCAAG |
| | | AGCACCCTGTTTCTGGACATGAGCA |
| | | ACTTGAGAGCCGAGGACACGGCCCT |
| | | GTATTACTGTGCCCTATGGCTACAA |
| | | GTGCACTTTGACAACTGGGGCCAGG |
| | | GAACCCTGGTCACCGTCTCCTCAG |
| 27 | VL | GATATTGTGATGACCCAGACTCCAC |
| | (κ) | TCTCCTCACCTGTCGCCCTTGGACA |
| | | GTCGGCCTCCATCTCCTGCAGGTCT |
| | | AGTGAAGTCCTCGTCCACAGTGATG |
| | | GAAACACCTACATGACTTGGTTTCA |
| | | CCAGAGGCCAGGCCAGCCTCCAAGA |
| | | CTCCTTATTTATAAGATTTCTAGGC |
| | | GATTCTCTGGGGTCCCAGACAGATT |
| | | CAGTGGCAGTGGGGCAGGGACAGAT |
| | | TTCACCCTGAAAATCAGCAGGGTGG |
| | | AGGCTGAGGATGTCGGGGTTTATTA |
| | | CTGCATGCAGGTTACACATTTTCCG |
| | | TGGACGTTCGGCCGAGGGACCAAGG |
| | | TGGAAATCAAAC |

TABLE 7
CDR sequences of anti-AM antibody A1AM23
(CDR nomenclature using IMGT).

| SEQ | | |
|---|---|---|
| ID | Fea- | Amino acid or nucleic acid |
| NO: | ture | sequence |
| 28 | CDRH1 | GFTLDDYG |
| 29 | CDRH2 | ISWNGGLT |
| 30 | CDRH3 | ARDWYRNDYYYGMDV |
| 31 | CRDL1 | QSVSSN |
| n/a | CDRL2 | GAS |
| 32 | CDRL3 | QQYHIWYT |
| 33 | VH | EVQLVESGGGVVRPGGSLRLSCAAS |
| | (CDRs | GFTLDDYGISWVRQAPGKGLEWVSG |
| | in | ISWNGGLTGYADSVKGRFTISRDSA |
| | bold) | KKSLYLQMNSLRAEDTALYHCARDW |
| | | YRNDYYYGMDVWGQGTTVTVSS |
| 34 | VL | EIVLTQSPATLSLSPGERATLSCRA |
| | (κ) | SQSVSSNLAWYQQKPGQTPRLLIYG |
| | (CDRs | ASTRATGIPARFSGSGSGTEFTLAI |
| | in | SSLQSEDFAVYYCQQYHIWYTFGQG |
| | bold) | TKVEIK |
| 35 | VH | GAGGTGCAGCTGGTGGAGTCTGGGG |
| | | GAGGTGTGGTACGGCCTGGGGGGTC |
| | | CCTGAGACTCTCCTGTGCAGCCTCT |
| | | GGATTCACCTTGGATGATTATGGCA |
| | | TAAGCTGGGTCCGCCAAGCTCCAGG |
| | | GAAGGGGCTGGAGTGGGTCTCTGGT |
| | | ATTAGCTGGAATGGTGGTCTCACAG |
| | | GTTATGCAGACTCTGTTAAGGGGCG |
| | | ATTCACCATCTCCAGGGACAGCGCC |
| | | AAGAAGTCCCTGTATCTGCAAATGA |
| | | ACAGTCTGAGAGCCGAGGACACGGC |
| | | CTTGTATCACTGTGCGAGAGATTGG |
| | | TACAGGAACGACTACTACTACGGTA |
| | | TGGACGTCTGGGGCCAAGGGACCAC |
| | | GGTCACCGTCTCCTCA |
| 36 | VL | GAAATTGTGTTGACGCAGTCTCCAG |
| | (κ) | CCACCCTGTCTCTGTCTCCAGGGGA |
| | | AAGAGCCACCCTCTCCTGCAGGGCC |
| | | AGTCAGAGTGTTAGCAGCAACTTGG |
| | | CCTGGTACCAGCAGAAACCTGGCCA |
| | | GACTCCCAGGCTCCTCATCTATGGT |
| | | GCTTCCACCAGGGCCACTGGTATCC |
| | | CAGCCAGGTTCAGTGGCAGTGGGTC |
| | | TGGGACAGAGTTTACTCTCGCCATC |
| | | AGTAGCCTGCAATCTGAAGACTTTG |
| | | CAGTTTATTACTGTCAGCAGTATCA |
| | | TATCTGGTACACTTTTGGCCAGGGG |
| | | ACCAAGGTGGAAATCAAAC |

TABLE 8
CDR sequences of anti-AM antibody T1AM65
(CDR nomenclature using IMGT).

| SEQ | | |
|---|---|---|
| ID | Fea- | Amino acid or nucleic acid |
| NO: | ture | sequence |
| 37 | CDRH1 | GFPFSERG |
| 38 | CDRH2 | INDNGANT |
| 39 | CDRH3 | AGWLFNHFDL |
| 40 | CRDL1 | ESLVHSDGKTY |
| n/a | CDRL2 | EVS |
| 41 | CDRL3 | LQATHFPWS |
| 42 | VH | EVQLLESGGELVQPGGSLRLSCAAS |
| | (CDRs | GFPFSERGMSWVRQAQGKGLEWVST |
| | in | INDNGANTHYADSVMGRFTISRDNS |
| | bold) | KNTVYLQMNSLRDEDTGIYYCAGWL |
| | | FNHFDLWGQGALVTVSS |
| 43 | VL | DIVMTQTPLSSTVILGQPASISCKS |
| | (κ) | SESLVHSDGKTYVSWFHQRPGQPPR |
| | (CDRs | LLIYEVSKRLSGVPDRFSGGGAATD |
| | in | YTLRISRVEAEDVGIYYCLQATHFP |
| | bold) | WSFGHGTKVEIK |
| 44 | VH | GAGGTGCAGCTGTTGGAGTCTGGGG |
| | | GAGAGTTGGTACAGCCCGGGGGGTC |
| | | CCTGAGACTCTCCTGTGCAGCCTCT |
| | | GGATTCCCCTTTAGTGAACGTGGCA |
| | | TGAGTTGGGTTCGCCAGGCTCAAGG |
| | | GAAGGGGCTGGAGTGGGTCTCCACT |
| | | ATCAATGATAATGGCGCCAACACAC |
| | | ACTACGCAGACTCCGTGATGGGCCG |
| | | CTTCACCATCTCCAGAGACAATTCC |
| | | AAGAACACAGTATATCTACAAATGA |
| | | ATAGCCTGAGAGACGAGGACACGGG |
| | | CATTTATTACTGTGCGGGGTGGTTG |
| | | TTCAATCACTTTGACCTCTGGGGCC |
| | | AGGGAGCCCTGGTCACCGTCTCCTC |
| | | AG |
| 45 | VL | GATATTGTGATGACCCAGACTCCAC |
| | (κ) | TCTCCTCAACTGTCATCCTTGGACA |
| | | GCCGGCCTCCATCTCCTGTAAGTCT |
| | | AGTGAAAGCCTCGTTCACAGTGATG |
| | | GAAAAACCTACGTGAGTTGGTTTCA |
| | | CCAGAGGCCAGGCCAGCCTCCAAGA |
| | | CTCCTCATTTATGAGGTTTCTAAGC |
| | | GGTTGTCTGGGGTCCCAGACAGATT |
| | | CAGTGGCGGTGGGGCAGCGACAGAT |
| | | TACACACTGAGAATCAGCAGGGTGG |
| | | AGGCTGAGGACGTCGGCATTTATTA |
| | | CTGCTTGCAAGCCACACATTTCCCG |
| | | TGGTCGTTCGGCCACGGGACCAAGG |
| | | TGGAAATCAAAC |

TABLE 9
CDR sequences of anti-AM antibody A2AM30
(CDR nomenclature using IMGT).

| SEQ | | |
|---|---|---|
| ID | Fea- | Amino acid or nucleic acid |
| NO: | ture | sequence |
| 46 | CDRH1 | AFPFGTFG |
| 47 | CDRH2 | ISNSGKNT |
| 48 | CDRH3 | TNDCCGDSPLFNS |
| 49 | CRDL1 | QSLKHENGNTY |
| n/a | CDRL2 | KIS |
| 50 | CDRL3 | MQATHFPRT |
| 51 | VH | EVQLLESGGRLIQPGGSLGLSCAAS |
| | (CDRs | AFPFGTFGMSWVRQAPGKGLEWVST |
| | in | ISNSGKNTHYADSVKGRFIVSRDNS |
| | bold) | KNTLYLQMNNLRAEDTALYYCTNDC |
| | | CGDSPLFNSWGRGTLVTVSS |
| 52 | VL | DIVMTQTPLSSPVTLGQSATISCRS |
| | (κ) | SQSLKHENGNTYVSWLQQRPGQPPR |
| | (CDRs | LLIYKISNRVSGVPARFSGGGAGTD |
| | in | FTLKISRVEAEDVGIYYCMQATHFP |
| | bold) | RTFGQGTKVEIK |
| 53 | VH | GAGGTGCAGCTGTTGGAGTCTGGGG |
| | | GACGCCTGATACAGCCGGGGGGGTC |
| | | CCTGGGACTCTCCTGTGCAGCCTCT |
| | | GCGTTCCCCTTTGGTACCTTTGGCA |
| | | TGAGTTGGGTCCGCCAGGCTCCAGG |
| | | CAAGGGCCTGGAGTGGGTCTCAACT |
| | | ATTAGTAATAGTGGTAAAAACACAC |
| | | ACTACGCAGACTCCGTGAAGGGCCG |
| | | TTTCATCGTCTCCAGAGACAACTCC |
| | | AAGAACACATTGTATTTGCAAATGA |
| | | ACAACCTGAGAGCCGAAGACACGGC |
| | | CTTATATTACTGTACGAACGACTGC |
| | | TGTGGTGACTCACCCCTCTTTAACT |
| | | CCTGGGGCCGGGGAACTCTGGTCAC |
| | | CGTCTCCTCAG |
| 54 | VL | GATATTGTGATGACCCAGACTCCAC |
| | (κ) | TCTCCTCACCTGTCACCCTAGGACA |
| | | GTCGGCTACCATCTCCTGCAGGTCT |
| | | AGTCAAAGCCTCAAACACGAAAATG |
| | | GGAACACCTACGTGAGTTGGCTTCA |
| | | GCAGAGGCCAGGCCAGCCCCCAAGA |
| | | CTCCTAATTTACAAGATTTCAAACC |
| | | GGGTCTCTGGGGTTCCAGCCAGATT |
| | | CAGTGGCGGTGGGGCAGGGACAGAT |
| | | TTCACACTGAAAATCAGCAGGGTGG |
| | | AAGCTGAGGATGTCGGCATTTACTA |
| | | CTGCATGCAAGCTACACATTTTCCT |
| | | CGGACGTTCGGCCAAGGGACCAAGG |
| | | TGGAGATCAAAC |

TABLE 10
CDR sequences of anti-AM antibody A2AM10
(CDR nomenclature using IMGT).

| SEQ | | |
|---|---|---|
| ID | Fea- | Amino acid or nucleic acid |
| NO: | ture | sequence |
| 161 | CDRH1 | GFTFSHSD |
| 162 | CDRH2 | IGTAHDT |
| 163 | CDRH3 | ARVAFSWRCPDF |
| 164 | CRDL1 | SSNIGSNS |
| | CDRL2 | KSD |
| 165 | CDRL3 | AAWDDSLNGHLV |
| 166 | VH | EVQLVDSGGGLVQPGGSLRLSCVAS |
| | (CDRs | GFTFSHSDMHWVRQVTGKGLEWVAS |
| | in | IGTAHDTWYAGSVKGRFTISRENAK |
| | bold) | NSLYLQMNSLTVEDTAVYYCARVAF |
| | | SWRCPDFWGQGTLVTVSS |
| 167 | VL | QSVLTQPPSASGTPGQRVTISCSGS |
| | (λ) | SSNIGSNSVTWYQQVPGTVPKLLIY |
| | (CDRs | KSDQRPSGVPDRFSGSKSGSSASLA |
| | in | ISGLQSEDEADYYCAAWDDSLNGHL |
| | bold) | VFGGGTKLTVL |
| 168 | VH | gaggtgcagctggtggactctgggg |
| | | gaggcttggtacagcctggggggtc |
| | | cctgagactctcctgtgtagcctct |
| | | ggattcaccttcagtcactccgaca |
| | | tgcactgggtccgccaggttacggg |
| | | aaagggtctggagtgggtcgcatct |
| | | attggtactgctcatgacacgtggt |
| | | atgcaggctccgtgaagggccgatt |
| | | caccatctccagagaaaatgccaag |
| | | aactccttgtatcttcaaatgaaca |
| | | gcctgacagtcgaggacacggccgt |
| | | atattactgtgcaagagttgccttc |
| | | agctggcggtgccctgacttctggg |
| | | gccagggaaccctggtcaccgtctc |
| | | ctcag |
| 169 | VL | cagtctgtgctgactcagccaccct |
| | (λ) | cagcgtctgggacccccgggcagag |
| | | ggtcaccatctcttgttctggaagc |
| | | agctccaatatcggaagtaatagtg |
| | | taacctggtaccagcaggtcccagg |
| | | aacagtccccaaactcctcatctat |
| | | aagagtgatcagcggccctcagggg |
| | | tccctgaccgattctctggctccaa |
| | | gtctggctcctcagcctccctggcc |
| | | atcagtgggctccagtctgaggatg |
| | | aggctgattattattgtgcagcatg |
| | | ggatgacagtctgaatggtcatctg |
| | | gttttcggcggagggaccaaactga |
| | | ccgtcctag |

TABLE 11
Sequences for heavy and light constant regions
for anti-AM antibodies T2AM02, T2AM11, T2AM15,
A1AM23, T1AM65, A2AM30, and A2AM10. T2AM02,
T2AM11, and A2AM10 comprise λ light chains.
T2AM15, A1AM23, T1AM65, and A2AM30 comprise
κ light chains.

| SEQ | | |
|---|---|---|
| ID | | |
| NO | Description | Sequence |
| 56 | Human | RSTKGPSVFPLAPSSKSTSGG |
| | immunoglobulin | TAALGCLVKDYFPEPVTVSWN |
| | gamma-1 heavy | VSGALTSGHTFPAVLQSSGLY |
| | chain (heavy | SLSSVVTVPSSSLGTQTYICN |
| | chain constant | VNHKPSNTKVDKKVEPKSCDK |
| | region) from | THTCPPCPAPELLGGPSVFLF |
| | cloning vector | PPKPKDTLMISRTPEVTCVVV |
| | Ab Vec-hIgG1 | DVSHEDPEVKFNWYVDGVEVH |
| | (GenBank: | NAKTKPREEQYNSTYRVVSVL |
| | FJ475055.1) | TVLHQDWLNGKEYKCKVSNKA |
| | | LPAPIEKTISKAKGQPREPQV |
| | | YTLPPSRDELTKNQVSLTCLV |
| | | KGFYPSDIAVEWESNGQPENN |
| | | YKTTPPVLDSDGSFFLYSKLT |
| | | VDKSRWQQGNVFSCSVMHEAL |
| | | HNHYTQKSLSLSPGK |
| 57 | Human | SSEELQANKATLVCLISDFYP |
| | immunoglobulin | GAVTVAWKADSSPVKAGVETT |
| | C-lambda-2 | TPSKQSNNKYAASSYLSLTPE |
| | (light chain | QWKSHRSYSCQVTHEGSTVEK |
| | constant | TVAPTECS |
| | region) from | |
| | pBR322 based | |
| | IG-lambda | |
| | expression | |
| | vector | |
| | (GenBank: | |
| | FJ517647.1) | |
| 58 | Human | TVAAPSVFIFPPSDEQLKSGT |
| | immunoglobulin | ASVVCLLNNFYPREAKVQWKV |
| | kappa (light | DNALQSGNSQESVTEQDSKDS |
| | chain constant | TYSLSSTLTLSKADYEKHKVY |
| | region)from | ACEVTHQGLSSPVTKSFNRGE |
| | cloning vector | C |
| | Ab Vec- | |
| | hIgKappa | |
| | (GenBank: | |
| | FJ475056.1) | |

### Example 2: Isolated Anti-AM Antibodies Bind to Capsular AM, Surface LAM, and to Whole Cells

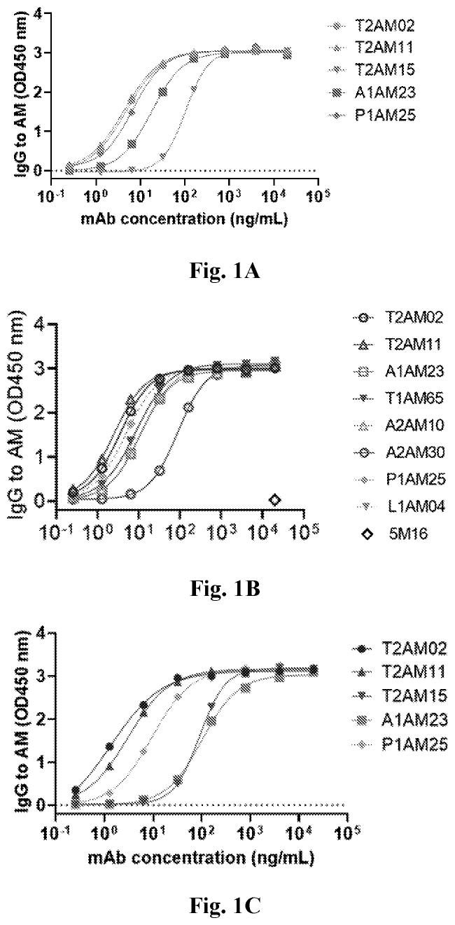

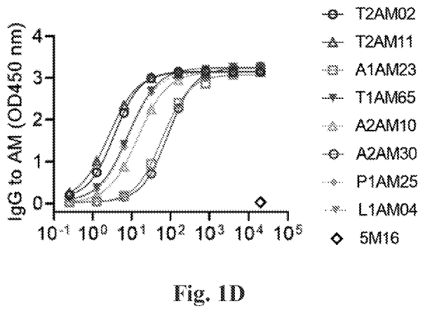

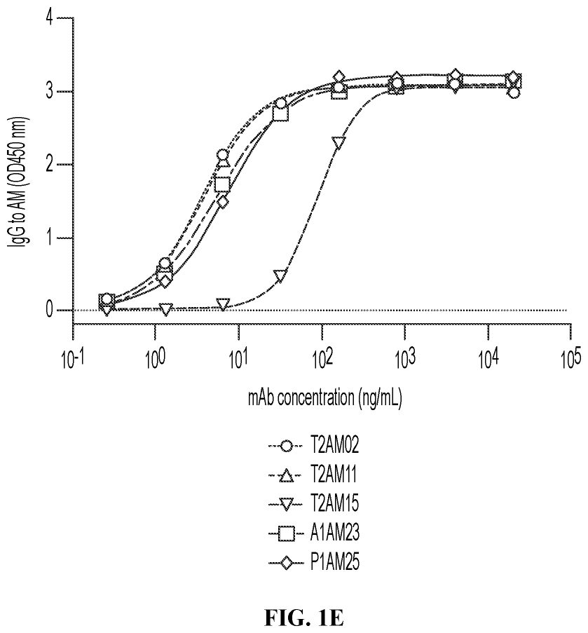

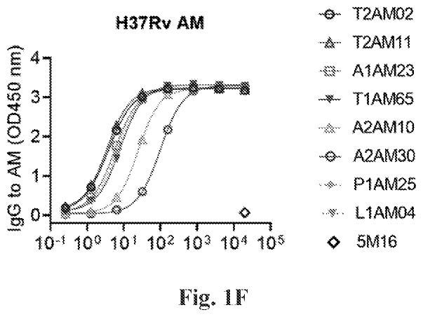

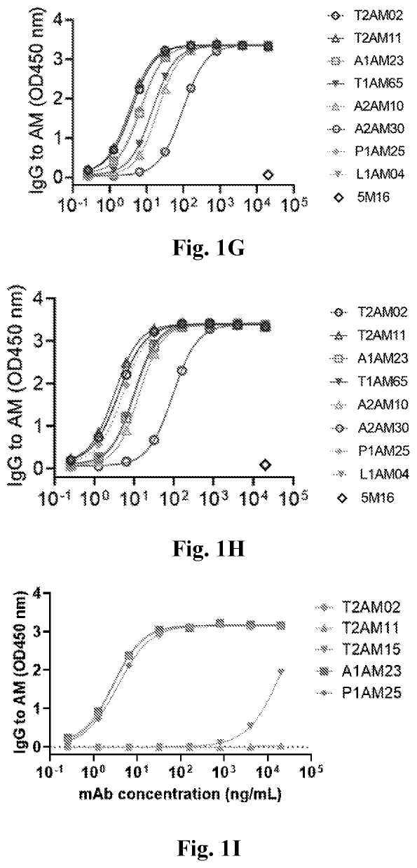

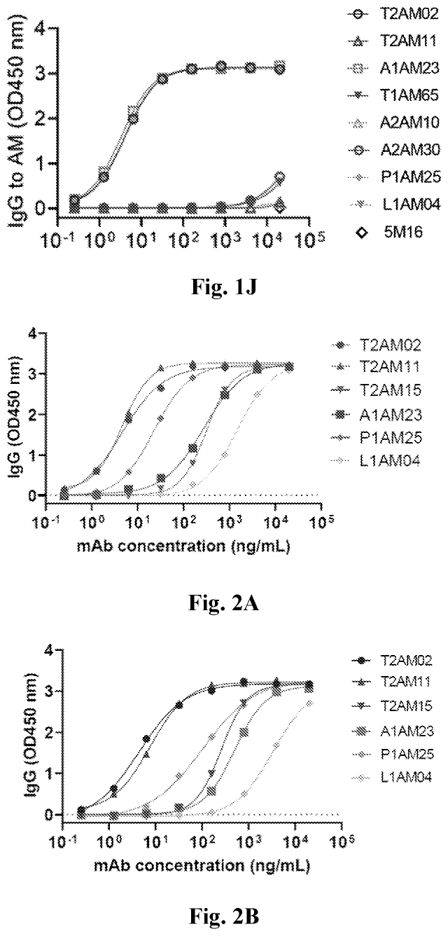

Binding of the isolated antibodies to capsular AiM isolated from Mtb andM. abscessus was determined by ELISA. Antibodies were titrated against AMV (10.g/ml) from clinical Mtb strains CDC1551 (virulent, FIGS. 1A and 11B), Beijing HN878 (virulent, FIGS. 1C and 1D), laboratory Mtb strain H37Rv (virulent, FIGS. 1E and 1F), avirulent strain H37Ra (FIG. 1G), avirulent M. bovis strain BCG (FIG. 111), and NTM strain M. abscessus (FIGS. 1I and 1J). Antibodies LIAM04 and PIAM25 served as controls.

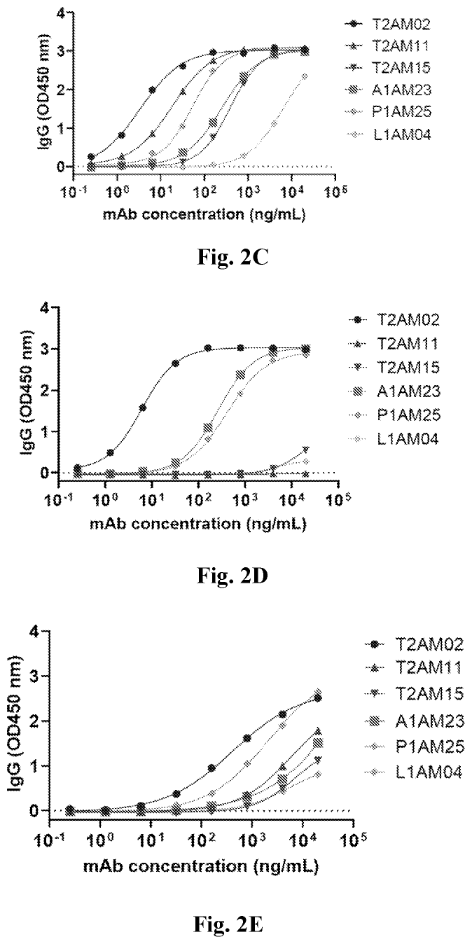

Binding of the isolated antibodies to whole cells of Mtb or NTM strains was determined by ELISA. Anti-AM mAbs were titrated against whole cells of clinical strains CDC1551 (FIG. 2A, Table 12), Beijing (FIG. 21B, Table 12), and a laboratory Mtb strain H37Rv (FIG. 2C, Table 12). Alternatively, anti-AM mAbs were titrated against whole cells of NTM strains M. abscessus (FIG. 2D, Table 12) andM. avium (FIG. 2E, Table 12). Strains were cultured in 7H9 in absence of detergent (to preserve the capsule) and were fixed in 2% paraformaldehyde. Bacterial cells coated based on 200 μg of total protein content per well. Five-fold serial diluted mAbs were applied as primary antibodies and the binding was detected using an HRP-conjugated anti-human IgG. Additional strains tested included

As shown in FIGS. 1 and 2 as well as Table 12, the isolated antibodies bound strongly to both capsular AM and whole cells. Of the tested antibodies, only T2AM02 and A1AM23 (as well as reference antibody P1Am25) bound to M. abscessus. With respect to binding to whole cells (FIG. 2), a broad range of concentrations for half-maximal binding was observed (EC50; Table 12), indicating variations in AM/LAM structures between mycobacterial strains. For example, T2AM02 showed strong binding to all mycobacteria, demonstrating that Mtb and NTM strains tested here contained α-arabinose. On the other hand, binding differences were observed for mAbs recognizing β-arabinose (P1AM25 and A1AM23). Moreover, A1AM23, recognizing non-capped β-arabinose, with weak to no binding to caps containing 2-3 mannose residues with or without MTX, bound weaker to Mtb clinical strains HN878 (Beijing; lineage 2) and CDC1551 (lineage 4) compared to Mtb lab strains Erdman and H37Rv (lineage 4), suggesting that more ManLAM capping is present in clinical than in lab strains. Anti-mannose mAbs bound much stronger to Mtb than NTM strains, indicating the absence of tri-(recognized by T1AM65) and tetra-mannoside epitopes (recognized by T2AM11 and A2AM10) as well as little presence of di-mannoside epitopes (recognized by A2AM30) in M. abscessus strains and little presence in M. avium and M smegmatis.

TABLE 12
Binding of anti-AM mAbs to whole bacteria of various mycobacterial strains
was assessed by ELISA. Bacteria were grown stationary and without detergent
to preserve the capsule. Binding curves were obtained by four-fold serial
dilution of mAbs starting at 20 μg/ml. The table shows log of EC50 values,
indicating the binding affinity of anti-AM mAbs to whole bacteria. N represents
no binding. Lower numbers indicate higher binding.

| | P1 | A1 | T2 | T2 | A2 | T1 | A2 |
|---|---|---|---|---|---|---|---|
| Strain | AM25 | AM23 | AM02 | AM11 | AM10 | AM65 | AM30 |
| Beijing HN878 | 2.7 | 3.4 | 1.1 | 1.5 | 2.3 | 2.7 | 2.7 |
| CDC1551 | 2.5 | 3.3 | 1.0 | 1.3 | 2.0 | 2.6 | 2.7 |
| Erdman | 1.4 | 2.6 | 0.9 | 0.9 | 1.9 | 1.6 | 2.3 |
| H37Rv | 2.3 | 2.8 | 1.1 | 1.5 | 2.3 | 2.7 | 2.7 |
| H37Ra | 2.1 | 2.9 | 0.9 | 1.5 | 2.3 | 2.7 | 2.6 |
| BCG | 2.7 | 3.7 | 1.4 | 2.5 | 2.8 | 3.4 | 3.2 |
| M. avium | 1.8 | 3.0 | 1.8 | 3.7 | 2.8 | 3.5 | 2.9 |
| M. abscessus (S) | 2.3 | 3.1 | 1.3 | N | N | N | 3.4 |
| M. abscessus (R) | 2.8 | 2.4 | 1.4 | N | N | N | 4.3 |
| M. smegmatis | 1.5 | 2.5 | 0.8 | 3.3 | 3.1 | 4.1 | 3.9 |

### Example 3: Isolated Anti-AM Antibodies Bind to Capsular AM with High Affinity as Determined by Bio-Layer Interferometry (BLI)

Kinetic binding curves of mAbs to AM (H37Rv) were determined by BLI. Cuves were fitting according to a 1:1 binding model. The kinetic binding constants are shown in Table 13. While all mAbs showed fast association patterns to Mtb (H37Rv) AM, they differed in their dissociation patterns. For instance, T2AM02 dissociated from AM extremely slowly, whereas A1AM23 dissociated very fast. Nevertheless, aside from A2AM30, all mAbs exhibited a high affinity with K_{D} values in the low nanomolar to picomolar range (10⁻⁹ to 10⁻¹⁰ M).

TABLE 13
Kinetic binding constants of antibodies analyzed.

| Antibody | K_{D, app} (M) | kₒₙ (1/Ms) | k_{dis} (1/s) |
|---|---|---|---|
| T2AM02 | 4.48E−10 | 1.78E+05 | 6.06E−05 |
| T2AM11 | 1.71E−09 | 2.44E+05 | 4.05E−04 |
| A1AM23 | 3.85E−09 | 7.40E+05 | 2.77E−03 |
| T1AM65 | 6.36E−09 | 1.44E+05 | 8.88E−04 |
| A2AM10 | 1.27E−08 | 3.12E+05 | 3.91E−03 |
| A2AM30 | 6.11E−09 | 1.55E+05 | 9.43E−04 |

### Example 4: Determination of Epitopes Targeted by Isolated Anti-AM Antibodies

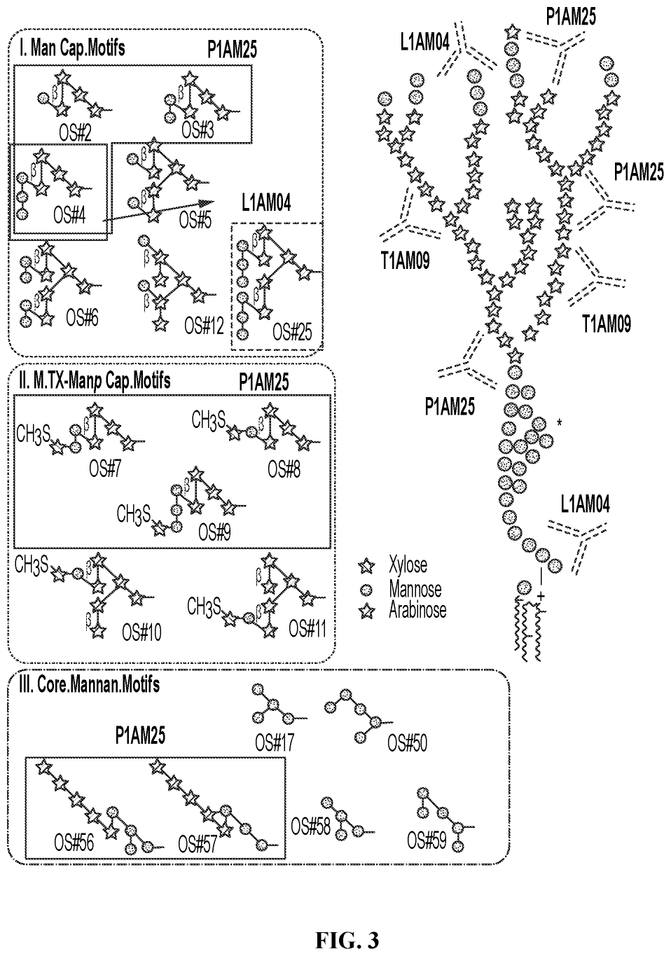

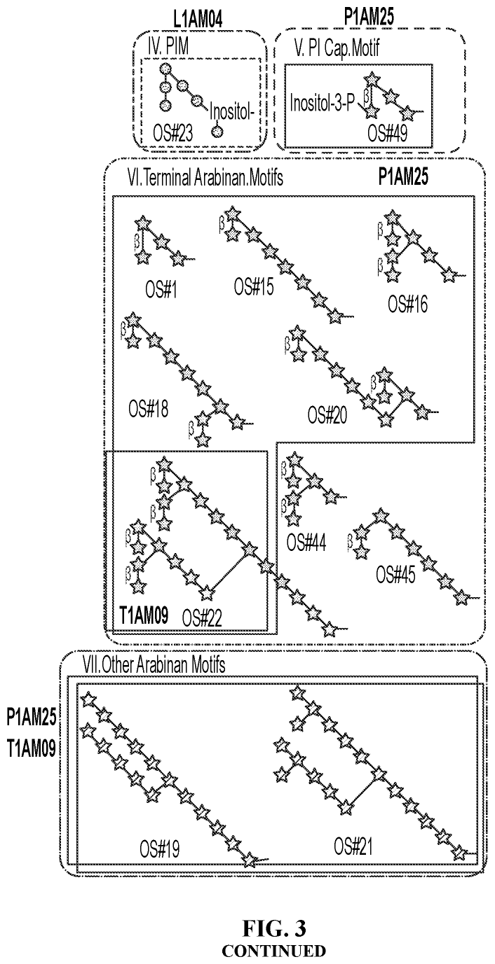

The reactivity of anti-AM mAbs to AM oligosaccharide (OS) motifs/epitopes was assessed using a glycan array containing 30 synthetically generated AM OS corresponding to structurally defined AM/LAM motifs (FIG. 3).

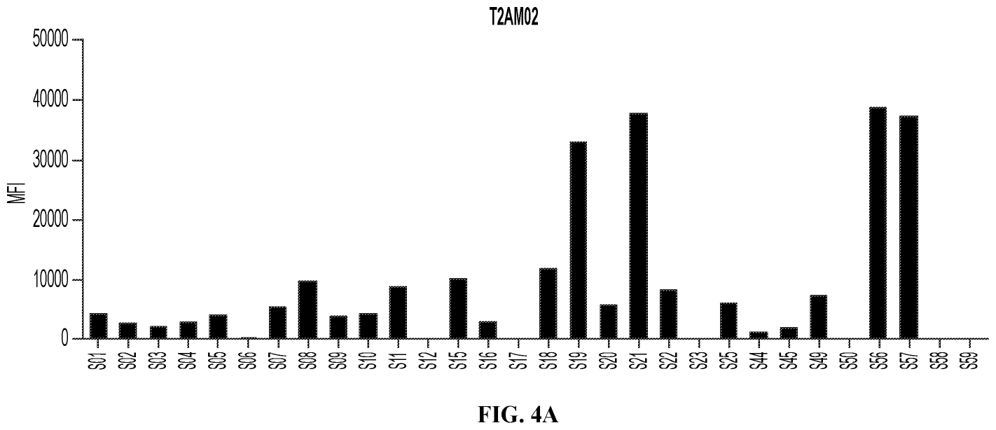

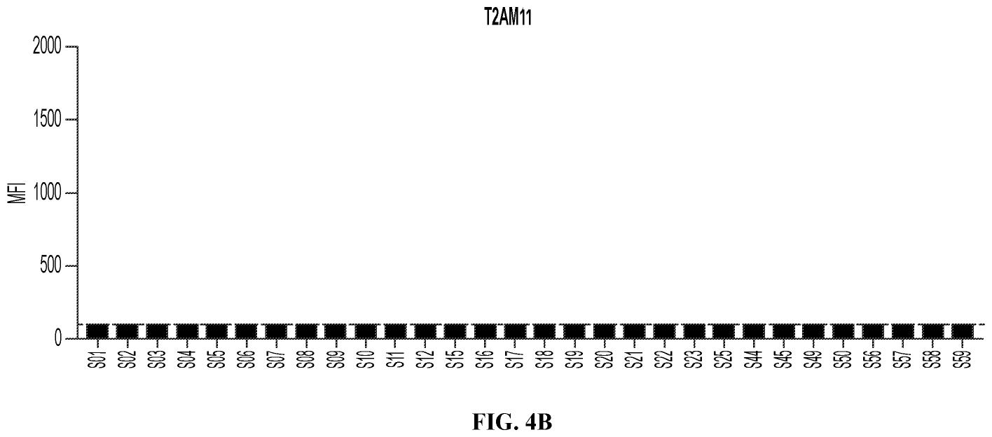

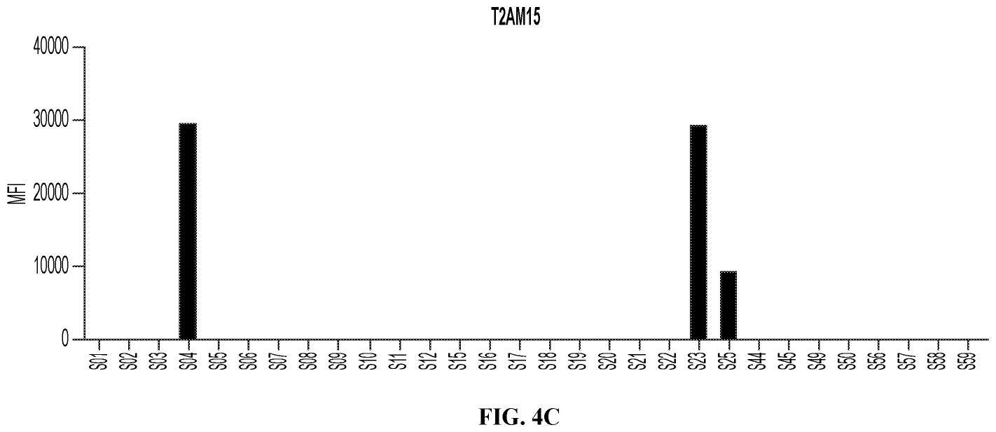

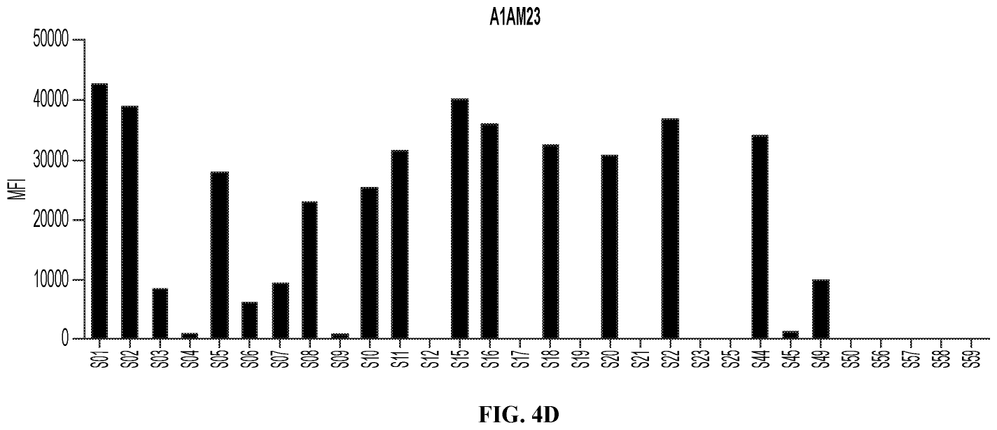

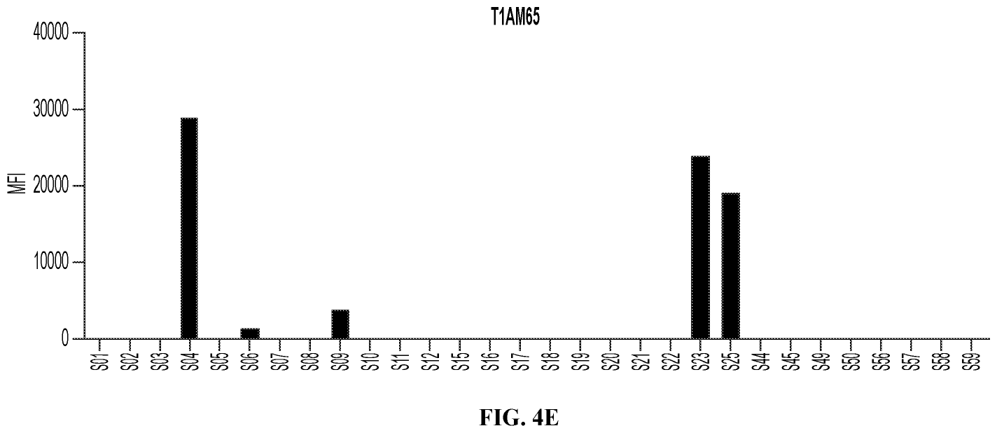

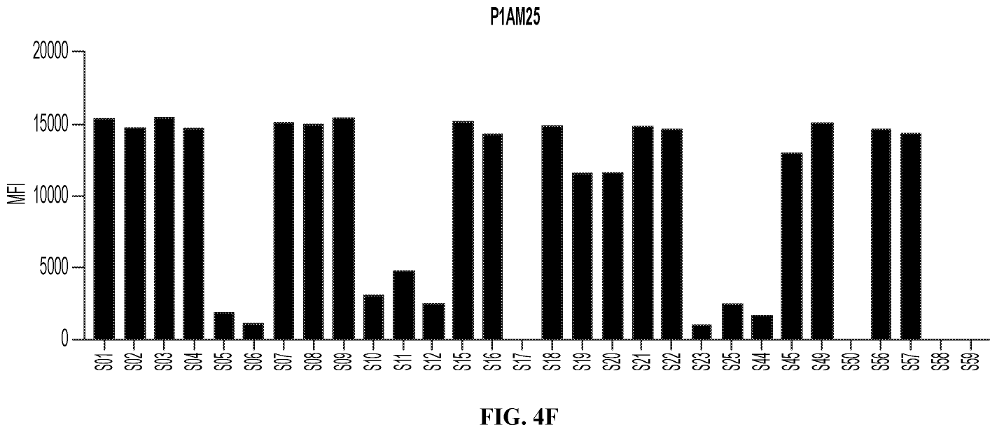

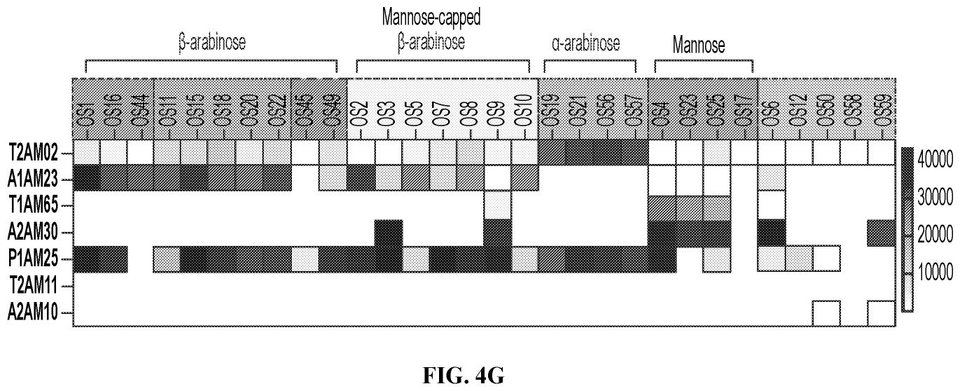

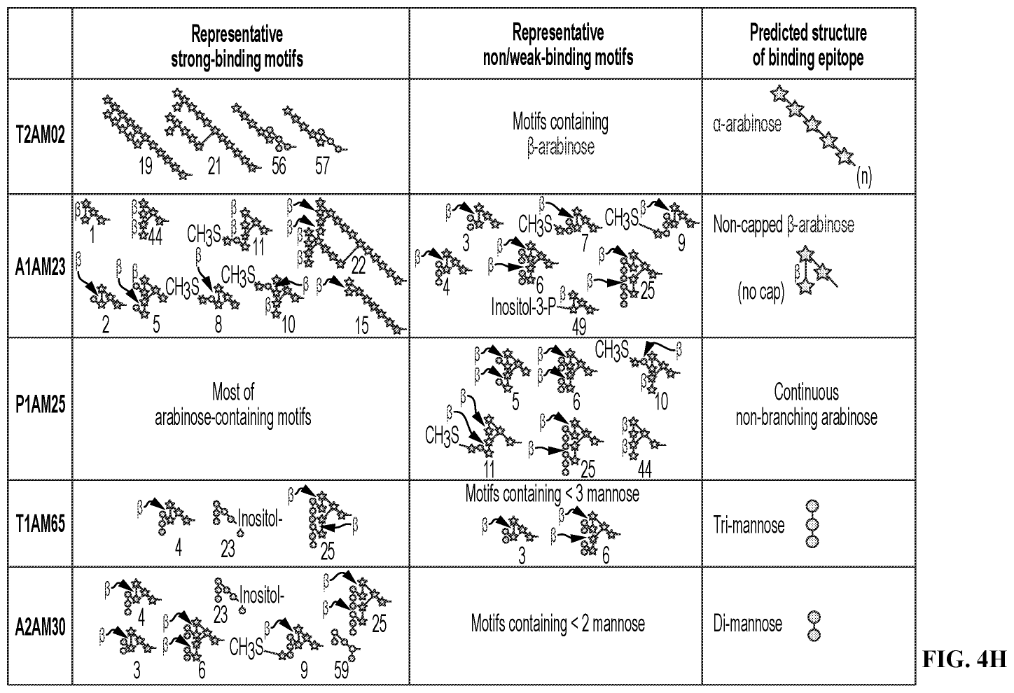

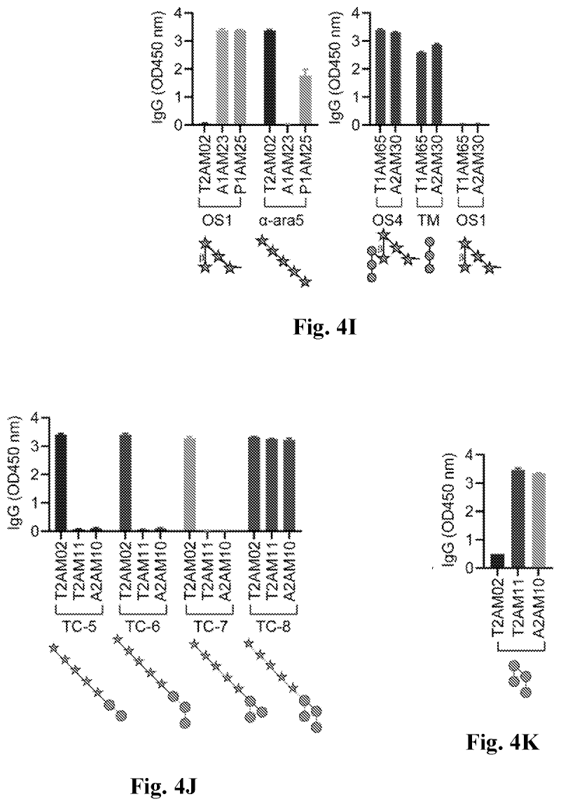

As shown generally in FIG. 4 and summarized in FIG. 4G, the isolated mAbs showed diverse reactivity to AM OS motifs. Antibodies T2AM15 and T1AM65 bound to same OS motifs as compared to control antibody L 1AM04 (FIGS. 3, 4C, and 4E), but with higher affinity. Antibodies T2AM02 and T2AM11 (and A2AM10) recognized different AM OS motifs that are further distinct from other known anti-AM antibodies (FIG. 4). T2AM02 exclusively bound to motifs in the α-arabinose group, A1AM23 recognized many but not all motifs containing 0-arabinose, while T1AM65 and A2AM30 bound to motifs containing mannose residues. Moreover, T2AM11 and A2AM10 recognized a newly identified tetra-mannoside epitope. A comparison of the structural differences between OS motifs that mAbs reacted with strongly (versus those they did not or only weakly reacted with) allowed a prediction of the structure of glycan epitopes the mAb recognized (FIG. 4H). Combining this approach with targeted evaluation of binding to truncated synthetic AM OS motifs demonstrated that T2AM02 binds to an epitope composed of α-arabinose, A1AM23 binds to a short non-capped β-arabinose, T1AM65 binds exclusively to a terminal tri-mannose whereas A2AM30 binds to a minimum of two mannose residues at the terminus (FIG. 4G). FIG. 4I shows verification of predicted epitopes by OS ELISA. Interestingly, whereas control antibody P1AM25, shared binding to β-arabinose structures with A1AM23, it also recognized, albeit to a much weaker extent, α-arabinose and thus also overlapped in part with T2AM02. Moreover, in contrast to A1AM32, P1AM25 reacted more broadly with range of ManLAM caps and showed little binding interference when arabinose structures contained more than one mannose residue (FIG. 4G).

Despite strong binding toMtb AM (FIGS. 1 and 2), antibodies T2AM11 and A2AM10 did not bind to any of the synthetic AM OS motifs printed on the glycan array. Using recently identified mannose containing AM/LAM motifs and the synthetic generation of related new fragments, it was shown that T2AM11 and A2AM10 recognized a tetrasacharide motif composed of four mannose residues (FIGS. 4J and 4K). These two mAbs were isolated from two different individuals with high anti-AM serum IgG titers, one with TB (Al) and one TST+(T2) subject, indicating that this newly identified tetra-mannoside motif is highly immunogenic.

### Example 5: Antibody Competition Experiments Provide Further Insights into AM Epitopes for the Isolated Anti-AM Antibodies

To further validate the predicted AM epitopes and investigate further how their relative position influences mAb binding competition, epitope binning experiments were performed with BLI. The isolated mAbs and control antibody P1AM25 were characterized as either arabinose- or mannose-binding. In the competition experiments, antibody binding to native Mtb AM (H37Rv) as assessed (Tables 14 and 15). Antibodies T2AM11 and A2AM10, both binding to a tetra-mannoside epitope, competed for binding to AM. However, antibodies T2AM11 and A2AM10 did not compete with antibody T1AM65, a tri-mannose binding mAb, or A2AM30, a di-mannose binding mAb, indicating that this tetra-mannoside epitope is distinct from the other mannose-based AM epitopes. On the other hand, antibody T1AM65 competed with antibody A2AM30 for binding to AM, whereas antibody A2AM30 competed only partially with antibody T1AM65, indicating that the di-mannoside epitope that antibody A2AM30 binds to is more present and accessible within Mtb AM than the tri-mannose that antibody T1AM65 binds to. Among the three mAbs targeting arabinose, antibodies A1AM23 and P1AM25 did not compete with antibody T2AM02 for binding to AM, confirming their distinct binding β-linked arabinose versus α-arabinose binding of antibody T2AM02, respectively. When antibody T2AM02 was used as the second mAb, some borderline partial competition with antibody P1AM25 was observed, suggesting that the binding of antibody P1AM25 include not just β-linked arabinose but also shorter fragments of α-arabinose as seen with our AM OS ELISA. Based on their binding to β-arabinose components, partial competition between antibodies A1AM23 and P1AM25 was observed. P_{GP34},T₁

TABLE 14
Results of two-phase binding experiment assessing competition
among anti-mannose mAbs and among anti-arabinose mAbs.

| | | Ab2 | | | |
|---|---|---|---|---|---|
| | | T2AM11 | A2AM10 | T1AM65 | A2AM30 |
| Ab1 | T2AM11 | 1.0 | 0.6 | 8.6 | 7.6 |
| | A2AM10 | 0.5 | 1.0 | 3.8 | 3.5 |
| | T1AM65 | 3.5 | 3.4 | 1.0 | 1.4 |
| | A2AM30 | 2.8 | 2.8 | 0.8 | 1.0 |

Antibody competition was assessed by determining the ratio of shift in wavelength of antibody 2 (Ab2) association to shift in wavelength of the same mAb at antibody 1 (Ab1) association, normalized by self-competition. Values in the table >=2 fold indicate o competition between antibodies. Values <=1 fold indicate competition between antibodies.

TABLE 15
Results of two-phase binding experiment assessing competition
among anti-mannose mAbs and among anti-arabinose mAbs.

| | | Ab2 | | |
|---|---|---|---|---|
| | | T2AM02 | A1AM23 | P1AM25 |
| Ab1 | T2AM02 | 1.0 | 6.1 | 4.6 |
| | A1AM23 | 2.0 | 1.0 | 1.6 |
| | P1AM25 | 1.8 | 1.5 | 1.0 |

Antibody competition was assessed by determining the ratio of shift in wavelength of antibody 2 (Ab2) association to shift in wavelength of the same mAb at antibody 1 (Ab1) association, normalized by self-competition. Values in the table >=2 fold indicate o competition between antibodies. Values <=1 fold indicate competition between antibodies.

### Example 6: Anti-AM Antibodies Promote Uptake of Mtb Bacteria by Neutrophils

Human neutrophils were extracted from the heparinized venous blood of healthy donors using a combination of dextran sedimentation and Ficoll-Hypaque gradient centrifugation techniques, as described in Maqbool M, Vidyadaran S, George E, Ramasamy R. Optimisation of laboratory procedures for isolating human peripheral blood derived neutrophils. Med J Malaysia 2011; 66:296-9. Freshly isolated neutrophils were adjusted to a concentration of 1×10⁶ cells/ml and infected with fluorescein isothiocyanate-(FITC) labeled Mtb strain H37Ra at a MOI of 5 and 10 for a duration of 1 hour in the presence of 10 μg/ml anti-LAM IgG1 mAbs and 10% heat-inactivated (HI) fetal bovine serum (FBS). Subsequently, the cells were washed multiple times and treated with trypan blue to extinguish the fluorescence emitted by noninternalized membrane-bound FITC-labeled bacteria. The rate of Mtb phagocytosis was assessed using flow cytometry. Human IgG1 monoclonal antibodies (5M16 and C144) targeting a non-Mtb antigens served as negative controls.

Co-incubation of anti-AM mAbs with bacteria increases uptake of bacteria (avirulent Mtb strain H37Ra) into neutrophils (FIG. 5). Compared to isotype matched control mAbs, the indicated anti-AM mAbs increase Mtb uptake in neutrophils.

### Example 7: An Anti-AM mAb Pair Shows High Sensitivity for Urinary LAM Detection

To illustrate the utility of antibodies disclosed herein to detect LAM with high sensitivity in biological samples, an ELISA was performed, using A1AM23 was as the capture antibody for LAM and P1AM25 was as the detection antibody. Over 20 mAb pair combinations were screened using capture ELISA with spiked LAM isolated from the clinical Mtb strain CDC1551.

A1AM23 and P1AM25 served as a capture and detection pair for detecting LAM in human urine. Initially, capture antibody A1AM23 at a concentration of 10 g/mL in PBS was applied to a 96-well polystyrene plate (Coming Costar) and incubated at 4° C. overnight. The plate underwent a triple wash with TBST before being blocked with 1% BSA/PBS for 1 hour at 25° C. After blocking, the plate underwent another triple wash with TBST, and human urine samples obtained from HIV, culture confirmed TB patients were introduced, followed by a 90-minute incubation at 25° C. Urine from a healthy volunteer residing in a non-TB endemic region was spiked with cultured CDC1551 LAM to create a standard curve. All urine samples were treated with proteinase K prior to use in ELISA. Following a third wash (5×with TBST), the detection antibody, biotinylated P1AM25, was introduced at a concentration of 250 ng/mL in 1% BSA/PBS and incubated for 90 minutes at 25° C. The plate was then washed again (10× with TBST) before adding a 1:200 dilution of streptavidin-horseradish peroxidase (R&D systems) in 1% BSA/PBS for 25 minutes at 25° C. A final wash was performed (10×with TBST), followed by the addition of 100 uL per well of TMB (ThermoFisher Scientific). After 30 minutes, the reaction was stopped with 2N sulfuric acid, and the absorbance was measured at 450 nm.

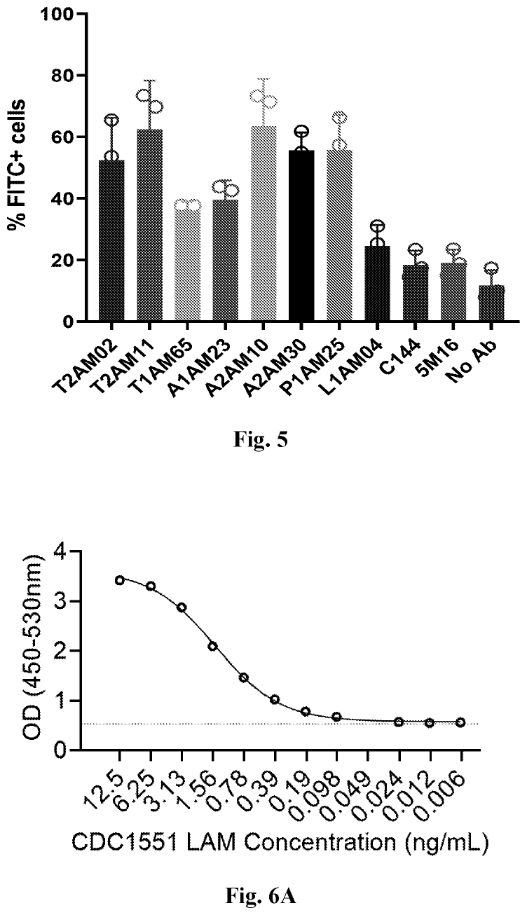

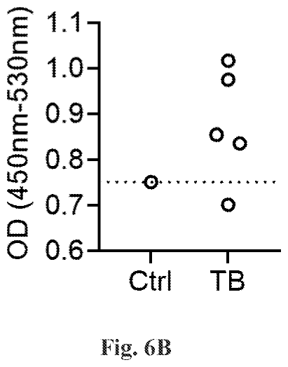

The antibody pair allowed for detection of cultured LAM from Mtb CDC 1551 spiked into urine with high sensitivity at concentrations as low as 30 μg/ml (FIG. 6A). Importantly, the antibody pair detected LAM in 80% ofurine samples from TB patients (FIG. 6B). As discussed herein, this detection rate is significantly better than the detection rate of the Alere Determine TB LAM Ag test or the FujiLAM test.

### Example 8: Generation of Anti-GroEL2 Antibodies

Recombinant Mtb protein GroEL2 was produced and purified using the Mycobacterium smegmatis expression system. The plasmid containing the sequence of Mtb GroEL2 protein followed by a polyhistidine tag was transformed to M. smegmatis. Single colonies were picked for small scale culture to screen positive colonies. Bacteria were then grown in a large scale. Recombinant protein was isolated by Ni-NTA (nickel-nitrilotriacetic acid) agarose from cell lysate, and further purified by a size exclusion chromatography. Five Balb/c mice (females, 6 weeks old) were immunized each with 100 μg of recombinant Groel2 intraperitoneal with a booster of 100 μg of recombinant Groel2 intraperitoneal after 4 weeks. Mice were bled 3 weeks after first and second dose to determine anti-Groel2 antibody titers. IgG titers to Groel2 after second immunization were high and mice were sacrificed.

Antigen specific B-cells from the spleen of the immunized mice were fused with myeloma cells to produce hybridoma cells. After screening, one clone from each hybridoma was selected for further study based on high antibody titers in culture supernatant. The sequences for the highest affinity mAbs, which had no or little competition with each other for binding to GroEL2 (antibodies 63.4, 80.8, 103.11, 111.6, and 238.11), are shown in Tables 16-20.

TABLE 16
CDR sequences of anti-GroEL2 antibody 63.4 (CDR nomenclature
using IMGT).

| SEQ | | |
|---|---|---|
| ID | | |
| NO: | Feature | Amino acid or nucleic acid sequence |
| 59 | CDRH1 | GFTFSSYA |
| 60 | CDRH2 | IDIGGDYT |
| 61 | CDRH3 | AYGHFAY |
| 62 | CRDL1 | QSLLDSDGKTY |
| n/a | CDRL2 | LVS |
| 63 | CDRL3 | WQGIYFPWT |
| 64 | VH | EVQLVESGGGLVKPGGSLKLSCAASGFTFSSYAMSWVRQTPE |
| | (CDRs in | KRLEWVATIDIGGDYTFYPDSVKGRFTISRDNAKNTLYLQMS |
| | bold) | SLRSEDTAMFYCAYGHFAYWGQGTLVTVSA |
| 65 | VL (κ) | DVVMTQTPLTLSVTIGQPASISCKSSQSLLDSDGKTYLNWLL |
| | (CDRs in | QRPGQSPKRLIYLVSKLDSGVPDRFTGSGSGTDFTLKISRVEAE |
| | bold) | DLGVYYCWQGIYFPWTFGGGTKLEIK |
| 66 | VH | GAAGTGCAGCTGGTGGAGTCTGGGGGAGGCTTAGTGAAGC |
| | | CTGGAGGGTCCCTGAAACTCTCCTGTGCAGCCTCTGGATTC |
| | | ACTTTCAGTAGCTATGCCATGTCTTGGGTTCGCCAGACTCC |
| | | GGAGAAGAGGCTGGAGTGGGTCGCAACCATTGATATTGGT |
| | | GGTGATTACACCTTCTATCCAGACAGTGTGAAGGGTCGATT |
| | | CACCATCTCCAGAGACAATGCCAAGAACACCCTGTACCTGC |
| | | AAATGAGCAGTCTGAGGTCTGAGGACACGGCCATGTTTTAC |
| | | TGTGCATATGGTCACTTTGCTTACTGGGGCCAGGGGACTCT |
| | | GGTCACTGTCTCTGCAG |
| 67 | VL (κ) | GATGTTGTGATGACCCAGACTCCACTCACTTTGTCGGTAAC |
| | | CATTGGACAACCAGCCTCCATCTCTTGCAAGTCAAGTCAGA |
| | | GCCTCTTGGATAGTGATGGAAAGACTTATTTGAATTGGTTG |
| | | TTACAGAGGCCAGGGCAGTCTCCAAAGCGCCTAATCTATCT |
| | | GGTGTCTAAACTGGACTCTGGAGTCCCTGACAGGTTCACTG |
| | | GCAGTGGATCAGGGACAGATTTCACACTGAAAATCAGCAG |
| | | AGTGGAGGCTGAGGATTTGGGAGTTTATTATTGCTGGCAAG |
| | | GTATATATTTTCCGTGGACGTTCGGTGGAGGAACCAAGCTG |
| | | GAAATCAAAC |
| 68 | Heavy | MNFVLSLIFLALILKGVQCEVQLVESGGGLVKPGGSLKLSC |
| | chain | AASGFTFSSYAMSWVRQTPEKRLEWVATIDIGGDYTFYPD |
| | (VH in | SVKGRFTISRDNAKNTLYLQMSSLRSEDTAMFYCAYGHFA |
| | bold; | YWGQGTLVTVSAAKTTPPSVYPLAPGCGDTTGSSVTLGCLV |
| | signal | KGYFPESVTVTWNSGSLSSGVHTFPAVLQSDLYTLSSSVTVPS |
| | sequence | STWPSETVTCNVAHPASSTKVDKKIVPRDCGCKPCICTVSEVS |
| | underlined) | SVFIFPPKPKDVLTITLTPKVTCVVVDISKDDPEVQFSWFVDDV |
| | | EVHTAQTQPREEQFNSTFRSVSELPIMHQDWLNGKEFKCRVN |
| | | SAAFPAPIEKTISKTKGRPKAPQVYTIPPPKEQMAKDKVSLTC |
| | | MITDFFPEDITVEWQWNGQPAENYKNTQPIMDTDGSYFVYSK |
| | | LNVQKSNWEAGNTFICSVLHEGLHNHHTEKSLSHSPGK |
| 69 | Light | MMSPAQFLFLLVLWIRETNGDVVMTQTPLTLSVTIGQPASIS |
| | chain | CKSSQSLLDSDGKTYLNWLLQRPGQSPKRLIYLVSKLDSG |
| | (VL in | VPDRFTGSGSGTDFTLKISRVEAEDLGVYYCWQGIYFPWT |
| | bold) | FGGGTKLEIKRADAAPTVSIFPPSSEQLTSGGASVVCFLNNFY |
| | | PKDINVKWKIDGSERQNGVLNSWTDQDSKDSTYSMSSTLTLT |
| | | KDEYERHNSYTCEATHKTSTSPIVKSFNRDEC |
| 70 | Heavy | ATGAACTTTGTGCTCAGCTTGATTTTCCTTGCCCTCATTTTA |
| | chain | AAAGGTGTCCAGTGTGAAGTGCAGCTGGTGGAGTCTGGG |
| | (VH in | GGAGGCTTAGTGAAGCCTGGAGGGTCCCTGAAACTCTC |
| | bold) | CTGTGCAGCCTCTGGATTCACTTTCAGTAGCTATGCCAT |
| | | GTCTTGGGTTCGCCAGACTCCGGAGAAGAGGCTGGAGT |
| | | GGGTCGCAACCATTGATATTGGTGGTGATTACACCTTCT |
| | | ATCCAGACAGTGTGAAGGGTCGATTCACCATCTCCAGA |
| | | GACAATGCCAAGAACACCCTGTACCTGCAAATGAGCAG |
| | | TCTGAGGTCTGAGGACACGGCCATGTTTTACTGTGCAT |
| | | ATGGTCACTTTGCTTACTGGGGCCAGGGGACTCTGGTC |
| | | ACTGTCTCTGCAGCCAAAACAACACCCCCATCAGTCTATC |
| | | CACTGGCCCCTGGGTGTGGAGATACAACTGGTTCCTCCGTG |
| | | ACTCTGGGATGCCTGGTCAAGGGCTACTTCCCTGAGTCAGT |
| | | GACTGTGACCTGGAACTCTGGATCCCTGTCCAGCGGTGTGC |
| | | ACACCTTCCCAGCTGTCCTGCAGTCTGACCTCTACACTCTG |
| | | AGCAGCTCAGTGACTGTCCCCTCCAGCACCTGGCCCAGCGA |
| | | GACCGTCACCTGCAACGTTGCCCACCCGGCCAGCAGCACC |
| | | AAGGTGGACAAGAAAATTGTGCCCAGGGATTGTGGTTGTA |
| | | AGCCTTGCATATGTACAGTCTCAGAAGTATCATCTGTCTTC |
| | | ATCTTCCCCCCAAAGCCCAAGGATGTGCTCACCATTACTCT |
| | | GACTCCTAAGGTCACGTGTGTTGTGGTAGACATCAGCAAGG |
| | | ATGATCCCGAGGTCCAGTTCAGCTGGTTTGTAGATGATGTG |
| | | GAGGTGCACACAGCTCAGACGCAACCCCGGGAGGAGCAGT |
| | | TCAACAGCACTTTCCGCTCAGTCAGTGAACTTCCCATCATG |
| | | CACCAGGACTGGCTCAATGGCAAGGAGTTCAAATGCAGGG |
| | | TCAACAGTGCAGCTTTCCCTGCCCCCATCGAGAAAACCATC |
| | | TCCAAAACCAAAGGCAGACCGAAGGCTCCACAGGTGTACA |
| | | CCATTCCACCTCCCAAGGAGCAGATGGCCAAGGATAAAGT |
| | | CAGTCTGACCTGCATGATAACAGACTTCTTCCCTGAAGACA |
| | | TTACTGTGGAGTGGCAGTGGAATGGGCAGCCAGCGGAGAA |
| | | CTACAAGAACACTCAGCCCATCATGGACACAGATGGCTCTT |
| | | ACTTCGTCTACAGCAAGCTCAATGTGCAGAAGAGCAACTG |
| | | GGAGGCAGGAAATACTTTCATCTGCTCTGTGTTACATGAGG |
| | | GCCTGCACAACCACCATACTGAGAAGAGCCTCTCCCACTCT |
| | | CCTGGTAAATGA |
| 71 | Light | ATGATGAGTCCTGCCCAGTTCCTGTTTCTGTTAGTGCTCTGG |
| | chain (κ) | ATTCGGGAAACCAACGGTGATGTTGTGATGACCCAGACT |
| | (VL in | CCACTCACTTTGTCGGTAACCATTGGACAACCAGCCTCC |
| | bold) | ATCTCTTGCAAGTCAAGTCAGAGCCTCTTGGATAGTGAT |
| | | GGAAAGACTTATTTGAATTGGTTGTTACAGAGGCCAGG |
| | | GCAGTCTCCAAAGCGCCTAATCTATCTGGTGTCTAAACT |
| | | GGACTCTGGAGTCCCTGACAGGTTCACTGGCAGTGGAT |
| | | CAGGGACAGATTTCACACTGAAAATCAGCAGAGTGGAG |
| | | GCTGAGGATTTGGGAGTTTATTATTGCTGGCAAGGTAT |
| | | ATATTTTCCGTGGACGTTCGGTGGAGGAACCAAGCTGG |
| | | AAATCAAACGGGCTGATGCTGCACCAACTGTATCCATCTTC |
| | | CCACCATCCAGTGAGCAGTTAACATCTGGAGGTGCCTCAGT |
| | | CGTGTGCTTCTTGAACAACTTCTACCCCAAAGACATCAATG |
| | | TCAAGTGGAAGATTGATGGCAGTGAACGACAAAATGGCGT |
| | | CCTGAACAGTTGGACTGATCAGGACAGCAAAGACAGCACC |
| | | TACAGCATGAGCAGCACCCTCACGTTGACCAAGGACGAGT |
| | | ATGAACGACATAACAGCTATACCTGTGAGGCCACTCACAA |
| | | GACATCAACTTCACCCATTGTCAAGAGCTTCAACAGGGATG |
| | | AGTGTTAG |

TABLE 17
CDR sequences of anti-GroEL2 antibody 80.8 (CDR nomenclature
using IMGT).

| SEQ | | |
|---|---|---|
| ID | | |
| NO: | Feature | Amino acid or nucleic acid sequence |
| 72 | CDRH1 | GFNIRDYY |
| 73 | CDRH2 | IDPDNGDT |
| 74 | CDRH3 | ARSPIGPAH |
| 75 | CRDL1 | SSVTN |
| n/a | CDRL2 | DTS |
| 76 | CDRL3 | FQGSGYPLT |
| 77 | VH | EVQLQQSGAALVRPGALVKLSCKSSGFNIRDYYIHWVKQRPE |
| | (CDRs in | QGLEWVGWIDPDNGDTMYDPKFQGKATLTADTYSNTAYLQ |
| | bold) | LSSLAFEDSAVYYCARSPIGPAHWGQGTLVTVSA |
| 78 | VL (κ) | ENVLTQSPAIMSASPGDKVTMTCSASSSVTNMHWYQQKSTTS |
| | (CDRs in | PKLWIYDTSKLASGVPGRFSGSGSGNSYSLTISSMEAEDVATY |
| | bold) | FCFQGSGYPLTFGGGTKLEIK |
| 79 | VH | GAGGTTCAACTGCAGCAGTCTGGGGCTGCGCTTGTGAGGCC |
| | | AGGGGCCTTAGTCAAGTTGTCCTGCAAATCTTCTGGCTTCA |
| | | ACATTAGAGACTACTATATACACTGGGTGAAGCAGAGGCC |
| | | TGAACAGGGCCTGGAGTGGGTTGGATGGATTGATCCTGAC |
| | | AATGGTGATACTATGTATGACCCGAAGTTCCAGGGCAAGG |
| | | CCACTTTAACAGCAGACACATATTCCAACACCGCCTACCTG |
| | | CAGCTCAGCAGCCTGGCATTCGAGGACAGTGCCGTCTATTA |
| | | CTGTGCTAGATCCCCAATTGGACCTGCTCACTGGGGCCAAG |
| | | GGACTCTGGTCACTGTCTCTGCAG |
| 80 | VL (κ) | GAAAATGTTCTCACCCAGTCTCCAGCAATCATGTCTGCATC |
| | | TCCAGGGGACAAGGTCACCATGACCTGTAGTGCCAGCTCA |
| | | AGTGTAACTAACATGCACTGGTACCAGCAGAAGTCAACCA |
| | | CCTCCCCCAAACTCTGGATTTATGACACATCCAAACTGGCT |
| | | TCTGGAGTCCCAGGTCGCTTCAGTGGCAGTGGGTCTGGAAA |
| | | CTCTTACTCTCTCACGATCAGCAGCATGGAGGCTGAAGATG |
| | | TTGCCACTTATTTCTGTTTTCAGGGGAGTGGGTACCCACTC |
| | | ACGTTCGGAGGGGGGACCAAGCTGGAAATAAAAC |
| 81 | Heavy | MKCSWVIFFLMAVVTGVNSEVQLQQSGAALVRPGALVKLS |
| | chain | CKSSGFNIRDYYIHWVKQRPEQGLEWVGWIDPDNGDTMY |
| | (VH in | DPKFQGKATLTADTYSNTAYLQLSSLAFEDSAVYYCARSPI |
| | bold; | GPAHWGQGTLVTVSAAKTTPPSVYPLAPGSAAQTNSMVTLG |
| | signal | CLVKGYFPEPVTVTWNSGSLSSGVHTFPAVLQSDLYTLSSSVT |
| | sequence | VPSSTWPSETVTCNVAHPASSTKVDKKIVPRDCGCKPCICTVP |
| | underlined) | EVSSVFIFPPKPKDVLTITLTPKVTCVVVDISKDDPEVQFSWFV |
| | | DDVEVHTAQTQPREEQFNSTFRSVSELPIMHQDWLNGKEFKC |
| | | RVNSAAFPAPIEKTISKTKGRPKAPQVYTIPPPKEQMAKDKVS |
| | | LTCMITDFFPEDITVEWQWNGQPAENYKNTQPIMDTDGSYFV |
| | | YSKLNVQKSNWEAGNTFTCSVLHEGLHNHHTEKSLSHSPGK |
| 82 | Light | MDFQVQIFSFLLISASVIMSRGENVLTQSPAIMSASPGDKVT |
| | chain | MTCSASSSVTNMHWYQQKSTTSPKLWIYDTSKLASGVPGR |
| | (VL in | FSGSGSGNSYSLTISSMEAEDVATYFCFQGSGYPLTFGGGT |
| | bold; | KLEIKRADAAPTVSTFPPSSEQLTSGGASVVCFLNNFYPKDIN |
| | signal | VKWKIDGSERQNGVLNSWTDQDSKDSTYSMSSTLTLTKDEY |
| | sequence | ERHNSYTCEATHKTSTSPIVKSFNRNEC |
| | underlined) | |
| 83 | Heavy | ATGAAATGCAGCTGGGTCATCTTCTTCCTGATGGCAGTGGT |
| | chain | TACAGGGGTCAATTCAGAGGTTCAACTGCAGCAGTCTGG |
| | (VH in | GGCTGCGCTTGTGAGGCCAGGGGCCTTAGTCAAGTTGT |
| | bold) | CCTGCAAATCTTCTGGCTTCAACATTAGAGACTACTATA |
| | | TACACTGGGTGAAGCAGAGGCCTGAACAGGGCCTGGAG |
| | | TGGGTTGGATGGATTGATCCTGACAATGGTGATACTAT |
| | | GTATGACCCGAAGTTCCAGGGCAAGGCCACTTTAACAG |
| | | CAGACACATATTCCAACACCGCCTACCTGCAGCTCAGC |
| | | AGCCTGGCATTCGAGGACAGTGCCGTCTATTACTGTGC |
| | | TAGATCCCCAATTGGACCTGCTCACTGGGGCCAAGGGA |
| | | CTCTGGTCACTGTCTCTGCAGCCAAAACGACACCCCCAT |
| | | CTGTCTATCCACTGGCCCCTGGATCTGCTGCCCAAACTAAC |
| | | TCCATGGTGACCCTGGGATGCCTGGTCAAGGGCTATTTCCC |
| | | TGAGCCAGTGACAGTGACCTGGAACTCTGGATCCCTGTCCA |
| | | GCGGTGTGCACACCTTCCCAGCTGTCCTGCAGTCTGACCTC |
| | | TACACTCTGAGCAGCTCAGTGACTGTCCCCTCCAGCACCTG |
| | | GCCCAGCGAGACCGTCACCTGCAACGTTGCCCACCCGGCC |
| | | AGCAGCACCAAGGTGGACAAGAAAATTGTGCCCAGGGATT |
| | | GTGGTTGTAAGCCTTGCATATGTACAGTCCCAGAAGTATCA |
| | | TCTGTCTTCATCTTCCCCCCAAAGCCCAAGGATGTGCTCAC |
| | | CATTACTCTGACTCCTAAGGTCACGTGTGTTGTGGTAGACA |
| | | TCAGCAAGGATGATCCCGAGGTCCAGTTCAGCTGGTTTGTA |
| | | GATGATGTGGAGGTGCACACAGCTCAGACGCAACCCCGGG |
| | | AGGAGCAGTTCAACAGCACTTTCCGCTCAGTCAGTGAACTT |
| | | CCCATCATGCACCAGGACTGGCTCAATGGCAAGGAGTTCA |
| | | AATGCAGGGTCAACAGTGCAGCTTTCCCTGCCCCCATCGAG |
| | | AAAACCATCTCCAAAACCAAAGGCAGACCGAAGGCTCCAC |
| | | AGGTGTACACCATTCCACCTCCCAAGGAGCAGATGGCCAA |
| | | GGATAAAGTCAGTCTGACCTGCATGATAACAGACTTCTTCC |
| | | CTGAAGACATTACTGTGGAGTGGCAGTGGAATGGGCAGCC |
| | | AGCGGAGAACTACAAGAACACTCAGCCCATCATGGACACA |
| | | GATGGCTCTTACTTCGTCTACAGCAAGCTCAATGTGCAGAA |
| | | GAGCAACTGGGAGGCAGGAAATACTTTCACCTGCTCTGTGT |
| | | TACATGAGGGCCTGCACAACCACCATACTGAGAAGAGCCT |
| | | CTCCCACTCTCCTGGTAAATGA |
| 84 | Light | ATGGATTTTCAAGTGCAGATTTTCAGCTTCCTGCTAATCAG |
| | chain (κ) | TGCCTCAGTCATAATGTCCAGAGGAGAAAATGTTCTCACC |
| | (VL in | CAGTCTCCAGCAATCATGTCTGCATCTCCAGGGGACAA |
| | bold) | GGTCACCATGACCTGTAGTGCCAGCTCAAGTGTAACTA |
| | | ACATGCACTGGTACCAGCAGAAGTCAACCACCTCCCCC |
| | | AAACTCTGGATTTATGACACATCCAAACTGGCTTCTGGA |
| | | GTCCCAGGTCGCTTCAGTGGCAGTGGGTCTGGAAACTC |
| | | TTACTCTCTCACGATCAGCAGCATGGAGGCTGAAGATG |
| | | TTGCCACTTATTTCTGTTTTCAGGGGAGTGGGTACCCAC |
| | | TCACGTTCGGAGGGGGGACCAAGCTGGAAATAAAACGG |
| | | GCTGATGCTGCACCAACTGTATCCACCTTCCCACCATCCAG |
| | | TGAGCAGTTAACATCTGGAGGTGCCTCAGTCGTGTGCTTCT |
| | | TGAACAACTTCTACCCCAAAGACATCAATGTCAAGTGGAA |
| | | GATTGATGGCAGTGAACGACAAAATGGCGTCCTGAACAGT |
| | | TGGACTGATCAGGACAGCAAAGACAGCACCTACAGCATGA |
| | | GCAGCACCCTCACGTTGACCAAGGACGAGTATGAACGACA |
| | | TAACAGCTATACCTGTGAGGCCACTCACAAGACATCAACTT |
| | | CACCCATTGTCAAGAGCTTCAACAGGAATGAGTGTTAG |

TABLE 18
CDR sequences of anti-GroEL2 antibody 103.11 (CDR nomenclature
using IMGT).

| SEQ | | |
|---|---|---|
| ID | | |
| NO: | Feature | Amino acid or nucleic acid sequence |
| 85 | CDRH1 | GFSLSTSGMG |
| 86 | CDRH2 | IYWDDDK |
| 87 | CDRH3 | ARTHYTYDVYAMDY |
| 88 | CRDL1 | SSVSSSY |
| n/a | CDRL2 | SAS |
| 89 | CDRL3 | HQYHRSPWT |
| 90 | VH | QVTLKESGPGILQPSQTLSLTCSFSGFSLSTSGMGVSWIRQPSG |
| | (CDRs in | KGLEWLAHIYWDDDKRYNPSLKSRLTISKDTSTNQVFLKITS |
| | bold) | VDTADTATYYCARTHYTYDVYAMDYWGQGTSVTVSS |
| 91 | VL (κ) | QIVLTQSPAIMSASLGERVTMTCTASSSVSSSYLHWYRQKPGS |
| | (CDRs in | SPKLWIYSASNLASGVPPRFSGSGSGTSYSLTISNMEAEDAAT |
| | bold) | YYCHQYHRSPWTFGGGTKLEIK |
| 92 | VH | CAGGTTACTCTGAAAGAGTCTGGCCCTGGGATATTGCAGCC |
| | | CTCCCAGACCCTCAGTCTGACTTGTTCTTTCTCTGGGTTTTC |
| | | ACTGAGCACTTCTGGTATGGGTGTGAGCTGGATTCGTCAGC |
| | | CTTCAGGAAAGGGTCTGGAGTGGCTGGCACACATTTACTGG |
| | | GATGATGACAAGCGCTATAACCCATCCCTGAAGAGCCGGC |
| | | TCACAATCTCCAAGGATACCTCCACAAACCAGGTATTCCTC |
| | | AAGATCACCAGTGTGGACACTGCAGATACTGCCACATACT |
| | | ACTGTGCTCGAACCCACTATACTTACGACGTTTATGCTATG |
| | | GACTACTGGGGTCAAGGAACCTCAGTCACCGTCTCCTCAG |
| 93 | VL (κ) | CAAATTGTTCTCACCCAGTCTCCAGCAATCATGTCTGCATC |
| | | TCTAGGGGAACGGGTCACCATGACCTGCACTGCCAGCTCA |
| | | AGTGTTAGTTCCAGTTACTTGCACTGGTACCGCCAGAAGCC |
| | | AGGATCCTCCCCCAAACTCTGGATTTATAGCGCATCCAACC |
| | | TGGCTTCTGGAGTCCCACCTCGCTTCAGTGGCAGTGGGTCT |
| | | GGGACCTCTTACTCTCTCACAATCAGCAACATGGAGGCTGA |
| | | AGATGCTGCCACTTATTACTGCCACCAGTATCATCGTTCCC |
| | | CGTGGACGTTCGGTGGAGGCACCAAGCTGGAAATCAAAC |
| 94 | Heavy | MDRLTSSFLLLIVPAYVLSQVTLKESGPGILQPSQTLSLTCSF |
| | chain | SGFSLSTSGMGVSWIRQPSGKGLEWLAHIYWDDDKRYNPS |
| | (VH in | LKSRLTISKDTSTNQVFLKITSVDTADTATYYCARTHYTYD |
| | bold; | VYAMDYWGQGTSVTVSSAKTTPPSVYPLAPGSAAQTNSMV |
| | signal | TLGCLVKGYFPEPVTVTWNSGSLSSGVHTFPAVLQSDLYTLSS |
| | sequence | SVTVPSSTWPSETVTCNVAHPASSTKVDKKIVPRDCGCKPCIC |
| | underlined) | TVPEVSSVFIFPPKPKDVLTITLTPKVTCVVVDISKDDPEVQFS |
| | | WFVDDVEVHTAQTQPREEQFNSTFRSVSELPIMHQDWLNGKE |
| | | FKCRVNSAAFPAPIEKTISKTKGRPKAPQVYTIPPPKEQMAKD |
| | | KVSLTCMITDFFPEDITVEWQWNGQPAENYKNTQPIMDTDGS |
| | | YFVYSKLNVQKSNWEAGNTFTCSVLHEGLHNHHTEKSLSHSP |
| | | GK |
| 95 | Light | MDFQVQIFSFLLISASVIMSRGQIVLTQSPAIMSASLGERVTM |
| | chain | TCTASSSVSSSYLHWYRQKPGSSPKLWIYSASNLASGVPPR |
| | (VL in | FSGSGSGTSYSLTISNMEAEDAATYYCHQYHRSPWTFGGG |
| | bold; | TKLEIKRADAAPTVSIFPPSSEQLTSGGASVVCFLNNFYPKDIN |
| | signal | VKWKIDGSERQNGVLNSWTDQDSKDSTYSMSSTLTLTKDEY |
| | sequence | ERHNSYTCEATHKTSTSPIVKSFNRNEC |
| | underlined) | |
| 96 | Heavy | ATGGACAGGCTTACTTCCTCATTCCTGCTGCTGATTGTCCCT |
| | chain | GCATATGTCCTTTCCCAGGTTACTCTGAAAGAGTCTGGCC |
| | (VH in | CTGGGATATTGCAGCCCTCCCAGACCCTCAGTCTGACTT |
| | bold) | GTTCTTTCTCTGGGTTTTCACTGAGCACTTCTGGTATGG |
| | | GTGTGAGCTGGATTCGTCAGCCTTCAGGAAAGGGTCTG |
| | | GAGTGGCTGGCACACATTTACTGGGATGATGACAAGCG |
| | | CTATAACCCATCCCTGAAGAGCCGGCTCACAATCTCCAA |
| | | GGATACCTCCACAAACCAGGTATTCCTCAAGATCACCA |
| | | GTGTGGACACTGCAGATACTGCCACATACTACTGTGCT |
| | | CGAACCCACTATACTTACGACGTTTATGCTATGGACTAC |
| | | TGGGGTCAAGGAACCTCAGTCACCGTCTCCTCAGCCAA |
| | | AACGACACCCCCATCTGTCTATCCACTGGCCCCTGGATCTG |
| | | CTGCCCAAACTAACTCCATGGTGACCCTGGGATGCCTGGTC |
| | | AAGGGCTATTTCCCTGAGCCAGTGACAGTGACCTGGAACTC |
| | | TGGATCCCTGTCCAGCGGTGTGCACACCTTCCCAGCTGTCC |
| | | TGCAGTCTGACCTCTACACTCTGAGCAGCTCAGTGACTGTC |
| | | CCCTCCAGCACCTGGCCCAGCGAGACCGTCACCTGCAACGT |
| | | TGCCCACCCGGCCAGCAGCACCAAGGTGGACAAGAAAATT |
| | | GTGCCCAGGGATTGTGGTTGTAAGCCTTGCATATGTACAGT |
| | | CCCAGAAGTATCATCTGTCTTCATCTTCCCCCCAAAGCCCA |
| | | AGGATGTGCTCACCATTACTCTGACTCCTAAGGTCACGTGT |
| | | GTTGTGGTAGACATCAGCAAGGATGATCCCGAGGTCCAGTT |
| | | CAGCTGGTTTGTAGATGATGTGGAGGTGCACACAGCTCAG |
| | | ACGCAACCCCGGGAGGAGCAGTTCAACAGCACTTTCCGCT |
| | | CAGTCAGTGAACTTCCCATCATGCACCAGGACTGGCTCAAT |
| | | GGCAAGGAGTTCAAATGCAGGGTCAACAGTGCAGCTTTCC |
| | | CTGCCCCCATCGAGAAAACCATCTCCAAAACCAAAGGCAG |
| | | ACCGAAGGCTCCACAGGTGTACACCATTCCACCTCCCAAGG |
| | | AGCAGATGGCCAAGGATAAAGTCAGTCTGACCTGCATGAT |
| | | AACAGACTTCTTCCCTGAAGACATTACTGTGGAGTGGCAGT |
| | | GGAATGGGCAGCCAGCGGAGAACTACAAGAACACTCAGCC |
| | | CATCATGGACACAGATGGCTCTTACTTCGTCTACAGCAAGC |
| | | TCAATGTGCAGAAGAGCAACTGGGAGGCAGGAAATACTTT |
| | | CACCTGCTCTGTGTTACATGAGGGCCTGCACAACCACCATA |
| | | CTGAGAAGAGCCTCTCCCACTCTCCTGGTAAATGA |
| 97 | Light | ATGGATTTTCAGGTGCAGATTTTCAGCTTCCTGCTAATCAG |
| | chain (κ) | TGCCTCAGTCATAATGTCCAGAGGACAAATTGTTCTCACC |
| | (VL in | CAGTCTCCAGCAATCATGTCTGCATCTCTAGGGGAACG |
| | bold) | GGTCACCATGACCTGCACTGCCAGCTCAAGTGTTAGTT |
| | | CCAGTTACTTGCACTGGTACCGCCAGAAGCCAGGATCC |
| | | TCCCCCAAACTCTGGATTTATAGCGCATCCAACCTGGCT |
| | | TCTGGAGTCCCACCTCGCTTCAGTGGCAGTGGGTCTGG |
| | | GACCTCTTACTCTCTCACAATCAGCAACATGGAGGCTGA |
| | | AGATGCTGCCACTTATTACTGCCACCAGTATCATCGTTC |
| | | CCCGTGGACGTTCGGTGGAGGCACCAAGCTGGAAATCA |
| | | AACGGGCTGATGCTGCACCAACTGTATCCATCTTCCCACCA |
| | | TCCAGTGAGCAGTTAACATCTGGAGGTGCCTCAGTCGTGTG |
| | | CTTCTTGAACAACTTCTACCCCAAAGACATCAATGTCAAGT |
| | | GGAAGATTGATGGCAGTGAACGACAAAATGGCGTCCTGAA |
| | | CAGTTGGACTGATCAGGACAGCAAAGACAGCACCTACAGC |
| | | ATGAGCAGCACCCTCACGTTGACCAAGGACGAGTATGAAC |
| | | GACATAACAGCTATACCTGTGAGGCCACTCACAAGACATC |
| | | AACTTCACCCATTGTCAAGAGCTTCAACAGGAATGAGTGTT |
| | | A |

TABLE 19
CDR sequences of anti-GroEL2 antibody 111.6 (CDR nomenclature
using IMGT).

| SEQ | | |
|---|---|---|
| ID | | |
| NO: | Feature | Amino acid or nucleic acid sequence |
| 98 | CDRH1 | GNSFTGHN |
| 99 | CDRH2 | VDPHYGSS |
| 100 | CDRH3 | ARSGRIHPLFFDV |
| 101 | CRDL1 | ESVDSYGIAF |
| n/a | CDRL2 | AAS |
| 102 | CDRL3 | QQTKEIPYT |
| 103 | VH | EVQLQQSGPELEKPGASVKISCKASGNSFTGHNINWVKQRNG |
| | (CDRs in | EALEWIGNVDPHYGSSDYNQKFKDKATLTADKSSSTAFLQFV |
| | bold) | SLTSEDSAVYYCARSGRIHPLFFDVWGAGTTVTVSS |
| 104 | VL (κ) | DIVLTQSPASLAVSLGQRATIFCRASESVDSYGIAFMNWFQQK |
| | (CDRs in | PGQPPKLLIYAASNQGSGVPARFSGSGSGTDFSLNIHPMEEDD |
| | bold) | TAMYFCQQTKEIPYTFGGGTRLEIK |
| 105 | VH | GAGGTCCAGCTGCAACAGTCTGGACCTGAGCTGGAGAAGC |
| | | CTGGCGCTTCAGTGAAGATATCCTGCAAGGCTTCTGGTAAC |
| | | TCATTCACTGGCCACAACATAAACTGGGTGAAACAAAGGA |
| | | ATGGAGAGGCCCTTGAGTGGATTGGAAATGTTGATCCCCAC |
| | | TATGGTAGTAGTGACTACAACCAGAAGTTTAAGGACAAGG |
| | | CCACATTGACTGCAGACAAATCCTCCAGCACAGCCTTCTTG |
| | | CAATTCGTGAGCCTGACATCTGAGGACTCTGCAGTCTATTA |
| | | CTGTGCAAGATCCGGACGAATTCATCCCTTATTCTTCGATG |
| | | TCTGGGGCGCCGGGACCACGGTCACCGTCTCCTCAG |
| 106 | VL (κ) | GACATTGTGCTGACCCAATCTCCAGCTTCTTTGGCTGTGTCT |
| | | CTAGGGCAGAGGGCCACCATCTTCTGCAGAGCCAGCGAAA |
| | | GTGTTGATAGTTATGGCATTGCTTTTATGAACTGGTTCCAA |
| | | CAGAAACCAGGACAGCCACCCAAACTCCTCATCTATGCTGC |
| | | ATCCAACCAAGGATCCGGGGTCCCTGCCAGGTTTAGTGGCA |
| | | GTGGGTCTGGGACAGACTTCAGCCTCAACATCCATCCTATG |
| | | GAGGAGGATGATACTGCAATGTATTTCTGTCAACAAACTAA |
| | | GGAGATTCCGTACACGTTCGGAGGCGGGACCAGGCTGGAA |
| | | ATAAAAC |
| 107 | Heavy | MGWTWIFILMLSVTTGVHSEVQLQQSGPELEKPGASVKISC |
| | chain | KASGNSFTGHNINWVKQRNGEALEWIGNVDPHYGSSDYN |
| | (VH in | QKFKDKATLTADKSSSTAFLQFVSLTSEDSAVYYCARSGRI |
| | bold; | HPLFFDVWGAGTTVTVSSAKATPPSVYPLAPGSAAQTNSMV |
| | signal | TLGCLVKGYFPVPVTVTWNSGSLSSGVHTFPAVLQSDLYTLSS |
| | sequence | SVTVPSSTWPSETVTCNVAHPASSTKVDKKIVPRDCGCKPCIC |
| | underlined) | TVPEVSSVFIFPPKPKDVLTITLTPKVTCVVVDISKDDPEVQFS |
| | | WFVDDVEVHTAQTQPREEQFNSTFRSVSELPIMHQDWLNGKE |
| | | FKCRVNSAAFPAPIEKTISKTKGRPKAPQVYTIPPPKEQMAKD |
| | | KVSLTCMITDFFPEDITVEWQWNGQPAENYKNTQPIMDTDGS |
| | | YFVYSKLNVQKSNWEAGNTFTCSVLHEGLHNHHTEKSLSHSP |
| | | GK |
| 108 | Light | MEKDTLLLWVLLLWVPGSTGDIVLTQSPASLAVSLGQRATI |
| | chain | FCRASESVDSYGIAFMNWFQQKPGQPPKLLIYAASNQGSG |
| | (VL in | VPARFSGSGSGTDFSLNIHPMEEDDTAMYFCQQTKEIPYTF |
| | bold; | GGGTRLEIKRADAAPTVSIFPPSSEQLTSGGASVVCFLNNFYP |
| | signal | KDINVKWKIDGSERQNGVLNSWTDQDSKDSTYSMSSTLTLTK |
| | sequence | DEYERHNSYTCEATHKTSTSPIVKSFNRNEC |
| | underlined) | |
| 109 | Heavy | ATGGGATGGACCTGGATCTTTATTTTAATGCTGTCAGTCAC |
| | chain | TACAGGTGTCCACTCTGAGGTCCAGCTGCAACAGTCTGG |
| | (VH in | ACCTGAGCTGGAGAAGCCTGGCGCTTCAGTGAAGATAT |
| | bold) | CCTGCAAGGCTTCTGGTAACTCATTCACTGGCCACAACA |
| | | TAAACTGGGTGAAACAAAGGAATGGAGAGGCCCTTGAG |
| | | TGGATTGGAAATGTTGATCCCCACTATGGTAGTAGTGA |
| | | CTACAACCAGAAGTTTAAGGACAAGGCCACATTGACTG |
| | | CAGACAAATCCTCCAGCACAGCCTTCTTGCAATTCGTGA |
| | | GCCTGACATCTGAGGACTCTGCAGTCTATTACTGTGCAA |
| | | GATCCGGACGAATTCATCCCTTATTCTTCGATGTCTGGG |
| | | GCGCCGGGACCACGGTCACCGTCTCCTCAGCCAAAGCG |
| | | ACACCCCCATCTGTCTATCCACTGGCCCCTGGATCTGCTGC |
| | | CCAAACTAACTCCATGGTGACCCTGGGATGCCTGGTCAAGG |
| | | GCTATTTCCCTGTGCCAGTGACAGTGACCTGGAACTCTGGA |
| | | TCCCTGTCCAGCGGTGTGCACACCTTCCCAGCTGTCCTGCA |
| | | GTCTGACCTCTACACTCTGAGCAGCTCAGTGACTGTCCCCT |
| | | CCAGCACCTGGCCCAGCGAGACCGTCACCTGCAACGTTGCC |
| | | CACCCGGCCAGCAGCACCAAGGTGGACAAGAAAATTGTGC |
| | | CCAGGGATTGTGGTTGTAAGCCTTGCATATGTACAGTCCCA |
| | | GAAGTATCATCTGTCTTCATCTTCCCCCCAAAGCCCAAGGA |
| | | TGTGCTCACCATTACTCTGACTCCTAAGGTCACGTGTGTTGT |
| | | GGTAGACATCAGCAAGGATGATCCCGAGGTCCAGTTCAGC |
| | | TGGTTTGTAGATGATGTGGAGGTGCACACAGCTCAGACGC |
| | | AACCCCGGGAGGAGCAGTTCAACAGCACTTTCCGCTCAGTC |
| | | AGTGAACTTCCCATCATGCACCAGGACTGGCTCAATGGCAA |
| | | GGAGTTCAAATGCAGGGTCAACAGTGCAGCTTTCCCTGCCC |
| | | CCATCGAGAAAACCATCTCCAAAACCAAAGGCAGACCGAA |
| | | GGCTCCACAGGTGTACACCATTCCACCTCCCAAGGAGCAG |
| | | ATGGCCAAGGATAAAGTCAGTCTGACCTGCATGATAACAG |
| | | ACTTCTTCCCTGAAGACATTACTGTGGAGTGGCAGTGGAAT |
| | | GGGCAGCCAGCGGAGAACTACAAGAACACTCAGCCCATCA |
| | | TGGACACAGATGGCTCTTACTTCGTCTACAGCAAGCTCAAT |
| | | GTGCAGAAGAGCAACTGGGAGGCAGGAAATACTTTCACCT |
| | | GCTCTGTGTTACATGAGGGCCTGCACAACCACCATACTGAG |
| | | AAGAGCCTCTCCCACTCTCCTGGTAAATGA |
| 110 | Light | ATGGAGAAAGACACACTCCTGCTATGGGTCCTGCTTCTCTG |
| | chain (κ) | GGTTCCAGGTTCCACAGGTGACATTGTGCTGACCCAATCT |
| | (VL in | CCAGCTTCTTTGGCTGTGTCTCTAGGGCAGAGGGCCAC |
| | bold) | CATCTTCTGCAGAGCCAGCGAAAGTGTTGATAGTTATG |
| | | GCATTGCTTTTATGAACTGGTTCCAACAGAAACCAGGAC |
| | | AGCCACCCAAACTCCTCATCTATGCTGCATCCAACCAAG |
| | | GATCCGGGGTCCCTGCCAGGTTTAGTGGCAGTGGGTCT |
| | | GGGACAGACTTCAGCCTCAACATCCATCCTATGGAGGA |
| | | GGATGATACTGCAATGTATTTCTGTCAACAAACTAAGGA |
| | | GATTCCGTACACGTTCGGAGGCGGGACCAGGCTGGAAA |
| | | TAAAACGGGCTGATGCTGCACCAACTGTATCCATCTTCCCA |
| | | CCATCCAGTGAGCAGTTAACATCTGGAGGTGCCTCAGTCGT |
| | | GTGCTTCTTGAACAACTTCTACCCCAAAGACATCAATGTCA |
| | | AGTGGAAGATTGATGGCAGTGAACGACAAAATGGCGTCCT |
| | | GAACAGTTGGACTGATCAGGACAGCAAAGACAGCACCTAC |
| | | AGCATGAGCAGCACCCTCACGTTGACCAAGGACGAGTATG |
| | | AACGACATAACAGCTATACCTGTGAGGCCACTCACAAGAC |
| | | ATCAACTTCACCCATTGTCAAGAGCTTCAACAGGAATGAGT |
| | | GTTA |

TABLE 20
CDR sequences of anti-GroEL2 antibody 238.11 (CDR nomenclature
using IMGT).

| SEQ | | |
|---|---|---|
| ID | | |
| NO: | Feature | Amino acid or nucleic acid sequence |
| 111 | CDRH1 | GFTFSTYV |
| 112 | CDRH2 | ISSGGFYT |
| 113 | CDRH3 | ARLTAAPYYFDF |
| 114 | CRDL1 | SSVSSSN |
| n/a | CDRL2 | GTS |
| 115 | CDRL3 | QQWSSYPWT |
| 116 | VH | EVMLVESGGGLVKPGGSLKLSCAASGFTFSTYVMSWVRQTP |
| | (CDRs in | EKKLEWVATISSGGFYTYYPDSVKGRFTISRDNAENTLFLQV |
| | bold) | RSLRSEDTAMYYCARLTAAPYYFDFWGQGTSLTVSS |
| 117 | VL (κ) | EIVLTQSPALVAASPGEKVTITCSVSSSVSSSNLHWYQQKSETS |
| | (CDRs in | PKPWIYGTSNLASGVPTRFSGSGSGTSYSLTISSMEAEDAATY |
| | bold) | YCQQWSSYPWTFGGGTKVEIK |
| 118 | VH | GAAGTGATGCTGGTGGAGTCTGGGGGAGGCTTAGTGAAGC |
| | | CTGGAGGGTCCCTGAAACTCTCCTGTGCAGCCTCTGGATTC |
| | | ACTTTCAGTACCTATGTCATGTCTTGGGTTCGCCAGACTCC |
| | | GGAGAAGAAGCTGGAGTGGGTCGCAACCATTAGTAGTGGT |
| | | GGTTTTTACACCTACTATCCAGACAGTGTGAAGGGGCGTTT |
| | | CACCATCTCCAGAGACAATGCCGAGAACACCCTGTTCCTGC |
| | | AAGTGCGCAGTCTGAGGTCTGAGGACACGGCCATGTATTA |
| | | CTGTGCAAGACTTACTGCGGCCCCTTACTACTTTGACTTCTG |
| | | GGGCCAAGGCACCTCTCTCACAGTCTCCTCAG |
| 119 | VL (κ) | GAAATTGTACTCACCCAATCTCCAGCACTCGTGGCTGCATC |
| | | TCCAGGGGAGAAGGTCACCATCACCTGCAGTGTCAGCTCA |
| | | AGTGTAAGTTCCAGCAACTTACACTGGTACCAGCAGAAGTC |
| | | AGAAACTTCCCCCAAACCCTGGATTTATGGCACATCCAACC |
| | | TGGCTTCTGGAGTCCCTACTCGCTTCAGTGGCAGTGGATCT |
| | | GGGACCTCTTATTCTCTCACAATCAGCAGCATGGAGGCTGA |
| | | AGATGCTGCCACTTATTACTGTCAACAGTGGAGTAGTTACC |
| | | CGTGGACGTTCGGTGGAGGCACCAAGGTGGAAATCAAAC |
| 120 | Heavy | MNFGLSLIFLVLVLKGVQCEVMLVESGGGLVKPGGSLKLSC |
| | chain | AASGFTFSTYVMSWVRQTPEKKLEWVATISSGGFYTYYPD |
| | (VH in | SVKGRFTISRDNAENTLFLQVRSLRSEDTAMYYCARLTAA |
| | bold; | PYYFDFWGQGTSLTVSSAKTTPPSVYPLAPGSAAQTNSMVT |
| | signal | LGCLVKGYFPEPVTVTWNSGSLSSGVHTFPAVLQSDLYTLSSS |
| | sequence | VTVPSSTWPSETVTCNVAHPASSTKVDKKIVPRDCGCKPCICT |
| | underlined) | VPEVSSVFIFPPKPKDVLTITLTPKVACVVVDISKDDPEVQFSW |
| | | FVDDVEVHTAQTQPREEQFNSTFRSVSELPIMHQDWLNGKEF |
| | | KCRVNSAAFPAPIEKTISKTKGRPKAPQVYTIPPPKEQMAKDK |
| | | VSLTCMITDFFPEDITVEWQWNGQPAENYKNTQPIMDTDGSY |
| | | FVYSKLNVQKSNWEAGNTFTCSVLHEGLHNHHTEKSLSHSPG |
| | | K |
| 121 | Light | DFHVQIFSFMLISVTVTLSSGEIVLTQSPALVAASPGEKVTIT |
| | chain | CSVSSSVSSSNLHWYQQKSETSPKPWIYGTSNLASGVPTRF |
| | (VL in | SGSGSGTSYSLTISSMEAEDAATYYCQQWSSYPWTFGGGT |
| | bold; | KVEIKRADAAPTVSIFPPSSEQLTSGGASVVCFLNNFYPKDINV |
| | signal | KWKIDGSERQNGVLNSWTDQDSKDSTYSMSSTLTLTKDEYER |
| | sequence | HNSYTCEATHKTSTSPIVKSFNRNEC |
| | underlined) | |
| 122 | Heavy | ATGAACTTCGGGCTCAGCTTGATTTTCCTTGTCCTTGTTTTA |
| | chain | AAAGGTGTCCAGTGTGAAGTGATGCTGGTGGAGTCTGGG |
| | (VH in | GGAGGCTTAGTGAAGCCTGGAGGGTCCCTGAAACTCTC |
| | bold) | CTGTGCAGCCTCTGGATTCACTTTCAGTACCTATGTCAT |
| | | GTCTTGGGTTCGCCAGACTCCGGAGAAGAAGCTGGAGT |
| | | GGGTCGCAACCATTAGTAGTGGTGGTTTTTACACCTACT |
| | | ATCCAGACAGTGTGAAGGGGCGTTTCACCATCTCCAGA |
| | | GACAATGCCGAGAACACCCTGTTCCTGCAAGTGCGCAG |
| | | TCTGAGGTCTGAGGACACGGCCATGTATTACTGTGCAA |
| | | GACTTACTGCGGCCCCTTACTACTTTGACTTCTGGGGCC |
| | | AAGGCACCTCTCTCACAGTCTCCTCAGCCAAAACGACAC |
| | | CCCCATCTGTCTATCCACTGGCCCCTGGATCTGCTGCCCAA |
| | | ACTAACTCCATGGTGACCCTGGGATGCCTGGTCAAGGGCTA |
| | | TTTCCCTGAGCCAGTGACAGTGACCTGGAACTCTGGATCCC |
| | | TGTCCAGCGGTGTGCACACCTTCCCAGCTGTCCTGCAGTCT |
| | | GACCTCTACACTCTGAGCAGCTCAGTGACTGTCCCCTCCAG |
| | | CACCTGGCCCAGCGAGACCGTCACCTGCAACGTTGCCCACC |
| | | CGGCCAGCAGCACCAAGGTGGACAAGAAAATTGTGCCCAG |
| | | GGATTGTGGTTGTAAGCCTTGCATATGTACAGTCCCAGAAG |
| | | TATCATCTGTCTTCATCTTCCCCCCAAAGCCCAAGGATGTG |
| | | CTCACCATTACTCTGACTCCTAAGGTCGCGTGTGTTGTGGT |
| | | AGACATCAGCAAGGATGATCCCGAGGTCCAGTTCAGCTGG |
| | | TTTGTAGATGATGTGGAGGTGCACACAGCTCAGACGCAAC |
| | | CCCGGGAGGAGCAGTTCAACAGCACTTTCCGCTCAGTCAGT |
| | | GAACTTCCCATCATGCACCAGGACTGGCTCAATGGCAAGG |
| | | AGTTCAAATGCAGGGTCAACAGTGCAGCTTTCCCTGCCCCC |
| | | ATCGAGAAAACCATCTCCAAAACCAAAGGCAGACCGAAGG |
| | | CTCCACAGGTGTACACCATTCCACCTCCCAAGGAGCAGATG |
| | | GCCAAGGATAAAGTCAGTCTGACCTGCATGATAACAGACT |
| | | TCTTCCCTGAAGACATTACTGTGGAGTGGCAGTGGAATGGG |
| | | CAGCCAGCGGAGAACTACAAGAACACTCAGCCCATCATGG |
| | | ACACAGATGGCTCTTACTTCGTCTACAGCAAGCTCAATGTG |
| | | CAGAAGAGCAACTGGGAGGCAGGAAATACTTTCACCTGCT |
| | | CTGTGTTACATGAGGGCCTGCACAACCACCATACTGAGAA |
| | | GAGCCTCTCCCACTCTCCTGGTAAATGA |
| 123 | Light | ATGGATTTTCATGTGCAGATTTTCAGCTTCATGCTAATCAGT |
| | chain (κ) | GTCACAGTCACATTGTCCAGTGGAGAAATTGTACTCACCC |
| | (VL in | AATCTCCAGCACTCGTGGCTGCATCTCCAGGGGAGAAG |
| | bold) | GTCACCATCACCTGCAGTGTCAGCTCAAGTGTAAGTTCC |
| | | AGCAACTTACACTGGTACCAGCAGAAGTCAGAAACTTC |
| | | CCCCAAACCCTGGATTTATGGCACATCCAACCTGGCTTC |
| | | TGGAGTCCCTACTCGCTTCAGTGGCAGTGGATCTGGGA |
| | | CCTCTTATTCTCTCACAATCAGCAGCATGGAGGCTGAAG |
| | | ATGCTGCCACTTATTACTGTCAACAGTGGAGTAGTTACC |
| | | CGTGGACGTTCGGTGGAGGCACCAAGGTGGAAATCAAA |
| | | CGGGCTGATGCTGCACCAACTGTATCCATCTTCCCACCATC |
| | | CAGTGAGCAGTTAACATCTGGAGGTGCCTCAGTCGTGTGCT |
| | | TCTTGAACAACTTCTACCCCAAAGACATCAATGTCAAGTGG |
| | | AAGATTGATGGCAGTGAACGACAAAATGGCGTCCTGAACA |
| | | GTTGGACTGATCAGGACAGCAAAGACAGCACCTACAGCAT |
| | | GAGCAGCACCCTCACGTTGACCAAGGACGAGTATGAACGA |
| | | CATAACAGCTATACCTGTGAGGCCACTCACAAGACATCAA |
| | | CTTCACCCATCGTCAAGAGCTTCAACAGGAATGAGTGTTAG |

### Example 9: Isolated Anti-GroEL2 Antibodies Bind to GroEL2, Mtb Fractions, and Whole Mycobacterial Cells

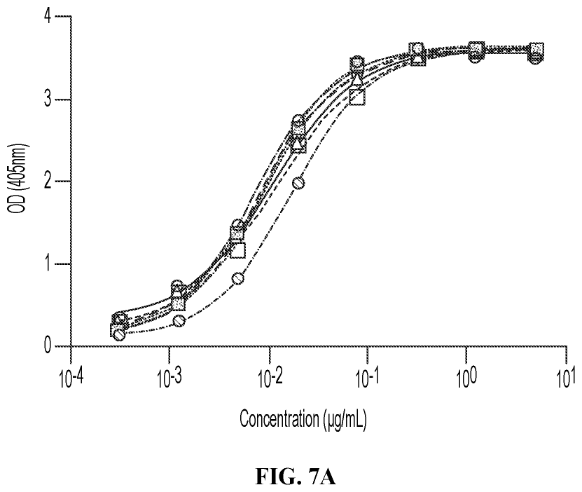

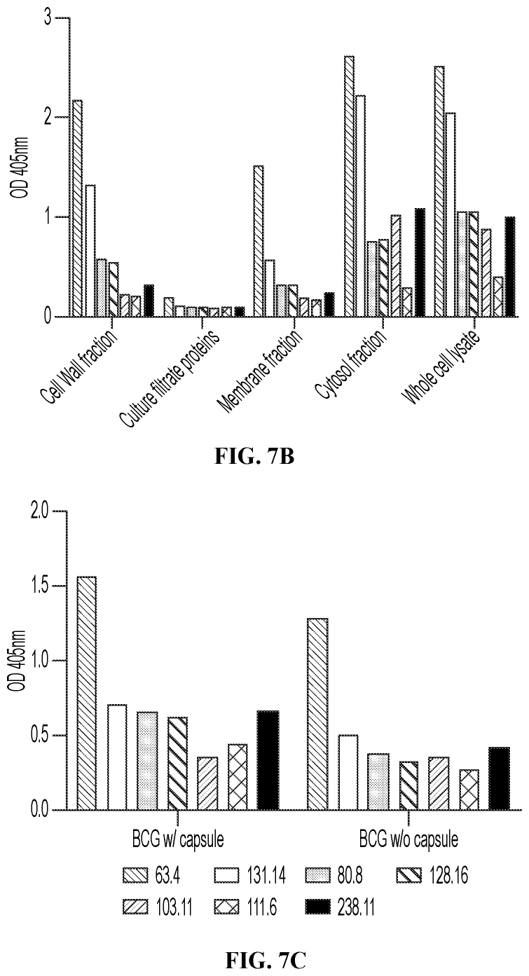

ELISA experiments were used to determine binding and estimate affinity of mAbs to recombinant Mtb protein GroEL2, Mtb fractions, as well whole cells of BCG. Antibodies were titrated against Mtb proteins (4 g/ml) of H37Rv (FIGS. 7A and B) or whole cells of BCG (FIG. 7C) grown in 7H9 with or without detergent. Bacterial cells were coated at 2×10⁷ bacteria per well based on GD value. Serial diluted mAbs were applied as primary antibodies and the binding was detected using an AP-conjugated anti-mouse IgG.

Anti-GroEL2 antibodies, including antibodies 63.4, 80.8, 103.11, 111.6, and 238.11, bound to recombinant GroEL2 (FIG. 7A), different Mtb fractions (FIG. 71B), and whole cells of M. bovis Bacillus Calmette-Guerin (BCG) (FIG. 7C).

### Example 10: Competition Assays with Isolated Anti-GroEL2 Antibodies

To obtain more information on the binding epitopes in GroEL2 for the isolated anti-GroEL2 antibodies, a competition assay for binding to GroEL2 was performed. Biotinylated anti-GroEL2 mAb (0.04 μg/ml) and serial diluted competing mAb were added to GroEL2 coated (4 μg/ml) plates the same time. The binding of biotinylated mAb to GroEL2 was detected by HIRP-conjugated streptavidin.

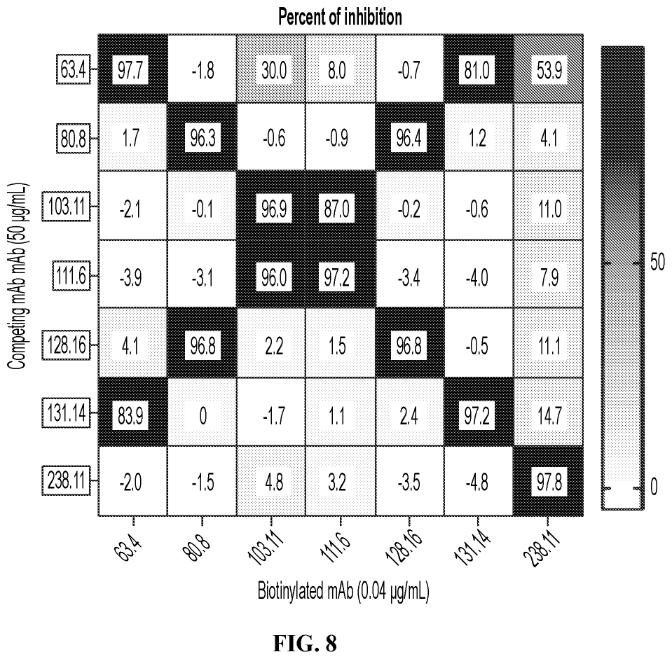

The competition assay revealed that the following groups of antibodies bound similar antibodies, that were different from the epitopes of other antibodies: (1) 63.4 and 131.14; (2) 80.8 and 128.16; (3) 103.11 and 111.6. Antibody 238.11 had a unique epitope and did not compete with any of the other antibodies for binding to GroEL2 (FIG. 8).

### Example 11: Polyclonal Anti-GroEL2 Antibody Reduces Mycobacterial Burden in Mtb-Infected Mice

It was demonstrated that the passive transfer of a purified human anti-GroEL2 polyclonal IgG reduces lung mycobacterial burden in Mtb-infected mice. For this, FcγR-humanized mice were treated i.p. with (1) normal saline (NS), (2) 20 μg control human IgG, or (3) 20 μg purified polyclonal anti-GroEL2 IgG isolated from two volunteers. After 24 hrs, the mice were then subjected to an aerosolized low-dose of Mtb, resulting in infection (Erdman; mean lung colony forming units (CFU) 60 one day post infection). Seven days after the infection, the mice were treated with (1) NS, (2) 10 μg control human IgG, or (3) 10 μg purified polyclonal anti-GroEL2 IgG. Lung CFU (median and interquartile) 2 weeks post infection were determined.

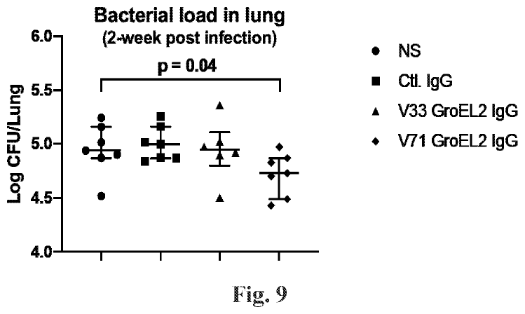

Mice receiving treatment with anti-Groel2 polyclonal IgG from volunteer V71 showed a decreased number of Mtb CFU as compared to the control (FIG. 9).

### Example 12: Isolation of an Anti-LprG Antibody

Recombinant Mtb protein LprG was produced and purified using the Mycobacterium smegmatis expression system. The plasmid containing the sequence of Mtb LprG protein followed by a polyhistidine tag was transformed to M. smegmatis. Single colonies were picked for small scale culture to screen positive colonies. Bacteria were then grown in a large scale. Recombinant protein was isolated by Ni-NTA (nickel-nitrilotriacetic acid) agarose from cell lysate, and further purified by a size exclusion chromatography. Five Balb/c mice (females, 6 weeks old) were immunized each with 100 ug of recombinant LprG intraperitoneal with a booster of 100 ug of recombinant LprG intraperitoneal after 4 weeks. Mice were bled 3 weeks after first and second dose to determine anti-LprG antibody titers. IgG titers to LprG after second immunization were high and mice were sacrificed. Antigen specific B-cells from the spleen of the immunized mice were fused with myeloma cells to produce hybridoma cells. After screening, one clone from each hybridoma was selected for further study based on high antibody titer in culture supernatant. The sequence for the highest affinity mAb to LprG (antibody 469.8) is shown in Table 21.

TABLE 21
CDR sequences of anti-LprG antibody 469.8 (CDR nomenclature
using IMGT).

| SEQ | | |
|---|---|---|
| ID | | |
| NO: | Feature | Amino acid or nucleic acid sequence |
| 124 | CDRH1 | GYTFTEYT |
| 125 | CDRH2 | INPNNGGS |
| 126 | CDRH3 | ASNGGY |
| 127 | CRDL1 | QSLLDSDGKTY |
| n/a | CDRL2 | LVS |
| 128 | CDRL3 | WQGTHYPWT |
| 129 | VH | EVQLQQSGPELVKPGASVKISCKTSGYTFTEYTMHWVKQSH |
| | (CDRs in | GKSLEWIGGINPNNGGSSYNQKFKGKATLTVNKSSSTAYMEL |
| | bold) | RSLTSEDSAVYYCASNGGYWGQGTTLTVSS |
| 130 | VL (κ) | DVVMTQTPLTLSVTIGQPASISCKSSQSLLDSDGKTYLNWLL |
| | (CDRs in | QRPGQSPERLIYLVSKLDSGVPDRFTGSGSGTDFTLKISRVEAE |
| | bold) | DLGVYHCWQGTHYPWTFGGGTKLEIK |
| 131 | VH | GAGGTCCAGCTGCAACAGTCTGGACCTGAGCTGGTGAAGC |
| | | CTGGGGCTTCAGTGAAGATATCCTGCAAGACTTCTGGATAC |
| | | ACATTCACTGAATACACCATGCACTGGGTGAAGCAGAGCC |
| | | ATGGAAAGAGCCTTGAGTGGATTGGAGGTATTAATCCTAA |
| | | CAATGGTGGTTCTAGCTACAACCAGAAGTTCAAGGGCAAG |
| | | GCCACATTGACTGTAAACAAGTCCTCCAGCACAGCCTACAT |
| | | GGAGCTCCGCAGCCTGACATCTGAGGATTCTGCAGTCTATT |
| | | ACTGTGCAAGCAATGGGGGCTACTGGGGCCAAGGCACCAC |
| | | TCTCACAGTCTCCTCAG |
| 132 | VL (κ) | GATGTTGTGATGACCCAGACTCCACTCACTTTGTCGGTTAC |
| | | CATTGGACAACCAGCCTCCATCTCTTGCAAGTCAAGTCAGA |
| | | GCCTCTTAGATAGTGATGGAAAGACATATTTGAATTGGTTG |
| | | TTACAGAGGCCAGGCCAGTCTCCAGAGCGCCTAATCTATCT |
| | | GGTGTCTAAATTGGACTCTGGAGTCCCTGACAGGTTCACTG |
| | | GCAGTGGCTCAGGGACAGATTTCACACTGAAAATTAGCAG |
| | | AGTGGAGGCTGAGGATTTGGGAGTTTATCATTGCTGGCAAG |
| | | GTACACATTATCCGTGGACGTTCGGTGGAGGCACCAAGCTG |
| | | GAAATCAAAC |
| 133 | Heavy | MGWSWIFLELLSGTAGVLSEVQLQQSGPELVKPGASVKISC |
| | chain | KTSGYTFTEYTMHWVKQSHGKSLEWIGGINPNNGGSSYN |
| | (VH in | QKFKGKATLTVNKSSSTAYMELRSLTSEDSAVYYCASNGG |
| | bold; | YWGQGTTLTVSSAKTTPPSVYPLAPGSAAQTNSMVTLGCLV |
| | signal | KGYFPEPVTVTWNSGSLSSGVHTFPAVLQSDLYTLSSSVTVPS |
| | sequence | STWPSETVTCNVAHPASSTKVDKKIVPRDCGCKPCICTVPEVS |
| | 128 | SVFIFPPKPKDVLTITLTPKVTCVVVDISKDDPEVQFSWFVDDV |
| | nderline) | EVHTAQTQPREEQFNSTFRSVSELPIMHQDWLNGKEFKCRVN |
| | | SAAFPAPIEKTISKTKGRPKAPQVYTIPPPKEQMAKDKVSLTC |
| | | MITDFFPEDITVEWQWNGQPAENYKNTQPIMDTDGSYFVYSK |
| | | LNVQKSNWEAGNTFTCSVLHEGLHNHHTEKSLSHSPGK |
| 134 | Light | MMSPAQFLFLLVLWIREINGDVVMTQTPLTLSVTIGQPASIS |
| | chain | CKSSQSLLDSDGKTYLNWLLQRPGQSPERLIYLVSKLDSG |
| | (VL in | VPDRFTGSGSGTDFTLKISRVEAEDLGVYHCWQGTHYPW |
| | bold; | TFGGGTKLEIKRADAAPTVSIFPPSSEQLTSGGASVVCFLNNF |
| | signal | YPKDINVKWKIDGSERQNGVLNSWTDQDSKDSTYSMSSTLTL |
| | sequence | TKDEYERHNSYTCEATHKTSTSPIVKSFNRNEC |
| | underlined) | |
| 135 | Heavy | ATGGGATGGAGCTGGATCTTTCTCTTTCTCCTGTCAGGAAC |
| | chain | TGCAGGTGTCCTCTCTGAGGTCCAGCTGCAACAGTCTGG |
| | (VH in | ACCTGAGCTGGTGAAGCCTGGGGCTTCAGTGAAGATAT |
| | bold) | CCTGCAAGACTTCTGGATACACATTCACTGAATACACCA |
| | | TGCACTGGGTGAAGCAGAGCCATGGAAAGAGCCTTGAG |
| | | TGGATTGGAGGTATTAATCCTAACAATGGTGGTTCTAGC |
| | | TACAACCAGAAGTTCAAGGGCAAGGCCACATTGACTGT |
| | | AAACAAGTCCTCCAGCACAGCCTACATGGAGCTCCGCA |
| | | GCCTGACATCTGAGGATTCTGCAGTCTATTACTGTGCAA |
| | | GCAATGGGGGCTACTGGGGCCAAGGCACCACTCTCACA |
| | | GTCTCCTCAGCCAAAACGACACCCCCATCTGTCTATCCAC |
| | | TGGCCCCTGGATCTGCTGCCCAAACTAACTCCATGGTGACC |
| | | CTGGGATGCCTGGTCAAGGGCTATTTCCCTGAGCCAGTGAC |
| | | AGTGACCTGGAACTCTGGATCCCTGTCCAGCGGTGTGCACA |
| | | CCTTCCCAGCTGTCCTGCAGTCTGACCTCTACACTCTGAGC |
| | | AGCTCAGTGACTGTCCCCTCCAGCACCTGGCCCAGCGAGAC |
| | | CGTCACCTGCAACGTTGCCCACCCGGCCAGCAGCACCAAG |
| | | GTGGACAAGAAAATTGTGCCCAGGGATTGTGGTTGTAAGC |
| | | CTTGCATATGTACAGTCCCAGAAGTATCATCTGTCTTCATCT |
| | | TCCCCCCAAAGCCCAAGGATGTGCTCACCATTACTCTGACT |
| | | CCTAAGGTCACGTGTGTTGTGGTAGACATCAGCAAGGATG |
| | | ATCCCGAGGTCCAGTTCAGCTGGTTTGTAGATGATGTGGAG |
| | | GTGCACACAGCTCAGACGCAACCCCGGGAGGAGCAGTTCA |
| | | ACAGCACTTTCCGCTCAGTCAGTGAACTTCCCATCATGCAC |
| | | CAGGACTGGCTCAATGGCAAGGAGTTCAAATGCAGGGTCA |
| | | ACAGTGCAGCTTTCCCTGCCCCCATCGAGAAAACCATCTCC |
| | | AAAACCAAAGGCAGACCGAAGGCTCCACAGGTGTACACCA |
| | | TTCCACCTCCCAAGGAGCAGATGGCCAAGGATAAAGTCAG |
| | | TCTGACCTGCATGATAACAGACTTCTTCCCTGAAGACATTA |
| | | CTGTGGAGTGGCAGTGGAATGGGCAGCCAGCGGAGAACTA |
| | | CAAGAACACTCAGCCCATCATGGACACAGATGGCTCTTACT |
| | | TCGTCTACAGCAAGCTCAATGTGCAGAAGAGCAACTGGGA |
| | | GGCAGGAAATACTTTCACCTGCTCTGTGTTACATGAGGGCC |
| | | TGCACAACCACCATACTGAGAAGAGCCTCTCCCACTCTCCT |
| | | GGTAAATGA |
| 136 | Light | ATGATGAGTCCTGCCCAGTTCCTGTTTCTGTTAGTGCTCTGG |
| | chain (κ) | ATTCGGGAAACCAACGGTGATGTTGTGATGACCCAGACT |
| | (VL in | CCACTCACTTTGTCGGTTACCATTGGACAACCAGCCTCC |
| | bold) | ATCTCTTGCAAGTCAAGTCAGAGCCTCTTAGATAGTGAT |
| | | GGAAAGACATATTTGAATTGGTTGTTACAGAGGCCAGG |
| | | CCAGTCTCCAGAGCGCCTAATCTATCTGGTGTCTAAATT |
| | | GGACTCTGGAGTCCCTGACAGGTTCACTGGCAGTGGCT |
| | | CAGGGACAGATTTCACACTGAAAATTAGCAGAGTGGAG |
| | | GCTGAGGATTTGGGAGTTTATCATTGCTGGCAAGGTAC |
| | | ACATTATCCGTGGACGTTCGGTGGAGGCACCAAGCTGG |
| | | AAATCAAACGGGCTGATGCTGCACCAACTGTATCCATCTTC |
| | | CCACCATCCAGTGAGCAGTTAACATCTGGAGGTGCCTCAGT |
| | | CGTGTGCTTCTTGAACAACTTCTACCCCAAAGACATCAATG |
| | | TCAAGTGGAAGATTGATGGCAGTGAACGACAAAATGGCGT |
| | | CCTGAACAGTTGGACTGATCAGGACAGCAAAGACAGCACC |
| | | TACAGCATGAGCAGCACCCTCACGTTGACCAAGGACGAGT |
| | | ATGAACGACATAACAGCTATACCTGTGAGGCCACTCACAA |
| | | GACATCAACTTCACCCATTGTCAAGAGCTTCAACAGGAATG |
| | | AGTGTTAG |

Tables 22 and 23 provide an overview of exemplary anti-AM antibody sequences disclosed herein.

Tables 24 and 25 provide an overview of exemplary anti-GroEL2 antibody sequences disclosed herein.

Tables 25 and 27 provide an overview of exemplary anti-LprG antibody sequences disclosed herein.

TABLE 22
Overview amino acid sequences for anti-AM antibodies.

| Type of | T2 | T2 | T2 | A1 | T1 | A2 | A2 |
|---|---|---|---|---|---|---|---|
| sequence | AM02 | AM11 | AM15 | AM23 | AM65 | AM30 | AM10 |
| CDRH1 | 1 | 10 | 19 | 28 | 37 | 46 | 161 |
| CDRH2 | 2 | 11 | 20 | 29 | 38 | 47 | 162 |
| CDRH3 | 3 | 12 | 21 | 30 | 39 | 48 | 163 |
| CRDL1 | 4 | 13 | 22 | 31 | 40 | 49 | 164 |
| CDRL2 | AVT | DVT | KIS | GAS | EVS | KIS | KSD |
| CDRL3 | 5 | 14 | 23 | 32 | 41 | 50 | 165 |
| VH | 6 | 15 | 24 | 33 | 42 | 51 | 166 |
| VL (λ) | 7 | 16 | n/a | n/a | n/a | n/a | 167 |
| VL (κ) | n/a | n/a | 25 | 34 | 43 | 52 | n/a |

TABLE 23
Overview nucleic acid sequences for anti-AM antibodies.

| Type of | T2 | T2 | T2 | A1 | T1 | A2 | A2 |
|---|---|---|---|---|---|---|---|
| sequence | AM02 | AM11 | AM15 | AM23 | AM65 | AM30 | AM10 |
| VH | 8 | 17 | 26 | 35 | 44 | 53 | 168 |
| VL (λ) | 9 | 18 | n/a | n/a | n/a | n/a | 169 |
| VL (κ) | n/a | n/a | 27 | 36 | 45 | 54 | n/a |

TABLE 24
Overview amino acid sequences for anti-GroEL2 antibodies.

| Type of sequence | 63.4 | 80.8 | 103.11 | 111.6 | 238.11 |
|---|---|---|---|---|---|
| CDRH1 | 59 | 72 | 85 | 98 | 111 |
| CDRH2 | 60 | 73 | 86 | 99 | 112 |
| CDRH3 | 61 | 74 | 87 | 100 | 113 |
| CRDL1 | 62 | 75 | 88 | 101 | 114 |
| CDRL2 | LVS | DTS | SAS | AAS | GTS |
| CDRL3 | 63 | 76 | 89 | 102 | 115 |
| VH | 64 | 77 | 90 | 103 | 116 |
| VL (κ) | 65 | 78 | 91 | 104 | 117 |
| Heavy chain | 68 | 81 | 94 | 107 | 120 |
| Light chain | 69 | 82 | 95 | 108 | 121 |

TABLE 25
Overview nucleic acid sequences for anti-GroEL2 antibodies.

| Type of sequence | 63.4 | 80.8 | 103.11 | 111.6 | 238.11 |
|---|---|---|---|---|---|
| VH | 66 | 79 | 92 | 105 | 118 |
| VL (κ) | 67 | 80 | 93 | 106 | 119 |
| Heavy chain | 70 | 83 | 96 | 109 | 122 |
| Light chain | 71 | 84 | 97 | 110 | 123 |

TABLE 26
Overview amino acid sequences for anti-LprG antibody.

| Type of sequence | 469.8 |
|---|---|
| CDRH1 | 124 |
| CDRH2 | 125 |
| CDRH3 | 126 |
| CRDL1 | 127 |
| CDRL2 | LVS |
| CDRL3 | 128 |
| VH | 129 |
| VL (κ) | 130 |
| Heavy chain | 133 |
| Light chain | 134 |

TABLE 27
Overview nucleic acid sequences for anti-LprG antibody.

| Type of sequence | 469.8 |
|---|---|
| VH | 131 |
| VL (κ) | 132 |
| Heavy chain | 135 |
| Light chain | 136 |

What is claimed is:

1. An isolated anti-Mycobacterium tuberculosis arabinomannan (anti-Mtb AM) antibody, or Mycobacterium tuberculosis arabinomannan-binding fragment (Mtb AM-binding fragment) thereof, wherein the anti-Mtb AM antibody, or Mtb AM-binding fragment thereof, comprises a heavy chain variable region and a light chain variable region, wherein each of the heavy chain and the light chain variable regions comprises a CDR1, CDR2, and CDR3, and wherein:
(a) CDR1H comprises SEQ ID NO:1, CDR2H comprises SEQ ID NO:2, CDR3H comprises SEQ ID NO:3, CDR1L comprises SEQ ID NO:4, CDR2L comprises sequence AVT, and CDR3L comprises SEQ ID NO:5;
(b) CDR1H comprises SEQ ID NO:10, CDR2H comprises SEQ ID NO:11, CDR3H comprises SEQ ID NO:12, CDR1L comprises SEQ ID NO:13, CDR2L comprises sequence DVT, and CDR3L comprises SEQ ID NO:14;
(c) CDR1H comprises SEQ ID NO:19, CDR2H comprises SEQ ID NO:20, CDR3H comprises SEQ ID NO:21, CDR1L comprises SEQ ID NO:22, CDR2L comprises sequence KIS, and CDR3L comprises SEQ ID NO:23;
(d) CDR1H comprises SEQ ID NO:28, CDR2H comprises SEQ ID NO:29, CDR3H comprises SEQ ID NO:30, CDR1L comprises SEQ ID NO:31, CDR2L comprises sequence GAS, and CDR3L comprises SEQ ID NO:32;
(e) CDR1H comprises SEQ ID NO:37, CDR2H comprises SEQ ID NO:38, CDR3H comprises SEQ ID NO:39, CDR1L comprises SEQ ID NO:40, CDR2L comprises sequence EVS, and CDR3L comprises SEQ ID NO:41; or
(f) CDR1H comprises SEQ ID NO:161, CDR2H comprises SEQ ID NO:162, CDR3H comprises SEQ ID NO:163, CDR1L comprises SEQ ID NO:164, CDR2L comprises sequence KSD, and CDR3L comprises SEQ ID NO:165.

2. The anti-Mtb AM antibody, or Mtb AM-binding fragment thereof, of claim 1, wherein the anti-Mtb AM antibody, or Mtb AM-binding fragment thereof, comprises:
(a) a heavy chain variable region that comprises a sequence that is at least 80% identical to SEQ ID NO:6 and a light chain variable region that comprises a sequence that is at least 80% identical to SEQ ID NO:7;
(b) a heavy chain variable region that comprises a sequence that is at least 80% identical to SEQ ID NO:15 and a light chain variable region that comprises a sequence that is at least 80% identical to SEQ ID NO:16;
(c) a heavy chain variable region that comprises a sequence that is at least 80% identical to SEQ ID NO:24 and a light chain variable region that comprises a sequence that is at least 80% identical to SEQ ID NO:25;
(d) a heavy chain variable region that comprises a sequence that is at least 80% identical to SEQ ID NO:33 and a light chain variable region that comprises a sequence that is at least 80% identical to SEQ ID NO:34;
(e) a heavy chain variable region that comprises a sequence that is at least 80% identical to SEQ ID NO:42 and a light chain variable region that comprises a sequence that is at least 80% identical to SEQ ID NO:43; or
(f) a heavy chain variable region that comprises a sequence that is at least 80% identical to SEQ ID NO:166 and a light chain variable region that comprises a sequence that is at least 80% identical to SEQ ID NO:167.

3. The anti-Mtb AM antibody, or Mtb AM-binding fragment thereof, of claim 2, wherein the anti-Mtb AM antibody, or Mtb AM-binding fragment thereof, comprises:
(a) a heavy chain variable region that comprises a sequence that is at least 90% identical to SEQ ID NO:6 and a light chain variable region that comprises a sequence that is at least 90% identical to SEQ ID NO:7;
(b) a heavy chain variable region that comprises a sequence that is at least 90% identical to SEQ ID NO:15 and a light chain variable region that comprises a sequence that is at least 90% identical to SEQ ID NO:16;
(c) a heavy chain variable region that comprises a sequence that is at least 90% identical to SEQ ID NO:24 and a light chain variable region that comprises a sequence that is at least 90% identical to SEQ ID NO:25;
(d) a heavy chain variable region that comprises a sequence that is at least 90% identical to SEQ ID NO:33 and a light chain variable region that comprises a sequence that is at least 90% identical to SEQ ID NO:34;
(e) a heavy chain variable region that comprises a sequence that is at least 90% identical to SEQ ID NO:42 and a light chain variable region that comprises a sequence that is at least 90% identical to SEQ ID NO:43; or
(f) a heavy chain variable region that comprises a sequence that is at least 90% identical to SEQ ID NO:166 and a light chain variable region that comprises a sequence that is at least 90% identical to SEQ ID NO:167.

4. The anti-Mtb AM antibody, or Mtb AM-binding fragment thereof, of claim 3, wherein the anti-Mtb AM antibody, or Mtb AM-binding fragment thereof, comprises:
(a) a heavy chain variable region that comprises SEQ ID NO:6 and a light chain variable region that comprises SEQ ID NO:7;
(b) a heavy chain variable region that comprises SEQ ID NO:15 and a light chain variable region that comprises SEQ ID NO:16;
(c) a heavy chain variable region that comprises SEQ ID NO:24 and a light chain variable region that comprises SEQ ID NO:25;
(d) a heavy chain variable region that comprises SEQ ID NO:33 and a light chain variable region that comprises SEQ ID NO:34;
(e) a heavy chain variable region that comprises SEQ ID NO:42 and a light chain variable region that comprises SEQ ID NO:43;
(f) a heavy chain variable region that comprises SEQ ID NO:166 and a light chain variable region that comprises SEQ ID NO:167.

5. The anti-Mtb AM antibody, or Mtb AM-binding fragment thereof, of any of one of claims 1-4, wherein the antibody is a monoclonal antibody.

6. The anti-Mtb AM antibody, or Mtb AM-binding fragment thereof, of any one of claims 1-4, wherein the anti-Mtb AM antibody is a recombinant antibody.

7. The anti-Mtb AM antibody, or Mtb AM-binding fragment thereof, of any one of claims 1-6, wherein the anti-Mtb AM antibody, or Mtb AM-binding fragment thereof, comprises a human framework region or a modified human framework region.

8. The anti-Mtb AM antibody, or Mtb AM-binding fragment thereof, of any one of claims 1-7, wherein the anti-Mtb AM antibody, or Mtb AM-binding fragment thereof, comprises a human constant region or a modified human constant region.

9. The Mtb AM-binding fragment of any one of claims 1-8, wherein the Mtb AM-binding fragment is an is an scFv, Fv, Fab′, Fab, F(ab′)₂, or diabody.

10. The anti-Mtb AM antibody, or Mtb AM-binding fragment thereof, of any one of claims 1-9, wherein the anti-Mtb AM antibody, or Mtb AM-binding fragment thereof, is deglycosylated.

11. The anti-Mtb AM antibody, or Mtb AM-binding fragment thereof, of any one of claims 1-10, wherein the anti-Mtb AM antibody, or Mtb AM-binding fragment thereof, is conjugated to one or more of a cytotoxin, a fluorescent label, and an imaging agent.

12. A nucleic acid molecule encoding the anti-Mtb AM antibody, or Mtb AM-binding fragment thereof, of any one of claims 1-10.

13. The nucleic acid molecule of claim 12, wherein the nucleic acid molecule comprises:
(a) SEQ ID NO:8 and/or SEQ ID NO:9;
(b) SEQ ID NO:17 and/or SEQ ID NO: 18;
(c) SEQ ID NO:26 and/or SEQ ID NO:27;
(d) SEQ ID NO:35 and/or SEQ ID NO:36;
(e) SEQ ID NO:44 and/or SEQ ID NO:45; or
(f) SEQ ID NO:168 and/or SEQ ID NO: 169.

14. A vector or set of vectors comprising the nucleic acid molecule of any one of claims 12 or 13.

15. A host cell comprising the nucleic acid molecule of any one of claims 12 or 13 or the vector or set of vectors of claim 14.

16. A method of producing an anti-Mtb AM antibody, or Mtb AM-binding fragment thereof, comprising culturing the host cell of claim 15 under conditions wherein the anti-Mtb AM antibody, or Mtb AM-binding fragment thereof, is produced by the host cell.

17. A pharmaceutical composition comprising an anti-Mtb AM antibody, or Mtb AM-binding fragment thereof, of any one of claims 1-11, and a pharmaceutically acceptable excipient.

18. A method of reducing an activity of Mycobacterium tuberculosis AM in a subject in need thereof, the method comprising administering to said subject the anti-Mtb AM antibody, or Mtb AM-binding fragment thereof, of any one of claims 1-11, or the pharmaceutical composition of claim 17.

19. A method of treating a Mycobacterium tuberculosis infection in a subject, the method comprising administering to the subject the anti-Mtb AM antibody, or Mtb AM-binding fragment thereof, of any one of claims 1-11, or the pharmaceutical composition of claim 17.

20. A method of reducing the likelihood of a Mycobacterium tuberculosis infection in a subject who does not have a Mycobacterium tuberculosis infection, the method comprising administering to the subject the anti-Mtb AM antibody, or Mtb AM-binding fragment thereof, of any one of claims 1-11, or the pharmaceutical composition of claim 17.

21. A method of treating a disease, disorder, or condition mediated by, or related to increased activity of Mycobacterium tuberculosis in a subject, the method comprising administering to said subject the anti-Mtb AM antibody, or Mtb AM-binding fragment thereof, of any one of claims 1-11, or the pharmaceutical composition of claim 17.

22. An assay device for selectively detecting AM, an AM fragment, LAM and/or an AM-comprising fragment of LAM in a biological sample comprising:
(a) a first portion comprising a first plurality of anti-Mtb AM antibodies, or Mtb AM-binding fragments thereof, of any of claims 1-11, wherein the anti-Mtb AM antibodies, or Mtb AM-binding fragments thereof, are attached to a reporting entity; and
(b) a second portion comprising a second plurality of anti-Mtb AM antibodies, or Mtb AM-binding fragments thereof, or anti-mycobacterial AM-antibodies of antigen-binding fragments thereof.

23. An assay device for selectively detecting MTC AM, an AM fragment, LAM and/or an AM-comprising fragment of LAM and/or one or more bacteria from the Mycobacterium tuberculosis complex (MTC) group in a biological sample comprising:
(a) a first portion comprising a first plurality of anti-Mtb AM antibodies, or Mtb AM-binding fragments thereof, of any of claims 1-11, wherein the anti-Mtb AM antibodies, or Mtb AM-binding fragments thereof, are attached to a reporting entity; and
(b) second portion comprising a second plurality of anti-Mtb AM antibodies, or Mtb AM-binding fragments thereof, or anti-mycobacterial AM-antibodies of antigen-binding fragments thereof.

24. The device of claim 22 or 23, wherein the reporting entity comprises a gold nanoparticle.

25. The device of any one of claims 22-24, wherein the reporting entity comprises an enzyme.

26. The device of claim 25, wherein the enzyme is horseradish peroxidase (lRP) or alkaline phosphatase (AP).

27. The device of any one of claims 22-26, wherein the second plurality of anti-Mtb AM antibodies, or Mtb AM-binding fragments thereof, or anti-mycobacterial AM-antibodies, or antigen-binding fragments thereof, is affixed to a solid support of the device.

28. The device of any of claims 22-27, wherein the first plurality of anti-Mtb AM antibodies, or Mtb AM-binding fragments thereof is not affixed to a solid support of the device.

29. The device of claim 27, wherein the solid support comprises nitrocellulose.

30. The device of any of claims 22-29, further comprising a fluid sample pad prior in sequential order to the first and second portions.

31. The device of any of claims 22-30, further comprising a control portion subsequent in sequential order to the first and second portions.

32. The device of claim 31, wherein the control portion comprises a third plurality of antibodies, immobilized on a solid support of the device, and which third plurality of antibodies are capable of binding the first plurality of anti-Mtb AM antibodies, or Mtb AM-binding fragments thereof each attached to their own reporting molecule.

33. The device of any of claims 22-32, further comprising a fluid-absorbent wicking pad subsequent in sequential order to the first and second portions, and third portion if present.

34. The device of any of claims 22-33, wherein the device is a lateral flow assay device.

35. A method of detecting AM, an AM fragment, LAM and/or an AM-comprising fragment of LAM in a biological sample comprising:
(a) contacting the device of any of claims 22, or 24-34 with the biological sample; and
(b) observing if the AM, AM fragment, LAM and/or AM-comprising fragment of LAM bind to the second plurality of anti-Mtb AM antibodies, or Mtb AM-binding fragments thereof, or anti-mycobacterial AM-antibodies, or antigen-binding fragments thereof;
wherein if the AM, AM fragment, LAM and/or AM-comprising fragment of LAM bind to the second plurality of anti-Mtb AM antibodies, or Mtb AM-binding fragments thereof, or anti-mycobacterial AM-antibodies, or antigen-binding fragments thereof, then AM, AM fragment, LAM and/or AM-comprising fragment of LAM have been detected in the biological sample; and
wherein if no AM, AM fragment, LAM and/or AM-comprising fragment of LAM bind to the second plurality of anti-Mtb AM antibodies, or Mtb AM-binding fragments thereof, or anti-mycobacterial AM-antibodies, or antigen-binding fragments thereof, then AM, AM fragment, LAM and/or AM-comprising fragment of LAM have not been detected in the biological sample.

36. A method of detecting MTC AM, an AM fragment, LAM and/or an AM-comprising fragment of LAM and/or one or more bacteria from the MTC group in a biological sample comprising:
(a) contacting the device of any of claims 23-34 with the biological sample; and
(b) observing if MTC AM, an AM fragment, LAM and/or an AM-comprising fragment of LAM and/or one or more bacteria from the MTC group bind to the second plurality of anti-Mtb AM antibodies, or Mtb AM-binding fragments thereof, or anti-mycobacterial AM-antibodies, or antigen-binding fragments thereof,
wherein if MTC AM, an AM fragment, LAM and/or an AM-comprising fragment of LAM and/or one or more bacteria from the MTC group bind to the second plurality of anti-Mtb AM antibodies, or Mtb AM-binding fragments thereof, or anti-mycobacterial AM-antibodies, or antigen-binding fragments thereof, then MTC AM, an AM fragment, LAM and/or an AM-comprising fragment of LAM and/or one or more bacteria from the MTC group bind have been detected in the biological sample; and
wherein if no MTC AM, an AM fragment, LAM and/or an AM-comprising fragment of LAM and no bacteria from the MTC group bind to the second plurality of anti-Mtb AM antibodies, or Mtb AM-binding fragments thereof, or anti-mycobacterial AM-antibodies, or antigen-binding fragments thereof, then MTC AM, an AM fragment, LAM and/or an AM-comprising fragment of LAM and bacteria from the MTC group have not been detected in the biological sample.

37. The method of claim 35 or 36, further comprising obtaining the sample from asubject.

38. The method of any one of claims 35-37, wherein the sample is urine, cerebrospinal fluid, pleural fluid, peritoneal fluid, sputum, exhaled breath condensate, saliva, mucosal lining fluid, whole blood, serum, plasma, breath, a tissue sample, or a fine-needle aspirate.

39. The method of any one of claims 18-21 or 37-38, wherein the subject is a mammal.

40. The method of claim 39, wherein the mammal is a human.

41. An isolated anti-GroEL2 antibody, or GroEL2-binding fragment thereof, wherein the anti-GroEL2 antibody, or GroEL2-binding fragment thereof, comprises a heavy chain variable region and a light chain variable region, wherein each of the heavy chain and the light chain variable regions comprises a CDR1, CDR2, and CDR3, and wherein:
(a) CDR1H comprises SEQ ID NO:59, CDR2H comprises SEQ ID NO:60, CDR3H comprises SEQ ID NO:61, CDR1L comprises SEQ ID NO:62, CDR2L comprises sequence LVS, and CDR3L comprises SEQ ID NO:63;
(b) CDR1H comprises SEQ ID NO:72, CDR2H comprises SEQ ID NO:73, CDR3H comprises SEQ ID NO:74, CDR1L comprises SEQ ID NO:75, CDR2L comprises sequence DTS, and CDR3L comprises SEQ ID NO:76;
(c) CDR1H comprises SEQ ID NO:85, CDR2H comprises SEQ ID NO:86, CDR3H comprises SEQ ID NO:87, CDR1L comprises SEQ ID NO:88, CDR2L comprises sequence SAS, and CDR3L comprises SEQ ID NO:89;
(d) CDR1H comprises SEQ ID NO:98, CDR2H comprises SEQ ID NO:99, CDR3H comprises SEQ ID NO:100, CDR1L comprises SEQ ID NO:101, CDR2L comprises sequence AAS, and CDR3L comprises SEQ ID NO:102; or
(e) CDR1H comprises SEQ ID NO:111, CDR2H comprises SEQ ID NO:112, CDR3H comprises SEQ ID NO:113, CDR1L comprises SEQ ID NO:114, CDR2L comprises sequence GTS, and CDR3L comprises SEQ ID NO:115.

42. The anti-GroEL2 antibody, or GroEL2-binding fragment thereof, of claim 41, wherein the anti-GroEL2 antibody, or GroEL2-binding fragment thereof, of comprises:
(a) a heavy chain variable region that comprises a sequence that is at least 80% identical to SEQ ID NO:64 and a light chain variable region that comprises a sequence that is at least 80% identical to SEQ ID NO:65;
(b) a heavy chain variable region that comprises a sequence that is at least 80% identical to SEQ ID NO:77 and a light chain variable region that comprises a sequence that is at least 80% identical to SEQ ID NO:78;
(c) a heavy chain variable region that comprises a sequence that is at least 80% identical to SEQ ID NO:90 and a light chain variable region that comprises a sequence that is at least 80% identical to SEQ ID NO:91;
(d) a heavy chain variable region that comprises a sequence that is at least 80% identical to SEQ ID NO:103 and a light chain variable region that comprises a sequence that is at least 80% identical to SEQ ID NO:104; or
(e) a heavy chain variable region that comprises a sequence that is at least 80% identical to SEQ ID NO:116 and a light chain variable region that comprises a sequence that is at least 80% identical to SEQ ID NO: 117.

43. The anti-GroEL2 antibody, or GroEL2-binding fragment thereof, of claim 42, wherein the anti-GroEL2 antibody, or GroEL2-binding fragment thereof, of comprises:
(a) a heavy chain variable region that comprises a sequence that is at least 90% identical to SEQ ID NO:64 and a light chain variable region that comprises a sequence that is at least 90% identical to SEQ ID NO:65;
(b) a heavy chain variable region that comprises a sequence that is at least 90% identical to SEQ ID NO:77 and a light chain variable region that comprises a sequence that is at least 90% identical to SEQ ID NO:78;
(c) a heavy chain variable region that comprises a sequence that is at least 90% identical to SEQ ID NO:90 and a light chain variable region that comprises a sequence that is at least 90% identical to SEQ ID NO:91;
(d) a heavy chain variable region that comprises a sequence that is at least 90% identical to SEQ ID NO:103 and a light chain variable region that comprises a sequence that is at least 90% identical to SEQ ID NO:104; or
(e) a heavy chain variable region that comprises a sequence that is at least 90% identical to SEQ ID NO:116 and a light chain variable region that comprises a sequence that is at least 90% identical to SEQ ID NO:117.

44. The anti-GroEL2 antibody, or GroEL2-binding fragment thereof, of claim 43, wherein the anti-GroEL2 antibody, or GroEL2-binding fragment thereof, of comprises:
(a) a heavy chain variable region that comprises SEQ ID NO:64 and a light chain variable region that comprises SEQ ID NO:65;
(b) a heavy chain variable region that comprises SEQ ID NO:77 and a light chain variable region that comprises SEQ ID NO:78;
(c) a heavy chain variable region that comprises SEQ ID NO:90 and a light chain variable region that comprises SEQ ID NO:91;
(d) a heavy chain variable region that comprises SEQ ID NO:103 and a light chain variable region that comprises SEQ ID NO:104; or
(e) a heavy chain variable region that comprises SEQ ID NO:116 and a light chain variable region that comprises SEQ ID NO:117.

45. The anti-GroEL2 antibody, or GroEL2-binding fragment thereof, of any of one of claims 41-44, wherein the anti-GroEL2 antibody is a monoclonal antibody.

46. The anti-GroEL2 antibody, or GroEL2-binding fragment thereof, of any one of claims 41-44, wherein anti-GroEL2 antibody is a recombinant antibody.

47. The anti-GroEL2 antibody, or GroEL2-binding fragment thereof, of any one of claims 41-46, wherein the anti-GroEL2 antibody, or GroEL2-binding fragment thereof, comprises a human framework region or a modified human framework region.

48. The anti-GroEL2 antibody, or GroEL2-binding fragment thereof, of any one of claims 41-47, wherein the anti-GroEL2 antibody, or GroEL2-binding fragment thereof, comprises a human constant region or a modified human constant region.

49. The GroEL2-binding fragment of any one of claims 41-48, wherein the GroEL2-binding fragment is an is an scFv, Fv, Fab′, Fab, F(ab′)₂, or diabody.

50. The anti-GroEL2 antibody, or GroEL2-binding fragment thereof, of any one of claims 41-49, wherein the anti-GroEL2 antibody, or GroEL2-binding fragment thereof, is deglycosylated.

51. The anti-GroEL2 antibody, or GroEL2-binding fragment thereof, of any one of claims 41-50, wherein the anti-GroEL2 antibody, or GroEL2-binding fragment thereof, is conjugated to one or more of a cytotoxin, a fluorescent label, and an imaging agent.

52. A nucleic acid molecule encoding the anti-GroEL2 antibody, or GroEL2-binding fragment thereof, of any one of claims 41-50.

53. The nucleic acid molecule of claim 52, wherein the nucleic acid molecule comprises
(a) SEQ ID NO:66 and/or SEQ ID NO: 67;
(b) SEQ ID NO:79 and/or SEQ ID NO: 80;
(c) SEQ ID NO:92 and/or SEQ ID NO: 93;
(d) SEQ ID NO:105 and/or SEQ ID NO: 106; or
(e) SEQ ID NO:118 and/or SEQ ID NO: 119.

54. A vector or set of vectors comprising the nucleic acid molecule of any one of claims 52 or 53.

55. A host cell comprising the nucleic acid molecule of any one of claims 52 or 53 or the vector or set of vectors of claim 54.

56. A method of producing an anti-GroEL2 antibody, or GroEL2-binding fragment thereof, comprising culturing the host cell of claim 55, under conditions wherein the anti-GroEL2 antibody, or GroEL2-binding fragment thereof, is produced by the host cell.

57. A pharmaceutical composition comprising an anti-GroEL2 antibody, or GroEL2-binding fragment thereof, of any one of claims 41-51, and a pharmaceutically acceptable excipient.

58. An isolated anti-LprG antibody, or LprG-binding fragment thereof, wherein the anti-LprG antibody, or LprG-binding fragment thereof, comprises a heavy chain variable region and a light chain variable region, wherein each of the heavy chain and the light chain variable regions comprises a CDR1, CDR2, and CDR3, and wherein CDR1H comprises SEQ ID NO:124, CDR2H comprises SEQ ID NO:125, CDR3H comprises SEQ ID NO:126, CDR1L comprises SEQ ID NO:127, CDR2L comprises sequence LVS, and CDR3L comprises SEQ ID NO:128.

59. The anti-LprG antibody, or LprG-binding fragment thereof, of claim 58, wherein the anti-LprG antibody, or LprG-binding fragment thereof, of comprises a heavy chain variable region that comprises a sequence that is at least 80% identical to SEQ ID NO:129 and a light chain variable region that comprises a sequence that is at least 80% identical to SEQ ID NO:130.

60. The anti-LprG antibody, or LprG-binding fragment thereof, of claim 59, wherein the anti-LprG antibody, or LprG-binding fragment thereof, of comprises a heavy chain variable region that comprises a sequence that is at least 90% identical to SEQ ID NO:129 and a light chain variable region that comprises a sequence that is at least 90% identical to SEQ ID NO:130.

61. The anti-LprG antibody, or LprG-binding fragment thereof, of claim 60, wherein the anti-LprG antibody, or LprG-binding fragment thereof, of comprises a heavy chain variable region that comprises SEQ ID NO:129 and a light chain variable region that comprises SEQ ID NO:130.

62. The anti-LprG antibody, or LprG-binding fragment thereof, of any of one of claims 58-61, wherein the antibody is a monoclonal antibody.

63. The anti-LprG antibody, or LprG-binding fragment thereof, of any one of claims 58-61, wherein the anti-LprG antibody is a recombinant antibody.

64. The anti-LprG antibody, or LprG-binding fragment thereof, of any one of claims 58-63, wherein the anti-LprG antibody, or LprG-binding fragment thereof, comprises a human framework region or a modified human framework region.

65. The anti-LprG antibody, or LprG-binding fragment thereof, of any one of claims 58-64, wherein the anti-LprG antibody, or LprG-binding fragment thereof, comprises a human constant region or a modified human constant region.

66. The LprG-binding fragment of any one of claims 58-65, wherein the LprG-binding fragment is an is an scFv, Fv, Fab′, Fab, F(ab′)₂, or diabody.

67. The anti-LprG antibody, or LprG-binding fragment thereof, of any one of claims 58-66, wherein the anti-LprG antibody, or LprG-binding fragment thereof, is deglycosylated.

68. The anti-LprG antibody, or LprG-binding fragment thereof, of any one of claims 58-67, wherein the anti-LprG antibody, or LprG-binding fragment thereof, is conjugated to one or more of a cytotoxin, a fluorescent label, and an imaging agent.

69. A nucleic acid molecule encoding the anti-LprG antibody, or LprG-binding fragment thereof, of any one of claims 58-67.

70. The nucleic acid molecule of claim 69, wherein the nucleic acid molecule comprises SEQ ID NO:131 and/or SEQ ID NO:132.

71. A vector or set of vectors comprising the nucleic acid molecule of any one of claims 69 or 70.

72. A host cell comprising the nucleic acid molecule of any one of claims 69 or 70 or the vector or set of vectors of claim 71.

73. A method of producing an anti-LprG antibody, or LprG-binding fragment thereof, comprising culturing the host cell of claim 72, under conditions wherein the anti-LprG antibody, or LprG-binding fragment thereof, is produced by the host cell.

74. A pharmaceutical composition comprising an anti-LprG antibody, or LprG-binding fragment thereof, of any one of claims 58-68, and a pharmaceutically acceptable excipient.


SEQUENCE LISTING (169 sequences)

SEQ ID NO: 1 - protein, 8 aa, Homo sapiens
GFRFSDYW

SEQ ID NO: 2 - protein, 8 aa, Homo sapiens
IKQDGSER

SEQ ID NO: 3 - protein, 14 aa, Homo sapiens
ARDFGMATPGGMFY

SEQ ID NO: 4 - protein, 9 aa, Homo sapiens
SSDVGRYNL

SEQ ID NO: 5 - protein, 10 aa, Homo sapiens
FSYAGGDNWM

SEQ ID NO: 6 - protein, 121 aa, Homo sapiens
EVQLVESGGDLVQPGGSLRLSCVASGFRFSDYWMTWVRQAPGKGLEWVANIKQDGSERHYVDSVKGRFTVSRDNAENSVYLQMNSLRAEDTAMYYCARDFGMATPGGMFYWGQGALVTVSS

SEQ ID NO: 7 - protein, 110 aa, Homo sapiens
QSALTQPPSASGSPGQSVTISCSGTSSDVGRYNLVSWFQRHPGKAPKLMIYAVTKRPSGVPARFSGSKSGNTASLTVSGLQADDEGDYYCFSYAGGDNWMFGGGTKLTVL

SEQ ID NO: 8 - protein, 364 aa, Homo sapiens
GAGGTGCAGCTGGTGGAGTCAGGGGGAGACTTGGTCCAGCCGGGGGGGTCCCTGAGACTCTCCTGTGTTGCCTCTGGATTCAGGTTTAGTGACTACTGGATGACGTGGGTCCGCCAGGCTCCAGGGAAGGGGCTGGAGTGGGTGGCCAACATAAAGCAAGATGGAAGTGAGAGGCACTATGTGGACTCTGTGAAGGGCCGGTTCACCGTCTCCAGAGACAACGCCGAGAACTCAGTCTATCTACAAATGAACAGCCTGAGAGCCGAAGACACGGCTATGTATTATTGTGCGCGAGATTTTGGTATGGCCACCCCGGGAGGGATGTTCTACTGGGGCCAGGGAGCCCTGGTCACCGTCTCCTCAG

SEQ ID NO: 9 - protein, 331 aa, Homo sapiens
CAGTCTGCCCTGACTCAGCCTCCCTCCGCGTCCGGGTCTCCTGGACAGTCAGTCACCATCTCCTGCTCTGGAACCAGCAGTGACGTTGGTCGTTATAACCTTGTCTCCTGGTTCCAGCGGCACCCAGGCAAAGCCCCCAAACTCATGATTTATGCGGTCACTAAGCGGCCCTCAGGGGTCCCTGCTCGCTTCTCTGGCTCCAAGTCTGGCAACACGGCCTCCCTGACCGTCTCTGGGCTCCAGGCTGACGATGAGGGTGATTATTACTGCTTCTCATATGCCGGCGGCGACAATTGGATGTTCGGCGGGGGGACCAAACTGACCGTCCTGG

SEQ ID NO: 10 - protein, 8 aa, Homo sapiens
GFTFTNAW

SEQ ID NO: 11 - protein, 10 aa, Homo sapiens
VKSEGDGGTT

SEQ ID NO: 12 - protein, 9 aa, Homo sapiens
ATGGLKYEA

SEQ ID NO: 13 - protein, 9 aa, Homo sapiens
SSDVGGHNL

SEQ ID NO: 14 - protein, 11 aa, Homo sapiens
SSYRSSDTLVV

SEQ ID NO: 15 - protein, 118 aa, Homo sapiens
EVQLVESGGGLVKPGGSLRLSCVVSGFTFTNAWMNWVRQAPGKGLEWVGRVKSEGDGGTTEYAASVKGRFIISRDDSKDTLYLQMNSLKREDTALYYCATGGLKYEAWGQGTMVTVSS

SEQ ID NO: 16 - protein, 111 aa, Homo sapiens
QSALTQPASVSGSPGQSITISCTGTSSDVGGHNLVAWYQQHPGDAPKLMIFDVTRRPSGVSNRFSGSKSGNTASLTISGLQAEDEADYYCSSYRSSDTLVVLGGGTRLTVL

SEQ ID NO: 17 - protein, 355 aa, Homo sapiens
GAGGTGCAGCTGGTGGAGTCGGGGGGAGGCTTGGTCAAGCCTGGGGGGTCCCTTAGACTCTCCTGTGTAGTCTCTGGATTCACTTTCACTAACGCCTGGATGAACTGGGTCCGCCAGGCTCCAGGGAAGGGGCTGGAGTGGGTTGGCCGTGTTAAAAGTGAAGGTGACGGTGGGACAACAGAGTACGCTGCATCCGTGAAAGGCAGATTCATCATCTCAAGAGATGATTCAAAAGACACTCTGTATCTGCAAATGAACAGTCTGAAAAGGGAGGACACAGCCCTGTATTATTGTGCCACAGGAGGGTTAAAATACGAAGCCTGGGGCCAAGGGACAATGGTCACCGTCTCTTCAG

SEQ ID NO: 18 - protein, 334 aa, Homo sapiens
CAGTCTGCCCTGACTCAGCCTGCCTCCGTGTCTGGGTCTCCTGGACAGTCGATCACCATCTCCTGCACTGGAACCAGCAGTGACGTTGGAGGGCATAACCTCGTCGCCTGGTACCAACAACACCCAGGCGATGCCCCCAAACTCATGATTTTTGATGTCACTAGACGGCCCTCAGGGGTTTCTAATCGCTTCTCTGGCTCCAAGTCTGGCAACACGGCCTCCCTGACCATCTCTGGGCTCCAGGCTGAGGACGAGGCTGATTATTATTGCAGCTCATATAGAAGCAGCGACACTCTCGTGGTGCTCGGCGGAGGGACCAGGTTGACCGTCCTAG

SEQ ID NO: 19 - protein, 8 aa, Homo sapiens
GFAFVSGM

SEQ ID NO: 20 - protein, 7 aa, Homo sapiens
SGDGHAT

SEQ ID NO: 21 - protein, 10 aa, Homo sapiens
ALWLQVHFDN

SEQ ID NO: 22 - protein, 11 aa, Homo sapiens
EVLVHSDGNTY

SEQ ID NO: 23 - protein, 9 aa, Homo sapiens
MQVTHFPWT

SEQ ID NO: 24 - protein, 116 aa, Homo sapiens
EVQLLESGGALVQPGGSLRLSCAASGFAFVSGMSWVRQAPGKGLEWVSTISGDGHATHYADSVKGRFTISREDSKSTLFLDMSNLRAEDTALYYCALWLQVHFDNWGQGTLVTVSS

SEQ ID NO: 25 - protein, 112 aa, Homo sapiens
DIVMTQTPLSSPVALGQSASISCRSSEVLVHSDGNTYMTWFHQRPGQPPRLLIYKISRRFSGVPDRFSGSGAGTDFTLKISRVEAEDVGVYYCMQVTHFPWTFGRGTKVEIK

SEQ ID NO: 26 - protein, 349 aa, Homo sapiens
GAGGTGCAGCTGTTGGAGTCTGGGGGAGCCTTGGTACAGCCTGGGGGGTCCCTGAGACTCTCCTGTGCAGCCTCTGGATTCGCCTTTGTGTCTGGCATGAGTTGGGTCCGCCAGGCTCCAGGGAAGGGGCTGGAGTGGGTCTCAACTATTAGTGGTGATGGTCATGCTACACATTATGCAGACTCCGTGAAGGGCCGGTTCACCATCTCCAGAGAGGATTCCAAGAGCACCCTGTTTCTGGACATGAGCAACTTGAGAGCCGAGGACACGGCCCTGTATTACTGTGCCCTATGGCTACAAGTGCACTTTGACAACTGGGGCCAGGGAACCCTGGTCACCGTCTCCTCAG

SEQ ID NO: 27 - protein, 337 aa, Homo sapiens
GATATTGTGATGACCCAGACTCCACTCTCCTCACCTGTCGCCCTTGGACAGTCGGCCTCCATCTCCTGCAGGTCTAGTGAAGTCCTCGTCCACAGTGATGGAAACACCTACATGACTTGGTTTCACCAGAGGCCAGGCCAGCCTCCAAGACTCCTTATTTATAAGATTTCTAGGCGATTCTCTGGGGTCCCAGACAGATTCAGTGGCAGTGGGGCAGGGACAGATTTCACCCTGAAAATCAGCAGGGTGGAGGCTGAGGATGTCGGGGTTTATTACTGCATGCAGGTTACACATTTTCCGTGGACGTTCGGCCGAGGGACCAAGGTGGAAATCAAAC

SEQ ID NO: 28 - protein, 8 aa, Homo sapiens
GFTLDDYG

SEQ ID NO: 29 - protein, 8 aa, Homo sapiens
ISWNGGLT

SEQ ID NO: 30 - protein, 15 aa, Homo sapiens
ARDWYRNDYYYGMDV

SEQ ID NO: 31 - protein, 6 aa, Homo sapiens
QSVSSN

SEQ ID NO: 32 - protein, 8 aa, Homo sapiens
QQYHIWYT

SEQ ID NO: 33 - protein, 122 aa, Homo sapiens
EVQLVESGGGVVRPGGSLRLSCAASGFTLDDYGISWVRQAPGKGLEWVSGISWNGGLTGYADSVKGRFTISRDSAKKSLYLQMNSLRAEDTALYHCARDWYRNDYYYGMDVWGQGTTVTVSS

SEQ ID NO: 34 - protein, 106 aa, Homo sapiens
EIVLTQSPATLSLSPGERATLSCRASQSVSSNLAWYQQKPGQTPRLLIYGASTRATGIPARFSGSGSGTEFTLAISSLQSEDFAVYYCQQYHIWYTFGQGTKVEIK

SEQ ID NO: 35 - protein, 366 aa, Homo sapiens
GAGGTGCAGCTGGTGGAGTCTGGGGGAGGTGTGGTACGGCCTGGGGGGTCCCTGAGACTCTCCTGTGCAGCCTCTGGATTCACCTTGGATGATTATGGCATAAGCTGGGTCCGCCAAGCTCCAGGGAAGGGGCTGGAGTGGGTCTCTGGTATTAGCTGGAATGGTGGTCTCACAGGTTATGCAGACTCTGTTAAGGGGCGATTCACCATCTCCAGGGACAGCGCCAAGAAGTCCCTGTATCTGCAAATGAACAGTCTGAGAGCCGAGGACACGGCCTTGTATCACTGTGCGAGAGATTGGTACAGGAACGACTACTACTACGGTATGGACGTCTGGGGCCAAGGGACCACGGTCACCGTCTCCTCA

SEQ ID NO: 36 - protein, 319 aa, Homo sapiens
GAAATTGTGTTGACGCAGTCTCCAGCCACCCTGTCTCTGTCTCCAGGGGAAAGAGCCACCCTCTCCTGCAGGGCCAGTCAGAGTGTTAGCAGCAACTTGGCCTGGTACCAGCAGAAACCTGGCCAGACTCCCAGGCTCCTCATCTATGGTGCTTCCACCAGGGCCACTGGTATCCCAGCCAGGTTCAGTGGCAGTGGGTCTGGGACAGAGTTTACTCTCGCCATCAGTAGCCTGCAATCTGAAGACTTTGCAGTTTATTACTGTCAGCAGTATCATATCTGGTACACTTTTGGCCAGGGGACCAAGGTGGAAATCAAAC

SEQ ID NO: 37 - protein, 8 aa, Homo sapiens
GFPFSERG

SEQ ID NO: 38 - protein, 8 aa, Homo sapiens
INDNGANT

SEQ ID NO: 39 - protein, 10 aa, Homo sapiens
AGWLFNHFDL

SEQ ID NO: 40 - protein, 11 aa, Homo sapiens
ESLVHSDGKTY

SEQ ID NO: 41 - protein, 9 aa, Homo sapiens
LQATHFPWS

SEQ ID NO: 42 - protein, 117 aa, Homo sapiens
EVQLLESGGELVQPGGSLRLSCAASGFPFSERGMSWVRQAQGKGLEWVSTINDNGANTHYADSVMGRFTISRDNSKNTVYLQMNSLRDEDTGIYYCAGWLFNHFDLWGQGALVTVSS

SEQ ID NO: 43 - protein, 112 aa, Homo sapiens
DIVMTQTPLSSTVILGQPASISCKSSESLVHSDGKTYVSWFHQRPGQPPRLLIYEVSKRLSGVPDRFSGGGAATDYTLRISRVEAEDVGIYYCLQATHFPWSFGHGTKVEIK

SEQ ID NO: 44 - protein, 352 aa, Homo sapiens
GAGGTGCAGCTGTTGGAGTCTGGGGGAGAGTTGGTACAGCCCGGGGGGTCCCTGAGACTCTCCTGTGCAGCCTCTGGATTCCCCTTTAGTGAACGTGGCATGAGTTGGGTTCGCCAGGCTCAAGGGAAGGGGCTGGAGTGGGTCTCCACTATCAATGATAATGGCGCCAACACACACTACGCAGACTCCGTGATGGGCCGCTTCACCATCTCCAGAGACAATTCCAAGAACACAGTATATCTACAAATGAATAGCCTGAGAGACGAGGACACGGGCATTTATTACTGTGCGGGGTGGTTGTTCAATCACTTTGACCTCTGGGGCCAGGGAGCCCTGGTCACCGTCTCCTCAG

SEQ ID NO: 45 - protein, 337 aa, Homo sapiens
GATATTGTGATGACCCAGACTCCACTCTCCTCAACTGTCATCCTTGGACAGCCGGCCTCCATCTCCTGTAAGTCTAGTGAAAGCCTCGTTCACAGTGATGGAAAAACCTACGTGAGTTGGTTTCACCAGAGGCCAGGCCAGCCTCCAAGACTCCTCATTTATGAGGTTTCTAAGCGGTTGTCTGGGGTCCCAGACAGATTCAGTGGCGGTGGGGCAGCGACAGATTACACACTGAGAATCAGCAGGGTGGAGGCTGAGGACGTCGGCATTTATTACTGCTTGCAAGCCACACATTTCCCGTGGTCGTTCGGCCACGGGACCAAGGTGGAAATCAAAC

SEQ ID NO: 46 - protein, 8 aa, Homo sapiens
AFPFGTFG

SEQ ID NO: 47 - protein, 8 aa, Homo sapiens
ISNSGKNT

SEQ ID NO: 48 - protein, 13 aa, Homo sapiens
TNDCCGDSPLFNS

SEQ ID NO: 49 - protein, 11 aa, Homo sapiens
QSLKHENGNTY

SEQ ID NO: 50 - protein, 9 aa, Homo sapiens
MQATHFPRT

SEQ ID NO: 51 - protein, 120 aa, Homo sapiens
EVQLLESGGRLIQPGGSLGLSCAASAFPFGTFGMSWVRQAPGKGLEWVSTISNSGKNTHYADSVKGRFIVSRDNSKNTLYLQMNNLRAEDTALYYCTNDCCGDSPLFNSWGRGTLVTVSS

SEQ ID NO: 52 - protein, 112 aa, Homo sapiens
DIVMTQTPLSSPVTLGQSATISCRSSQSLKHENGNTYVSWLQQRPGQPPRLLIYKISNRVSGVPARFSGGGAGTDFTLKISRVEAEDVGIYYCMQATHFPRTFGQGTKVEIK

SEQ ID NO: 53 - protein, 361 aa, Homo sapiens
GAGGTGCAGCTGTTGGAGTCTGGGGGACGCCTGATACAGCCGGGGGGGTCCCTGGGACTCTCCTGTGCAGCCTCTGCGTTCCCCTTTGGTACCTTTGGCATGAGTTGGGTCCGCCAGGCTCCAGGCAAGGGCCTGGAGTGGGTCTCAACTATTAGTAATAGTGGTAAAAACACACACTACGCAGACTCCGTGAAGGGCCGTTTCATCGTCTCCAGAGACAACTCCAAGAACACATTGTATTTGCAAATGAACAACCTGAGAGCCGAAGACACGGCCTTATATTACTGTACGAACGACTGCTGTGGTGACTCACCCCTCTTTAACTCCTGGGGCCGGGGAACTCTGGTCACCGTCTCCTCAG

SEQ ID NO: 54 - protein, 337 aa, Homo sapiens
GATATTGTGATGACCCAGACTCCACTCTCCTCACCTGTCACCCTAGGACAGTCGGCTACCATCTCCTGCAGGTCTAGTCAAAGCCTCAAACACGAAAATGGGAACACCTACGTGAGTTGGCTTCAGCAGAGGCCAGGCCAGCCCCCAAGACTCCTAATTTACAAGATTTCAAACCGGGTCTCTGGGGTTCCAGCCAGATTCAGTGGCGGTGGGGCAGGGACAGATTTCACACTGAAAATCAGCAGGGTGGAAGCTGAGGATGTCGGCATTTACTACTGCATGCAAGCTACACATTTTCCTCGGACGTTCGGCCAAGGGACCAAGGTGGAGATCAAAC

SEQ ID NO: 55 - protein, 5 aa, Homo sapiens
SYAMS

SEQ ID NO: 56 - protein, 330 aa, Homo sapiens
RSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 92 aa, Homo sapiens
SSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 58 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 59 - protein, 8 aa, Homo sapiens
GFTFSSYA

SEQ ID NO: 60 - protein, 8 aa, Homo sapiens
IDIGGDYT

SEQ ID NO: 61 - protein, 7 aa, Homo sapiens
AYGHFAY

SEQ ID NO: 62 - protein, 11 aa, Homo sapiens
QSLLDSDGKTY

SEQ ID NO: 63 - protein, 9 aa, Homo sapiens
WQGIYFPWT

SEQ ID NO: 64 - protein, 114 aa, Homo sapiens
EVQLVESGGGLVKPGGSLKLSCAASGFTFSSYAMSWVRQTPEKRLEWVATIDIGGDYTFYPDSVKGRFTISRDNAKNTLYLQMSSLRSEDTAMFYCAYGHFAYWGQGTLVTVSA

SEQ ID NO: 65 - protein, 112 aa, Homo sapiens
DVVMTQTPLTLSVTIGQPASISCKSSQSLLDSDGKTYLNWLLQRPGQSPKRLIYLVSKLDSGVPDRFTGSGSGTDFTLKISRVEAEDLGVYYCWQGIYFPWTFGGGTKLEIK

SEQ ID NO: 66 - protein, 343 aa, Homo sapiens
GAAGTGCAGCTGGTGGAGTCTGGGGGAGGCTTAGTGAAGCCTGGAGGGTCCCTGAAACTCTCCTGTGCAGCCTCTGGATTCACTTTCAGTAGCTATGCCATGTCTTGGGTTCGCCAGACTCCGGAGAAGAGGCTGGAGTGGGTCGCAACCATTGATATTGGTGGTGATTACACCTTCTATCCAGACAGTGTGAAGGGTCGATTCACCATCTCCAGAGACAATGCCAAGAACACCCTGTACCTGCAAATGAGCAGTCTGAGGTCTGAGGACACGGCCATGTTTTACTGTGCATATGGTCACTTTGCTTACTGGGGCCAGGGGACTCTGGTCACTGTCTCTGCAG

SEQ ID NO: 67 - protein, 337 aa, Homo sapiens
GATGTTGTGATGACCCAGACTCCACTCACTTTGTCGGTAACCATTGGACAACCAGCCTCCATCTCTTGCAAGTCAAGTCAGAGCCTCTTGGATAGTGATGGAAAGACTTATTTGAATTGGTTGTTACAGAGGCCAGGGCAGTCTCCAAAGCGCCTAATCTATCTGGTGTCTAAACTGGACTCTGGAGTCCCTGACAGGTTCACTGGCAGTGGATCAGGGACAGATTTCACACTGAAAATCAGCAGAGTGGAGGCTGAGGATTTGGGAGTTTATTATTGCTGGCAAGGTATATATTTTCCGTGGACGTTCGGTGGAGGAACCAAGCTGGAAATCAAAC

SEQ ID NO: 68 - protein, 457 aa, Homo sapiens
MNFVLSLIFLALILKGVQCEVQLVESGGGLVKPGGSLKLSCAASGFTFSSYAMSWVRQTPEKRLEWVATIDIGGDYTFYPDSVKGRFTISRDNAKNTLYLQMSSLRSEDTAMFYCAYGHFAYWGQGTLVTVSAAKTTPPSVYPLAPGCGDTTGSSVTLGCLVKGYFPESVTVTWNSGSLSSGVHTFPAVLQSDLYTLSSSVTVPSSTWPSETVTCNVAHPASSTKVDKKIVPRDCGCKPCICTVSEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFICSVLHEGLHNHHTEKSLSHSPGK

SEQ ID NO: 69 - protein, 239 aa, Homo sapiens
MMSPAQFLFLLVLWIRETNGDVVMTQTPLTLSVTIGQPASISCKSSQSLLDSDGKTYLNWLLQRPGQSPKRLIYLVSKLDSGVPDRFTGSGSGTDFTLKISRVEAEDLGVYYCWQGIYFPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRDEC

SEQ ID NO: 70 - DNA, 1374 bp, Homo sapiens
  source 1..1374: mol_type = unassigned DNA
atgaactttgtgctcagcttgattttccttgccctcattttaaaaggtgtccagtgtgaagtgcagctggtggagtctgggggaggcttagtgaagcctggagggtccctgaaactctcctgtgcagcctctggattcactttcagtagctatgccatgtcttgggttcgccagactccggagaagaggctggagtgggtcgcaaccattgatattggtggtgattacaccttctatccagacagtgtgaagggtcgattcaccatctccagagacaatgccaagaacaccctgtacctgcaaatgagcagtctgaggtctgaggacacggccatgttttactgtgcatatggtcactttgcttactggggccaggggactctggtcactgtctctgcagccaaaacaacacccccatcagtctatccactggcccctgggtgtggagatacaactggttcctccgtgactctgggatgcctggtcaagggctacttccctgagtcagtgactgtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtctcagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcatctgctctgtgttacatgagggcctgcacaaccaccatactgagaagagcctctcccactctcctggtaaatga

SEQ ID NO: 71 - DNA, 720 bp, Homo sapiens
  source 1..720: mol_type = unassigned DNA
atgatgagtcctgcccagttcctgtttctgttagtgctctggattcgggaaaccaacggtgatgttgtgatgacccagactccactcactttgtcggtaaccattggacaaccagcctccatctcttgcaagtcaagtcagagcctcttggatagtgatggaaagacttatttgaattggttgttacagaggccagggcagtctccaaagcgcctaatctatctggtgtctaaactggactctggagtccctgacaggttcactggcagtggatcagggacagatttcacactgaaaatcagcagagtggaggctgaggatttgggagtttattattgctggcaaggtatatattttccgtggacgttcggtggaggaaccaagctggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacagggatgagtgttag

SEQ ID NO: 72 - protein, 8 aa, Homo sapiens
GFNIRDYY

SEQ ID NO: 73 - protein, 8 aa, Homo sapiens
IDPDNGDT

SEQ ID NO: 74 - protein, 9 aa, Homo sapiens
ARSPIGPAH

SEQ ID NO: 75 - protein, 5 aa, Homo sapiens
SSVTN

SEQ ID NO: 76 - protein, 9 aa, Homo sapiens
FQGSGYPLT

SEQ ID NO: 77 - protein, 116 aa, Homo sapiens
EVQLQQSGAALVRPGALVKLSCKSSGFNIRDYYIHWVKQRPEQGLEWVGWIDPDNGDTMYDPKFQGKATLTADTYSNTAYLQLSSLAFEDSAVYYCARSPIGPAHWGQGTLVTVSA

SEQ ID NO: 78 - protein, 106 aa, Homo sapiens
ENVLTQSPAIMSASPGDKVTMTCSASSSVTNMHWYQQKSTTSPKLWIYDTSKLASGVPGRFSGSGSGNSYSLTISSMEAEDVATYFCFQGSGYPLTFGGGTKLEIK

SEQ ID NO: 79 - DNA, 349 bp, Homo sapiens
  source 1..349: mol_type = unassigned DNA
gaggttcaactgcagcagtctggggctgcgcttgtgaggccaggggccttagtcaagttgtcctgcaaatcttctggcttcaacattagagactactatatacactgggtgaagcagaggcctgaacagggcctggagtgggttggatggattgatcctgacaatggtgatactatgtatgacccgaagttccagggcaaggccactttaacagcagacacatattccaacaccgcctacctgcagctcagcagcctggcattcgaggacagtgccgtctattactgtgctagatccccaattggacctgctcactggggccaagggactctggtcactgtctctgcag

SEQ ID NO: 80 - DNA, 319 bp, Homo sapiens
  source 1..319: mol_type = unassigned DNA
gaaaatgttctcacccagtctccagcaatcatgtctgcatctccaggggacaaggtcaccatgacctgtagtgccagctcaagtgtaactaacatgcactggtaccagcagaagtcaaccacctcccccaaactctggatttatgacacatccaaactggcttctggagtcccaggtcgcttcagtggcagtgggtctggaaactcttactctctcacgatcagcagcatggaggctgaagatgttgccacttatttctgttttcaggggagtgggtacccactcacgttcggaggggggaccaagctggaaataaaac

SEQ ID NO: 81 - protein, 459 aa, Homo sapiens
MKCSWVIFFLMAVVTGVNSEVQLQQSGAALVRPGALVKLSCKSSGFNIRDYYIHWVKQRPEQGLEWVGWIDPDNGDTMYDPKFQGKATLTADTYSNTAYLQLSSLAFEDSAVYYCARSPIGPAHWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 82 - protein, 235 aa, Homo sapiens
MDFQVQIFSFLLISASVIMSRGENVLTQSPAIMSASPGDKVTMTCSASSSVTNMHWYQQKSTTSPKLWIYDTSKLASGVPGRFSGSGSGNSYSLTISSMEAEDVATYFCFQGSGYPLTFGGGTKLEIKRADAAPTVSTFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 83 - DNA, 1380 bp, Homo sapiens
  source 1..1380: mol_type = unassigned DNA
atgaaatgcagctgggtcatcttcttcctgatggcagtggttacaggggtcaattcagaggttcaactgcagcagtctggggctgcgcttgtgaggccaggggccttagtcaagttgtcctgcaaatcttctggcttcaacattagagactactatatacactgggtgaagcagaggcctgaacagggcctggagtgggttggatggattgatcctgacaatggtgatactatgtatgacccgaagttccagggcaaggccactttaacagcagacacatattccaacaccgcctacctgcagctcagcagcctggcattcgaggacagtgccgtctattactgtgctagatccccaattggacctgctcactggggccaagggactctggtcactgtctctgcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 84 - DNA, 708 bp, Homo sapiens
  source 1..708: mol_type = unassigned DNA
atggattttcaagtgcagattttcagcttcctgctaatcagtgcctcagtcataatgtccagaggagaaaatgttctcacccagtctccagcaatcatgtctgcatctccaggggacaaggtcaccatgacctgtagtgccagctcaagtgtaactaacatgcactggtaccagcagaagtcaaccacctcccccaaactctggatttatgacacatccaaactggcttctggagtcccaggtcgcttcagtggcagtgggtctggaaactcttactctctcacgatcagcagcatggaggctgaagatgttgccacttatttctgttttcaggggagtgggtacccactcacgttcggaggggggaccaagctggaaataaaacgggctgatgctgcaccaactgtatccaccttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 85 - protein, 10 aa, Homo sapiens
GFSLSTSGMG

SEQ ID NO: 86 - protein, 7 aa, Homo sapiens
IYWDDDK

SEQ ID NO: 87 - protein, 14 aa, Homo sapiens
ARTHYTYDVYAMDY

SEQ ID NO: 88 - protein, 7 aa, Homo sapiens
SSVSSSY

SEQ ID NO: 89 - protein, 9 aa, Homo sapiens
HQYHRSPWT

SEQ ID NO: 90 - protein, 122 aa, Homo sapiens
QVTLKESGPGILQPSQTLSLTCSFSGFSLSTSGMGVSWIRQPSGKGLEWLAHIYWDDDKRYNPSLKSRLTISKDTSTNQVFLKITSVDTADTATYYCARTHYTYDVYAMDYWGQGTSVTVSS

SEQ ID NO: 91 - protein, 108 aa, Homo sapiens
QIVLTQSPAIMSASLGERVTMTCTASSSVSSSYLHWYRQKPGSSPKLWIYSASNLASGVPPRFSGSGSGTSYSLTISNMEAEDAATYYCHQYHRSPWTFGGGTKLEIK

SEQ ID NO: 92 - DNA, 367 bp, Homo sapiens
  source 1..367: mol_type = unassigned DNA
caggttactctgaaagagtctggccctgggatattgcagccctcccagaccctcagtctgacttgttctttctctgggttttcactgagcacttctggtatgggtgtgagctggattcgtcagccttcaggaaagggtctggagtggctggcacacatttactgggatgatgacaagcgctataacccatccctgaagagccggctcacaatctccaaggatacctccacaaaccaggtattcctcaagatcaccagtgtggacactgcagatactgccacatactactgtgctcgaacccactatacttacgacgtttatgctatggactactggggtcaaggaacctcagtcaccgtctcctcag

SEQ ID NO: 93 - DNA, 325 bp, Homo sapiens
  source 1..325: mol_type = unassigned DNA
caaattgttctcacccagtctccagcaatcatgtctgcatctctaggggaacgggtcaccatgacctgcactgccagctcaagtgttagttccagttacttgcactggtaccgccagaagccaggatcctcccccaaactctggatttatagcgcatccaacctggcttctggagtcccacctcgcttcagtggcagtgggtctgggacctcttactctctcacaatcagcaacatggaggctgaagatgctgccacttattactgccaccagtatcatcgttccccgtggacgttcggtggaggcaccaagctggaaatcaaac

SEQ ID NO: 94 - protein, 465 aa, Homo sapiens
MDRLTSSFLLLIVPAYVLSQVTLKESGPGILQPSQTLSLTCSFSGFSLSTSGMGVSWIRQPSGKGLEWLAHIYWDDDKRYNPSLKSRLTISKDTSTNQVFLKITSVDTADTATYYCARTHYTYDVYAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 95 - protein, 237 aa, Homo sapiens
MDFQVQIFSFLLISASVIMSRGQIVLTQSPAIMSASLGERVTMTCTASSSVSSSYLHWYRQKPGSSPKLWIYSASNLASGVPPRFSGSGSGTSYSLTISNMEAEDAATYYCHQYHRSPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 96 - DNA, 1398 bp, Homo sapiens
  source 1..1398: mol_type = unassigned DNA
atggacaggcttacttcctcattcctgctgctgattgtccctgcatatgtcctttcccaggttactctgaaagagtctggccctgggatattgcagccctcccagaccctcagtctgacttgttctttctctgggttttcactgagcacttctggtatgggtgtgagctggattcgtcagccttcaggaaagggtctggagtggctggcacacatttactgggatgatgacaagcgctataacccatccctgaagagccggctcacaatctccaaggatacctccacaaaccaggtattcctcaagatcaccagtgtggacactgcagatactgccacatactactgtgctcgaacccactatacttacgacgtttatgctatggactactggggtcaaggaacctcagtcaccgtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 97 - DNA, 713 bp, Homo sapiens
  source 1..713: mol_type = unassigned DNA
atggattttcaggtgcagattttcagcttcctgctaatcagtgcctcagtcataatgtccagaggacaaattgttctcacccagtctccagcaatcatgtctgcatctctaggggaacgggtcaccatgacctgcactgccagctcaagtgttagttccagttacttgcactggtaccgccagaagccaggatcctcccccaaactctggatttatagcgcatccaacctggcttctggagtcccacctcgcttcagtggcagtgggtctgggacctcttactctctcacaatcagcaacatggaggctgaagatgctgccacttattactgccaccagtatcatcgttccccgtggacgttcggtggaggcaccaagctggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgtta

SEQ ID NO: 98 - protein, 8 aa, Homo sapiens
GNSFTGHN

SEQ ID NO: 99 - protein, 8 aa, Homo sapiens
VDPHYGSS

SEQ ID NO: 100 - protein, 13 aa, Homo sapiens
ARSGRIHPLFFDV

SEQ ID NO: 101 - protein, 10 aa, Homo sapiens
ESVDSYGIAF

SEQ ID NO: 102 - protein, 9 aa, Homo sapiens
QQTKEIPYT

SEQ ID NO: 103 - protein, 120 aa, Homo sapiens
EVQLQQSGPELEKPGASVKISCKASGNSFTGHNINWVKQRNGEALEWIGNVDPHYGSSDYNQKFKDKATLTADKSSSTAFLQFVSLTSEDSAVYYCARSGRIHPLFFDVWGAGTTVTVSS

SEQ ID NO: 104 - protein, 111 aa, Homo sapiens
DIVLTQSPASLAVSLGQRATIFCRASESVDSYGIAFMNWFQQKPGQPPKLLIYAASNQGSGVPARFSGSGSGTDFSLNIHPMEEDDTAMYFCQQTKEIPYTFGGGTRLEIK

SEQ ID NO: 105 - DNA, 361 bp, Homo sapiens
  source 1..361: mol_type = unassigned DNA
gaggtccagctgcaacagtctggacctgagctggagaagcctggcgcttcagtgaagatatcctgcaaggcttctggtaactcattcactggccacaacataaactgggtgaaacaaaggaatggagaggcccttgagtggattggaaatgttgatccccactatggtagtagtgactacaaccagaagtttaaggacaaggccacattgactgcagacaaatcctccagcacagccttcttgcaattcgtgagcctgacatctgaggactctgcagtctattactgtgcaagatccggacgaattcatcccttattcttcgatgtctggggcgccgggaccacggtcaccgtctcctcag

SEQ ID NO: 106 - DNA, 334 bp, Homo sapiens
  source 1..334: mol_type = unassigned DNA
gacattgtgctgacccaatctccagcttctttggctgtgtctctagggcagagggccaccatcttctgcagagccagcgaaagtgttgatagttatggcattgcttttatgaactggttccaacagaaaccaggacagccacccaaactcctcatctatgctgcatccaaccaaggatccggggtccctgccaggtttagtggcagtgggtctgggacagacttcagcctcaacatccatcctatggaggaggatgatactgcaatgtatttctgtcaacaaactaaggagattccgtacacgttcggaggcgggaccaggctggaaataaaac

SEQ ID NO: 107 - protein, 463 aa, Homo sapiens
MGWTWIFILMLSVTTGVHSEVQLQQSGPELEKPGASVKISCKASGNSFTGHNINWVKQRNGEALEWIGNVDPHYGSSDYNQKFKDKATLTADKSSSTAFLQFVSLTSEDSAVYYCARSGRIHPLFFDVWGAGTTVTVSSAKATPPSVYPLAPGSAAQTNSMVTLGCLVKGYFPVPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 108 - protein, 238 aa, Homo sapiens
MEKDTLLLWVLLLWVPGSTGDIVLTQSPASLAVSLGQRATIFCRASESVDSYGIAFMNWFQQKPGQPPKLLIYAASNQGSGVPARFSGSGSGTDFSLNIHPMEEDDTAMYFCQQTKEIPYTFGGGTRLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 109 - DNA, 1392 bp, Homo sapiens
  source 1..1392: mol_type = unassigned DNA
atgggatggacctggatctttattttaatgctgtcagtcactacaggtgtccactctgaggtccagctgcaacagtctggacctgagctggagaagcctggcgcttcagtgaagatatcctgcaaggcttctggtaactcattcactggccacaacataaactgggtgaaacaaaggaatggagaggcccttgagtggattggaaatgttgatccccactatggtagtagtgactacaaccagaagtttaaggacaaggccacattgactgcagacaaatcctccagcacagccttcttgcaattcgtgagcctgacatctgaggactctgcagtctattactgtgcaagatccggacgaattcatcccttattcttcgatgtctggggcgccgggaccacggtcaccgtctcctcagccaaagcgacacccccatctgtctatccactggcccctggatctgctgcccaaactaactccatggtgaccctgggatgcctggtcaagggctatttccctgtgccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 110 - DNA, 716 bp, Homo sapiens
  source 1..716: mol_type = unassigned DNA
atggagaaagacacactcctgctatgggtcctgcttctctgggttccaggttccacaggtgacattgtgctgacccaatctccagcttctttggctgtgtctctagggcagagggccaccatcttctgcagagccagcgaaagtgttgatagttatggcattgcttttatgaactggttccaacagaaaccaggacagccacccaaactcctcatctatgctgcatccaaccaaggatccggggtccctgccaggtttagtggcagtgggtctgggacagacttcagcctcaacatccatcctatggaggaggatgatactgcaatgtatttctgtcaacaaactaaggagattccgtacacgttcggaggcgggaccaggctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgtta

SEQ ID NO: 111 - protein, 8 aa, Homo sapiens
GFTFSTYV

SEQ ID NO: 112 - protein, 8 aa, Homo sapiens
ISSGGFYT

SEQ ID NO: 113 - protein, 12 aa, Homo sapiens
ARLTAAPYYFDF

SEQ ID NO: 114 - protein, 7 aa, Homo sapiens
SSVSSSN

SEQ ID NO: 115 - protein, 9 aa, Homo sapiens
QQWSSYPWT

SEQ ID NO: 116 - protein, 119 aa, Homo sapiens
EVMLVESGGGLVKPGGSLKLSCAASGFTFSTYVMSWVRQTPEKKLEWVATISSGGFYTYYPDSVKGRFTISRDNAENTLFLQVRSLRSEDTAMYYCARLTAAPYYFDFWGQGTSLTVSS

SEQ ID NO: 117 - protein, 108 aa, Homo sapiens
EIVLTQSPALVAASPGEKVTITCSVSSSVSSSNLHWYQQKSETSPKPWIYGTSNLASGVPTRFSGSGSGTSYSLTISSMEAEDAATYYCQQWSSYPWTFGGGTKVEIK

SEQ ID NO: 118 - DNA, 358 bp, Homo sapiens
  source 1..358: mol_type = unassigned DNA
gaagtgatgctggtggagtctgggggaggcttagtgaagcctggagggtccctgaaactctcctgtgcagcctctggattcactttcagtacctatgtcatgtcttgggttcgccagactccggagaagaagctggagtgggtcgcaaccattagtagtggtggtttttacacctactatccagacagtgtgaaggggcgtttcaccatctccagagacaatgccgagaacaccctgttcctgcaagtgcgcagtctgaggtctgaggacacggccatgtattactgtgcaagacttactgcggccccttactactttgacttctggggccaaggcacctctctcacagtctcctcag

SEQ ID NO: 119 - DNA, 325 bp, Homo sapiens
  source 1..325: mol_type = unassigned DNA
gaaattgtactcacccaatctccagcactcgtggctgcatctccaggggagaaggtcaccatcacctgcagtgtcagctcaagtgtaagttccagcaacttacactggtaccagcagaagtcagaaacttcccccaaaccctggatttatggcacatccaacctggcttctggagtccctactcgcttcagtggcagtggatctgggacctcttattctctcacaatcagcagcatggaggctgaagatgctgccacttattactgtcaacagtggagtagttacccgtggacgttcggtggaggcaccaaggtggaaatcaaac

SEQ ID NO: 120 - protein, 462 aa, Homo sapiens
MNFGLSLIFLVLVLKGVQCEVMLVESGGGLVKPGGSLKLSCAASGFTFSTYVMSWVRQTPEKKLEWVATISSGGFYTYYPDSVKGRFTISRDNAENTLFLQVRSLRSEDTAMYYCARLTAAPYYFDFWGQGTSLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVACVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 121 - protein, 236 aa, Homo sapiens
DFHVQIFSFMLISVTVTLSSGEIVLTQSPALVAASPGEKVTITCSVSSSVSSSNLHWYQQKSETSPKPWIYGTSNLASGVPTRFSGSGSGTSYSLTISSMEAEDAATYYCQQWSSYPWTFGGGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 122 - DNA, 1389 bp, Homo sapiens
  source 1..1389: mol_type = unassigned DNA
atgaacttcgggctcagcttgattttccttgtccttgttttaaaaggtgtccagtgtgaagtgatgctggtggagtctgggggaggcttagtgaagcctggagggtccctgaaactctcctgtgcagcctctggattcactttcagtacctatgtcatgtcttgggttcgccagactccggagaagaagctggagtgggtcgcaaccattagtagtggtggtttttacacctactatccagacagtgtgaaggggcgtttcaccatctccagagacaatgccgagaacaccctgttcctgcaagtgcgcagtctgaggtctgaggacacggccatgtattactgtgcaagacttactgcggccccttactactttgacttctggggccaaggcacctctctcacagtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcgcgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 123 - DNA, 714 bp, Homo sapiens
  source 1..714: mol_type = unassigned DNA
atggattttcatgtgcagattttcagcttcatgctaatcagtgtcacagtcacattgtccagtggagaaattgtactcacccaatctccagcactcgtggctgcatctccaggggagaaggtcaccatcacctgcagtgtcagctcaagtgtaagttccagcaacttacactggtaccagcagaagtcagaaacttcccccaaaccctggatttatggcacatccaacctggcttctggagtccctactcgcttcagtggcagtggatctgggacctcttattctctcacaatcagcagcatggaggctgaagatgctgccacttattactgtcaacagtggagtagttacccgtggacgttcggtggaggcaccaaggtggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccatcgtcaagagcttcaacaggaatgagtgttag

SEQ ID NO: 124 - protein, 8 aa, Homo sapiens
GYTFTEYT

SEQ ID NO: 125 - protein, 8 aa, Homo sapiens
INPNNGGS

SEQ ID NO: 126 - protein, 6 aa, Homo sapiens
ASNGGY

SEQ ID NO: 127 - protein, 11 aa, Homo sapiens
QSLLDSDGKTY

SEQ ID NO: 128 - protein, 9 aa, Homo sapiens
WQGTHYPWT

SEQ ID NO: 129 - protein, 113 aa, Homo sapiens
EVQLQQSGPELVKPGASVKISCKTSGYTFTEYTMHWVKQSHGKSLEWIGGINPNNGGSSYNQKFKGKATLTVNKSSSTAYMELRSLTSEDSAVYYCASNGGYWGQGTTLTVSS

SEQ ID NO: 130 - protein, 112 aa, Homo sapiens
DVVMTQTPLTLSVTIGQPASISCKSSQSLLDSDGKTYLNWLLQRPGQSPERLIYLVSKLDSGVPDRFTGSGSGTDFTLKISRVEAEDLGVYHCWQGTHYPWTFGGGTKLEIK

SEQ ID NO: 131 - DNA, 340 bp, Homo sapiens
  source 1..340: mol_type = unassigned DNA
gaggtccagctgcaacagtctggacctgagctggtgaagcctggggcttcagtgaagatatcctgcaagacttctggatacacattcactgaatacaccatgcactgggtgaagcagagccatggaaagagccttgagtggattggaggtattaatcctaacaatggtggttctagctacaaccagaagttcaagggcaaggccacattgactgtaaacaagtcctccagcacagcctacatggagctccgcagcctgacatctgaggattctgcagtctattactgtgcaagcaatgggggctactggggccaaggcaccactctcacagtctcctcag

SEQ ID NO: 132 - DNA, 337 bp, Homo sapiens
  source 1..337: mol_type = unassigned DNA
gatgttgtgatgacccagactccactcactttgtcggttaccattggacaaccagcctccatctcttgcaagtcaagtcagagcctcttagatagtgatggaaagacatatttgaattggttgttacagaggccaggccagtctccagagcgcctaatctatctggtgtctaaattggactctggagtccctgacaggttcactggcagtggctcagggacagatttcacactgaaaattagcagagtggaggctgaggatttgggagtttatcattgctggcaaggtacacattatccgtggacgttcggtggaggcaccaagctggaaatcaaac

SEQ ID NO: 133 - protein, 456 aa, Homo sapiens
MGWSWIFLFLLSGTAGVLSEVQLQQSGPELVKPGASVKISCKTSGYTFTEYTMHWVKQSHGKSLEWIGGINPNNGGSSYNQKFKGKATLTVNKSSSTAYMELRSLTSEDSAVYYCASNGGYWGQGTTLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 134 - protein, 239 aa, Homo sapiens
MMSPAQFLFLLVLWIRETNGDVVMTQTPLTLSVTIGQPASISCKSSQSLLDSDGKTYLNWLLQRPGQSPERLIYLVSKLDSGVPDRFTGSGSGTDFTLKISRVEAEDLGVYHCWQGTHYPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 135 - DNA, 1371 bp, Homo sapiens
  source 1..1371: mol_type = unassigned DNA
atgggatggagctggatctttctctttctcctgtcaggaactgcaggtgtcctctctgaggtccagctgcaacagtctggacctgagctggtgaagcctggggcttcagtgaagatatcctgcaagacttctggatacacattcactgaatacaccatgcactgggtgaagcagagccatggaaagagccttgagtggattggaggtattaatcctaacaatggtggttctagctacaaccagaagttcaagggcaaggccacattgactgtaaacaagtcctccagcacagcctacatggagctccgcagcctgacatctgaggattctgcagtctattactgtgcaagcaatgggggctactggggccaaggcaccactctcacagtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 136 - DNA, 720 bp, Homo sapiens
  source 1..720: mol_type = unassigned DNA
atgatgagtcctgcccagttcctgtttctgttagtgctctggattcgggaaaccaacggtgatgttgtgatgacccagactccactcactttgtcggttaccattggacaaccagcctccatctcttgcaagtcaagtcagagcctcttagatagtgatggaaagacatatttgaattggttgttacagaggccaggccagtctccagagcgcctaatctatctggtgtctaaattggactctggagtccctgacaggttcactggcagtggctcagggacagatttcacactgaaaattagcagagtggaggctgaggatttgggagtttatcattgctggcaaggtacacattatccgtggacgttcggtggaggcaccaagctggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 137 - protein, 17 aa, Homo sapiens
TLSDSGGATHYADSVQG

SEQ ID NO: 138 - protein, 13 aa, Homo sapiens
DVPPKTAGPMFDY

SEQ ID NO: 139 - protein, 16 aa, Homo sapiens
RSSESLVHSNGNTYLS

SEQ ID NO: 140 - protein, 8 aa, Homo sapiens
EISKRYSG

SEQ ID NO: 141 - protein, 9 aa, Homo sapiens
AQVSHFPRT

SEQ ID NO: 142 - protein, 5 aa, Homo sapiens
TYWIH

SEQ ID NO: 143 - protein, 17 aa, Homo sapiens
WIIPKSGGTNYAQKFQG

SEQ ID NO: 144 - protein, 12 aa, Homo sapiens
GILLNGIGAFDY

SEQ ID NO: 145 - protein, 11 aa, Homo sapiens
RTSQTVSSNLN

SEQ ID NO: 146 - protein, 7 aa, Homo sapiens
GISDLHS

SEQ ID NO: 147 - protein, 9 aa, Homo sapiens
QQSYSLPRT

SEQ ID NO: 148 - protein, 5 aa, Homo sapiens
SYYLS

SEQ ID NO: 149 - protein, 16 aa, Homo sapiens
YMFHRGSTNRNPSLRS

SEQ ID NO: 150 - protein, 14 aa, Homo sapiens
HLGIVGVTNAAFDI

SEQ ID NO: 151 - protein, 13 aa, Homo sapiens
SGSSSNIGNNYVS

SEQ ID NO: 152 - protein, 7 aa, Homo sapiens
QNDKRPS

SEQ ID NO: 153 - protein, 11 aa, Homo sapiens
GTWDRSLNADV

SEQ ID NO: 154 - protein, 122 aa, Homo sapiens
EVQLLESGGGLVQPGGSQRLFCEASGFTFASYAMSWVRLAPGKGLEWVSTLSDSGGATHYADSVQGRFTISRDNSNNVVYLQMNSLRAEDTAVYYCARDVPPKTAGPMFDYWGLGTLVTVSS

SEQ ID NO: 155 - protein, 112 aa, Homo sapiens
DIVMTQTPLSSPVSLGQPASISCRSSESLVHSNGNTYLSWLHQRPGQPPRLLIYEISKRYSGVSDRFSGSGAGTDFTLKISRVEAEDVGIYFCAQVSHFPRTFGQGTKVEIK

SEQ ID NO: 156 - protein, 121 aa, Homo sapiens
QVQLVESGAEVKKPGASVKVSCKASGYTFSTYWIHWMRQAPGQGPEWMGWIIPKSGGTNYAQKFQGRVAMTRDTSLNTVYMELSRLTSDDTAVYYCARGILLNGIGAFDYWGQGTLVTVSS

SEQ ID NO: 157 - protein, 121 aa, Homo sapiens
QVQLVESGAEVKKPGASVKVSCKASGYTFATYWIHWMRQAPGQGPEWMGWIIPKSGGTNYAQKFQGRVAMTRDTSLNTVYMELSRLTSDDTAVYYCARGILLNGIGAFDYWGQGTLVTVSS

SEQ ID NO: 158 - protein, 107 aa, Homo sapiens
DIVMTQSPSSLSASVGDRVTITCRTSQTVSSNLNWYQQRPGKAPKLLISGISDLHSGVPSRFSGSGSGTDFTLTISSLQPEDSATYYCQQSYSLPRTFGQGTKVEIK

SEQ ID NO: 159 - protein, 122 aa, Homo sapiens
QVQLQESGPGLVEPSETLSLTCTVSGGSIRSYYLSWIRQSPKKGLEWIGYMFHRGSTNRNPSLRSRVTMSLDTSKNQFSLKLTSVTAADTAVYYCATHLGIVGVTNAAFDIWGQGTMVTVSS

SEQ ID NO: 160 - protein, 110 aa, Homo sapiens
QSVLTQPPSVSAAPGQKVTISCSGSSSNIGNNYVSWYQLLPGTAPKLLIYQNDKRPSDIPDRFSGSKSGTSATLGITGLQTGDEADYYCGTWDRSLNADVFGGGTKLTVL

SEQ ID NO: 161 - protein, 8 aa, Homo sapiens
GFTFSHSD

SEQ ID NO: 162 - protein, 7 aa, Homo sapiens
IGTAHDT

SEQ ID NO: 163 - protein, 12 aa, Homo sapiens
ARVAFSWRCPDF

SEQ ID NO: 164 - protein, 8 aa, Homo sapiens
SSNIGSNS

SEQ ID NO: 165 - protein, 12 aa, Homo sapiens
AAWDDSLNGHLV

SEQ ID NO: 166 - protein, 118 aa, Homo sapiens
EVQLVDSGGGLVQPGGSLRLSCVASGFTFSHSDMHWVRQVTGKGLEWVASIGTAHDTWYAGSVKGRFTISRENAKNSLYLQMNSLTVEDTAVYYCARVAFSWRCPDFWGQGTLVTVSS

SEQ ID NO: 167 - protein, 111 aa, Homo sapiens
QSVLTQPPSASGTPGQRVTISCSGSSSNIGSNSVTWYQQVPGTVPKLLIYKSDQRPSGVPDRFSGSKSGSSASLAISGLQSEDEADYYCAAWDDSLNGHLVFGGGTKLTVL

SEQ ID NO: 168 - DNA, 355 bp, Homo sapiens
  source 1..355: mol_type = unassigned DNA
gaggtgcagctggtggactctgggggaggcttggtacagcctggggggtccctgagactctcctgtgtagcctctggattcaccttcagtcactccgacatgcactgggtccgccaggttacgggaaagggtctggagtgggtcgcatctattggtactgctcatgacacgtggtatgcaggctccgtgaagggccgattcaccatctccagagaaaatgccaagaactccttgtatcttcaaatgaacagcctgacagtcgaggacacggccgtatattactgtgcaagagttgccttcagctggcggtgccctgacttctggggccagggaaccctggtcaccgtctcctcag

SEQ ID NO: 169 - DNA, 334 bp, Homo sapiens
  source 1..334: mol_type = unassigned DNA
cagtctgtgctgactcagccaccctcagcgtctgggacccccgggcagagggtcaccatctcttgttctggaagcagctccaatatcggaagtaatagtgtaacctggtaccagcaggtcccaggaacagtccccaaactcctcatctataagagtgatcagcggccctcaggggtccctgaccgattctctggctccaagtctggctcctcagcctccctggccatcagtgggctccagtctgaggatgaggctgattattattgtgcagcatgggatgacagtctgaatggtcatctggttttcggcggagggaccaaactgaccgtcctag